SEQUENCE LISTING (37 sequences)

SEQ ID NO: 1 - DNA, 21 bp, Mus musculus
ggtggcatctctcttccaatt

SEQ ID NO: 2 - DNA, 21 bp, Mus musculus
ccaaggacactctacgtatct

SEQ ID NO: 3 - DNA, 21 bp, Mus musculus
ggaaccagtttcgtacatgtt

SEQ ID NO: 4 - DNA, 21 bp, Mus musculus
gccaatatgtgaaacatttct

SEQ ID NO: 5 - protein, 43 aa, Artificial Sequence
  Description of Artificial Sequence Synthetic polypeptide
CXXXXXXXCXXXXXCXXXXXXXXXXXXXCXCXXXXXXXXGXRC

SEQ ID NO: 6 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 7 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 8 - protein, 461 aa, Homo sapiens; identical to SEQ ID NO: 7

SEQ ID NO: 9 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 11 - protein, 212 aa, Homo sapiens; identical to SEQ ID NO: 9

SEQ ID NO: 12 - protein, 1210 aa, Homo sapiens [sequence omitted]

SEQ ID NO: 13 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 14 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 15 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 16 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 17 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 18 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 19 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 20 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 21 - protein, 352 aa, Homo sapiens [sequence omitted]

SEQ ID NO: 22 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 23 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 24 - protein, 352 aa, Homo sapiens [sequence omitted]

SEQ ID NO: 25 - protein, 92 aa, Homo sapiens
MKLCVTVLSLLMLVAAFCSPALSAPMGSDPPTACCFSYTARKLPRNFVVDYYETSSLCSQPAVVFQTKRSKQVCADPSESWVQEYVYDLELN

SEQ ID NO: 26 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 27 - protein, 352 aa, Homo sapiens [sequence omitted]

SEQ ID NO: 28 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 29 - protein, 976 aa, Homo sapiens [sequence omitted]

SEQ ID NO: 30 - protein, 92 aa, Homo sapiens; identical to SEQ ID NO: 22

SEQ ID NO: 31 - protein, 355 aa, Homo sapiens; identical to SEQ ID NO: 26

SEQ ID NO: 32 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 33 - protein, 360 aa, Homo sapiens; identical to SEQ ID NO: 23

SEQ ID NO: 34 - protein, 352 aa, Homo sapiens [sequence omitted]

SEQ ID NO: 35 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 36 - protein, 976 aa, Homo sapiens [sequence omitted]

SEQ ID NO: 37 - protein, 337 aa, Homo sapiens
MRPTLLWSLLLLLGVFAAAAAAPPDPLSQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQVILISVGTFSLLSVLAGACFFLVLKYRGLIKYWFHTPPSIPLQIEEYLKDPTQPILEALDKDSSPKDDVWDSVSIISFPEKEQEDVLQTL

(19) United States
(12) Patent Application Publication
(10) Pub. No.: US 2015/0140009 A1
(43) Pub. Date: May 21, 2015

(54) COMPOSITIONS AND METHODS FOR THE TREATMENT OF RADIATION EXPOSURE

(71) Applicant: GeneSys Research Institute, Brighton, MA (US)

(72) Inventor: David A. Goukassian, West Newton, MA (US)

(21) Appl. No.: 14/463,324

(22) Filed: Aug. 19, 2014

Related U.S. Application Data
(60) Provisional application No. 61/867,279, filed on Aug. 19, 2013.

(51) Int. Cl. C12N 15/113

(52) U.S. Cl. CPC C12N 15/113; C12N 2310/531; C12N 2310/14; C12N 2310/11
USPC 424/145.1; 514/44 A; 424/172.1; 424/158.1; 424/152.1

(57) ABSTRACT

The invention provides methods for the treatment of radiation exposure featuring agents that interfere with the expression, production, release, accumulation, or activity of a TNFα, IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide or fragment thereof.

### RELATED APPLICATIONS

This application claims the benefit of priority under 35 U.S.C. §119(e) to U.S. Provisional Application No. 61/867,279, filed Aug. 19, 2013, which is incorporated herein by reference in its entirety.

### BACKGROUND OF THE INVENTION

Cardiovascular (CV) morbidity may occur within months and years after radiation exposure, and cardiovascular mortality may occur within decades after initial radiation exposure. Previous epidemiologic data from studies of A-bomb survivors, accidental exposures (Chernobyl), astronauts, and cancer patients undergoing radiotherapy have demonstrated CV effects due to exposure to radiation. Without being bound to a particular theory, there is evidence that unirradiated cells exhibit irradiated effects as a result of signals received from irradiated cells (radiation-induced bystander effect). Thus, in cancer patients undergoing radiotherapy, a significant risk may exist for the development of CV diseases following exposure to therapeutic radiation.

In astronauts, who are exposed to cosmic radiation during space missions, the physiological effects of space travel have been documented. To date the majority of space flight-associated risks identified for the CV system were determined shortly after space missions (days and weeks) and include serious cardiac rhythm problems, compromised CV response to orthostatic and stress stimuli that may compromise cardiac function and manifestation of previously asymptomatic CV disease. Echocardiographic measurements obtained from astronauts revealed a 19-23% lower stroke volume and a 15-23% reduction in cardiac size post-mission compared to pre-mission. MRI measurements obtained from four astronauts following a 10-day space mission revealed ˜12% decrease in left ventricular mass. However, the effect of exposure to space radiation during and after space flights on molecular, cellular, and tissue levels has not been studied. At present, these adverse CV symptoms were determined to be the consequence of adaptation to microgravity, ameliorated by exercise, rather than risk factors causally related to space radiation.

Longitudinal studies of low-dose proton and heavy ion (HZE) radiation are warranted to determine long-term risk for CV diseases due to radiation exposure. In particular, radiation exposure may increase the risk for CV diseases by affecting the survival or mobilization of BM-derived EPCs (e.g., DNA damage response). Thus, a need exists for effective treatments of subjects exposed to radiation including accidental exposures, cancer patients undergoing radiotherapy, astronauts, and civilian space travelers. Accordingly, methods of treating the effects of radiation exposure are required.

### SUMMARY OF THE INVENTION

As described below, the present invention features compositions and methods for inhibiting the expression or activity of one or more of TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide or fragment thereof for the treatment or prevention of diseases and conditions associated with the effects of radiation exposure, particularly radiation exposure associated with space travel.

In one aspect the invention provides a method of ameliorating the effects of radiation exposure, including radiation-induced non-targeted effects, on a cell, the method involving contacting the cell with an agent that selectively reduces the expression or activity of one or more of a p75 TNF-α, p55 TNF-α, IL6, EGF, IL1-beta, G-CSF, MCP-1, MIP-1, SCF, or RANTES receptor in the cell relative to an untreated control cell, thereby ameliorating the effects of radiation exposure or radiation-induced non-targeted effects on the cell.

In another aspect the invention provides, a method of ameliorating the effects of radiation exposure, including radiation-induced non-targeted effects, on a cell, the method involving contacting the cell with an agent that selectively reduces the expression or activity of one or more of a p75 TNF-α, p55 TNF-α, IL6, EGF, IL1-alpha, IL1-beta, G-CSF, MCP-1, MIP-1, SCF, or RANTES peptide or fragment thereof in the cell relative to an untreated control cell, thereby ameliorating the effects of radiation exposure or radiation-induced non-targeted effects on the cell.

In yet another aspect, the invention provides a method of ameliorating the effects of radiation exposure on a subject (e.g., in a cell, tissue, or organ in a mammalian subject), the method involving administering to the subject an agent that selectively reduces the expression or activity of one or more of a receptor for p75 TNF-α, p55 TNF-α, IL6, EGF, IL1-beta, G-CSF, MCP-1, MIP-1, SCF, or RANTES in a cell relative to an untreated control cell, thereby ameliorating the effects of radiation exposure on the subject.

In still another aspect, the invention provides a method of ameliorating the effects of radiation exposure on a subject (e.g., in a cell, tissue, or organ in a mammalian subject), the method involving administering to the subject an agent that selectively reduces the expression or activity of one or more of a TNF-α, IL6, EGF, IL1-beta, G-CSF, MCP-1, MIP-1, SCF, or RANTES peptide or fragment thereof in a cell relative to an untreated control cell, thereby ameliorating the effects of radiation exposure on the subject.

In one aspect, the invention provides a pharmaceutical composition for the treatment of radiation exposure, the composition containing an effective amount of two or more agents that selectively reduce the expression or activity of two or more of a p75 TNF-α, p55 TNF-α, IL6, EGF, IL1-beta, G-CSF, MCP-1, MIP-1, SCF, or RANTES receptor in a cell, relative to a reference cell.

In another aspect, the invention provides a pharmaceutical composition for the treatment of radiation exposure, the composition containing an effective amount of two or more agents that selectively reduce the expression or activity of two or more of a TNF-α, IL6, EGF, IL1-beta, G-CSF, MCP-1, MIP-1, SCF, or RANTES peptide or fragment thereof in a cell, relative to a reference cell.

In yet another aspect, the invention provides a kit for treating radiation exposure containing an effective amount of an agent that selectively reduces the expression or activity of a p75 TNF-α, p55 TNF-α, IL6, EGF, IL1-beta, G-CSF, MCP-1, MIP-1, SCF, or RANTES receptor; or a TNF-α, IL6, EGF, IL1-beta, G-CSF, MCP-1, MIP-1, SCF, or RANTES peptide or fragment thereof in a cell and instructions for using the kit to treat radiation exposure.

In various embodiments of any of the aspects delineated herein, the cell is an endothelial progenitor cell, BM-derived endothelial progenitor cell, hemangioblast, hematopoietic stem cell, endothelial cell, cardiac cell, cardiomyocyte, muscle cell, vascular smooth muscle cell, satellite-cell, myoblast, or differentiated skeletal muscle cell. In various embodiments of any of the aspects delineated herein, the cell is contacted with a cytokine produced by radiation exposure. In various embodiments, the cytokine is one or more of TNF-α, IL6, EGF, IL1-beta, G-CSF, MCP-1, MIP-1, SCF, and RANTES. In various embodiments of any of the aspects delineated herein, the cell is not exposed to radiation.

In various embodiments of any of the aspects delineated herein, the cell is contacted with a cell or product of a cell that has been exposed to radiation. In particular embodiments, the contacting is via a gap junction. In various embodiments of any of the aspects delineated herein, the cell is in the immediate vicinity of a cell that has been exposed to radiation and not in direct contact with a cell that has been exposed to radiation. In various embodiments of any of the aspects delineated herein, the cell and cell exposed to radiation are present in a subject (e.g., a human, mouse, or other mammal). In various embodiments of any of the aspects delineated herein, the cell exposed to radiation is a BM-derived endothelial progenitor cell, hemangioblast, hematopoietic stem cells, an endothelial cell, a cardiomyocyte, a smooth muscle cell in the vascular wall, or a satellite-cell, myoblast, differentiated skeletal muscle cell.

In various embodiments of any of the aspects delineated herein, the radiation is one or more of low-dose or high-dose of terrestrial (e.g., X-ray, gamma, etc) or hadron (e.g., proton, neutron, etc.,) and high charge and energy (HZE) heavy ion (e.g., carbon, oxygen, silicon, iron, etc.) particle radiation (e.g., ionizing radiation, space, environmental, or therapeutic radiation). In various embodiments of any of the aspects delineated herein, the effect of radiation exposure is direct or indirect. In various embodiments of any of the aspects delineated herein, the effect of radiation exposure is one or more of a DNA double-strand break, gene inactivating mutation in a somatic or stem cell, increase in cytoplasmic Ca²⁺ signaling, reduction in mitochondrial action potential, decreased ATP production, increased production of reactive oxygen and nitrogen species (ROS and NOS), decreased cardiomyocyte or skeletal muscle contractility, or decreased cardiac and skeletal muscle function (e.g., in a subject). In various embodiments of any of the aspects delineated herein, the effect occurs within 24 hrs, 1-28 days, 1-24 months 1-40 years after radiation exposure. In various embodiments of any of the aspects delineated herein, the method, composition, or agent reduces DNA damage (e.g., DNA double-strand break, oxidative damage), or increases DNA repair.

In various embodiments of any of the aspects delineated herein, the agent is an inhibitory nucleic acid molecule that is complementary to at least a portion of a p75 TNF-α, p55 TNF-α, IL6, EGF, IL1-beta, G-CSF, MCP-1, MIP-1, SCF, or RANTES receptor nucleic acid molecule; or a TNF-α, EGF, IL1-alpha, IL1-beta, G-CSF, MCP-1, MIP-1, SCF, or RANTES nucleic acid molecule. In particular embodiments, the inhibitory nucleic acid molecule is one or more of an antisense molecule, an siRNA, or an shRNA. In specific embodiments, the inhibitory nucleic acid molecule includes or consists essentially of a nucleic acid molecule with a sequence selected from the group consisting SEQ ID NO: 1, SEQ ID NO: 2, SEQ ID NO: 3, and SEQ ID NO: 4.

In various embodiments of any of the aspects delineated herein, the agent is an antibody or fragment thereof that selectively binds to a p75 TNF-α, p55 TNF-α, IL6, EGF, IL1-beta, G-CSF, MCP-1, MIP-1, SCF, or RANTES receptor; or a TNF-α, EGF, IL1-alpha, IL1-beta, G-CSF, MCP-1, MIP-1, SCF, or RANTES peptide. In various embodiments, the antibody is a monoclonal or polyclonal antibody.

The invention provides compositions and methods for the treatment of radiation exposure. Other features and advantages of the invention will be apparent from the detailed description, and from the claims.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “blood vessel formation” is meant the dynamic process that includes one or more steps of blood vessel development and/or maturation, such as angiogenesis, vasculogenesis, formation of an immature blood vessel network, blood vessel remodeling, blood vessel stabilization, blood vessel maturation, blood vessel differentiation, or establishment of a functional blood vessel network.

By “cardiac protective activity” is meant any biological activity that maintains or increases the survival or function of a cardiac cell or cardiac tissue in vitro or in vivo.

By “cardiac function” is meant the biological function of cardiac tissue or heart. In one embodiment, cardiac function refers to contractile function. Methods for measuring the biological function of the heart are standard in the art (e.g., Textbook of Medical Physiology, Tenth edition, (Guyton et al., W.B. Saunders Co., 2000) and are also described herein. By “increasing cardiac function” is meant an increase in a biological function of the heart by at least about 5%, 10%, 15%, 20%, 30%, 40%, 50%, 60%, 70%, 80%, 90%, or even 100% relative to the biological function present in a reference cardiac tissue or heart.

By “cell survival” is meant cell viability.

By “reducing cell death” is meant reducing the propensity or probability that a cell will die. Cell death can be apoptotic, necrotic, or by any other means.

By “reducing inflammation” is meant reducing the severity or symptoms of an inflammatory reaction in a tissue. An inflammatory reaction within tissue is generally characterized by leukocyte infiltration, edema, redness, pain, neovascularization (in advanced cases), and finally impairment of function. Inflammation can also be measured by analyzing levels of cytokines, C reactive protein, or any other inflammatory marker.

By “control” or “reference” is meant a standard of comparison. For example, the level of TNF-α peptide in a cell exposed to radiation may be compared to the level of TNF-α peptide in a corresponding normal or healthy cell.

By “disease” is meant any condition or disorder that damages or interferes with the normal function of a cell, tissue, or organ. Examples of diseases include any disease or injury that results in a reduction in cell number or biological function, including ischemic injury, such as stroke, myocardial infarction, or any other ischemic event that causes tissue damage.

By “effective amount” is meant the amount of a required to ameliorate the symptoms of a disease relative to an untreated patient. The effective amount of active compound(s) used to practice the present invention for therapeutic treatment of a ischemic injury varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. A fragment of a polypeptide or nucleic acid molecule may have the biological activity of the polypeptide or nucleic acid molecule

By “inhibitory nucleic acid molecule” is meant a polynucleotide that disrupts the expression of a target nucleic acid molecule or an encoded polypeptide. Exemplary inhibitory nucleic acid molecules include, but are not limited to, shRNAs, siRNAs, antisense nucleic acid molecules, and analogs thereof.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis. When a cellular factor is “isolated” from a cultured cell the cellular factor is typically separated from cells and cellular debris. It need not be purified to homogeneity.

By “marker” is meant any protein or polynucleotide having an alteration in expression level or activity that is associated with a disease or disorder.

By “neoplasia” is meant a disease that is caused by or results in inappropriately high levels of cell division, inappropriately low levels of cell death, or both.

As used herein, “obtaining” as in “obtaining an agent” includes synthesizing, purchasing, or otherwise acquiring the agent.

The term “portion” is meant to refer to a part of a polypeptide or nucleic acid molecule. This part contains, preferably, at least 10%, 20%, 30%, 40%, 50%, 60%, 70%, 80%, or 90% of the entire length of the reference nucleic acid molecule or polypeptide. A portion may contain 10, 20, 30, 40, 50, 60, 70, 80, 90, or 100, 200, 300, 400, 500, 600, 700, 800, 900, or 1000 nucleotides or amino acids.

By “subject” is meant a mammal, including, but not limited to, a human or non-human mammal, such as a bovine, equine, canine, ovine, or feline.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “radiation” is meant sub-atomic particles or electromagnetic waves that are energetic enough to detach electrons from atoms or molecules, therefore ionizing them. For example, DNA exposed to ionizing radiation is susceptible to double-strand breaks.

The term “reduce” or “increase” is meant to alter negatively or positively, respectively, by at least 5%. An alteration may be by 5%, 10%, 25%, 30%, 50%, 75%, or even by 100%.

By “reference” is meant a standard or control condition. In one embodiment, the function of a treated cardiac tissue is compared to the function of that tissue prior to treatment. In other embodiments, the function of a treated cardiac tissue is compared to the function of an untreated corresponding control tissue.

By “selectively” is meant the ability to affect the activity or expression of a target molecule without affecting the activity or expression of a non-target molecule. For example, a p75/TNF-α receptor inhibitory nucleic acid molecule selectively reduces the levels of the p75/TNF-α receptor without affecting the activity or expression of the p55/TNF-α receptor.

By “TNF-α” is meant a protein or fragment thereof having substantial identity to the amino acid sequence of TNF-α provided at GenBank Accession No. NP_{—}000585 that promotes angiogenesis or has binding activity to p55/TNF-α receptor or p75/TNF-α receptor.

By “p75/TNFR2 polypeptide” is meant a protein or fragment thereof having substantial identity to the amino acid sequence of p75/TNFR2 provided at GenBank Accession No. NP_{—}001057 that promotes angiogenesis or has TNF-α binding activity.

By “p75/TNFR2 nucleic acid molecule” is meant a polynucleotide that encodes a p75/TNFR2 polypeptide.

By “p55/TNFR1 polypeptide” is meant a protein or fragment thereof having substantial identity to the amino acid sequence of p55/TNFR1 provided at GenBank Accession No. NP_{—}001056 that promotes angiogenesis or has TNF-α binding activity.

By “p55/TNFR1 nucleic acid molecule” is meant a polynucleotide that encodes a p55/TNFR1 polypeptide.

By “p75/TNFR2 biological activity” is meant TNF binding activity, p75/TNFR2 trimerization, p75/TNFR2-mediated signal transduction, promotion of stem and/or progenitor cell survival and proliferation, mobilization of bone marrow derived progenitor cells, stimulation of antioxidative pathways, angiogenesis enhancing activity, or regulation of cell adhesion molecules, such as E-selectin, VCAM-1, and ICAM.

By “p55/TNFR1 biological activity” is meant TNF binding activity, p55/TNFR2 trimerization, or p55/TNFR1-mediated signal transduction, angiogenesis enhancing activity, induction of apoptosis, or mediation of inflammation, such as leukocyte infiltration.

By “IL6R polypeptide” is meant a protein or fragment thereof having substantial identity to the amino acid sequence of IL6R provided at GenBank Accession No. NP_{—}000556 that promotes inflammation or has IL6 binding activity.

By “IL6R nucleic acid molecule” is meant a polynucleotide that encodes an IL6R polypeptide.

By “IL6 biological activity” is meant IL6R binding activity, IL6R-mediated signal transduction, stem and/or progenitor cell survival and proliferation, mobilization of bone marrow derived progenitor cells, stimulation of antioxidative pathways, angiogenesis enhancing activity, or regulation of cell adhesion molecules, such as E-selectin, VCAM-1, ICAM.

By “EGF” is meant a protein or fragment thereof having substantial identity to the amino acid sequence of EGF provided at GenBank Accession No. NP_{—}001954 that promotes cell growth, proliferation, and differentiation.

By “EGFR polypeptide” is meant a protein or fragment thereof having substantial identity to the amino acid sequence of EGFR provided at GenBank Accession No. NP_{—}005219 that promotes cell growth, proliferation, and differentiation or has IL6EGF binding activity.

By “EGFR nucleic acid molecule” is meant a polynucleotide that encodes an EGFR polypeptide.

By “EGF biological activity” is meant EGFR binding activity, EGFR-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “IL1-alpha” is meant a protein or fragment thereof having substantial identity to the amino acid sequence of IL1-alpha provided at GenBank Accession No. NP_{—}000566 that promotes fibroblast, neutrophil, and lymphocyte proliferation or mobilization.

By “IL1-beta” is meant a protein or fragment thereof having substantial identity to the amino acid sequence of IL1-beta provided at GenBank Accession No. NP_{—}000567 that promotes inflammatory activity, cell proliferation, differentiation, or apoptosis.

By “IL1R polypeptide” is meant a protein or fragment thereof having substantial identity to the amino acid sequence of IL1-R provided at GenBank Accession Nos. NP_{—}000868 and NP_{—}004624 that promote fibroblast, neutrophil, and lymphocyte proliferation or mobilization, promote inflammatory activity, cell proliferation, differentiation, or apoptosis, or has IL1-alpha or IL1-beta binding activity.

By “IL1R nucleic acid molecule” is meant a polynucleotide that encodes an IL1R polypeptide.

By “IL1-alpha biological activity” is meant IL1R binding activity, IL1R-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “IL1-beta biological activity” is meant IL1R binding activity, IL1R-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “G-CSF” is meant a protein or fragment thereof having substantial identity to the amino acid sequence of G-CSF provided at GenBank Accession No. NP_{—}000750 that promotes proliferation and release of granulocytes and bone marrow derived progenitor cells.

By “G-CSF-R polypeptide” is meant a protein or fragment thereof having substantial identity to the amino acid sequence of G-CSFR provided at GenBank Accession No. NP_{—}000751 that promotes proliferation and release of bone marrow derived progenitor cells or has G-CSF binding activity.

By “G-CSF-R nucleic acid molecule” is meant a polynucleotide that encodes an G-CSFR polypeptide.

By “G-CSF biological activity” is meant G-CSFR binding activity, G-CSFR-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “MCP-1” is meant a protein or fragment thereof having substantial identity to the amino acid sequence of MCP-1 provided at GenBank Accession No. NP_{—}002973 that promotes monocyte, T cell, or dendritic cell recruitment.

By “MCP-1R polypeptide” is meant a protein or fragment thereof having substantial identity to the amino acid sequence of MCP-1R provided at GenBank Accession Nos. NP_{—}001116513 and NP_{—}000570 that promotes immune cell recruitment or has MCP-1 binding activity.

By “MCP-1R nucleic acid molecule” is meant a polynucleotide that encodes an MCP-1R polypeptide.

By “MCP-1 biological activity” is meant MCP-1R binding activity, MCP-1R-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “MIP-1alpha” is meant a protein or fragment thereof having substantial identity to the amino acid sequence of MIP-1 provided at GenBank Accession No. NP_{—}002974 that promotes immune cell recruitment.

By “MIP-1beta” is meant a protein or fragment thereof having substantial identity to the amino acid sequence of MIP-1 provided at GenBank Accession No. NP_{—}002975 that promotes immune cell recruitment.

By “MIP-1R polypeptide” is meant a protein or fragment thereof having substantial identity to the amino acid sequence of MIP-1R provided at GenBank Accession Nos. NP_{—}001286, NP_{—}005499, or NP_{—}000570 that promotes immune cell recruitment or has MIP-1 binding activity.

By “MIP-1R nucleic acid molecule” is meant a polynucleotide that encodes an MIP-1R polypeptide.

By “MIP-1 biological activity” is meant MIP-1R binding activity, MIP-1R-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “SCF” is meant a protein or fragment thereof having substantial identity to the amino acid sequence of SCF provided at GenBank Accession No. NP_{—}000890 that promotes recruitment of hematopoietic stem cells, melanocytes, and germ cells.

By “SCFR polypeptide” is meant a protein or fragment thereof having substantial identity to the amino acid sequence of SCFR provided at GenBank Accession No. NP_{—}000213 that promotes recruitment of hematopoietic stem cells, melanocytes, and germ cells or has SCF binding activity.

By “SCFR nucleic acid molecule” is meant a polynucleotide that encodes an SCFR polypeptide.

By “SCF biological activity” is meant SCFR binding activity, SCFR-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “RANTES” is meant a protein or fragment thereof having substantial identity to the amino acid sequence of RANTES provided at GenBank Accession No. NP_{—}000591 that promotes mobilization of T cells, eosinophils, and basophils; promotes leukocyte recruitment; or promotes proliferation of natural killer cells.

By “RANTES-R polypeptide” is meant a protein or fragment thereof having substantial identity to the amino acid sequence of RANTES-R provided at GenBank Accession Nos. NP_{—}001286, NP_{—}001828, NP_{—}0005499, or NP_{—}000570 that promotes mobilization of T cells, eosinophils, and basophils; promotes leukocyte recruitment; or promotes proliferation of natural killer cells or has RANTES binding activity.

By “RANTES-R nucleic acid molecule” is meant a polynucleotide that encodes an RANTES-R polypeptide.

By “RANTES biological activity” is meant RANTES-R binding activity, RANTES-R-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

By “IFN-γ” is meant a protein or fragment thereof having substantial identity to the amino acid sequence of RANTES provided at GenBank Accession No. NP_{—}000591 that promotes mobilization of leukocytes; or promotes activation of natural killer cells.

By “IFNGR polypeptide” is meant a protein or fragment thereof having substantial identity to the amino acid sequence of IFNGR provided at GenBank Accession Nos. NP_{—}000407 or NP_{—}005525 that promotes mobilization of leukocytes; or promotes activation of natural killer cells or has IFN-γ binding activity.

By “IFNGR nucleic acid molecule” is meant a polynucleotide that encodes an IFNGR polypeptide.

By “IFN-γ biological activity” is meant IFNGR binding activity, IFNGR-mediated signal transduction, stem and/or progenitor cell survival and proliferation, mobilization of bone marrow derived progenitor cells, stimulation of antioxidative pathways, angiogenesis enhancing activity, or regulation of cell adhesion molecules such as E-selectin, VCAM-1, ICAM.

The term “polynucleotide” or “nucleic acid” as used herein designates mRNA, RNA, cRNA, cDNA or DNA.

By the terms “polypeptide”, “peptide” and “protein” are meant to be used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “repair” is meant to ameliorate damage or disease in a tissue or organ.

By “tissue” is meant a collection of cells having a similar morphology and function.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1A-FIG. 1B depict homing of BM-derived EPCs to ischemic tissue. Confocal microscopy of HL tissue from operated ischemic border zone (FIG. 1A) and operated ischemic zone (FIG. 1B) twenty eight days after HL surgery. Operated limbs of GFP-labeled BM transplanted mice showed that BM-derived cells homed only into ischemic areas (FIG. 1A and FIG. 1B—green GFP positive cells). Dotted lines in FIG. 1A indicated the boundary between ischemic tissue and non-ischemic normal muscle (40× magnification). Homing and recruitment of endothelial lineage cells into ischemic areas was examined (FIG. 1A and FIG. 1B—Isolectin/B4, an EC/EPC marker, red positive cells).

FIG. 2A-FIG. 2C depicts γ-H2AX decay in BM-derived EPC. FIG. 2A shows representative images of γ-H2AX foci (yellow) in BM-derived EPCs, nuclei, stained with Propidium Iodide (PI-red), ×100 magn. FIG. 2B is a graph depicting the distribution of EPCs with N foci 30 min (black bars), 24 hrs (grey bars) and 7 days (white bars) after irradiation plus 60-hrs selection in EPC medium. FIG. 2C is a graph depicting mean number of foci/cell for 30 min, 24 hrs and 7 days plus 60 hrs of culture selection.

FIG. 3A-FIG. 3D depict γ-H2AX decay in heart resident EC and non-EC cells. FIG. 3A shows representative images of γ-H2AX foci (green), Isolectin/B4 (red) immunostaining in the heart tissue of control and 1 Gy gamma irradiated mice 30 min, 24 hrs and 7 days post-irradiation; nuclei were stained with TopRo-3 (blue), 40× magnification. In 1 Gy 30 min inset−squares=heart resident ECs; circles=all other cells. FIG. 3B is a graph depicting the distribution of heart ECs with N foci after 30 min (black bars), 24 hrs (grey bars) and 7 days (white bars). The graph in the background was plotted after excluding the cells with zero foci. FIG. 3C is a graph depicting the distribution of heart non-EC cells with N foci after 30 min (black bars), 24 hrs (grey bars) and 7 days (white bars). The graph in the background was plotted after excluding the cells with zero foci. FIG. 3D is a graph depicting mean number of foci/cell in cardiac ECs (black bars) and all other cells (clear bars) for 30 min, 24 hrs and 7 days.

FIG. 4A and FIG. 4B depict the effects of radiation on myocytes. FIG. 4A is a graph depicting the effects of radiation on [Ca2+]i. [Ca2+]i was assessed in myocytes from control and irradiated mice 1 hr and 7 days post-IR. 1 hr post-IR myocytes exhibited a substantial increase in [Ca²⁺]i, suggesting compromised cellular Ca²⁺ homeostasis. Compared to controls, in IR cells [Ca²⁺]i was elevated after 7 days, but lower than at 24 hr. FIG. 4B is a graph depicting the effects of radiation on ΔΨₘ. ΔΨₘ was assessed in myocytes post irradiation (1-hr and 7 days) and compared to myocytes from non-irradiated animals. Tetra-methylrhodamine ethyl ester (TMRE) fluorescence was used as a reporter of ΔΨₘ. Reduction in TMRE fluorescence was observed at both time points, which suggest that a large number of mitochondria had a depolarized membrane potential and possibly altered functionality.

FIGS. 5A-FIG. 5C show that BM-derived EPC demonstrated radiobiological bystander response in the medium transfer experiments. FIG. 5A depicts representative images of γ-H2AX foci (yellow) in non-irradiated BM-derived EPCs after 24-hr incubation with conditioned media collected from EPCs 30 min, 5 and 24 hrs after 1 Gy γ-irradiation, nuclei were stained with TopRo-3-red, 100× magnification. FIG. 5B is a graph depicting distribution of EPCs with N foci 24 hrs after incubation with control or conditioned EPC media 30 min (black bars), 5 hrs (grey bars) and 24 hrs (white bars). The distribution of EPCs with N foci second graph in the background was plotted after excluding the cells with zero foci. FIG. 5C is a graph depicting mean number of foci/cell for 30 min, 5 hrs and 24 hrs after incubation for 24 hrs in conditioned medium.

FIGS. 6A and 6B are graphs depicting mean number of p-γH2AX foci/cell in WT, p75KO, and p55KO EPC samples (non-irradiated control (CTRL) and 1 Gy irradiation at various time points (24 hrs post media transfer from respective genotype CTRL and 1 Gy irradiated EPCs). FIG. 6A depicts a graph all cells with or without p-γH2AX foci were counted. FIG. 6B depicts a graph in which EPCs with 0 p-γH2AX foci/cells were excluded from the graph (in all three genotypes at any given time point 60-80% of EPCs had 0 p-γH2AX foci and no statistical difference was observed between genotypes at any time point).

FIG. 7 presents graphs quantitating p-γH2AX foci distribution (>5 foci) in ex-vivo expanded BM-derived EPCs from WT, p55KO and p75KO mice treated with irradiated (1 Gy gamma) conditioned EPC medium from corresponding genotypes (WT, p55KO and p75KO). Please note: cells under normal conditions no-IR or IR medium transfer or any DNA damaging treatments may have 1-4 pH2AX foci. For clarity, the graphs are presented starting from cells with 5 foci. Panels (top-bottom) are graphs corresponding to control (CTRL), 5 hr, Day 1, Day 3, and Day 5 timepoints.

FIG. 8 are graphs depicting p-γH2AX foci distribution (1-4 foci) in ex-vivo expanded BM-derived EPCs from WT, p55KO and p75KO mice treated with irradiated (1 Gy gamma) conditioned EPC medium from corresponding genotypes (WT, p55KO and p75KO). Panels (top-bottom) are graphs corresponding to control (CTRL), 5 hr. Day 1, Day 3, and Day 5 timepoints.

FIGS. 9A-9P are graphs showing ELISA profiling of cytokines and growth factors in conditioned medium (1 Gy gamma-irradiated EPCs). FIG. 9A shows TNF-α levels in conditioned medium after irradiation of EPCs with 1 Gy γ radiation. FIG. 9B shows IGF1 levels in conditioned medium after irradiation of EPCs with 1 Gy γ radiation. FIG. 9C shows VEGF levels in conditioned medium after irradiation of EPCs with 1 Gy γ radiation. FIG. 9D shows IFNγ levels in conditioned medium after irradiation of EPCs with 1 Gy γ radiation. FIG. 9E shows FGFb levels in conditioned medium after irradiation of EPCs with 1 Gy γ radiation. FIG. 9F shows IL6 levels in conditioned medium after irradiation of EPCs with 1 Gy γ radiation. FIG. 9G shows leptin levels in conditioned medium after irradiation of EPCs with 1 Gy γ radiation. FIG. 9H shows EGF levels in conditioned medium after irradiation of EPCs with 1 Gy γ radiation. FIG. 9I shows IL1α levels in conditioned medium after irradiation of EPCs with 1 Gy γ radiation. FIG. 9J shows IL1β levels in conditioned medium after irradiation of EPCs with 1 Gy γ radiation. FIG. 9K shows G-CSF levels in conditioned medium after irradiation of EPCs with 1 Gy γ radiation. FIG. 9L shows GM-CSF levels in conditioned medium after irradiation of EPCs with 1 Gy γ radiation. FIG. 9M shows MCP-1 levels in conditioned medium after irradiation of EPCs with 1 Gy γ radiation. FIG. 9N shows MIP-1α levels in conditioned medium after irradiation of EPCs with 1 Gy γ radiation. FIG. 9O shows SCF levels in conditioned medium after irradiation of EPCs with 1 Gy γ radiation. FIG. 9P shows RANTES levels in conditioned medium after irradiation of EPCs with 1 Gy γ radiation. For all graphs, wild-type, ♦; p75KO, ▪; p55KO, ▴.

FIGS. 10A-10AF show sequences for a TNF-α polypeptide provided at GenBank Accession No. NP_{—}000585 (FIG. 10A); a p55/TNFR2 polypeptide provided at GenBank Accession No. NP_{—}001056 (FIG. 10B); a p75/TNFR2 polypeptide provided at GenBank Accession No. NP_{—}001057 (FIG. 10C); an IL6 polypeptide provided at GenBank Accession No. NP_{—}000591 (FIG. 10D); an IL6R polypeptide provided at GenBank Accession No. NP_{—}000556 (FIG. 10E); an EGF polypeptide provided at GenBank Accession No. NP_{—}001954 (FIG. 10F); an EGFR polypeptide provided at GenBank Accession No. NP_{—}005219 (FIG. 10G); an IL-1α polypeptide provided at GenBank Accession No. NP_{—}000566 (FIG. 10H); an IL-1β polypeptide provided at GenBank Accession No. NP_{—}000567 (FIG. 10I); an IL1R1 polypeptide provided at GenBank Accession No. NP_{—}000868 (FIG. 10J); an IL1R2 polypeptide provided at GenBank Accession No. NP_{—}004624 (FIG. 10K); a G-CSF polypeptide provided at GenBank Accession No. NP_{—}000750 (FIG. 10L); a G-CSF-R polypeptide provided at GenBank Accession No. NP_{—}000751 (FIG. 10M); an MCP-1 polypeptide provided at GenBank Accession No. NP_{—}002973 (FIG. 10N); an MCP-1-R (CCR4) polypeptide provided at GenBank Accession No. NP_{—}001116513 (FIG. 10O); an MCP-1-R (CCR5) polypeptide provided at GenBank Accession No. NP_{—}000570 (FIG. 10P); an MIP-1α polypeptide provided at GenBank Accession No. NP_{—}002974 (FIG. 10Q); an MIP-1α-R (CCR4) polypeptide provided at GenBank Accession No. NP_{—}005499 (FIG. 10R); for an MIP-1α-R (CCR5) polypeptide provided at GenBank Accession No. NP_{—}000570 (FIG. 10S); for an MIP-1β polypeptide provided at GenBank Accession No. NP_{—}002975 (FIG. 10T); an MIP-1β-R (CCR1) polypeptide provided at GenBank Accession No. NP_{—}001286 (FIG. 10U); for an MIP-1β-R (CCR5)) polypeptide provided at GenBank Accession No. NP_{—}000570 (FIG. 10V); an SCF polypeptide provided at GenBank Accession No. NP_{—}000890 (FIG. 10W); an SCFR polypeptide provided at GenBank Accession No. NP_{—}000213 (FIG. 10X); for a RANTES polypeptide provided at GenBank Accession No. NP_{—}002974 (FIG. 10Y); a RANTES-R (CCR1) polypeptide provided at GenBank Accession No. NP_{—}001286 (FIG. 10Z); a RANTES-R (CCR3) polypeptide provided at GenBank Accession No. NP_{—}001828 (FIG. 10AA); a RANTES-R (CCR4) polypeptide provided at GenBank Accession No. NP_{—}005499 (FIG. 10AB); a RANTES-R (CCR5) polypeptide provided at GenBank Accession No. NP_{—}000570 (FIG. 10AC); an IFN-7 polypeptide provided at GenBank Accession No. NP_{—}000610 (FIG. 10AD); IFNGR1 polypeptide provided at GenBank Accession No. NP_{—}000407 (FIG. 10AE); and for an IFNGR2 polypeptide provided at GenBank Accession No. NP_{—}005525 (FIG. 10AF), each of which sequence is incorporated herein by reference in its entirety.

FIG. 11A-FIG. 11D is a series of photomicrographs and a bar chart showing the characterization of bone marrow derived EPCs. (FIG. 11A) Top row—representative fluorescent confocal images of BM-derived EPCs triple stained with c-kit—green (stem/progenitor cell marker). Isolectin-B4—red (endothelial cell marker) and TopRo-3—blue (to visualize nuclei) on days 5 after initial plating. Bottom row—representative fluorescent confocal images of BM-derived EPCs double stained with Sca-1—green (stem/progenitor cell marker), Isolectin-B4—red and TopRo-3—blue (to visualize nuclei) on days 5 after initial plating. Far right in both rows are the triple overlay. These double c-kit/Isolectin-B4 (+) and Sca-1/Isolectin-B4 (+) cells, presumably EPCs, constituted nearly 100% of cells on day 5, respectively, indicating that by this time most if not all of BM-derived cells were identified as EPCs by double (+) staining with EC marker and two progenitor markers (c-kit and Sca-1). (FIG. 11B) Representative confocal images of BM-derived EPCs expanded ex vivo for 5 days and stained for hematopoeitic lineage markers—Gr1 (to identify neutrophils). F4/80 (to identify macrophages), Isolectin-B4 and the uptake of DiI-ac-LDL while in culture (to confirm EC lineage of the cells). Graphic representation of the percent (+) cells in BM-derived EPC cultures for Gr1, F4/80, Isolectin-B4 and DiI-ac-LDL. (FIG. 11C) Representative phase contrast (×20) images of ex vivo expanded BM-derived EPC cultures on days 3 and 5 to demonstrate colony-specific expansion of EPCs on day 3 and relative morphologic homogeneity of EPC cultures on days 3 and 5 after initial plating. (FIG. 11D) In vitro tube-like structure formation assay on VEGF reduced matrigel to confirm EC function of EPCs in BM-derived cultures.

FIG. 12A-FIG. 12F is a series of bar charts, line graphs, and photomicrographs showing slow decay and increased p-H2AX foci in ex-vivo EPCs by Day 7 after full body 1 Gy γ-irradiation and increased bystander responses demonstrated by BM-derived EPCs in-vitro with increase in mean p-H2AX foci/cell over time. (FIG. 12A) Graphic representation of mean p-H2AX foci/cell after 60 h in the selective EPC culture media from WT mice at 30 min (black bars), 24 h (grey bars) and 7 days (white bars) after full body irradiation with 1 Gy γ-IR compared to respective N-IR controls. In control N-IR ex-vivo expanded EPCs there was no change over 7 days in p-HA2X foci—0.27±0.15 vs. 0.27±0.15 and 0.29±0.08, p=NS, all comparisons. Graphs represent data pooled from 3 independent biological samples treated under similar conditions. (FIG. 12B) Foci distribution plot of % WT EPCs with an N of p-H2AX foci for 30 min, 24 h and Day 7 post-IR treatment. (FIG. 12C) Representative images for p-H2AX immunostaining (yellow) and Topro-3 stained nuclei (red) in naïve WT EPCs in-vitro, treated with IR-CM medium transferred at 30 min, 5 h and 24 h post-IR of WT EPCs with 1 Gy γ-IR compared to respective controls after 24 h treatment with CM. (FIG. 12D) graphic representation of mean p-H2AX foci/cell post 24 h treatment of naïve WT ECPs with IR-CM medium from WT EPCs at 30 min (black bars), 5 h (grey bars) and 24 h (white bars) post 1 Gy γ-IR. In control Non-IR CM-treated EPCs there was no change over 24 hr in p-HA2X foci—0.7±0.1 vs. 0.8±0.2 and 0.8±0.2, p=NS, all comparisons. In IR-CM treated EPCs there was a significant increase in p-H2AX foci when comparing 30 min vs. 24 h—1±0.2 vs. 3.2±0.5, p<0.0001, and 5 h vs. 24 h 1.4±0.3 vs. 3.2±0.46, p<0.001. Graphs represent data pooled from n=3 independent biological samples treated under similar conditions. (FIG. 12E) Foci distribution plot of % naïve WT EPCs after IR-CM transfer with a given number (N) of foci count from 0-15 foci for 30 min, 5 h and 24 h treatment conditions. (FIG. 12F) For better visualization of distribution of EPCs with N foci insert graph provided shows the distribution after excluding the cells with zero foci (1-15 foci).

FIG. 13A is a line graph showing that TNF ligand-receptor interactions modify formation of p-H2AX foci in BM-derived EPCs in vitro. The analysis of p-H2AX formation and decay was performed for every time point where we included all cells with ≧1 p-H2AX foci and mean foci/cell was plotted. Graphic representation of mean p-H2AX foci/cell 24 h after treatment of naïve WT, p55KO and p75KO EPCs with IR-CM medium collected from respective WT, p55KO and p75KO EPCs at 1 h, 5 h, 24 h, Day 3 and Day 5 after 1 Gy of γ-IR, pH2AX foci/cell treated with 1 h IR-CM in WT vs. p75KO and p55KO—4.3±0.4 vs. 6.9±1 and 8.5±0.9, p<0.002 and p<0.0001, respectively; pH2AX foci/cell treated with 5 h IR-CM in WT vs. p75KO and p55KO—6.9±0.9 vs. 7.7±0.6 and 8.5±0.9, p=NS, both comparisons; pH2AX foci/cell treated with 24 h IR-CM in WT vs. p75KO and p55KO—9±0.8 vs. 3.7±0.5 and 4.8±0.6, p<0.0001, both comparisons; pH2AX foci/cell treated with 3-day IR-CM in WT vs. p75KO and p55KO—7.6±0.8 vs. 7.3±1.5 and 7.8±0.7, p=NS, both comparisons; pH2AX foci/cell treated with 5-day IR-CM in WT vs. p75KO and p55KO—3.8±0.4 vs. 5.9±0.8 and 8.5±1, p<0.04 and p<0.0001, as well as p<0.03 when comparing p75KO vs. p55KO. There was a steady increase over time in the formation of p-H2AX foci in N-IR p55KO treated with 1, 3 and 5-days IR-CM collected from corresponding genotype EPCs—4.8±0.6 vs. 7.8±0.7 vs. 8.5±1; p<0.002—day 1 vs. day 3, p<0.0001—day 1 vs. day 5, p=N.S—day 3 vs. day 5. There was a similar increase, however, followed by decrease in p75KO EPCs—3.7±0.5 vs. 7.3±1.5 vs. 5.9±0.8; p<0.003—day 1 vs. day 3, p<0.04—day 1 vs. day 5, p=N.S—day 3 vs. day 5. Graphs represent data pooled from n=3 independent biological samples treated under similar conditions.

FIG. 14 is a line graph showing neutralization of TNF-α in IR-CM resulted in significantly decreased p-H2AX foci formation in TNFR1/p55KO and WT EPCs in vitro. Please note, for the clarity of the comparison between the formation p-H2AX foci in EPCs treated with IR-CM media with or without TNF neutralization data points for WT and p55KO EPCs from FIG. 3 are overplayed in FIG. 4 again. Graphic representation of mean p-H2AX foci/cell post 24 h treatment of naïve WT and p55KO with IR-CM medium collected and treated with TNF-α neutralizing antibody from respective WT and p55KO EPCs at 1 h, 5 h, 24 h, Day 3 and Day 5 post 1 Gy γ-IR. pH2AX foci/cell treated with 1 h IR-CM in WT vs. p55KO—2.4±0.2 vs. 3.7±0.6, p<0.05; pH2AX foci/cell treated with 5 h IR-CM in WT vs. p55KO—4.3±0.5 vs. 3.6±0.5, p=N.S; pH2AX foci/cell treated with 24 h IR-CM in WT vs. p55KO—5.0±0.5 vs. 4.2±0.6, p=N.S; pH2AX foci/cell treated with 3-day IR-CM in WT vs. p55KO—3.2±0.4 vs. 4.9±0.8, p<0.03; pH2AX foci/cell treated with 5-day IR-CM in WT vs. p55KO—3.5±0.3 vs. 3.5±0.3, p=N.S. Treatment of IR-CM with TNF neutralizing antibody decreased the formation of p-H2AX foci at all time point examined in both WT and p55KO EPCs. However, the most significant decreases for WT EPCs were observed at 5, 24 hours and 3 days and for p55KO EPCs at 1, 5 hours, and 3, 5 days. Graphs represent data pooled from n=3 independent biological samples treated under similar conditions.

FIG. 15A-FIG. 15F is a series of bar charts showing Foci distribution of naïve p55KO EPCs with N number of foci with and without TNF-α neutralization after 1 Gy γ-IR and media transfer at various time points—before (control), 1 h, 5 h, 24 h, Day 3 and Day 5. (FIG. 15A-FIG. 15F) Foci distribution of naïve p55KO EPCs with N number of foci upon treatment with IR-CM from p55KO (red bars and dashed lines) and with IR-CM from p55KO EPCs after TNF neutralization (green bars and dashed lines).

FIG. 16A-FIG. 16N is a series of line graphs showing radiation induced increase in the accumulation of cytokines, chemokines and growth factors in WT and p55KO EPCs in-vitro. (FIG. 16A) TNF-α levels (pg/ml) measured in IR-CM growth media from WT and p55KO EPCs after 1 Gy γ-IR at 1 h, 5 h, 24 h, day 3 and day 5. (FIG. 16B-FIG. 16G) Cytokine and chemokine concentrations (pg/ml) measured in IR-CM from WT and p55KO EPCs after 1 Gy γ-IR at 1 h, 5 h, 24 h, day 3 and day 5. (FIG. 16H-FIG. 16N), Growth factor concentrations (pg/ml) measured in IR-CM from WT and p55KO EPCs after 1 Gy γ-IR at 1 h, 5 h, 24 h, day 3 and day 5. Graphs represent data pooled from n=3 independent biological samples treated under similar conditions. Statistical significance between WT and p55KO EPCs at each time point is denoted as—*p<0.05-p<0.01, **p<0.009-p<0.001 and ***p<0.0009.

FIG. 17A-FIG. 17C is a series of bar charts showing p-H2AX foci formation increased in non-irradiated p55KO EPCs in-vitro after treatment with mouse recombinant TNF-α and IL-1α. (FIG. 17A) Graphic representation of mean p-H2AX foci/cell after 24 h treatment of naïve p55KO EPCs with various concentrations of mouse recombinant (rm) IL-1α (red bars), rmRantes (green bars), rmMCP-1 (blue bars) and rmTNF-α (yellow bars) compared to control p55KO EPCs (black bar). Graphs represent data pooled from identically treated 3 independent biological samples. (FIG. 17B) Foci distribution of naïve p55KO EPCs with N number of foci after treatment with various concentrations of rmTNF-α protein in vitro for 24 hr. Almost 0.5%-2.55% and 0.5-1% of cells treated with 100 pg/ml (red bars and dashed line), 1000 pg/ml (green bars and dashed line) and 40000 pg/ml (blue bars and dashed line) of TNF-α had 9-18 and 19-31 foci/cell respectively when compared to control p55KO EPCs (black bars and dashed line) which had no more than 0.5% cells with a maximum of 13 and 15 p-H2AX foci/cell. Treatment with 40000 pg/ml of TNF-α resulted in a maximum of 31 foci/cell in 0.5% of EPCs. (FIG. 17C) Foci distribution of naïve p55KO EPCs with N number of foci upon treatment with 290 pg/ml (green bars) and 580 pg/ml (red bars) concentrations of rmIL-1α protein in vitro for 24 h. Mouse recombinant IL-1α treatment resulted in >4% of cells with 9-18 foci/cell, whereas control p55KO EPCs (black bars) had no more than 0.5% cells with a maximum of 13 and 15 p-H2AX foci/cell. At the same time 0.5%-3% of p55KO EPCs had a maximum of 19-37 foci/cell in 580 pg/ml and 19-51 foci/cell in 290 pg/ml treatment conditions.

FIG. 18A and FIG. 18B are a series of photomicro graphs and a bar chart demonstrating foci distribution of naïve BM-EPCs with N number of foci post ¹H-IR and CM transfer at various time points. FIG. 18A is a series of photomicrographs showing reprehensive images of p-H2AX foci at the indicated time points. FIG. 18B shows a foci distribution plot for % of naïve BM-EPCs with a given number (N) of foci upon 24 h treatment with CM from 0.90 Gy ¹H-IR BM-EPCs at 2 h (red bars and dotted lines), 5 h (blue bars and dashed lines) and 24 h (green bars and dashed/dotted lines) compared to non-IR controls (black bars and solid lines). Graph provided shows the distribution after excluding the cells with zero foci for both ionizing IRs.

FIG. 19A-FIG. 19H is a series of line graphs demonstrating that inflammatory cytokines and chemokines are significantly increased in ¹H-IR Conditioned Medium. A graphic representation of radiation induced increase in the cumulative concentration (pg/mL) of cytokines, chemokines and growth factors in CM from 0.90 Gy ¹H-IR BM-EPCs in-vitro at 2 h, 5 h and 24 h post-IR for IL-1α (FIG. 19A), IL-1β (FIG. 19B), G-CSF (FIG. 19C), GM-CSF (FIG. 19D), MCP-1 (FIG. 19E), MIP-1α (FIG. 19F), SCF (FIG. 19G), and Rantes (FIG. 19H). Graphs represent data pooled from 3 independent biological samples treated under similar conditions.

FIG. 20A is a line graph demonstrating that full body ¹H-IR induces delayed decrease in BM-EPC Proliferation ex-vivo. FIG. 20A is a graphic representation of cell proliferation count performed using MTT proliferation assay on BM-EPCs cultured ex-vivo for 72 h from full-body irradiated mice at 2 h, 5 h, 24 h, 7 days, 14 days and 28 days post-IR with different initial seeding densities for full-body ¹H-IR mice. Graph represents data pooled from 5-6 independent biological samples treated under similar conditions depicted as mean±SEM values. Statistical significance was assigned when p<0.05. FIG. 20B is a line graph demonstrating that full-body ¹H-IR induces early (5-24 h) and delayed (28 days) apoptosis in BM-EPCs ex-vivo. FIG. 20B is a graphic representation of mean % change in Annexin V and P.I positive (dbl+ve) BM-EPCs cultured ex-vivo from 0.90 Gy ¹H full-body irradiated mice at 2 h, 5 h, 24 h, 7 days, 14 days and 28 days post-IR. Dbl+ve (Annexin V+/P.I+) BM-EPCs were analyzed by FACS and compared to non-IR control set at 100%. Graph represents data pooled from 5-6 independent biological samples treated under similar conditions depicted as mean±SEM values. Statistical significance was assigned when p<0.05.

FIG. 21A FIG. 21B are a series of photomicro graphs and a bar chart demonstrating foci distribution of naïve BM-EPCs with N number of foci post ⁵⁶Fe-IR and CM transfer at various time points. FIG. 21A is a series of photomicrographs showing reprehensive images of p-H2AX foci at the indicated time points. FIG. 21B shows a foci distribution plot for % of naïve BM-EPCs with a given number (N) of foci upon 24 h treatment with CM from 0.15 Gy ⁵⁶Fe-IR BM-EPCs at 2 h (red bars and dotted lines), 5 h (blue bars and dashed lines) and 24 h (green bars and dashed/dotted lines) compared to non-IR controls (black bars and solid lines). Graph provided shows the distribution after excluding the cells with zero foci for both ionizing IRs.

FIG. 22A-FIG. 22H is a series of line graphs demonstrating that inflammatory cytokines and chemokines are significantly increased in ⁵⁶Fe-IR Conditioned Medium. A graphic representation of radiation induced increase in the cumulative concentration (pg/mL) of cytokines, chemokines and growth factors in CM from 0.15 Gy ⁵⁶Fe-IR BM-EPCs in-vitro at 2 h, 5 h and 24 h post-IR for IL-1α (FIG. 22A), IL-1β (FIG. 22B). G-CSF (FIG. 22C), GM-CSF (FIG. 22D), MCP-1 (FIG. 22E), MIP-1α (FIG. 22F), SCF (FIG. 22G), and Rantes (FIG. 22H). Graphs represent data pooled from 3 independent biological samples treated under similar conditions.

FIG. 23A is a line graph demonstrating that full body ⁵⁶Fe-IR induces delayed decrease in BM-EPC Proliferation ex-vivo. FIG. 20A is a graphic representation of cell proliferation count performed using MTT proliferation assay on BM-EPCs cultured ex-vivo for 72 h from full-body irradiated mice at 2 h, 5 h, 24 h, 7 days, 14 days and 28 days post-IR with different initial seeding densities for full-body ⁵⁶Fe-IR mice. Graph represents data pooled from 5-6 independent biological samples treated under similar conditions depicted as mean±SEM values. Statistical significance was assigned when p<0.05. FIG. 23B is a line graph demonstrating that full-body ⁵⁶Fe-IR induces early (5-24 h) and delayed (28 days) apoptosis in BM-EPCs ex-vivo. FIG. 23B is a graphic representation of mean % change in Annexin V and P.I positive (dbl+ve) BM-EPCs cultured ex-vivo from 0.15 Gy ⁵⁶Fe full-body irradiated mice at 2 h, 5 h, 24 h, 7 days, 14 days and 28 days post-IR. Dbl+ve (Annexin V+/P.I+) BM-EPCs were analyzed by FACS and compared to non-IR control set at 100%. Graph represents data pooled from 5-6 independent biological samples treated under similar conditions depicted as mean±SEM values. Statistical significance was assigned when p<0.05.

### DETAILED DESCRIPTION OF THE INVENTION

The invention features compositions and methods that are useful for the treatment or prevention of radiation exposure featuring compositions that inhibit the expression or activity of one or more (e.g., 2, 3, 4, 5, 10, 20, or more) of TNF-α (p55 or p75), IL6, EGF, IL1-alpha, IL1-beta, G-CSF, MCP-1, MIP-1, SCF, or RANTES receptor, or a TNF-α, IL6, EGF, IL1-alpha, IL1-beta, G-CSF, MCP-1, MIP-1, SCF, or RANTES peptide, or fragment thereof. Additionally, agents useful in the invention include those that inhibit the pathways (e.g., signal transduction pathways) the receptors and peptides described herein are involved in (e.g., kinase inhibitors).

The invention is based, at least in part, on the discovery that interfering with the expression of one or more of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide is useful for the treatment of radiation exposure. As reported in more detail below, increased levels of one or more of TNF-α (p55 or p75), IL6, EGF, IL1-alpha, IL1-beta, G-CSF, MCP-1, MIP-1, SCF, or RANTES receptor were expressed after exposure of cells to radiation. These results indicate that inhibiting the expression of one or more of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide decrease radiation-induced delayed non-targeted effects (i.e., continues generation of DNA double strand breaks and increased oxidative trees). Thus, inhibiting the expression of one or more of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide increases cell growth or survival, reduce cell death, and/or decreases the risk of neoplastic transformation from radiation exposure. Without intending to be bound by theory, long-term degenerative risks to organ-systems such as heart and central nervous system (CNS) are minimized. Increased levels of one or more of IL6, EGF, IL1-alpha, IL1-beta, G-CSF, MCP-1, MIP-1, SCF, or RANTES peptide were expressed after exposure of p55KO/TNFR1 or p75KO/TNFR2 cells to radiation. Accordingly, treatment with an agent that reduces the expression or biological activity of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide is useful for increasing survival or proliferation of a cell (e.g., bone marrow stem and progenitor cells) exposed to radiation. In particular embodiments, methods of the invention prevent or treat a cardiovascular condition associated with radiation exposure.

Accordingly, the invention provides methods of treating radiation exposure and/or disorders or symptoms thereof which comprise administering a therapeutically effective amount of a pharmaceutical composition comprising a compound (e.g., an inhibitory nucleic acid molecule that disrupts p75 or p55 TNF-α receptor expression) described herein to a subject (e.g., a mammal such as a human). Thus, in one embodiment, the invention provides a method of treating a subject suffering from or susceptible to radiation exposure or disorder or symptom thereof. The method includes the step of administering to the mammal a therapeutic amount of a compound described herein sufficient to treat the disease or disorder or symptom thereof, under conditions such that the disease or disorder is treated.

In particular, inhibiting the expression of one or more of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide can be used to treat short- and long-term degenerative cardiovascular risks due to radiation exposure. Without being bound to a particular theory, low-dose space radiation-induced DNA damage repair (e.g., double strand breaks, oxidative damage) is inefficient in bone marrow (BM)-derived endothelial progenitor cells (EPCs), which can lead to increased mutagenesis with subsequent long-term loss of endothelial function of BM-derived EPCs. A growing body of evidence indicates that neovascularization involves not only proliferation of local ECs but also BM-derived EPCs³⁰. Consequently, EPCs are critical to endothelial maintenance and repair, and EPC dysfunction contributes to the pathogenesis of ischemic vascular diseases, as well as for maintenance of normal vascular homeostasis in the heart. Indeed, studies have demonstrated that in patients with CV risk factors, the number and migratory ability of EPCs isolated from peripheral blood is reduced³¹ and EPC function is impaired³². In addition, a strong inverse correlation was reported between the number of circulating EPCs, vascular function and the subject's combined Framingham cardiovascular factor score³³. Furthermore, measurements of flow-mediated brachial-artery reactivity also revealed a significant relation between endothelial function and the number of EPCs, supporting a role for EPCs in the maintenance of endothelial integrity³⁴. Without being bound to a particular theory, loss of endothelial function of BM-derived EPCs poses significant degenerative CV risk on physiologic homeostasis in the aging heart and on the regeneration and neovascularization processes in the heart under pathologic conditions such as acute myocardial infarction (AMI), and acute or chronic heart failure (AHF or CHF).

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test; enzyme or protein marker number of bone marrow-derived endothelial progenitor cells, hemangioblasts, or hematopoeitic stem cells; family history; and the like). The compounds herein may be also used in the treatment of any other disorders in which radiation exposure, or an increased expression of one or more of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide may be implicated.

### Tumor Necrosis Factor Alpha (TNF-α)

Tumor necrosis factor alpha (TNF-α) was first identified through the detection of antitumor activity in the sera of mice treated with endotoxin, and has been shown to be a member of a large family of related cytokines termed the TNF family. TNF family ligands are type II transmembrane proteins (intracellular N-terminus) that are biologically active as self assembling, non covalent bound, trimers. Some of these ligands, e.g. TNF-α, are active both as a membrane integrated form and as a soluble form released from the cell membrane after proteolytic cleavage, mainly by metalloproteinases induced by various stimuli.

Mature TNF-α is a 17 kDa polypeptide that specifically binds at least two cell surface receptors, p55 and p75 TNF-α receptors. These receptors have highly homologous extracellular domains, but show little homology between their intracellular domains. Without being tied to a particular theory, it is believed that p55 and p75 TNF-α receptors act through distinct signal transduction pathways. The p55 TNF-α receptor is generally responsible for signaling a variety of responses, including cytotoxicity and cytokine secretion, whereas the p75 TNF-α receptor is generally responsible for lymphoproliferative signals and the activation of T cells. TNF-α receptors are ubiquitously expressed on nearly all cell types, but the p75 TNF-α receptor is preferentially expressed by lymphoid cells, as well as other hematopoeitic and endothelial cells.

Depending on TNF-α concentration and duration of exposure, TNF-α can act as either a tumor necrosis factor or a tumor-promoting factor. Endogenous TNF-α, which is constitutively produced in the tumor microenvironment, enhances tumor growth and induces cytokines/chemokines involved in cancer progression. Ongoing studies and results have shown the direct role of TNF-α receptors in tumor growth and cancer propagation. In contrast. TNF-α administered locally at high-dose, is anti-angiogenic and displays a potent anti-tumor effect. However, administration of TNF-α also induces systemic toxicity. As reported herein, disruption of the TNF-α p75 receptor or the TNF-α p55 receptor is associated with a reduction in neoplastic transformation, cell survival or proliferation. Without intending to be bound to theory, non-targeted effects due to DNA damage lead to apoptosis. Increased apoptosis in neoplastic and endothelial cells and reduced angiogenesis in radiation exposure are observed when the expression of either the TNF-α p75 receptor or the TNF-α p55 receptor is inhibited. Accordingly, the invention provides methods that reduce the expression or biological activity of the TNF-α p75 receptor or the TNF-α p55 receptor for the prevention of delayed non-targeted effects of radiation exposure, including degerative risks to heart and CNS, and for the treatment for the effects of radiation exposure, including lung cancer and other human cancers.

### Interleukin 1 Alpha (IL-1α)

Interleukin-1 alpha possesses a wide spectrum of metabolic, physiological, haematopoietic activities, and plays one of the central roles in the regulation of the immune responses. IL-1α binds to the interleukin-1 receptor. IL-1α is also known as fibroblast-activating factor (FAF), lymphocyte-activating factor (LAF), B-cell-activating factor (BAF), leukocyte endogenous mediator (LEM), epidermal cell-derived thymocyte-activating factor (ETAF), serum amyloid A inducer of hepatocyte-stimulating factor (HSP), catabolin, hemopoetin-1 (H-1), endogenous pyrogen (EP), osteoclast-activating factor (OAF), and proteolysis-inducing factor (PIF).

IL-1α is a unique member in the cytokine family because the structure of its initially synthesized precursor does not contain a signal peptide fragment. Calpain, a calcium-activated cysteine protease, associated with the plasma membrane, is primarily responsible for the cleavage of the IL-1α precursor into a mature molecule. Both the 31 kDa precursor form of IL-1α and its 18 kDa mature form are biologically active. The 31 kDa IL-1α precursor is synthesized in association with cytoskeletal structures (micro-tubules), unlike most proteins, which are translated in the endoplasmic reticulum. Crystal structure analysis of the mature form of IL-1α shows that it has two sites for binding IL-1 receptor.

IL-1α is constitutively produced by epithelial cells, and is found in substantial amounts in normal human epidermis. A wide variety of other cells only upon stimulation can be induced to transcribe the IL-1α genes and produce the precursor form of IL-1α (including fibroblasts, macrophages, granulocytes, eosinophils, mast cells and basophils, endothelial cells, platelets, monocytes and myeloid cell lines, blood T-lymphocytes and B-lymphocytes, astrocytes, kidney mesangial cells, Langerhans cells, dermal dendritic cells, natural killer cells, large granular lymphocytes, microglia, blood neutrophils, lymph node cells, maternal placental cells and several other cell types). IL-1α function as an epidermal cytokine.

IL1-1α has been shown to interact with HAX1 and NDN. Although there are many interactions of IL-1α with other cytokines, the most consistent and most clinically relevant is its synergism with TNFα. However, there are a few examples in which the synergism between IL-1α and TNFα has not been demonstrated, including radioprotection, the Shwartzman reaction, PGE2 synthesis, sickness behavior, nitric oxide production, nerve growth factor synthesis, insulin resistance, loss of mean body mass, and IL-8 and chemokine synthesis.

### Interleukin 1 Alpha (IL-1β)

Interleukin-1 beta (IL-1β; catabolin) is a member of the interleukin 1 cytokine family. IL-1β precursor is cleaved by caspase 1 (interleukin 1 beta convertase). IL-1β is produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. The induction of cyclooxygenase-2 (PTGS2/COX2) by this cytokine in the central nervous system (CNS) is found to contribute to inflammatory pain hypersensitivity. This gene and eight other interleukin 1 family genes are from a cytokine gene cluster on chromosome 2.

### Interleukin 6 (IL-6)

IL-6 is an interleukin that acts as both a pro-inflammatory and anti-inflammatory cytokine. It is secreted by T cells and macrophages to stimulate immune response to trauma, especially burns or other tissue damage leading to inflammation. IL-6 is also a “myokine,” a cytokine produced from muscle, and is elevated in response to muscle contraction. It is significantly elevated with exercise, and precedes the appearance of other cytokines in the circulation. Additionally, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1γa and IL-10. IL-6 causes an IL-10-dependent inhibition of CD4 T-cell expansion and function by up-regulating PD-1 levels on monocytes which leads to IL-10 production by monocytes after binding of PD-1 by PD-L.

IL-6 is an important mediator of fever and of the acute phase response. It is capable of crossing the blood brain barrier and initiating synthesis of PGE2 in the hypothalamus, thereby changing the body's temperature setpoint. In the muscle and fatty tissue IL-6 stimulates energy mobilization which leads to increased body temperature.

IL-6 can be secreted by macrophages in response to specific microbial molecules, referred to as pathogen associated molecular patterns (PAMPs). These PAMPs bind to highly important group of detection molecules of the innate immune system, called pattern recognition receptors (PRRs), including Toll-like receptors (TLRs). These are present on the cell surface and intracellular compartments and induce intracellular signaling cascades that give rise to inflammatory cytokine production. IL-6 is also essential for hybridoma growth and is found in many supplemental cloning media such as briclone. Il-6 is also produced by adipocytes and is thought to be a reason why obese individuals have higher endogeneous levels of CRP. In a 2009 study, intranasally administered IL-6 was shown to improve sleep-associated consolidation of emotional memories.

IL-6 signals through a cell-surface type I cytokine receptor complex consisting of the ligand-binding IL-6Rα chain (CD126), and the signal-transducing component gp130 (also called CD130). CD130 is the common signal transducer for several cytokines including leukemia inhibitory factor (LIF), ciliary neurotropic factor, oncostatin M, IL-11 and cardiotrophin-1, and is almost ubiquitously expressed in most tissues. In contrast, the expression of CD126 is restricted to certain tissues. As IL-6 interacts with its receptor, it triggers the gp130 and IL-6R proteins to form a complex, thus activating the receptor. These complexes bring together the intracellular regions of gp130 to initiate a signal transduction cascade through certain transcription factors, Janus kinases (JAKs) and Signal Transducers and Activators of Transcription (STATs) (ref 5).

In addition to the membrane-bound receptor, a soluble form of IL-6R (sIL-6R) has been purified from human serum and urine. Many neuronal cells are unresponsive to stimulation by IL-6 alone, but differentiation and survival of neuronal cells can be mediated through the action of sIL-6R. The sIL-6R/IL-6 complex can stimulate neurites outgrowth promote survival of neurons, hence may be important in nerve regeneration through remyelination.

IL-6 is relevant to many disease processes such as diabetes, atherosclerosis, osteoporosis, depression, Alzheimer's Disease, systemic lupus erythematosus, prostate cancer, and rheumatoid arthritis. Advanced/metastatic cancer patients have higher levels of IL-6 in their blood. Hence there is an interest in developing anti-IL-6 agents as therapy against many of these diseases. The first such is tocilizumab which has been approved for rheumatoid arthritis. Another, ALD518, is in clinical trials. Inhibitors of IL-6 (including estrogen) are used to treat postmenopausal osteoporosis.

### Epidermal Growth Factor (EGF)

Epidermal growth factor or EGF is a growth factor that plays an important role in the regulation of cell growth, proliferation, and differentiation by binding to its receptor EGFR. Human EGF is a 6 kDa protein with 53 amino acid residues and three intramolecular disulfide bonds. Epidermal growth factor can be found in human platelets, macrophages, urine, saliva, milk, and plasma. Epidermal growth factor has been shown to interact with Epidermal growth factor receptor and PIK3R2.

EGF results in cellular proliferation, differentiation, and survival. EGF is a low-molecular-weight polypeptide first purified from the mouse submandibular gland, but since then found in many human tissues including submandibular gland, parotid gland. Salivary EGF, which seems also regulated by dietary inorganic iodine, also plays an important physiological role in the maintenance of oro-esophageal and gastric tissue integrity. The biological effects of salivary EGF include healing of oral and gastroesophageal ulcers, inhibition of gastric acid secretion, stimulation of DNA synthesis as well as mucosal protection from intraluminal injurious factors such as gastric acid, bile acids, pepsin, and trypsin and to physical, chemical and bacterial agents.

EGF activity is mediated through the MAPK/ERK pathway. EGF acts by binding with high affinity to epidermal growth factor receptor (EGFR) on the cell surface and stimulating the intrinsic protein-tyrosine kinase activity of the receptor. The tyrosine kinase activity, in turn, initiates a signal transduction cascade that results in a variety of biochemical changes within the cell—a rise in intracellular calcium levels, increased glycolysis and protein synthesis, and increases in the expression of certain genes including the gene for EGFR—that ultimately lead to DNA synthesis and cell proliferation.

EGF is the founding member of the EGF-family of proteins. Members of this protein family have highly similar structural and functional characteristics. Besides EGF itself other family members include: Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4). EGF family members contain one or more repeats of the conserved amino acid sequence: CX7CX4-5CX10-13CXCX8GXRC, where X represents any amino acid. This sequence contains 6 cysteine residues that form three intramolecular disulfide bonds. Disulfide bond formation generates three structural loops that are essential for high-affinity binding between members of the EGF-family and their cell-surface receptors.

Because of the increased risk of cancer by EGF, inhibiting it decreases cancer risk. Such medications are so far mainly based on inhibiting the EGF receptor. Monoclonal antibodies are potential substances for this purpose.

### Monocyte Chemotactic Protein-1 (MCP-1)

Monocyte chemotactic protein-1 (MCP-1) Chemokine (C-C motif) ligand 2 (CCL2) is a small cytokine belonging to the CC chemokine family that is also known as Chemokine (C-C motif) ligand 2 (CCL2) and small inducible cytokine A2. CCL2 recruits monocytes, memory T cells, and dendritic cells to sites of tissue injury, infection, and inflammation. The levels of CCL2 vary considerably between normal people. Multivariable-adjusted heritability of CCL2 concentrations in whites of European descent has been reported to be 0.37 in plasma and 0.44 in serum.

As with many other CC chemokines, CCL2 is located on chromosome 17 (17q 11.2-q21.1) in humans. The gene spans 1,927 bases and lies on the Watson (plus) strand. The gene has three exons and two introns. CCL2 is produced as a protein precursor containing signal peptide of 23 amino acids and a mature peptide of 76 amino acids with a predicted weight of 11.025 kiloDaltons (kDa). CCL2 is a monomeric polypeptide, with a molecular weight of approximately 13 kDa. It is tethered on endothelial cells by glycosaminoglycan side chains of proteoglycans. It is primarily secreted by monocytes, macrophages and dendritic cells. It is a platelet derived growth factor inducible gene. It is cleaved by the metalloproteinase MMP-12. Cell surface receptors that bind CCL2 include CCR2 and CCR4.

CCL2 displays chemotactic activity for monocytes and basophils but not for neutrophils or eosinophils. Deletion of the N-terminal residue converts it from an activator of basophils to an eosinophil chemoattractant. CCL2 causes the degranulation of basophils and mast cells, an effect potentiated by pre-treatment with IL-3 and other cytokines. It augments monocyte anti-tumor activity and is essential for granuloma formation

CCL2 is found at the site of tooth eruption and bone degradation. In the bone, CCL2 is expressed by mature osteoclasts and osteoblasts and is under the control of nuclear factor κB (NFκB). In human osteoclasts, it has been shown that CCL2 and RANTES (regulated on activation normal T cell expressed and secreted) are unregulated by RANKL (receptor activator of NFκB ligand). Both MCP-1 and RANTES were also shown to induce the formation of TRAP-positive, multinuclear cells from M-CSF-treated monocytes in the absence of RANKL, but produced osteoclasts that lacked cathepsin K expression and resorptive capacity. It is proposed that CCL2 and RANTES act as autocrine loop in human osteoclast differentiation.

The CCL2 chemokine is also expressed by neurons, astrocytes and microglia in nervous tissue. Neuronal expression of CCL2 is mainly found in the cerebral cortex, globus pallidus, hippocampus, paraventricular and supraoptic hypothalamic nuclei, lateral hypothalamus, substantia nigra, facial nuclei, motor and spinal trigeminal nuclei, gigantocellular reticular nucleus and in Purkinje cells in the cerebellum.

CCL2 has been implicated in the pathogenesis of diseases characterized by monocytic infiltrates, like psoriasis, rheumatoid arthritis and atherosclerosis. Administration of anti-MCP-1 antibodies in a model of glomerulonephritis have shown reduced macrophage and T cell infiltration, as well as reduced crescent formation, scarring and renal impairment. Recent data indicates an important role for CCL2 in the neuroinflammatory processes that takes place in various central nervous system (CNS) diseases characterized by neuronal degeneration. Its expression by glial cells is increased in epilepsy, brain ischemia, Alzheimer's disease, experimental autoimmune encephalomyelitis (EAE), and traumatic brain injury.

### Macrophage Inflammatory Protein-1α (MIP-1α)

Macrophage inflammatory protein-1α (MIP-1α), also known as Chemokine (C-C motif) ligand 3 (CCL3), is a cytokine belonging to the CC chemokine family that is involved in the acute inflammatory state in the recruitment and activation of polymorphonuclear leukocytes (Wolpe et al., 1988). Sherry et al. (1988) demonstrated 2 protein components of MIP1, called by them alpha and beta. CCL3 has been shown to interact with CCL4.

Macrophage Inflammatory Proteins (MIP) belong to the family of chemotactic cytokines known as chemokines. Macrophage inflammatory protein-1 (MIP-1), MIP-1 (CCL3) and MIP-1 (CCL4) are chemokines crucial for immune responses towards infection and inflammation. In humans, there are two major forms. MIP-1α and MIP-1β that are now officially named CCL3 and CCL4 respectively. Both are major factors produced by macrophages after they are stimulated with bacterial endotoxins. They activate human granulocytes (neutrophils, eosinophils and basophils) which can lead to acute neutrophilic inflammation. They also induce the synthesis and release of other pro-inflammatory cytokines such as interleukin 1 (IL-1), IL-6 and TNF-α from fibroblasts and macrophages. The genes for CCL3 and CCL4 are both located on human chromosome 17.

They are produced by many cells, particularly macrophages, dendritic cells, and lymphocytes. MIP-1 are best known for their chemotactic and proinflammatory effects but can also promote homoeostasis. Biophysical analyses and mathematical modelling has shown that MIP-1 reversibly forms a polydisperse distribution of rod-shaped polymers in solution. Polymerization buries receptor-binding sites of MIP-1, thus depolymerization mutations enhance MIP-1 to arrest monocytes onto activated human endothelium.

Chemokine (C-C motif) Ligand 5 (CCL5)

Chemokine (C-C motif) ligand 5 (also CCL5) is a protein which in humans is encoded by the CCL5 gene. It is also known as RANTES (Regulated upon Activation, Normal T-cell Expressed, and Secreted).

CCL5 is an 8 kDa protein classified as a chemotactic cytokine or chemokine. CCL5 is chemotactic for T cells, eosinophils, and basophils, and plays an active role in recruiting leukocytes into inflammatory sites. With the help of particular cytokines (i.e., IL-2 and IFN-γ) that are released by T cells, CCL5 also induces the proliferation and activation of certain natural-killer (NK) cells to form CHAK (CC-Chemokine-activated killer) cells. It is also an HIV-suppressive factor released from CD8+ T cells. This chemokine has been localized to chromosome 17 in humans. CCL5 has been shown to interact with CCR3, CCR5 and CCR1. CCL5 also activates the G-protein coupled receptor GPR75.

RANTES was first identified in a search for genes expressed “late” (3-5 days) after T cell activation. It was subsequently determined to be a CC chemokine and expressed in more than 100 human diseases. RANTES expression is regulated in T lymphocytes by Kruppel like factor 13 (KLF13). RANTES, along with the related chemokines MIP-1alpha and MIP-1beta, has been identified as a natural HIV-suppressive factor secreted by activated CD8+ T cells and other immune cells. Recently, the RANTES protein has been engineered for in vivo production by Lactobacillus bacteria, and this solution is being developed into a possible HIV entry-inhibiting topical microbicide.

### Stem Cell Factor (SCF)

Stem Cell Factor (also known as SCF, kit-ligand, KL, or steel factor) is a cytokine that binds to the c-Kit receptor (CD117). SCF can exist both as a transmembrane protein and a soluble protein. This cytokine plays an important role in hematopoiesis (formation of blood cells), spermatogenesis, and melanogenesis. The gene encoding stem cell factor (SCF) is found on the SI locus in mice and on chromosome 12q22-12q24 in humans.

Alternative splicing of the same RNA transcript produces soluble and transmembrane forms of stem cell factor (SCF). The soluble form of SCF contains a proteolytic cleavage site in exon 6. Cleavage at this site allows the extracellular portion of the protein to be released. The transmembrane form of SCF is formed by alternative splicing that excludes exon 6. Both forms of SCF bind to c-Kit and are biologically active. Soluble and transmembrane SCF is produced by fibroblasts and endothelial cells. Soluble SCF has a molecular weight is 18.5 KDa and forms a dimer. It is detected in normal human blood serum at 3.3 ng/mL. The presence of both soluble and transmembrane SCF is required for normal hematopoietic function. Mice that produce the soluble SCF but not transmembrane SCF suffer from anemia, are sterile, and lack pigmentation. This suggests that transmembrane SCF plays a special role in vivo that is separate from that of soluble SCF.

SCF binds to the c-Kit receptor (CD 117), a receptor tyrosine kinase, c-Kit is expressed in HSCs, mast cells, melanocytes, and germ cells. It is also expressed in hematopoietic progenitor cells including erythroblasts, myeloblasts, and megakaryocytes. However, with the exception of mast cells, expression decreases as these hematopoietic cells mature and c-Kit is not present when these cells are fully differentiated. SCF binding to c-Kit causes the receptor to homodimerize and auto-phosphorylate at tyrosine residues. The activation of c-Kit leads to the activation of multiple signaling cascades, including the RAS/ERK, PI3-Kinase, Src kinase, and JAK/STAT pathways.

SCF plays an important role in the hematopoiesis during embryonic development. Sites where hematopoiesis takes place, such as the fetal liver and bone marrow, all express SCF. Mice that do not express SCF die in utero from severe anemia. Mice that do not express the receptor for SCF (c-Kit) also die from anemia. SCF may serve as guidance cues that direct hematopoietic stem cells (HSCs) to their stem cell niche (the microenvironment in which a stem cell resides), and it plays an important role in HSC maintenance. Non-lethal point mutants on the c-Kit receptor can cause anemia, decreased fertility, and decreased pigmentation.

During development, the presence of the SCF also plays an important role in the localization of melanocytes, cells that produce melanin and control pigmentation. In melanogenisis, melanoblasts migrate from the neural crest to their appropriate locations in the epidermis. Melanoblasts express the Kit receptor, and it is believed that SCF guides these cells to their terminal locations. SCF also regulates survival and proliferation of fully differentiated melanocytes in adults. Fetal HSCs are more sensitive to SCF than HSCs from adults. In fact, fetal HSCs in cell culture are 6 times more sensitive to SCF than adult HSCs based on the concentration that allows maximum survival.

In spermatogenesis, c-Kit is expressed in primordial germ cells, spermatogonia, and in primordial oocytes. It is also expressed in the primordial germ cells of females. SCF is expressed along the pathways that the germ cells use to reach their terminal destination in the body. It is also expressed in the final destinations for these cells. Like for melanoblasts, this helps guide the cells to their appropriate locations in the body.

SCF plays a role in the regulation of HSCs in the stem cell niche in the bone marrow. SCF has been shown to increase the survival of HSCs in vitro and contributes to the self renewal and maintenance of HSCs in-vivo. HSCs at all stages of development express the same levels of the receptor for SCF (c-Kit). The stromal cells that surround HSCs are a component of the stem cell niche, and they release a number of ligands, including SCF. In the bone marrow, HSCs and hematopoietic progenitor cells are adjacent to stromal cells, such as fibroblasts and osteoblasts. These HSCs remain in the niche by adhering to ECM proteins and to the stromal cells themselves. SCF has been shown to increase adhesion and thus may play a large role in ensuring that HSCs remain in the niche. A small percentage of HSCs regularly leave the bone marrow to enter circulation and then return back to their niche in the bone marrow. It is believed that concentration gradients of SCF, along with the chemokine SDF-1, allow HSCs to find their way back to the niche.

In adult mice, the injection of the ACK2 anti-kit antibody, which binds to the c-Kit receptor and inactivates it, leads to severe problems in hematopoiesis. It causes a significant decrease in the number HSC and other hematopoietic progenitor cells in the bone marrow. This suggests that SCF and c-Kit plays an important role in hematopoietic function in adulthood. SCF also increases the survival of various hematopoietic progenitor cells, such as megakaryocyte progenitors, in vitro. In addition, it works with other cytokines to support the colony growth of BFU-E, CFU-GM, and CFU-GEMM4. Hematopoietic progenitor cells have also been shown to migrate towards a higher concentration gradient of SCF in vitro, which suggests that SCF is involved in chemotaxis for these cells.

Mast cells are the only terminally differentiated hematopoietic cells that express the c-Kit receptor. Mice with SCF or c-Kit mutations have severe defects in the production of mast cells, having less than 1% of the normal levels of mast cells. Conversely, the injection of SCF increases mast cell numbers near the site of injection by over 100 times. In addition, SCF promotes mast cell adhesion, migration, proliferation, and survival. It also promotes the release of histamine and tryptase, which are involved in the allergic response.

SCF may be used along with other cytokines to culture HSCs and hematopoietic progenitors. The expansion of these cells ex-vivo (outside the body) would allow advances in bone-marrow transplantation, in which HSCs are transferred to a patient to re-establish blood formation. One of the problems of injecting SCF for therapeutic purposes is that SCF activates mast cells. The injection of SCF has been shown to cause allergic-like symptoms and the proliferation of mast cells and melanocytes.

### Granulocyte Colony-Stimulating Factor (G-CSF; GCSF)

Granulocyte colony-stimulating factor (G-CSF or GCSF) is a colony-stimulating factor hormone. G-CSF is also known as colony-stimulating factor 3 (CSF 3). G-CSF is a glycoprotein, growth factor and cytokine produced by a number of different tissues to stimulate the bone marrow to produce granulocytes and stem cells. G-CSF then stimulates the bone marrow to release them into the blood.

G-CSF also stimulates the survival, proliferation, differentiation, and function of neutrophil precursors and mature neutrophils. G-CSF regulates them using Janus kinase (JAK)/signal transducer and activator of transcription (STAT) and Ras/mitogen-activated protein kinase (MAPK) and phosphatidylinositol 3-kinase (PI3K)/protein kinase B (Akt) signal transduction pathway.

G-CSF is produced by endothelium, macrophages, and a number of other immune cells. The gene for G-CSF is located on chromosome 17, locus q11.2-q12. The GCSF gene has 4 introns, and that 2 different polypeptides are synthesized from the same gene by differential splicing of mRNA. It is thought that stability of the G-CSF mRNA is regulated by an RNA element called the G-CSF factor stem-loop destabilising element. The 2 polypeptides differ by the presence or absence of 3 amino acids. Expression studies indicate that both have authentic GCSF activity. The natural human glycoprotein exists in two forms, a 174- and 180-amino-acid-long protein of molecular weight 19,600 grams per mole. The more-abundant and more-active 174-amino acid form has been used in the development of pharmaceutical products by recombinant DNA (rDNA) technology.

The G-CSF-receptor is present on precursor cells in the bone marrow, and, in response to stimulation by G-CSF, initiates proliferation and differentiation into mature granulocytes. G-CSF is also a potent inducer of HSCs mobilization from the bone marrow into the bloodstream, although it has been shown that it does not directly affect the hematopoietic progenitors that are mobilized.

Beside the effect on the hematopoietic system, G-CSF can also act on neuronal cells as a neurotrophic factor. Indeed, its receptor is expressed by neurons in the brain and spinal cord. The action of G-CSF in the central nervous system is to induce neurogenesis, to increase the neuroplasticity and to counteract apoptosis. These proprieties are currently under investigations for the development of treatments of neurological diseases such as cerebral ischemia.

G-CSF stimulates the production of white blood cells (WBC). In oncology and hematology, a recombinant form of G-CSF is used with certain cancer patients to accelerate recovery from neutropenia after chemotherapy, allowing higher-intensity treatment regimens. Chemotherapy can cause myelosuppression and unacceptably low levels of white blood cells, making patients prone to infections and sepsis.

G-CSF is also used to increase the number of hematopoietic stem cells in the blood of the donor before collection by leukapheresis for use in hematopoietic stem cell transplantation. It may also be given to the receiver, to compensate for conditioning regimens. G-CSF has been studied as a treatment for heart degeneration by injecting it into the blood-stream, plus SDF (stromal cell-derived factor) directly to the heart. G-CSF is currently under investigation for cerebral ischemia in a clinical phase IIb and several clinical pilot studies are published for other neurological disease such as amyotrophic lateral sclerosis. Sweet's syndrome is a known side effect of using this drug. A study in mice has shown that G-CSF may decrease bone mineral density.

G-CSF was first marketed by Amgen with the brand name Neupogen. Several bio-generic versions are now also available in markets such as Europe and Australia. The recombinant human G-CSF synthesised in an E. coli expression system is called filgrastim. The structure of filgrastim differs slightly from the structure of the natural glycoprotein. Most published studies have used filgrastim. Filgrastim (Neupogen) and PEG-filgrastim (Neulasta) are two commercially-available forms of rhG-CSF (recombinant human G-CSF). The PEG (polyethylene glycol) form has a much longer half-life, reducing the necessity of daily injections. Another form of recombinant human G-CSF called lenograstim is synthesised in Chinese Hamster Ovary cells (CHO cells). As this is a mammalian cell expression system, lenograstim is indistinguishable from the 174-amino acid natural human G-CSF. No clinical or therapeutic consequences of the differences between filgrastim and lenograstim have yet been identified, but there are no formal comparative studies.

### Interferon Gamma (IFN-γ)

Interferons (IFNs) are potent extracellular protein mediators of host defence and homoeostasis. They are cytokines produced by the cells of the immune system in response to challenges by foreign agents such as viruses, parasites and tumor cells. It is produced by a wide variety of cells in response to the presence of double-stranded RNA, a key indicator of viral infection. IFNs are divided into two major subgroups. Type I IFNs all bind to a type I IFN receptor, such as IFN-α and IFN-β. IFN-γ is the sole type II IFN, which binds to a distinct type II receptor IFN-γ receptor (IFNGR). Almost all cell types produce type I IFNs, while the type II IFN-r is produced in T cells and natural killer (NK) cells upon immunological stimulation. IFN-r coordinates a diverse array of cellular programs through transcriptional regulation of immunologically relevant genes. Cellular effects of IFN-r includes up-regulation of pathogen recognition, antigen processing and presentation, the antiviral state, inhibition of cellular proliferation and effects on apoptosis, activation of microbicidal effector functions, immunomodulation, and leukocyte trafficking.

Production of interferons predominantly occurs in response to microbes, such as viruses and bacteria, and their products. Binding of molecules uniquely found in microbes—viral glycoproteins, viral RNA, bacterial endotoxin (lipopolysaccharide), bacterial flagella, CpG motifs—by pattern recognition receptors, such as membrane bound Toll like receptors or the cytoplasmic receptors RIG-1 or MDA5, can trigger release of IFNs. Toll Like Receptor 3 (TLR3) is important for inducing interferon in response to the presence of double-stranded RNA viruses; the ligand for this receptor is double-stranded RNA (dsRNA). After binding dsRNA, this receptor activates the transcription factors IRF3 and NF-κB, which are important for initiating synthesis of many inflammatory proteins. Release of IFN from cells is also induced by mitogens. Other cytokines, such as interleukin 1, interleukin 2, interleukin-12, tumor necrosis factor and colony-stimulating factor, can also enhance interferon production.

### Radiation and Radiobiological Bystander Effect

Radiation is a process in which energetic particles or energy or waves travel through a medium or space, of which there are two distinct types: ionizing and non-ionizing. Ionizing radiation, including nuclear radiation and cosmic radiation, consists of sub-atomic particles or electromagnetic waves that are energetic enough to detach electrons from atoms or molecules, therefore ionizing them. Examples of ionizing particles are energetic alpha particles, beta particles, neutrons, and heavy ions (e.g., ⁵⁶Fe). Photons and particles with energies above a few electron volts (eV) are ionizing. Alpha particles, beta particles, gamma rays, X-ray radiation, and neutrons may all carry energy high enough to ionize atoms. This energy is usually higher than about two electron volts (eV). The ability of an electromagnetic wave (photons) to ionize an atom or molecule depends on its frequency. EM radiation on the short-wavelength end of the electromagnetic spectrum—high frequency ultraviolet, X-rays, and gamma rays—is ionizing. Ionizing electromagnetic radiation consists of photons having energies larger than about two electron volts (an energy of about 3.2×10⁻¹⁹ joules), which is a typical binding energy of an outer electron to an atom or organic molecule. This corresponds with a frequency of 4.8×10¹⁴ Hz, and a wavelength of 620 nm (approximately corresponding to red colored visible light).

Sources of ionizing radiation included radioactive materials. X-ray tubes, particle accelerators, and IR is present in the environment. It is invisible and not directly detectable by human senses, so instruments such as Geiger counters are usually required to detect its presence. In some cases, IR may lead to secondary emission of visible light upon interaction with matter, as in Cherenkov radiation and radioluminescence. Ionizing radiation has practical uses in medicine, research, and construction, but presents a potential health hazard. Exposure to radiation causes damage to living tissue, resulting in skin burns, radiation sickness and death at high doses and cancer, tumors and genetic damage at low doses. For example, DNA exposed to ionizing radiation is susceptible to double-strand breaks. Because cells and more importantly the DNA can be damaged, this ionization can result in an increased chance of cancer. The probability of ionizing radiation causing cancer is dependent upon the dose rate of the radiation and the sensitivity of the organism being irradiated.

Alpha (α) Radiation

Alpha decay is by far the most common form of cluster decay where an atom ejects a defined collection of nucleons, leaving another defined product behind (in nuclear fission, a number of different pairs of atoms of approximately equal size are formed). An alpha particle is the same as a helium-4 nucleus, and both mass number and atomic number are the same. Alpha particles have a typical kinetic energy of 5 MeV (that is, ≈0.13% of their total energy, i.e. 110 TJ/kg) and a speed of 15,000 km/s. There is small variation around this energy, due to the heavy dependence of the half-life of this process on the energy produced (see equations in the Geiger-Nuttall law). Because of their relatively large mass, +2 electric charge and relatively low velocity, alpha particles are very likely to interact with other atoms and lose their energy, so their forward motion is effectively stopped within a few centimeters of air.

Beta (β) Radiation

Beta-minus (β−) radiation consists of an energetic electron. It is more ionizing than alpha radiation, but less than gamma radiation. The electrons can often be stopped with a few centimeters of metal. It occurs when a neutron decays into a proton in a nucleus, releasing the beta particle and an antineutrino. Beta-plus (β+) radiation is the emission of positrons. Because these are antimatter particles, they annihilate any matter nearby, releasing gamma photons.

Neutron Radiation

Neutrons are categorized according to their speed. High-energy (high-speed) neutrons have the ability to ionize atoms and are able to deeply penetrate materials. Neutrons are the only type of ionizing radiation that can make other objects, or material, radioactive. This process, called neutron activation, is the primary method used to produce radioactive sources for use in medical, academic, and industrial applications. High-energy neutrons can travel great distances in air and typically require hydrogen rich shielding, such as concrete or water, to block them. A common source of neutron radiation occurs inside a nuclear reactor, where many feet of water is used as effective shielding.

X-Ray Radiation

X-rays are electromagnetic waves with a wavelength smaller than about 10 nanometres. A smaller wavelength corresponds to a higher energy according to the equation E=h·c/λ. (“E” is Energy; “h” is Planck's Constant; “c” is the speed of light; “λ” is wavelength.) A “packet” of electromagnetic waves is called a photon. When an X-ray photon collides with an atom, the atom may absorb the energy of the photon and boost an electron to a higher orbital level or if the photon is very energetic, it may knock an electron from the atom altogether, causing the atom to ionize. Generally, a larger atom is more likely to absorb an X-ray photon, since larger atoms have greater energy differences between orbital electrons. Soft tissue in the human body is composed of smaller atoms than the calcium atoms that make up bone, hence there is a contrast in the absorption of X-rays. X-ray machines are specifically designed to take advantage of the absorption difference between bone and soft tissue, allowing physicians to examine structure in the human body.

Gamma (γ) Radiation

Gamma (γ) radiation consists of photons with a frequency of greater than 1019 Hz. Gamma radiation occurs to rid the decaying nucleus of excess energy after it has emitted either alpha or beta radiation. Both alpha and beta particles have an electric charge and mass, and thus are quite likely to interact with other atoms in their path. Gamma radiation is composed of photons, and photons have neither mass nor electric charge. Gamma radiation penetrates much further through matter than either alpha or beta radiation. Gamma rays, which are highly energetic photons, penetrate deeply and are difficult to stop. They can be stopped by a sufficiently thick layer of material, where stopping power of the material per given area depends mostly (but not entirely) on its total mass, whether the material is of high or low density. However, as is the case with X-rays, materials with high atomic number such as lead or depleted uranium add a modest (typically 20% to 30%) amount of stopping power over an equal mass of less-dense and lower atomic weight materials (such as water or concrete).

Cosmic/Space Radiation

Cosmic radiation (radiation originating from outer space) consists of positively-charged ions from protons to iron nuclei (⁵⁶Fe). The energy of this radiation can far exceed that which can be created in a particle accelerator. In general, about 90% of all the incoming cosmic rays are protons, about 9% are helium nuclei (alpha particles) and nearly 1% are electrons. The remaining fraction is made up of the other heavier nuclei which are abundant end products of stars' nuclear synthesis. The cosmic-radiation dose rate on airplanes is so high that studies have indicated that airline flight crew workers receive more dose on average than any other worker, including those in nuclear power plants. Cosmic radiation interacts in the atmosphere to create secondary radiation, including x-rays, muons, protons, alpha particles, pions, electrons, and neutrons. The dose from cosmic radiation varies in different parts of the world based largely on the geomagnetic field, altitude, and solar cycle.

Different types of ionizing radiation behave in different ways, thus, resulting in use of different shielding techniques. Particle radiation consists of a stream of charged or neutral particles, both charged ions and subatomic elementary particles, including, solar wind, cosmic radiation, and neutron flux in nuclear reactors. Alpha particles (helium nuclei) are the least penetrating type of ionizing radiation (e.g., energetic alpha particles can be stopped by a single sheet of paper). Beta particles (electrons) are more penetrating, but still can be absorbed by a few millimeters of aluminum. However, in cases where high energy beta particles are emitted shielding must be accomplished with low density materials, e.g. plastic, wood, water or acrylic glass (Plexiglas. Lucite), to reduce generation of Bremsstrahlung X-rays. In the case of beta+ radiation (positrons), the gamma radiation from the electron-positron annihilation reaction poses additional concern. Neutron radiation is not as readily absorbed as charged particle radiation, which makes this type highly penetrating. Neutrons are absorbed by nuclei of atoms in a nuclear reaction. This most-often creates a secondary radiation hazard, as the absorbing nuclei transmute to the next-heavier isotope, many of which are unstable. Cosmic radiation is not a common concern, as the Earth's atmosphere absorbs it and the magnetosphere acts as a shield, but it poses a problem for satellites and astronauts and frequent fliers are also at a slight risk. Cosmic radiation is extremely high energy, and is very penetrating. Electromagnetic radiation consists of emissions of electromagnetic waves, the properties of which depend on the wavelength. X-ray and gamma radiation are best absorbed by atoms with heavy nuclei; the heavier the nucleus, the better the absorption. In some special applications, depleted uranium is used, but lead is much more common; several centimeters are often required. Barium sulfate is used in some applications too. However, when cost is important, almost any material can be used, but it must be far thicker. Most nuclear reactors use thick concrete shields to create a bioshield with a thin water cooled layer of lead on the inside to protect the porous concrete from the coolant inside. The concrete is also made with heavy aggregates, such as Baryte, to aid in the shielding properties of the concrete.

Previous studies of A-bomb survivors²⁴, accidental exposures (Chernobyl)^{25,26}, radiotherapy in breast^{27,28} and esophageal cancer patients²⁹ have demonstrated that cardiovascular morbidity may occur within months and years and that cardiovascular mortality may occur within decades after initial radiation exposure. Astronauts will be exposed to radiation composed of a spectrum of low-fluence protons and high energy (HZE) ionizing cosmic ray nuclei (e.g. ⁵⁶Fe). Accordingly, a significant risk may exist for the potential development of degenerative cardiovascular risks later in life following exposure to Galactic Cosmic Rays (GCR) and Solar Particle Events (SPE) during space travel. What remains is to determine the degree, and specifically establish the dose-response relationship bearing on eventual CV disease risk.

Full body low-dose radiation is known to induce apoptotic and immunological responses in the organ-tissues, including the heart⁴². The acute phase is usually characterized by a neutrophilic infiltrate of the affected area and macrophages are responsible for the phagocytic clearance of the apoptotic cells^{43,44}. It was shown that phagocytosis of radiation-induced apoptotic cells can activate macrophages, leading them to induce an inflammatory response in the surrounding tissue⁴⁵ by releasing various cytokines, superoxide and nitric oxide, which are capable of causing tissue damage⁴⁶. This can provide a potential feedback loop mechanism perpetuating inflammatory response leading to mitochondrial dysfunction in the heart (e.g., cardiomyocytes).

Radiobiological Bystander Effect

The Radiation-Induced Bystander Effect (Bystander Effect) is a phenomenon in which unirradiated cells exhibit irradiated effects as a result of signals received from nearby irradiated cells. There is evidence that targeted cytoplasmic irradiation results in mutation in the nucleus of the hit cells. Cells that that are not directly hit by an alpha particle, but are in the vicinity of one that is hit, also contribute to the genotoxic response of the cell population.

Similarly, when cells are irradiated, and the medium is transferred to unirradiated cells, these unirradiated cells show bystander responses when assayed for clonogenic survival and oncogenic transformation. This is also attributed to the bystander effect. The demonstration of a bystander effect in 3D human tissues and, more recently, in whole organisms have clear implication of the potential relevance of the non-targeted response to human health. This effect may also contribute to the final biological consequences of exposure to low and/or high doses of radiation.

### Endothelial Progenitor Cells (EPCs)

Endothelial progenitor cells are a population of cells that circulate in the blood with the ability to differentiate into endothelial cells, the cells that make up the lining of blood vessels. Endothelial progenitor cells expressing CD34 isolated from the blood of adult mice differentiate into endothelial cells in vitro. Endothelial progenitor cells function in de novo blood vessel formation (vasculogenesis). Most vasculogenesis occurs in during embryologic development. Pathologic angiogenesis, such as in retinopathy and tumor growth, also involves endothelial progenitor cells.

A growing body of evidence indicates that neovascularization does not exclusively rely on proliferation of local ECs but also involves BM-derived EPCs³⁰. Without being bound to a particular theory, if EPCs are critical to endothelial maintenance and repair, EPC dysfunction could contribute to the pathogenesis of ischemic vascular diseases, as well as for maintenance of normal vascular homeostasis in the heart. Indeed, studies have demonstrated that in patients with CV risk factors, the number and migratory ability of EPCs isolated from peripheral blood is reduced³¹ and EPC function is impaired³². In addition, a strong inverse correlation was reported between the number of circulating EPCs, vascular function and the subject's combined Framingham cardiovascular factor score³³. Furthermore, measurements of flow-mediated brachial-artery reactivity also revealed a significant relation between endothelial function and the number of EPCs, supporting a role for EPCs in the maintenance of endothelial integrity³⁴. Without being bound to a particular theory, numbers of myeloid and lymphoid bone marrow stem and progenitor cells are reduced to just one-half of their normal levels after space flight²³, suggesting that EPCs may be similarly reduced in the normal EPC population. Unfortunately, data on BM-derived EPCs survival or mobilization during and after space flights, or DNA damage responses of EPCs to space radiation, are not available.

Stem cells express at high levels genes associated with DNA repair and protection from stress, including oxidative stress^{35,36}. It has been shown that EPCs express lower levels of basal and stress-induced intracellular ROS than primary ECs because EPCs express higher levels of catalase, manganese superoxide dismutase (MnSOD) and glutathione peroxidase-1 (GPx-1)^{37,38}. It has further been shown that the collective inhibition of catalase, MnSOD, and GPx-1³⁹ increases ROS levels in EPCs and that this inhibition impairs EPC survival and migration³⁷. However, these in vitro findings have yet to be validated in a mouse model of ischemia- and/or radiation-induced oxidative stress⁴⁰. As ischemic/damaged tissue is characterized by high levels of inflammatory cytokines which activate ROS production⁴¹, it has been proposed that high levels of ROS metabolizing enzymes in EPCs are essential to maintain their survival during tissue regeneration after injury. Without being bound to a particular theory, these findings suggest that an imbalance in ROS can contribute to EPC dysfunction and that oxidative stress may impair neovascularization, thereby contributing to the pathogenesis and the progression of cardiovascular disease.

To obtain endothelial progenitor cells from peripheral blood about 5 ml to about 500 ml of blood is taken from a donor. Preferably, about 50 ml to about 200 ml of blood is taken. Endothelial progenitor cells are expanded in vivo by administration of recruitment growth factors, e.g., GM-CSF and IL-3, to the donor prior to removing the progenitor cells. Methods for obtaining and using hematopoietic progenitor cells in autologous transplantation are disclosed in U.S. Pat. No. 5,199,942, the disclosure of which is incorporated by reference. Alternatively, the cells are expanded ex vivo using, for example, the method disclosed by U.S. Pat. No. 5,541,103. Endothelial progenitor cells may be obtained from human mononuclear cells obtained from peripheral blood or bone marrow of the subject before treatment. Such cells may also be obtained from heterologous or autologous umbilical cord blood. In particular, endothelial progenitor cells may be obtained from the leukocyte fraction of peripheral blood. Endothelial progenitor cells may be isolated using antibodies that recognize endothelial progenitor cell specific antigens on immature human hematopoietic progenitor cells. For example, CD34 is commonly shared by endothelial progenitor cells and hematopoietic stem cells. CD34 is expressed by all hematopoietic stem cells but is lost by hematopoietic cells as they differentiate. Flk-1, a receptor for vascular endothelial growth factor (VEGF) is also expressed by both early hematopoietic stem cells and endothelial cells, but ceases to be expressed in the course of hematopoietic differentiation.

Transplantation of hematopoietic stem cells derived from peripheral blood can provide sustained hematopoietic recovery. (See, for example, Kessinger et al., Blood 77, 211 (1991); Sheridan et al., Lancet 339, 640 (1992); Shpall et al., J. Clin. Oncol. 12, 28 (1994). This observation is now being exploited clinically as an alternative to bone marrow transplantation. By using techniques similar to those employed for hematopoietic stem cells, endothelial progenitor cells can be isolated from circulating blood. Such cells, once isolated, can be expanded in vitro and engineered to express one or more heterologous nucleic acid molecules. The cells are then delivered back to the donor, or to another subject, to achieve a therapeutic result.

In vitro, endothelial progenitor cells differentiate into endothelial cells. Indeed, one can use a multipotentiate undifferentiated cell as long as it is still capable of becoming an endothelial cell in the presence of agents that promote its differentiation. In vivo, heterologous, homologous, and autologous endothelial cell progenitor grafts incorporate into sites of active angiogenesis or blood vessel injury by selectively migrating to such locations. Angiogenesis can be promoted in a subject by administering a potent angiogenesis factor, such as VEGF, alone or in combination with endothelial progenitor cells. Once the progenitor cells are obtained by a particular separation technique, they may be administered to a selected subject to treat a number of conditions including, for example, unregulated angiogenesis or blood vessel injury. The cells may also be stored in cryogenic conditions.

As used herein the term “endothelial cell mitogen” means any protein, polypeptide, variant or portion thereof that is capable of, directly or indirectly, inducing endothelial cell growth. Such proteins include, for example, acidic and basic fibroblast growth factors (aFGF) (GenBank Accession No. NP_{—}149127) and bFGF (GenBank Accession No. AAA52448), vascular endothelial growth factor (VEGF) (GenBank Accession No. AAA35789 or NP_{—}001020539), epidermal growth factor (EGF)(GenBank Accession No. NP_{—}001954), transforming growth factor α (TGF-α) (GenBank Accession No. NP_{—}003227) and transforming growth factor β (TFG-β) (GenBank Accession No. 1109243A), platelet-derived endothelial cell growth factor (PD-ECGF) (GenBank Accession No. NP_{—}001944), platelet-derived growth factor (PDGF) (GenBank Accession No. 1109245A), tumor necrosis factor α (TNF-α) (GenBank Accession No. CAA26669), hepatocyte growth factor (HGF) (GenBank Accession No. BAA14348), insulin like growth factor (IGF) (GenBank Accession No. P08833), erythropoietin (GenBank Accession No. P01588), colony stimulating factor (CSF), macrophage-CSF (M-CSF) (GenBank Accession No. AAB59527), granulocyte/macrophage CSF (GM-CSF) (GenBank Accession No. NP_{—}000749), monocyte chemotactic protein-1 (GenBank Accession No. P13500) and nitric oxide synthase (NOS) (GenBank Accession No. AAA36365). See, Klagsbrun, et al., Annu. Rev. Physiol., 53:217-239 (1991); Folkman, et al., J. Biol. Chem., 267:10931-10934 (1992) and Symes, et al., Current Opinion in Lipidology, 5:305-312 (1994). Variants or fragments of a mitogen may be used as long as they induce or promote endothelial cell or endothelial progenitor cell growth. Preferably, the endothelial cell mitogen contains a secretory signal sequence that facilitates secretion of the protein. Proteins having native signal sequences, e.g., VEGF, are preferred. Proteins that do not have native signal sequences, e.g., bFGF, can be modified to contain such sequences using routine genetic manipulation techniques. See, Nabel et al., Nature, 362:844 (1993).

The nucleotide sequence of numerous endothelial cell mitogens, are readily available through a number of computer data bases, for example, GenBank, EMBL and Swiss-Prot. Using this information, a DNA segment encoding the desired may be chemically synthesized or, alternatively, such a DNA segment may be obtained using routine procedures in the art, e.g, PCR amplification. A DNA encoding VEGF is disclosed in U.S. Pat. No. 5,332,671, the disclosure of which is herein incorporated by reference.

### Angiogenesis

Angiogenesis and/or vasculogenesis have been shown to play a role in maintaining or promoting neoplastic cell growth. By “angiogenesis” is meant the growth of new blood vessels originating from existing blood vessels. Methods for measuring angiogenesis are standard, and are described, for example, in Jain et al. (Nat. Rev. Cancer 2: 266-276, 2002). Angiogenesis can be assayed by measuring the number of non-branching blood vessel segments (number of segments per unit area), the functional vascular density (total length of perfused blood vessel per unit area), the vessel diameter, or the vessel volume density (total of calculated blood vessel volume based on length and diameter of each segment per unit area). By “vasculogenesis” is meant the development of new blood vessels originating from stem cells, angioblasts, or other precursor cells, such as endothelial progenitor cells (EPCs). These stem cells can be recruited from bone marrow endogenously or implanted therapeutically. As described herein, agents that reduce angiogenesis in a neoplasm are useful for the treatment of neoplasms.

### Cardiomyocytes

Cardiomyocytes, the cells that form cardiac muscle tissue are mononuclear, smooth muscle cells. Cardiac muscle is a type of involuntary striated muscle found in the walls and histologic foundation of the heart, specifically the myocardium. Coordinated contractions of cardiomyoctyes in the heart propel blood out of the atria and ventricles to the blood vessels of the left/body/systemic and right/lungs/pulmonary circulatory systems.

Cardiac muscle exhibits cross striations formed by alternating segments of thick and thin protein filaments. The primary structural proteins of cardiac muscle are actin and myosin. Histologically, the actin filaments are thin causing the lighter appearance of the I bands in striated muscle, while the myosin filament is thicker lending a darker appearance to the alternating A bands as observed with electron microscopy. In contrast to skeletal muscle, cardiac muscle cells may be branched instead of linear and longitudinal. Another histological difference between cardiac muscle and skeletal muscle is that the T-tubules in the cardiac muscle are larger, broader and run along the Z-Discs. There are fewer T-tubules in comparison with skeletal muscle. Additionally, cardiac muscle forms diads instead of the triads formed between the T-tubules and the sarcoplasmic reticulum in skeletal muscle. T-tubules play critical role in excitation-contraction coupling (ECC).

Intercalated discs (IDs) are complex adhering structures which connect single cardiac myocytes to an electrochemical syncytium (in contrast to the skeletal muscle, which becomes a multicellular syncytium during mammalian embryonic development) and are mainly responsible for force transmission during muscle contraction. Intercalated discs also support the rapid spread of action potentials and the synchronized contraction of the myocardium. IDs are described to consist of three different types of cell-cell junctions: the actin filament anchoring adherens junctions (fascia adherens), the intermediate filament anchoring desmosomes (macula adherens) and gap junctions. Gap junctions are responsible for electrochemical and metabolic coupling. They allow action potentials to spread between cardiac cells by permitting the passage of ions between cells, producing depolarization of the heart muscle.

Under light microscopy, intercalated discs appear as thin, typically dark-staining lines dividing adjacent cardiac muscle cells. The intercalated discs run perpendicular to the direction of muscle fibers. Under electron microscopy, an intercalated disc's path appears more complex. At low magnification, this may appear as a convoluted electron dense structure overlying the location of the obscured Z-line. At high magnification, the intercalated disc's path appears even more convoluted, with both longitudinal and transverse areas appearing in longitudinal section.

Cardiac muscle requires extracellular calcium ions for contraction to occur. Like skeletal muscle, the initiation and upshoot of the action potential in ventricular muscle cells is derived from the entry of sodium ions across the sarcolemma in a regenerative process. However, an inward flux of extracellular calcium ions through L-type calcium channels sustains the depolarization of cardiac muscle cells for a longer duration. The reason for the calcium dependence is due to the mechanism of calcium-induced calcium release (CICR) from the sarcoplasmic reticulum that must occur under normal excitation-contraction (EC) coupling to cause contraction. Once the intracellular concentration of calcium increases, calcium ions bind to the protein troponin, which initiate extracellular fluid and intracellular stores, and skeletal muscle, which is only activated by calcium stored in the sarcoplasmic reticulum.

Intracellular Ca²⁺ plays a fundamental role in numerous signaling pathways and serves as a mediator of pathological processes in muscle and non-muscle diseases. A Ca²⁺-dependent Excitation-Contraction (EC) coupling regulates cardiac muscle contraction and force generation. In this process, a rapid cascade of events is initiated by an action potential that activates Ca²⁺ influx through L-type Ca²⁺, which triggers massive release of Ca²⁺ from the sarcoplasmic reticulum (SR) via Ca²⁺ release channels residing in the ryanodine receptors (RyRs). The resulting release of Ca²⁺ produces a transient increase in intracellular [Ca²⁺]⁴⁷, which activates the contractile apparatus of muscle fibers. Muscle contraction is terminated when Ca²⁺ is sequestered back into SR via SR Ca²⁺ ATPase. Cardiac contractions consume large amounts of cellular energy and require an efficient mitochondrial oxidative phosphorylation for ATP generation. To meet cellular energy demands mitochondria require an increase in [Ca²⁺] within the mitochondrial matrix ([Ca²⁺]ₘ), which is achieved by transporting Ca²⁺ from the cytoplasm. Under normal physiological conditions Ca²⁺ exerts a positive effect on mitochondrial function^{48, 49}. Increase in [Ca²⁺]ₘ stimulates Ox-Phos by allosteric activation of numerous members of TCA cycle⁵⁵⁻⁵⁵. In contrast to beneficial aspects of mitochondrial Ca²⁺ transport, perturbations in [Ca²⁺]ₘ due to altered cytoplasmic Ca²⁺ homeostasis could have detrimental implications on cellular energy production as well as overall cellular function⁵⁶. A sustained elevation in resting [Ca²⁺]_{c} (also termed [Ca²⁺]ᵢ) leads to an overload of [Ca²⁺]ₘ. Mitochondrial Ca²⁺ overload can lead to opening of the permeability transition (PT) pore⁵⁷, which under normal physiological conditions, serves to release Ca²⁺ from the mitochondrial matrix by rapidly transitioning between open and closed states^{58,59}. A sustained elevation in resting [Ca²⁺]_{c} leads to an overload of [Ca²⁺]ₘ, which triggers a prolonged activation of the PT pore. This is accompanied by the loss of □ mitochondrial membrane potential □ΔΨₘ), expansion of the matrix, rupture of the outer mitochondrial membrane⁶⁰ and a burst in reactive oxygen species (ROS)^{61,62}. Without being bound to a particular theory, radiation has deleterious effects on Ca²⁺ entry through the L-type Ca²⁺ channels, RyR Ca²⁺ release and cause Ca²⁺ overload-mediated mitochondrial alterations, which could result in severe reduction in cardiomyocyte contractility.

### Neoplastic Cell Growth and Apoptosis

Neoplastic cell growth is not subject to the same regulatory mechanisms that govern the growth or proliferation of normal cells. Without being bound to a particular theory, chronic low-dose exposure to radiation induces DNA damage, leading to, e.g., cellular transformation, and induction of mutagenesis. Inefficient damage repair (e.g., in BM-derived hematopoeitic stem cells-HSC, hemangioblasts, EPCs) may lead to increased mutagenesis and increased risk of developing neoplasia or cancer (e.g., neoplastic transformation). Inefficient damage repair may also lead to long-term disfunction of these vessel forming cells.

Agents that reduce the survival of a neoplasia, that increase cell death (e.g., apoptosis) in a neoplasia, or that inhibit vasculogenic cell function of EPCs, hemangioblasts, and HSCs, are useful for the treatment of a neoplastic disease. Such compounds include agents, including but not limited to inhibitory nucleic acid molecules (e.g., antisense nucleic acid molecules, RNAi, siRNA, shRNA), that interfere with the expression or activity of oncogenes, angiogenic growth factors, chemokines, cytokines, or endothelial function of ECs and EPCs. Cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

Agents that reduce the growth or proliferation of a neoplasm are useful for the treatment of neoplasms. Methods of assaying cell growth and proliferation are known in the art. See, for example, Kittler et al. (Nature. 432 (7020):1036-40, 2004) and Miyamoto et al. (Nature 416(6883):865-9, 2002). Assays for cell proliferation generally involve the measurement of DNA synthesis during cell replication. In one embodiment, DNA synthesis is detected using labeled DNA precursors, such as ([³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650): 1581-4, 2003; Gu et al., Science 302 (5644):445-9, 2003).

Candidate compounds that reduce the survival of a neoplastic cell, ECs, and EPCs are also useful as anti-neoplasm therapeutics. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 0.1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Candidate compounds that increase neoplastic, EC, or EPC cell death (e.g., increase apoptosis) are also useful as anti-neoplasm therapeutics. Assays for measuring cell apoptosis are known to the skilled artisan. Apoptotic cells are characterized by characteristic morphological changes; including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Assaying Compounds and Extracts

The invention provides methods for treating radiation exposure by inhibiting one or more of a TNF-α (p55 or p75), IL6, EGF, IL1-alpha, IL1-beta, G-CSF, MCP-1, MIP-1, SCF, or RANTES receptor, or a TNF-α, IL6, EGF, IL1-alpha, IL1-beta, G-CSF, MCP-1, MIP-1, SCF, or RANTES peptide. In one embodiment, the method or agent increases cell survival or proliferation in a cell exposed to the effects of radiation exposure by reducing the expression or activity of one or more of a TNF-α (p55 or p75), IL6, EGF, IL1-alpha, IL1-beta, G-CSF, MCP-1, MIP-1, SCF, or RANTES receptor, or a TNF-α, IL6, EGF, IL1-alpha, IL1-beta, G-CSF, MCP-1, MIP-1, SCF, or RANTES peptide by at least about 10, 20, 30, 40, 50, 60, 70, 80, 90, or 100% relative to the expression of the corresponding receptor or peptide in an untreated control cell. In a particular embodiment, the cell is not “hit” by radiation directly but exposed to the effects of radiation exposure (e.g., via non-targeted bystander effect). In one embodiment, the method provides an inhibitory nucleic acid molecule (e.g., a siRNA, shRNA, or antisense nucleic acid molecule) that binds to or that is complementary to at least a portion of a one or a TNF-α (p55 or p75), IL6, EGF, IL1-alpha, IL1-beta, G-CSF, MCP-1, MIP-1, SCF, or RANTES receptor nucleic acid molecule; or a TNF-α, IL6, EGF, IL1-alpha, IL1-beta, G-CSF, MCP-1, MIP-1, SCF, or RANTES nucleic acid molecule. While the Examples described herein specifically discuss the use of shRNA technology, one skilled in the art understands that the methods of the invention are not so limited. Virtually any agent that reduces the expression or activity of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide may be employed in the methods of the invention. In another embodiment, the method provides an antibody or fragment thereof that binds to a p55/TNF-α receptor or p75/TNF-α receptor.

A selective receptor antagonist is one that reduces the expression of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide. Potential selective p55/TNF-α receptor or p75/TNF-α receptor antagonists include organic molecules, peptides, peptide mimetics, polypeptides (e.g., dominant negative polypeptides or proteins), nucleic acid ligands, aptamers, and antibodies that bind to a p55/TNF-α receptor or a p75/TNF-α receptor and selectively inhibit its activity. As used herein a “dominant negative polypeptide or protein” refers to a polypeptide or protein that adversely affects the normal, wild-type gene product when expressed within the same cell. This situation can occur if the product can interact with the same elements as the wild-type product, but block some aspect of its function. For example, a dominant negative fragment of either the p55/TNF-α receptor or p75/TNF-α receptor may contain the extracellular domain, but lack intracellular sequences. Trimerization between dominant negative polypeptides of either the p55/TNF-α receptor or p75/TNF-α receptor would affect signalling through their respective pathways because the dominant negative polypeptides lack the intracellular sequences necessary for signal transduction.

Methods of assaying the expression of a polypeptide or polynucleotide of interest are known in the art, and include co-immunoprecipitation, Western blotting, flow cytometry, immunocytochemistry, binding to magnetic and/or p55/TNF-α receptor- or p75/TNF-α receptor-specific antibody-coated beads, in situ hybridization, fluorescence in situ hybridization (FISH). ELISA, microarray analysis, RT-PCR, Northern blotting, or colorimetric assays, such as the Bradford Assay and Lowry Assay. Potential antagonists also include small molecules that bind to and reduce the activity of the p55/TNF-α receptor or p75/TNF-α receptor.

Such agents may be used, for example, as a therapeutic to combat radiation exposure in a subject. Optionally, agents identified in any of the assays described herein may be confirmed as useful in conferring protection against the development of radiobiological bystander effect in any standard animal model (e.g., full body irradiated mice) and, if successful, may be used as therapeutics to treat radiation exposure.

Agents that selectively reduce the expression or activity of the TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide are identified as useful in the methods of the invention. In one embodiment, an agent that reduces the expression or activity of the TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide, is isolated and tested for its activity on irradiated cell growth or survival in an in vitro assay. In another embodiment, the agent is isolated tested for its activity in an in vitro assay on cell growth or survival of cells exposed to the cellular products of an irradiated cell. One skilled in the art appreciates that the effects of a candidate agent on a cell is typically compared to a corresponding control cell not contacted with the candidate agent.

In one working example, one or more candidate agents are added at varying concentrations to culture medium containing a cell directly or indirectly exposed to the effects of radiation. An agent that suppresses the expression of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide expressed in the cell is considered useful in the invention; such an agent may be used, for example, as a therapeutic to prevent, delay, ameliorate, stabilize, or treat radiation exposure. Once identified, agents of the invention (e.g., agents that reduce the expression or activity of the TNF-α (p55 or p75), IL6, EGF, IL1-alpha, IL1-beta, G-CSF, MCP-1, MIP-1, SCF, or RANTES receptor, or TNF-α, IL6, EGF, IL1-alpha, IL1-beta, G-CSF, MCP-1, MIP-1, SCF, or RANTES peptide) may be used to reduce the effects of radiation exposure or radiobiological bystander effects in a patient in need thereof. Alternatively, an agent identified as useful to a method of the invention is locally or systemically delivered to increase cell proliferation or survival in situ.

In one example, a candidate compound that binds to a p55/TNF-α receptor or p75/TNF-α receptor may be identified using a chromatography-based technique. For example, a recombinant a p55/TNF-α receptor or p75/TNF-α receptor polypeptide of the invention may be purified by standard techniques from cells engineered to express the polypeptide, or may be chemically synthesized, once purified the peptide is immobilized on a column. A solution of candidate agents is then passed through the column, and an agent that specifically binds the p55/TNF-α receptor or p75/TNF-α receptor polypeptide or a fragment thereof is identified on the basis of its ability to bind to the p55/TNF-α receptor or p75/TNF-α receptor polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). Agents isolated by this approach may also be used, for example, as therapeutics to treat or prevent radiation exposure (e.g., radiobiological bystander effect). Compounds that are identified as reducing the expression or activity of the TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES polypeptide at least 10, 20, 30, 40, 50, 60, 70, 80, 90, or 100% are considered particularly useful in the invention.

Such agents may be used, for example, as a therapeutic to combat radiation exposure in a subject. Optionally, agents identified in any of the above-described assays may be confirmed as useful in conferring protection against the development of radiation exposure in any standard animal model (e.g., full body irradiation of mice) and, if successful, may be used as therapeutics for radiation exposure.

In general, TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES antagonists (e.g., agents that selectively reduce the expression or activity of the TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide) can be identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include those known as therapeutics for the treatment of radiation exposure. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK). Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Furthermore, if desired, any library or compound is readily modified using standard chemical, physical, or biochemical methods. Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have TNF-α (p55 or p75), IL6, EGF, IL1-alpha, IL1-beta, G-CSF, MCP-1, MIP-1, SCF, or RANTES receptor-; or a TNF-α, IL6, EGF, IL1-alpha, IL1-beta, G-CSF, MCP-1, MIP-1, SCF, or RANTES inhibitory activity further fractionation of the positive lead extract is necessary to isolate molecular constituents responsible for the observed effect. Thus, the goal of the extraction, fractionation, and purification process is the careful characterization and identification of a chemical entity within the crude extract that reduces the expression or activity of the TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES polypeptide. Methods of fractionation and purification of such heterogenous extracts are known in the art. Methods of assaying the expression or activity of the TNF-α (p55 or p75), IL6, EGF, IL1-alpha, IL1-beta, G-CSF, MCP-1, MIP-1, SCF, and RANTES receptors; and TNF-α, IL6, EGF, IL1-alpha, IL1-beta, G-CSF, MCP-1, MIP-1, SCF, and RANTES polypeptides are known in the art and can be used to determine whether an agent is selective for a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES polypeptide. If desired, compounds shown to be useful as therapeutics are chemically modified according to methods known in the art, e.g., to provide selectivity between the p55/TNF-α and p75/TNF-α receptors

### Inhibitory Nucleic Acids

Inhibitory nucleic acid molecules are those oligonucleotides that selectively inhibit the expression or activity of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES polypeptide or nucleic acid molecule. Such oligonucleotides include single and double stranded nucleic acid molecules (e.g., DNA, RNA, and analogs thereof) that are complementary to or that bind a nucleic acid molecule that encodes a TNF-α receptor polypeptide (e.g., antisense molecules, RNAi, siRNA, shRNA) as well as nucleic acid molecules that bind directly to a p55 or p75 TNF-α receptor polypeptide to modulate its biological activity (e.g., aptamers).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39, 2002).

Given the sequence of a target gene, siRNAs may be designed to inactivate that gene. Such siRNAs, for example, could be administered directly to an affected tissue, or administered systemically. The nucleic acid sequence of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES polypeptide gene can be used to design small interfering RNAs (siRNAs). The 21 to 25 nucleotide siRNAs may be used, for example, as therapeutics to treat radiation exposure or a disorder thereof.

The inhibitory nucleic acid molecules of the present invention may be employed as double-stranded RNAs for RNA interference (RNAi)-mediated knock-down of TNF-α (p55 or p75), IL6, EGF, IL1-alpha, IL1-beta, G-CSF, MCP-1, MIP-1, SCF, or RANTES receptor, or TNF-α, IL6, EGF, IL1-alpha, IL1-beta, G-CSF, MCP-1, MIP-1, SCF, or RANTES polypeptide expression. In one embodiment, TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide expression is reduced in an irradiated cell or an endothelial cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore. Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

### Ribozymes

Catalytic RNA molecules or ribozymes that include an antisense TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES sequence of the present invention can be used to inhibit expression of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide nucleic acid molecule or polypeptide in vivo. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591, 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

shRNA

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). In one embodiment of the invention, the shRNA molecule is made that includes between eight and twenty-one consecutive nucleobases of a TNF-α (p55 or p75), IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES gene. In specific embodiments, the shRNA comprises a sequence represented by SEQ ID NO: 1, SEQ ID NO: 2, SEQ ID NO: 3, or SEQ ID NO: 4.

TABLE 1
shRNA encoded on plasmids.

| Plasmid | Query | Sequence of shRNA | Description | GenBank | Symbol |
|---|---|---|---|---|---|
| 1 | KM03091 | GGTGGCATCTCTCTTCCAATT | TNFR2/p75 | NM_011610 | Tnfrsf1b |
| | | (SEQ ID NO: 1) | | | |
| 2 | KM03091 | CCAAGGACACTCTACGTATCT | TNFR2/p75 | NM_011610 | Tnfrsf1b |
| | | (SEQ ID NO: 2) | | | |
| 3 | KM03091 | GGAACCAGTTTCGTACATGTT | TNFR2/p75 | NM_011610 | Tnfrsf1b |
| | | (SEQ ID NO: 3) | | | |
| 4 | KM03091 | GCCAATATGTGAAACATTTCT | TNFR2/p75 | NM_011610 | Tnfrsf1b |
| | | (SEQ ID NO: 4) | | | |

For expression of shRNAs within cells, plasmid vectors containing either the polymerase III H1-RNA or U6 promoter, a cloning site for the stem-looped RNA insert, and a 4-5-thymidine transcription termination signal can be employed (e.g., pGeneClip Neomycin Vector; Promega Corporation). The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs.

For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Oligonucleotides and Other Nucleobase Oligomers

At least two types of oligonucleotides induce the cleavage of RNA by RNase H: polydeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. It is understood that the methods and reagents of the present invention may be used in conjunction with any technologies that may be developed, including covalently-closed multiple antisense (CMAS) oligonucleotides (Moon et al., Biochem J. 346:295-303, 2000; PCT Publication No. WO 00/61595), ribbon-type antisense (RiAS) oligonucleotides (Moon et al., J. Biol. Chem. 275:4647-4653, 2000; PCT Publication No. WO 00/61595), and large circular antisense oligonucleotides (U.S. Patent Application Publication No. US 2002/0168631 A1).

As is known in the art, a nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriest-ers, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. The nucleobase units are maintained for hybridization with a nucleic acid molecule encoding a p75/TNF-α receptor or p55/TNF-α receptor. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂.NH—O—CH₂—, —CH₂—N(CH₃)—O—CH₂— (known as a methylene(methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₙCH₃, O(CH₂)ₙOCH₃, O(CH₂)ₙNH₂, O(CH₂)ₙCH₃, O(CH₂)ₙONH₂, and O(CH₂)nON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy(2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy(2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and 0-6 substituted purines, including 2-aminopropyladenine, 5-propynyluracil and 5-propynylcytosine, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86:6553-6556, 1989), cholic acid (Manoharan et al., Bioorg. Med. Chem. Let, 4:1053-1060, 1994), a thioether, e.g., hexyl-S-tritylthiol (Manoharan et al., Ann. N.Y. Acad. Sci., 660:306-309, 1992; Manoharan et al., Bioorg. Med. Chem. Let., 3:2765-2770, 1993), a thiocholesterol (Oberhauser et al., Nucl. Acids Res., 20:533-538: 1992), an aliphatic chain, e.g., dodecandiol or undecyl residues (Saison-Behmoaras et al., EMBO J., 10:1111-1118, 1991; Kabanov et al., FEBS Lett., 259:327-330, 1990; Svinarchuk et al., Biochimie, 75:49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36:3651-3654, 1995; Shea et al., Nucl. Acids Res., 18:3777-3783, 1990), a polyamine or a polyethylene glycol chain (Manoharan et al., Nucleosides & Nucleotides, 14:969-973, 1995), or adamantane acetic acid (Manoharan et al., Tetrahedron Lett., 36:3651-3654, 1995), a palmityl moiety (Mishra et al., Biochim. Biophys. Acta, 1264:229-237, 1995), or an octadecylamine or hexylamino-carbonyl-oxycholesterol moiety (Crooke et al., J. Pharmacol. Exp. Ther., 277:923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see. e.g., U.S. Pat. Nos. 5,656,611, 5,753,613, 5,785,992, 6,120,798, 6,221,959, 6,346,613, and 6,353,055, each of which is hereby incorporated by reference).

### Antibodies

Antibodies that selectively bind a TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide and inhibit its activity are useful in the methods of the invention. In one embodiment, selective binding of antibody to a TNF-α (p55 or p75), IL6, EGF, IL1-alpha, IL1-beta, G-CSF, MCP-1, MIP-1, SCF, or RANTES receptor reduces biological activity of the receptor, respectively, e.g., as assayed by analyzing binding to a ligand for the receptor. In another embodiment, selective binding of antibody to TNF-α, IL6, EGF, IL1-alpha, IL1-beta, G-CSF, MCP-1, MIP-1, SCF, or RANTES peptide reduces the biological activity of the peptide. e.g., binding to its receptor.

Methods of preparing antibodies are well known to those of ordinary skill in the science of immunology. As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv), fusion polypeptides, and unconventional antibodies.

In one embodiment, an antibody that binds a TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide is monoclonal. Alternatively, the anti-TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES antibody is a polyclonal antibody. The preparation and use of polyclonal antibodies are known to the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing a TNF-α (p55 or p75), IL6, EGF, IL1-alpha, IL1-beta, G-CSF, MCP-1, MIP-1, SCF, or RANTES receptor, or TNF-α, IL6, EGF, IL1-alpha, IL1-beta, G-CSF, MCP-1, MIP-1, SCF, or RANTES peptide, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide, or immunogenic fragments thereof, can be expressed in cells in vitro, followed by isolation of the polypeptide and administration of the polypeptide to a suitable host in which antibodies are raised.

Alternatively, antibodies against a TNF-α (p55 or p75), IL6, EGF, IL1-alpha, IL1-beta, G-CSF, MCP-1, MIP-1, SCF, or RANTES receptor; or TNF-α, IL6, EGF, IL-alpha, IL1-beta, G-CSF, MCP-1, MIP-1, SCF, or RANTES peptide may, if desired, be derived from an antibody phage display library. A bacteriophage is capable of infecting and reproducing within bacteria, which can be engineered, when combined with human antibody genes, to display human antibody proteins. Phage display is the process by which the phage is made to ‘display’ the human antibody proteins on its surface. Genes from the human antibody gene libraries are inserted into a population of phage. Each phage carries the genes for a different antibody and thus displays a different antibody on its surface. Antibodies made by any method known in the art can then be purified from the host.

Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naïve histocompatible or immunotolerant mammal, especially a mouse. The mammal may be primed for ascites production by prior administration of a suitable composition (e.g., Pristane).

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205.

In other embodiments, the invention provides “unconventional antibodies.” Unconventional antibodies include, but are not limited to, nanobodies, linear antibodies (Zapata et al., Protein Eng. 8(10): 1057-1062, 1995), single domain antibodies, single chain antibodies, and antibodies having multiple valencies (e.g., diabodies, tribodies, tetrabodies, and pentabodies). Nanobodies are the smallest fragments of naturally occurring heavy-chain antibodies that have evolved to be fully functional in the absence of a light chain. Nanobodies have the affinity and specificity of conventional antibodies although they are only half of the size of a single chain Fv fragment. The consequence of this unique structure, combined with their extreme stability and a high degree of homology with human antibody frameworks, is that nanobodies can bind therapeutic targets not accessible to conventional antibodies. Recombinant antibody fragments with multiple valencies provide high binding avidity and unique targeting specificity to cancer cells. These multimeric scFvs (e.g., diabodies, tetrabodies) offer an improvement over the parent antibody since small molecules of ˜60-100 kDa in size provide faster blood clearance and rapid tissue uptake See Power et al., (Generation of recombinant multimeric antibody fragments for tumor diagnosis and therapy. Methods Mol Biol, 207, 335-50, 2003); and Wu et al. (Anti-carcinoembryonic antigen (CEA) diabody for rapid tumor targeting and imaging. Tumor Targeting, 4, 47-58, 1999).

Various techniques for making unconventional antibodies have been described. Bispecific antibodies produced using leucine zippers are described by Kostelny et al. (J. Immunol. 148(5):1547-1553, 1992). Diabody technology is described by Hollinger et al. (Proc. Natl. Acad. Sci. USA 90:6444-6448, 1993). Another strategy for making bispecific antibody fragments by the use of single-chain Fv (sFv) diners is described by Gruber et al. (J. Immunol. 152:5368, 1994). Trispecific antibodies are described by Tutt et al. (J. Immunol. 147:60, 1991). Single chain Fv polypeptide antibodies include a covalently linked VH::VL heterodimer which can be expressed from a nucleic acid including V_{H}- and V_{L}-encoding sequences either joined directly or joined by a peptide-encoding linker as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

### Therapy

Treatment for radiation exposure employing a p55/TNFα receptor antagonist or a p75/TNFα receptor antagonist (e.g., an agent that inhibits the expression or activity of one such receptor) is also provided by the invention. Therapy may be provided wherever treatment for radiation exposure is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind and/or dosage of the radiation exposure being treated, the age and condition of the patient, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of radiation exposure, the therapy can be used to reduce the risk of neoplasia or cancer, increase cell proliferation or survival, and decrease cell death or apoptosis, to relieve symptoms caused by radiation exposure, or to prevent radiation exposure in the first place.

An inhibitory nucleic acid described herein, or other selective inhibitor of TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide, may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be topical, parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for TNF-α (p55 or p75), IL6, EGF, IL1-alpha, IL1-beta, G-CSF, MCP-1, MIP-1, SCF, or RANTES receptor; or TNF-α, IL6, EGF, IL1-alpha, IL1-beta, G-CSF, MCP-1, MIP-1, SCF, or RANTES peptide modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. “Therapeutically effective amount” is intended to include an amount of a compound useful in the present invention or an amount of the combination of compounds claimed, e.g., to treat or prevent the disease or disorder, or to treat the symptoms of the disease or disorder, in a host. The combination of compounds is preferably a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is advantageously demonstrated at suboptimal concentrations of the compounds. Synergy can be in terms of lower cytotoxicity, increased activity, or some other beneficial effect of the combination compared with the individual components. The preferred dosage of an inhibitory nucleic acid of the invention is likely to depend on such variables as the type and extent of the disorder, the overall health status of the particular patient, the formulation of the compound excipients, and its route of administration.

For any of the methods of application described above, an agent of the invention is desirably administered intravenously or is applied to the site of radiation exposure (e.g., by injection). As described above, if desired, treatment with an agent of the invention may be combined with any other therapies for the treatment of radiation exposure.

### Methods for Evaluating Therapeutic Efficacy

In one approach, the efficacy of the treatment is evaluated by measuring, for example, the biological function of the treated organ (e.g., cardiac cell function). Such methods are standard in the art and are described, for example, in the Textbook of Medical Physiology, Tenth edition, (Guyton et al., W.B. Saunders Co., 2000). In particular, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%. Preferably, the tissue is cardiac tissue and, preferably, the organ is heart.

In another approach, the therapeutic efficacy of the methods of the invention is assayed by measuring an increase in cell number in the treated or transplanted tissue or organ as compared to a corresponding control tissue or organ (e.g., a tissue or organ that did not receive treatment). Preferably, cell number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described, for example, in Bonifacino et al., (Current Protocols in Cell Biology Loose-leaf, John Wiley and Sons, Inc., San Francisco, Calif.). For example, assays for cell proliferation may involve the measurement of DNA synthesis during cell replication. In one embodiment, DNA synthesis is detected using labeled DNA precursors, such as [³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650): 1581-4, 2003; Gu et al., Science 302 (5644):445-9, 2003).

In another approach, efficacy is measured by detecting an increase in the number of viable cells present in a tissue or organ relative to the number present in an untreated control tissue or organ, or the number present prior to treatment. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Alternatively, or in addition, therapeutic efficacy is assessed by measuring a reduction in apoptosis. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Methods for Evaluating Cardiac Function

Compositions of the invention may be used to enhance cardiac function in a subject having reduced cardiac function. Methods for measuring the biological function of the heart (e.g., contractile function) are standard in the art and are described, for example, in the Textbook of Medical Physiology, Tenth edition, (Guyton et al., W.B. Saunders Co., 2000). In the invention, cardiac function is increased by at least 5%, 10%, 15%, 20%, 30%, 40%, 50%, 60%, 70%, 80%, 90%, or even 100% relative to the cardiac function present in a naturally-occurring, corresponding tissue or organ. Most advantageously, cardiac function is enhanced or damage is reversed, such that the function is substantially normal (e.g., 85%, 90%, 95%, or 100% of the cardiac function of a healthy control subject). Reduced cardiac function may result from conditions such as cardiac hypertrophy, reduced systolic function, reduced diastolic function, maladaptive hypertrophy, heart failure with preserved systolic function, diastolic heart failure, hypertensive heart disease, aortic and mitral valve disease, pulmonary valve disease, hypertrophic cardiomyopathy (e.g., hypertrophic cardiomyopathy originating from a genetic or a secondary cause), post ischemic and post-infarction cardiac remodeling and cardiac failure.

Any number of standard methods are available for assaying cardiovascular function. Preferably, cardiovascular function in a subject (e.g., a human) is assessed using non-invasive means, such as measuring net cardiac ejection (ejection fraction, fractional shortening, and ventricular end-systolic volume) by an imaging method such echocardiography, nuclear or radiocontrast ventriculography, or magnetic resonance imaging, and systolic tissue velocity as measured by tissue Doppler imaging. Systolic contractility can also be measured non-invasively using blood pressure measurements combined with assessment of heart outflow (to assess power), or with volumes (to assess peak muscle stiffening). Measures of cardiovascular diastolic function include ventricular compliance, which is typically measured by the simultaneous measurement of pressure and volume, early diastolic left ventricular filling rate and relaxation rate (can be assessed from echoDoppler measurements). Other measures of cardiac function include myocardial contractility, resting stroke volume, resting heart rate, resting cardiac index (cardiac output per unit of time [L/minute], measured while seated and divided by body surface area [m²])) total aerobic capacity, cardiovascular performance during exercise, peak exercise capacity, peak oxygen (O₂) consumption, or by any other method known in the art or described herein. Measures of vascular function include determination of total ventricular afterload, which depends on a number of factors, including peripheral vascular resistance, aortic impedance, arterial compliance, wave reflections, and aortic pulse wave velocity, Methods for assaying cardiovascular function include any one or more of the following: Doppler echocardiography, 2-dimensional echo-Doppler imaging, pulse-wave Doppler, continuous wave Doppler, oscillometric arm cuff, tissue Doppler imaging, cardiac catheterization, magnetic resonance imaging, positron emission tomography, chest X-ray, X ray contrast ventriculography, nuclear imaging ventriculography, computed tomography imaging, rapid spiral computerized tomographic imaging, 3-D echocardiography, invasive cardiac pressures, invasive cardiac flows, invasive cardiac cardiac pressure-volume loops (conductance catheter), non-invasive cardiac pressure-volume loops.

### Kits

The invention provides kits for the treatment or prevention of radiation exposure. In one embodiment, the kit includes a therapeutic or prophylactic composition containing an effective amount of an agent described herein, such as an inhibitory nucleic acid described herein (e.g., an shRNA comprising SEQ ID NO:1, SEQ ID NO:2, SEQ ID NO:3 or SEQ ID NO:4) in unit dosage form. In some embodiments, the kit comprises a sterile container that contains a therapeutic or prophylactic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an agent of the invention is provided together with instructions for administering the agent to a subject having or at risk of developing radiation exposure. The instructions will generally include information about the use of the composition for the treatment or prevention of radiation exposure. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1

### DNA DSB Repair in BM-Derived EPCs is Inefficient or Delayed after Exposure to γ-Radiation

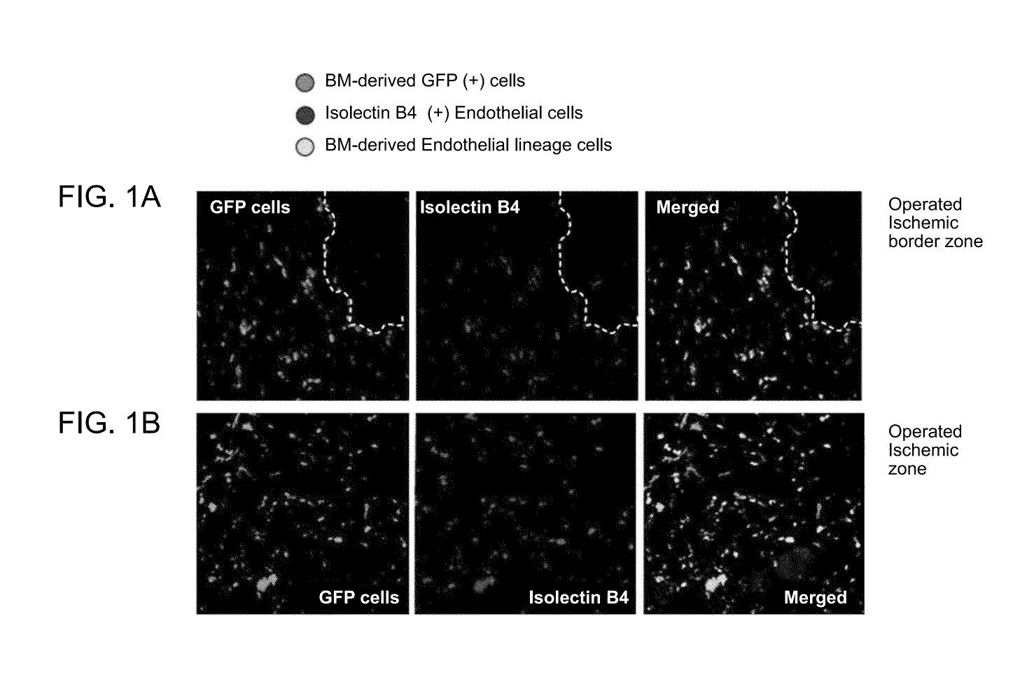

A chimeric animal model derived from green fluorescent protein (GFP) bone marrow (BM) transplanted into C57Bl6J mice¹¹ was used to study the effects of radiation exposure. In ischemic tissue [hindlimb ischemia (HLI) induced by surgical ligation and removal of the femoral artery] at 28 days post-surgery, 60-70% of ECs in the ischemic tissue were BM-derived EPCs (FIGS. 1A and 1B). These data indicated that BM-derived EPCs are recruited to the sites of ischemic injury in large numbers and that BM-derived EPCs substantially contribute to post-natal neovascularization.

To assess the effect of low-dose radiation in BM-derived EPC, exposure to a full body single dose 1 Gy γ-irradiation (low linear energy transfer (LET) type of radiation) on the formation of γ-H2AX foci was evaluated in BM-derived EPCs in C57/Bl6J mice. BM-derived EPCs were isolated 30 min, 24 hours and 7 days post-irradiation and expanded ex-vivo for 60 hrs in selective medium as described in an earlier publication by the PI¹¹. EPCs were then fixed and stained with γ-H2AX antibody to evaluate and quantify the DNA damage response, as DNA damage-induced γ-H2AX foci occur specifically at sites of DNA double-strand breaks (DSBs) and decay of γ-H2AX foci over time correlates well with DSB repair⁶³. Cells with apoptotic features or micronuclei were not considered for γ-H2AX analysis.

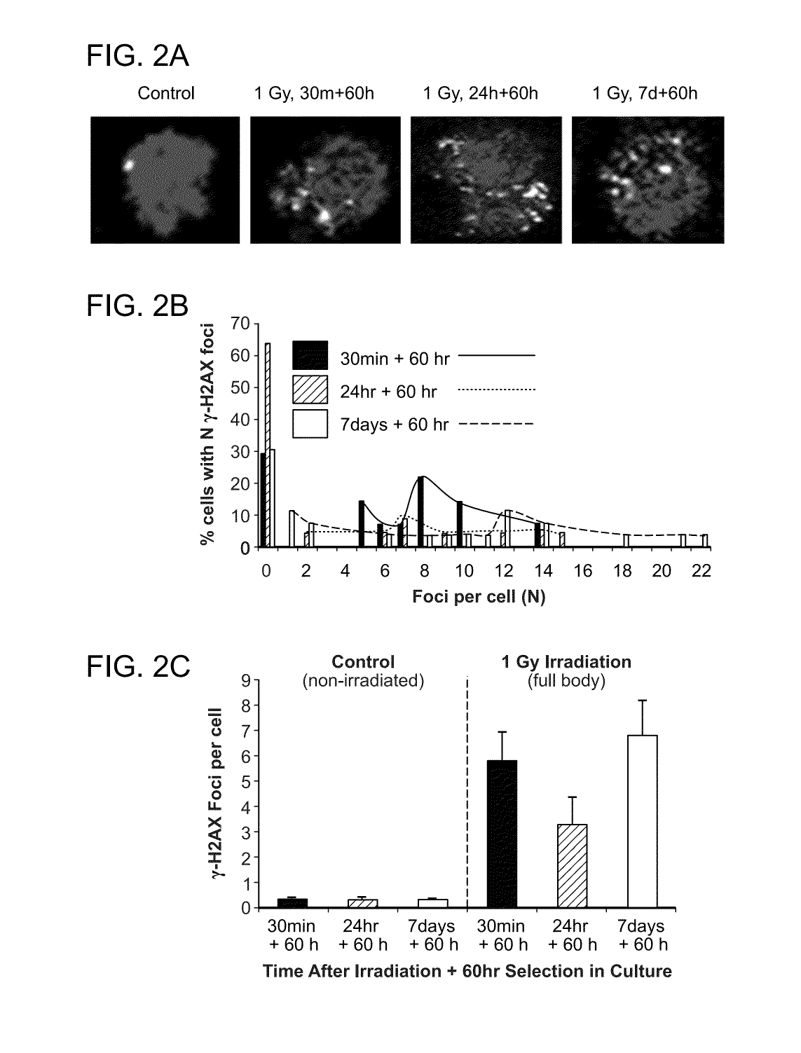

In control, non-irradiated (N-IR) EPCs there were a negligible number of γ-H2AX (+) EPCs, each with no more than 1-2 endogenous γ-H2AX foci (FIGS. 2A and 2C). Distribution of the EPCs with a given number of γ-H2AX foci showed an increase over time in the percentage of cells with γ-H2AX (+) foci (FIG. 2B). Compared to 1-hr samples, by 24 hrs there was about a two-fold decrease (p=NS) in the mean number of foci/cells. However, the number of foci/cell at 7 days was twice that at 24 hrs (p<0.06) (FIG. 2C, gray vs. clear bars).

The decay of γ-H2AX foci was slow in mouse EPCs 24 hrs after full body 1 Gy γ-radiation. These results are indicative of inefficient or delayed DNA DSB repair. Increase in the % of BM-derived EPCs with N γ-H2AX foci and increase of γ-H2AX per cell over 7 days post-radiation also indicated significant radiobiological bystander responses⁶⁴⁻⁶⁸.

To assess acute effects of low-dose radiation on mouse heart tissue, formation of γ-H2AX foci in the heart and resident cardiac ECs in C57/Bl6J mice was evaluated after full body single dose γ-irradiation (1 Gy). Animals were irradiated as described above. Mice were sacrificed 30 min. 24 hrs and 7 days after radiation and hearts were harvested and embedded in OCT compound, snap-frozen and later processed for triple immunostaining with Isolectin/B4 (EC marker), γ-H2AX and TopRo-3 (nuclei).

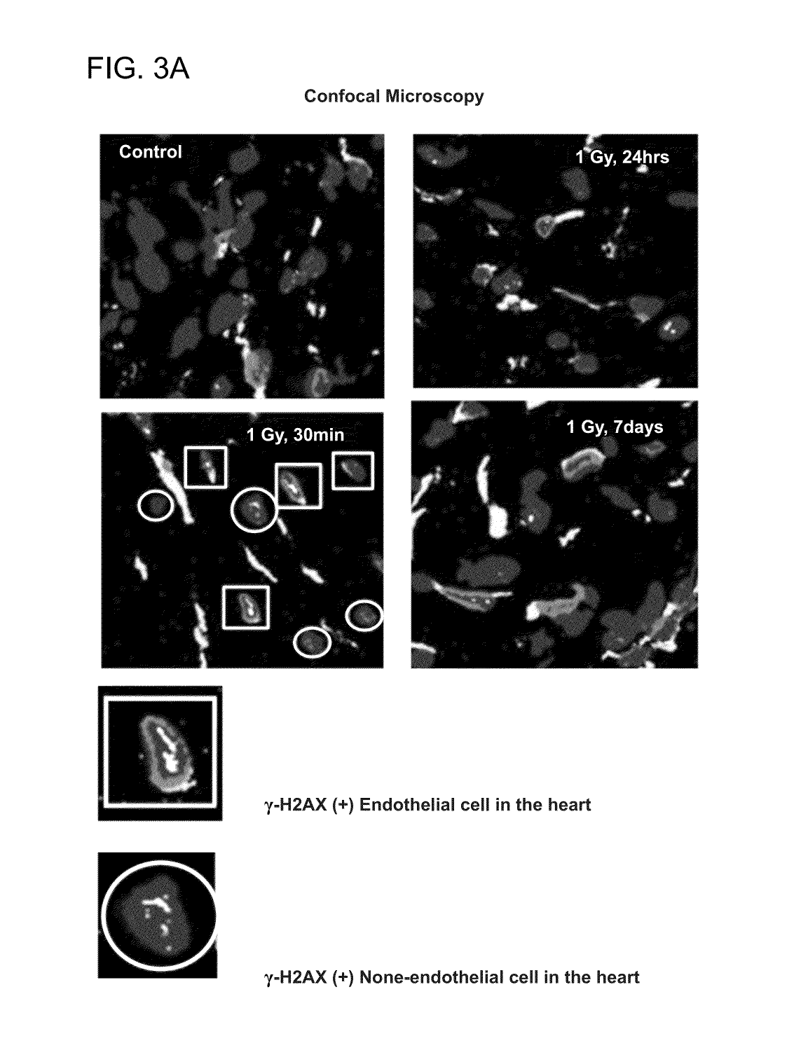

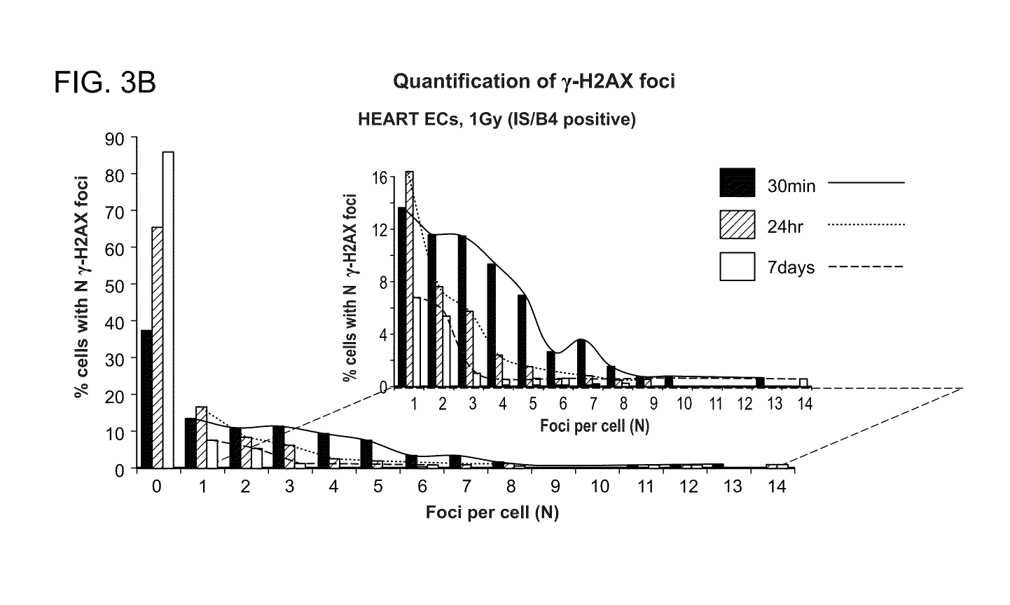

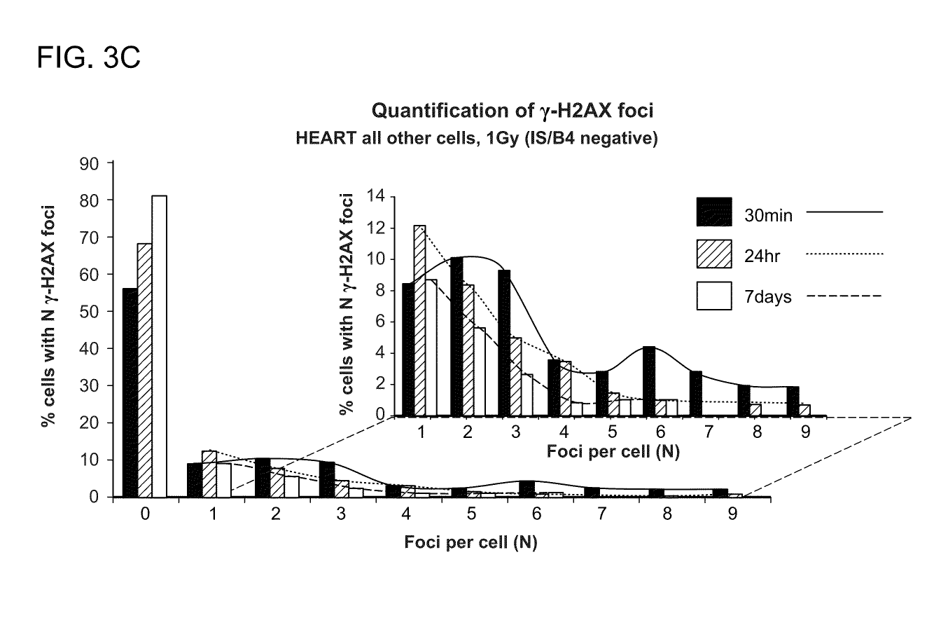

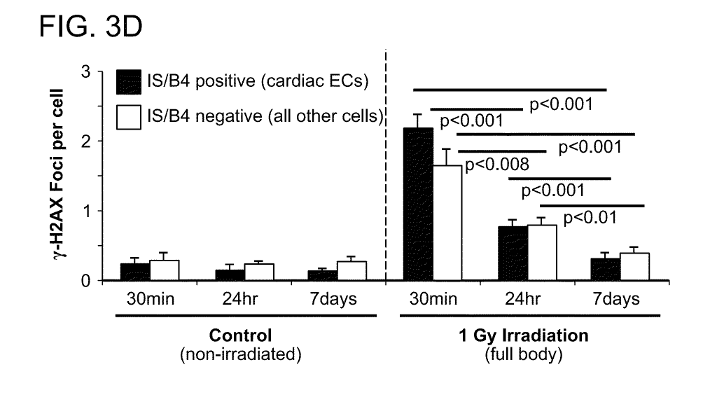

Distribution of the heart-resident EC nuclei (squares—in 1 Gy 30 min images) with a given number of γ-H2AX foci showed a gradual decrease over 7 days in the percentage of cells with γ-H2AX foci (FIGS. 3A and 3B). Distribution of the non-EC (i.e., cardiomyocyte, inflammatory cell) nuclei (circles—in 1 Gy 30 min images) in the heart having a given number of γ-H2AX foci showed a gradual decrease over 7 days in the % of cells with γ-H2AX foci (FIGS. 3A and 3C). The mean number of foci/cell showed that in the heart-resident ECs (black bars) there was approximately a 65% and 87% decay of γ-H2AX foci per cell over 24 hrs and 7 days, respectively, while in the non-EC cells (clear bars) the respective decays were about 51% and 76% (FIGS. 3A and 3D).

There was a significant decay of γ-H2AX foci in irradiated mouse heart resident EC and non-EC cells, which is indicative of considerable DNA DSB repair. However, the repair kinetics were slower than those reported for other primary cells, i.e., fibroblasts, leukocytes^{75,76}.

### Example 2

### DNA DSB Repair in BM-Derived EPCs is Inefficient or Delayed after Exposure to γ-Radiation

To assess the effect of low-dose radiation on BM-derived EPCs the effect of a full-body single dose (0.15 Gy, 1 Gev/n) Iron irradiation on the survival and proliferation of BM-derived EPCs over 28 days post-irradiation was evaluated. BM-derived EPCs were isolated and maintained in corresponding selective EBM2 medium (supplemented with growth factors) ex-vivo for 48 and 72 hours (a minimum time required to select EPC from total BM ex-vivo in the culture). The results revealed that 2, 5, and 24 hrs after full-body irradiation, there was 2-6-fold increase in EPC apoptosis ex-vivo (FACS analysis, subGo/G1 fraction of the cells after PI staining), with peak 6-fold increased apoptosis at 5 hrs (p<0.001). EPC apoptosis was gradually decreased below control non-irradiated EPC levels by day 14. However, by day 28 there was a second significant 4-fold increase (p<0.03) in EPC apoptosis. The data indicate a bimodal (early 5 hrs and delayed 28 days) increase in BM-derived EPC apoptosis after a single 0.15 Gy Iron radiation.

Ex-vivo proliferation of BM-derived EPCs after Iron irradiation was evaluated using CyQUAT cell proliferation assay kit. There was no significant cell proliferation up to 7 days post-irradiation. However there was ˜45 (p<0.005) increase in the rate of EPC proliferation on day 14, but the rate of EPC proliferation had dropped significantly (to 55% of 14 days, p<0.001) on day 28. Without intending to be bound by theory, the data indicate that early increase in BM-derived EPC apoptosis is a direct effect of radiation, and later increase in apoptosis and decrease in proliferation are the result of non-targeted effects. Single low dose of Iron irradiation has long-lasting effect on survival and proliferation of BM-derived EPCs and induces delayed non-targeted effects.

### Example 3

### Myocytes Exposed to γ-Radiation Sustained Increase in Cytoplasmic [Ca²⁺]i Concentration and Loss of Mitochondrial Membrane Potential

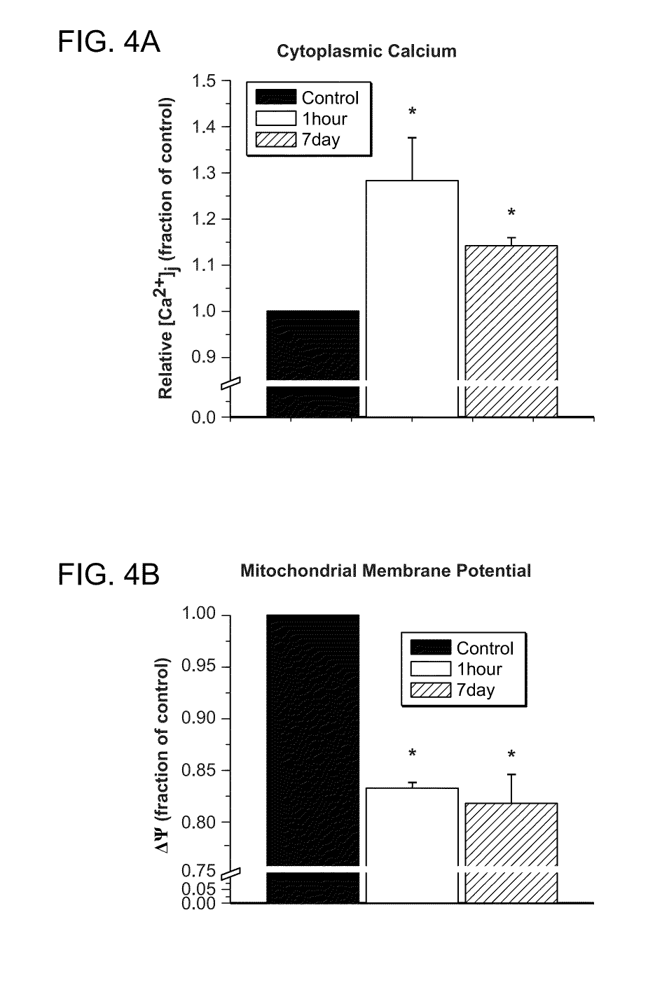

Studies demonstrated that exposure of myocytes to γ-radiation affected resting cytoplasmic [Ca²⁺] in myocytes. Preliminary results demonstrated that within 1 hr, γ-irradiation (1 Gy) of mice results in ˜28% (p<0.01) increase in [Ca²⁺]i. Compared to control, in N-IR myocytes resting intracellular Ca²⁺ levels remained 14% (p<0.03) higher 7 d post-irradiation (FIG. 4A). Thus, a radiation-induced increase in cytoplasmic [Ca²⁺ ]i concentration is sustained for long periods of time (i.e., at least 7 days), leading eventually to mitochondrial calcium overload triggering activation of the permeability transition (PT) pore.

Studies demonstrated that exposure of myocytes to γ-radiation affected the mitochondrial membrane potential (Δ•ₘ) in myocytes. A proper ΔΨₘ is essential for mitochondrial activity and is an indicator for the health of mitochondria. Within 1 hr following γ-irradiation (1 Gy) of mice, a substantial loss of ΔΨₘ in myocytes results. Furthermore, the loss of ΔΨₘ does not recover even at 7 days post-irradiation (FIG. 4B). Thus, radiation caused substantial loss of mitochondrial membrane potential for at least seven days. If radiation exposure is sustained for longer periods of time, mitochondrial membrane integrity is altered.

The effects of whole body irradiation on Ca²⁺ handling in isolated skeletal muscle fibers was investigated using fluorescent Ca²⁺ indicator dyes (Fura-2AM and magFluo-4AM). Mice (C57Bl6J: 9-10 month old) were irradiated with a single dose of proton or ⁵⁶Fe radiation. At set time points (24 hr, 72 hr, and 7 days), mice were sacrificed and single muscle fibers were prepared by enzymatic dissociation of dissected Flexor Digitorum Brevis (FDB) muscle. A ratiometric Fura-2AM dye was used to assess the effects of radiation on [Ca²⁺]i and magFluo-4AM was used to monitor Ca²⁺ release and subsequent clearance in response to stimulation by action potentials. Both proton and ⁵⁶Fe irradiation resulted in detectable increase in [Ca²⁺]i, as well as, reduction of action potential evoked Ca²⁺ release from the SR. There was no apparent correlation between the time course of changes in [Ca²⁺]i and stimulated Ca²⁺ release. Furthermore, it appears that the time course of the observed changes was dependent on the type of radiation. ⁵⁶Fe radiation produced an increase in [Ca²⁺]i at the 24 hr time point which then declined back to near normal levels. Alternatively, proton irradiation did not have an effect at the 24 hr or 48 hr time point, but resulted in a robust increase in [Ca²⁺]i by 72 hrs. Without intending to be bound to theory, ionizing radiation affects the functional state of skeletal muscle and does not produce histological changes.

### Example 4

### BM-Derived EPCs Demonstrated a Radiobiological Bystander Response

To determine if EPCs exhibit bystander responses, two identical confluent sets of ex-vivo expanded BM-derived EPCs were prepared. After 4 days in culture at 60-70% confluence one set was γ-irradiated (1 Gy). Media from irradiated (IR) (conditioned media) and control N-IR dishes were collected after 30 min, 5 hrs, and 24 hrs, which were passed through a 0.22 μm pore filter. The filtered media were added to the dishes with non-irradiated (N-IR) EPCs. After incubation (24 hr) with control and conditioned medium N-IR EPC were fixed and stained with γ-H2AX as described herein.

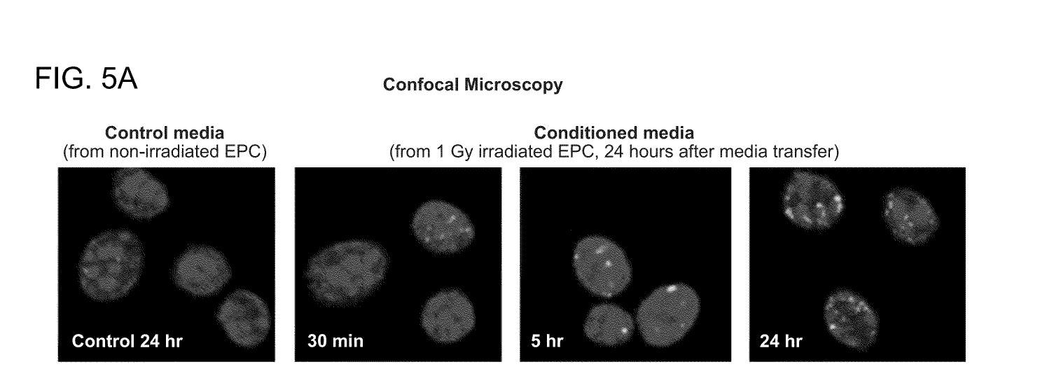

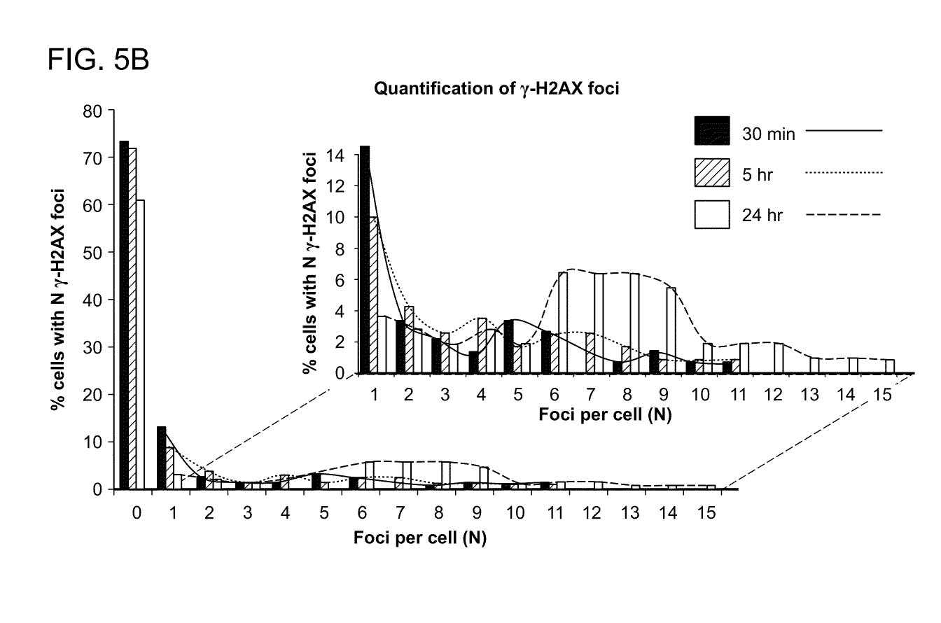

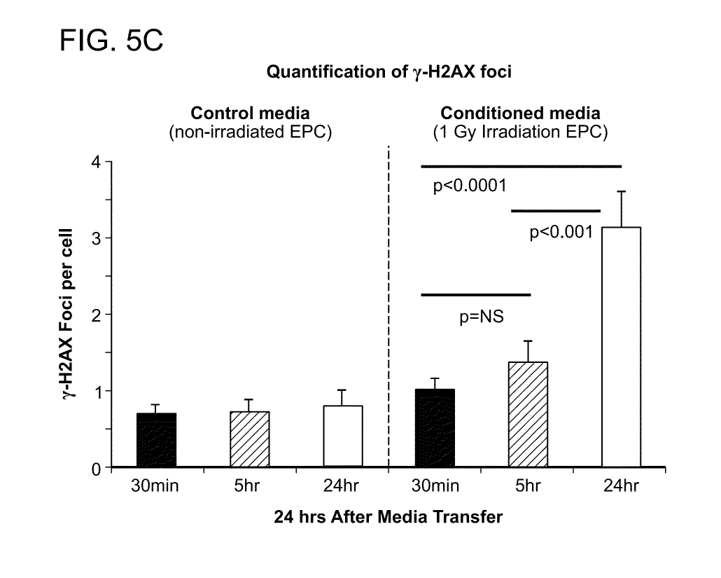

In control medium transferred EPCs there was a negligible number of γ-H2AX (+) EPCs, each with no more than 1-4 endogenous γ-H2AX foci (FIGS. 5A and 5C). Compared to control, 30 min, and 5 hr IR conditioned medium-treated cells, the distribution of the EPCs with a given number of γ-H2AX foci showed a significant increase in N-IR cells treated with 24 hr IR conditioned medium in the percentage of cells with γ-H2AX (+) foci (FIGS. 5A and 5B). These results indicated that bystander responses were mediated in BM-derived EPCs. Compared to all other samples, EPCs treated with 24-hrs conditioned medium had a 3-fold increase (p<0.0001) in the mean number of foci/cells (FIGS. 5A and 5C). Thus, BM-derived EPCs exhibited significant bystander responses in medium transfer experiments in vitro.

### Example 5

### Radiobiological Bystander Effects are Modified Due to Selective Inhibition of TNF Signaling Via Either TNFR1/p55 or TNFR2/75, Increasing Production, Release and Accumulation of IL6, EGF, IL-1α, IL-1β, G-CSF, GM-CSF, MCP1, MIP-1, SCF, and/or RANTES

The radiobiological bystander effect a biological process where irradiated cells transmit DNA damaging signals to naïve none-irradiated cells either by direct contact via gap junction or via release of growth factors or inflammatory cytokines that then mediate DNA damage in other organs (i.e., cardiomyocytes, skeletal muscle, satellite cells, bone marrow, i.e., HSC, hemangioblasts, EPCs). Experiments were performed to determine the radiobiological bystander effect of p75KO and p55KO cells. EPCs from WT, p75KO, and p55KO were treated with media from corresponding irradiated WT, p75KO, and p55KO EPC cultures.

EPCs from young (8-12 weeks) mice for WT, p75KO, and p55KO were obtained (crushed bone marrow), isolated, and plated into two 6-well dishes per mouse. Cells were grown on glass coverslips coated with 0.2% gelatin (one coverslip per well). Of the two 6-well dishes one served as the un-irradiated cells on to which irradiated/conditioned media transfer would be performed and the other dish was gamma irradiated at 1 Gy for all genotypes. Post seeding cells were cultured for 5 days before initiating the study to attain confluence.

For the bystander effect study, medium transfer experiments were performed. On the day of the study (5 days after initial plating), media in all wells was changed with fresh media (3 ml) including control (CTRL) wells. After change of media, cells were incubated for 1 hr prior to irradiation. For control (CTRL) wells, the coverslip was transferred into a 35 mm dish with fresh media. The rest of the cells in the 6-well dishes were irradiated at 1 Gy. At respective time points post irradiation (CTRL-medium from none irradiated cells, 5 hr, Day 1, Day 3 and Day 5—medium collected from irradiated cells), filtered media (0.22 μm filter) was transfer onto non-irradiated EPCs (˜2 ml of irradiated/conditioned media). Non-irradiated cells were incubated for 24 hrs in conditioned media before collecting the coverslips for staining for presence/decay p-γH2AX foci (p-γH2AX foci correlate well with repair of DNA double strand breaks and are indirect indicator of DSB repair/decay). Coverslips at each time point were fixed and stained for pH2AX+Topro-3 (nuclear staining). Results of pH2AX foci were confirmed by co-localization of the foci using another marker for DSB—p53BP1. 100× images obtained for all samples were analyzed using computer assisted image analysis for foci count. Statistical analysis was performed using ANOVA/ANCOVA Fisher's PLSD (StatView statistical package). Statistical significance was assigned when p<0.05.

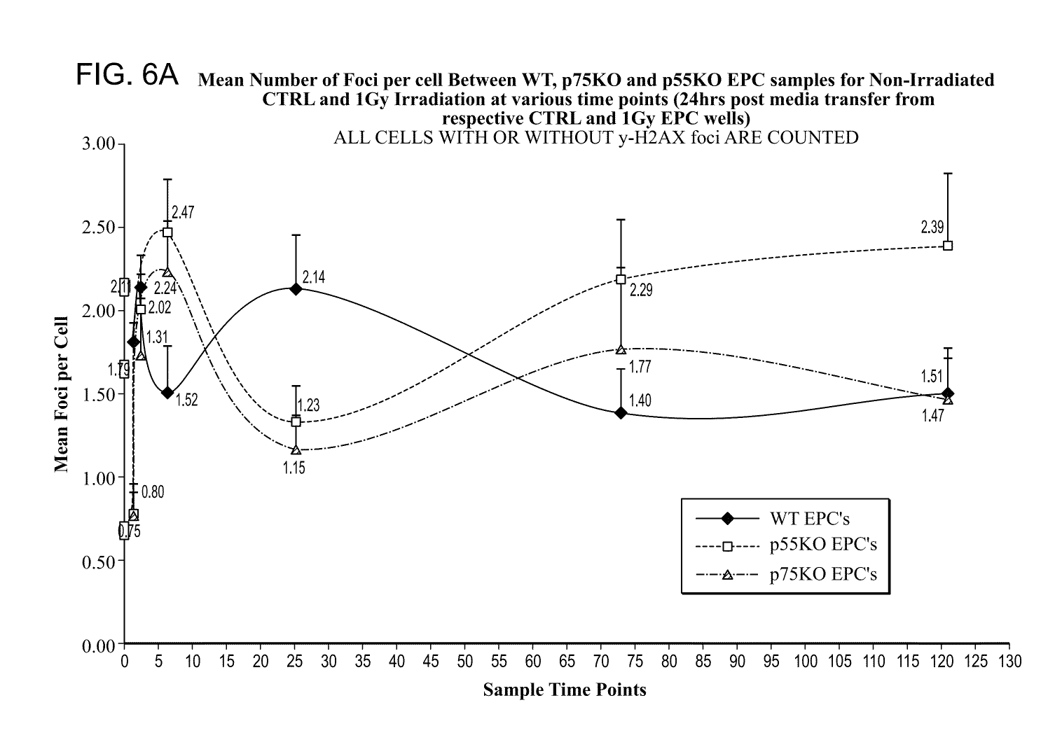

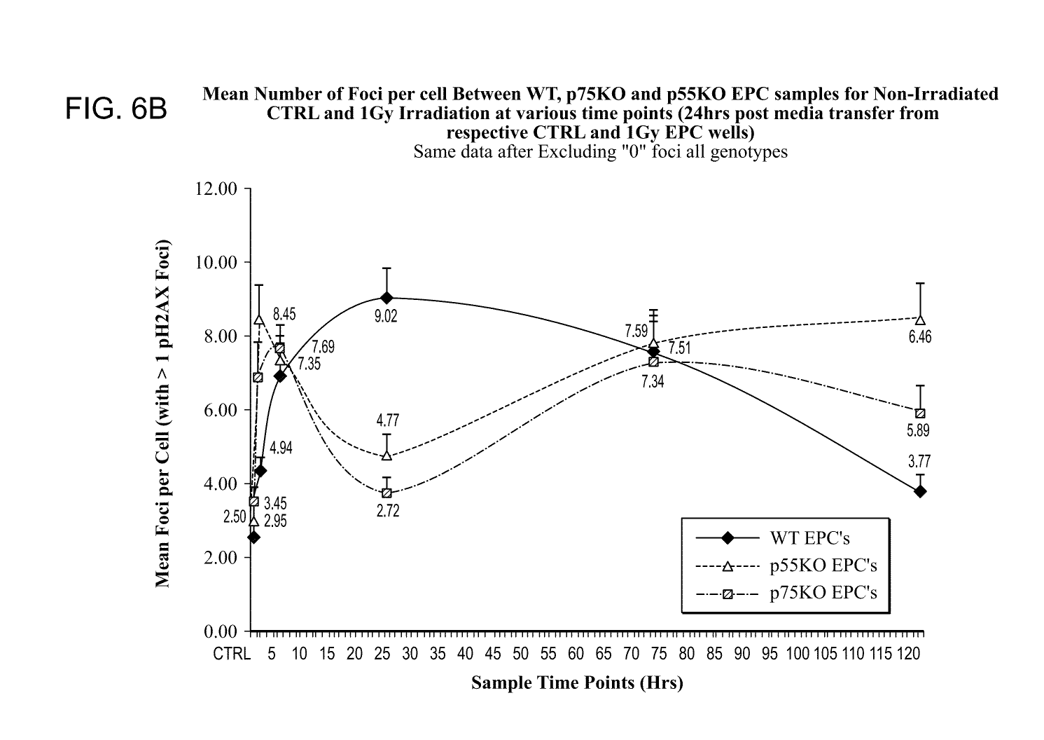

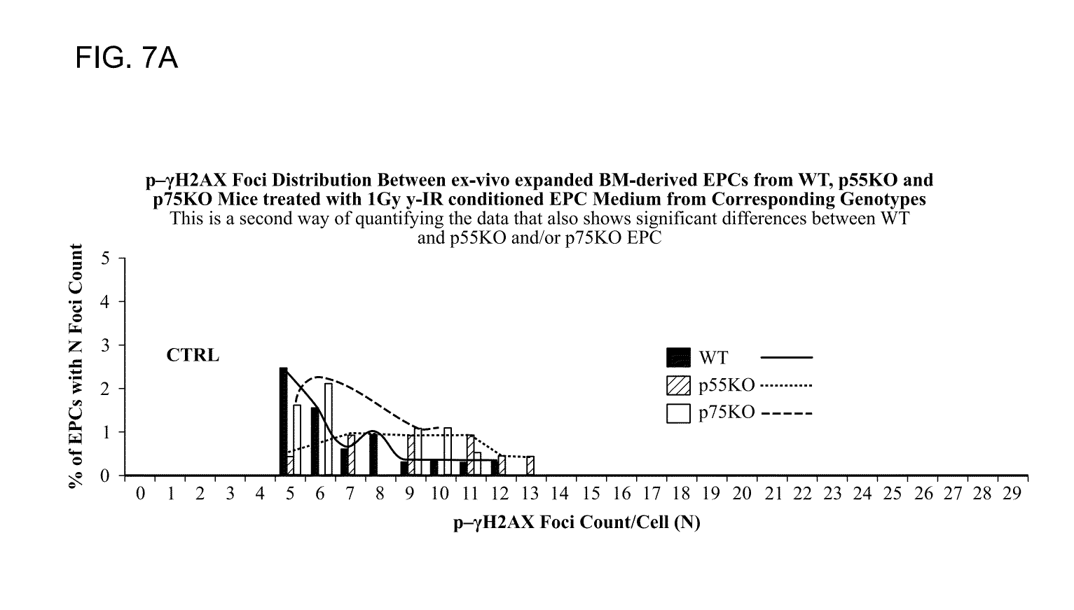

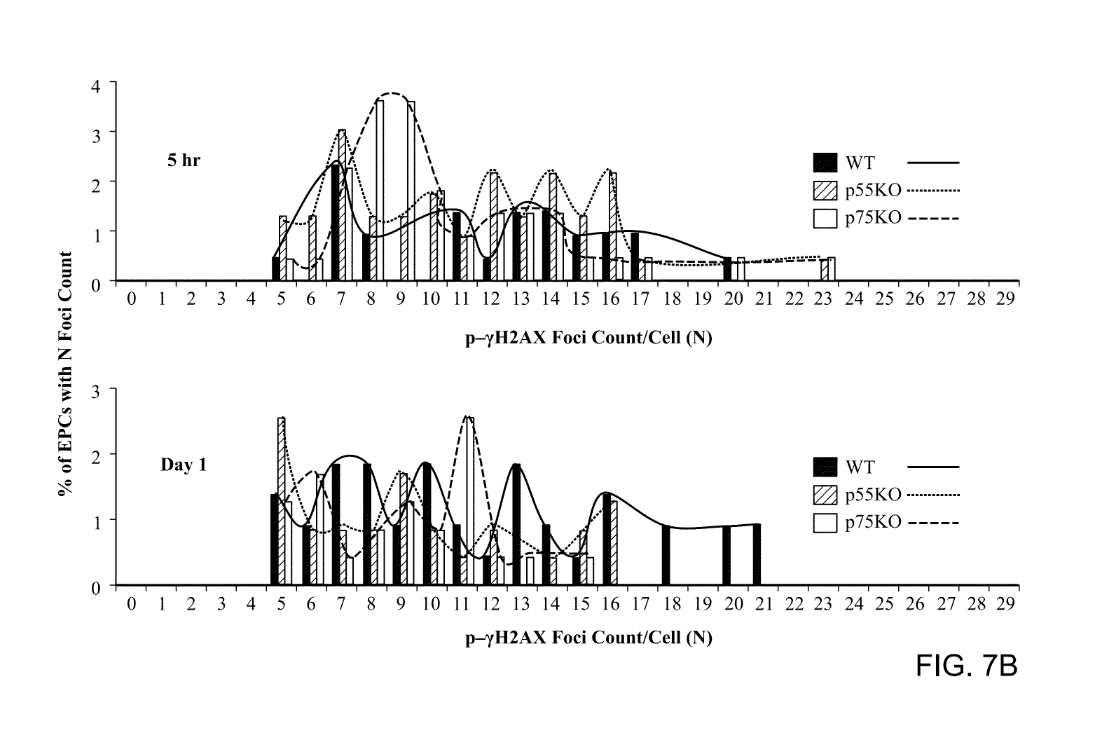

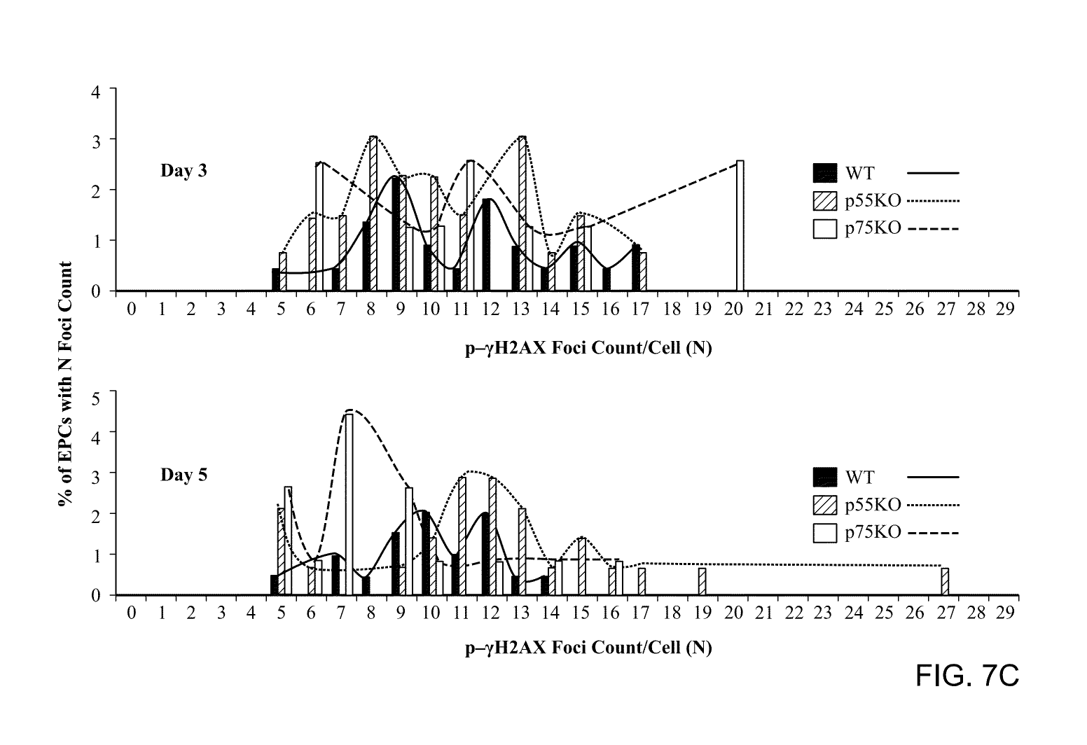

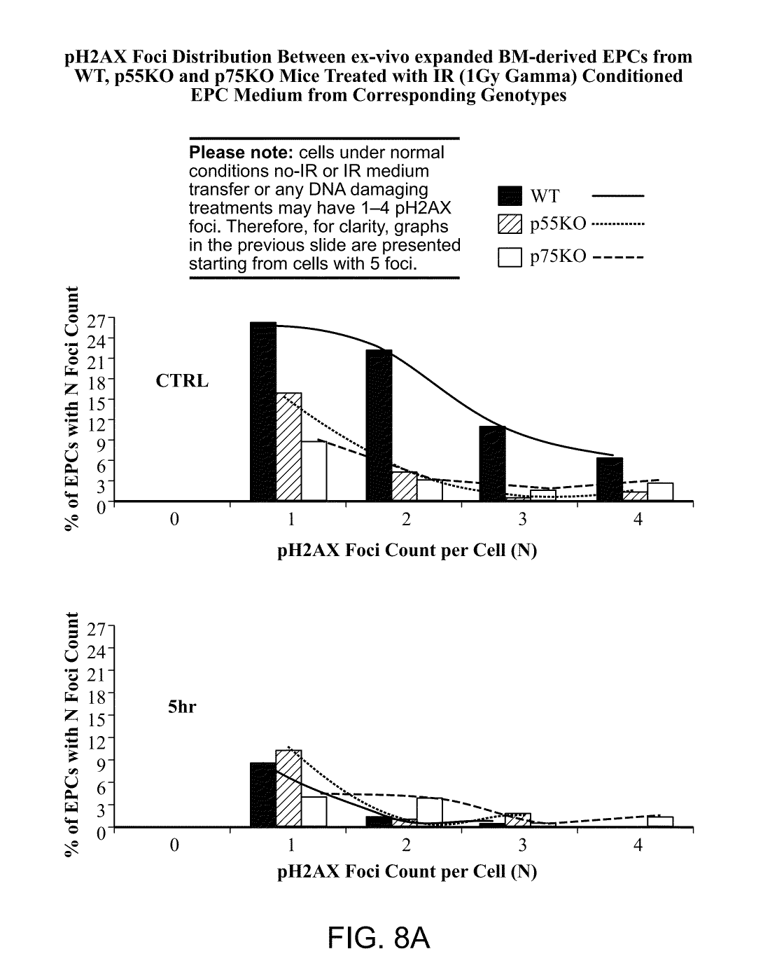

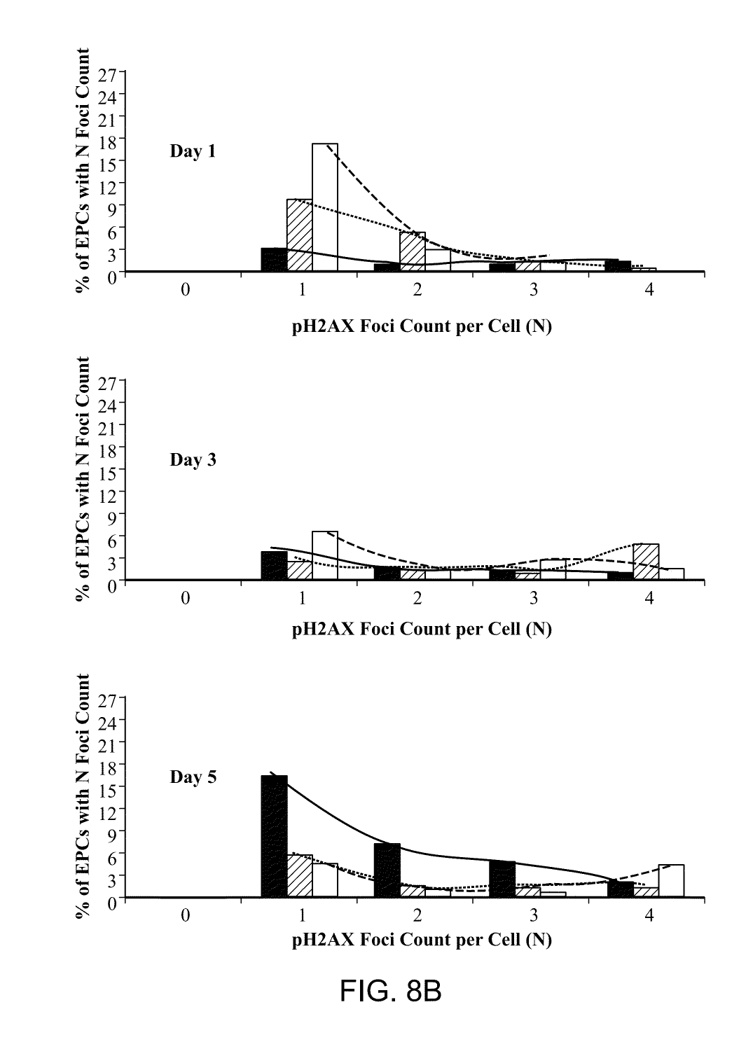

In the absence of either of TNF receptors (p55 or p75) there was a significant decrease, compared to WT, in the formation of p-γH2AX foci between 5-24 hrs after adding IR-conditioned medium to naïve BM-derived EPCs. This result indicated that TNF and signaling via either of TNF receptors was necessary for development of radiobiological bystander responses in non-irradiated BM-derived EPC (FIGS. 6A and 6B). However, in the absence of either of TNF receptors (p55 or p75) there was a significant increase, compared to WT, in the formation of p-γH2AX foci between 1-5 days after adding IR-conditioned medium to naïve BM-derived EPCs. This result indicated that TNF signaling via either of TNF receptors in the absence of the other receptor was delayed (FIG. 6A, FIG. 6B. FIG. 7, and FIG. 8). Thus, bystander responses in non-irradiated BM-derived EPCs were initially inhibited (within 24 hrs) but were then amplified (up to 5 days). Without being bound to a particular theory, a continuous increase in the number (N) of p-γH2AX foci/Cell between 1-5 days in naïve p55KO BM-derived EPCs is indicative that unopposed (by p55, mainly apoptotic) signaling via p75 (that is mainly survival signaling) in p55KO EPCs played an important role in delayed bystander responses. The same result was also observed for unopposed signaling by p75, although to a lesser degree.

Modification of TNF signaling via TNFR1/p55 or TNFR2/75 modulated radiobiological bystander effects. Understanding the roles of TNFR1/p55 or TNFR2/75 can be used to prevent delayed bystander effect-induced damage to naïve BM-derived EPC in normal tissue and in any other normal distant non-irradiated tissue in the mammalian organism that is perfused with blood containing cytokines and growth factors that may induce DNA double strand breaks in non “hit” cells.

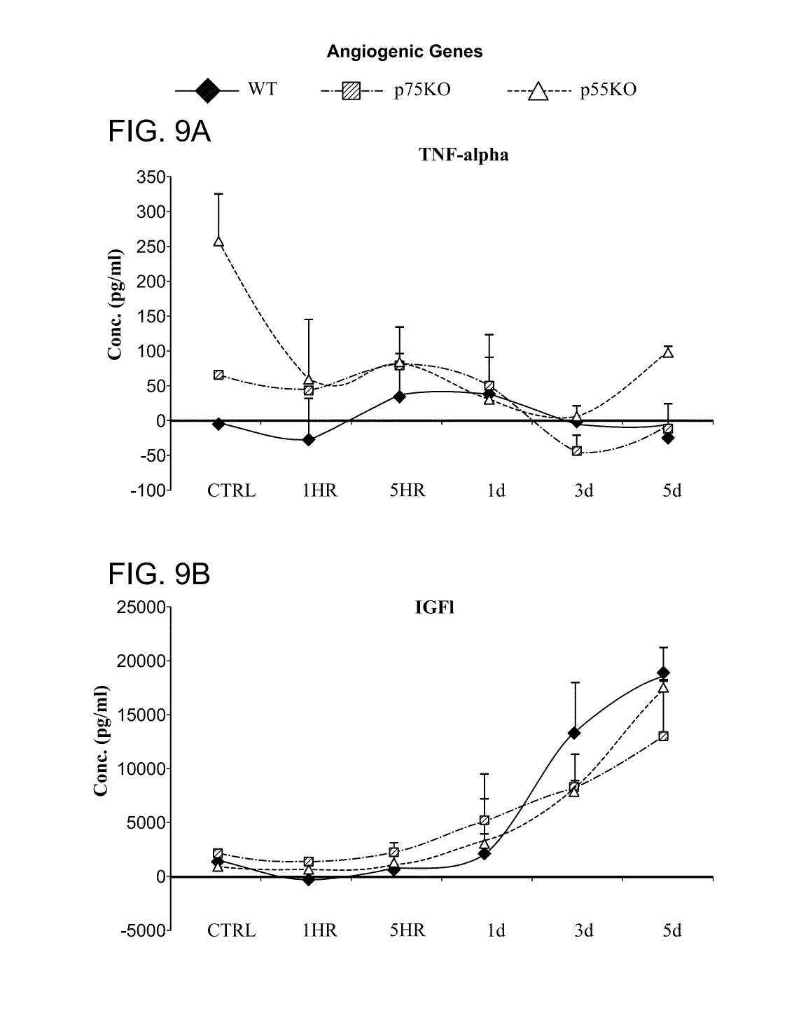

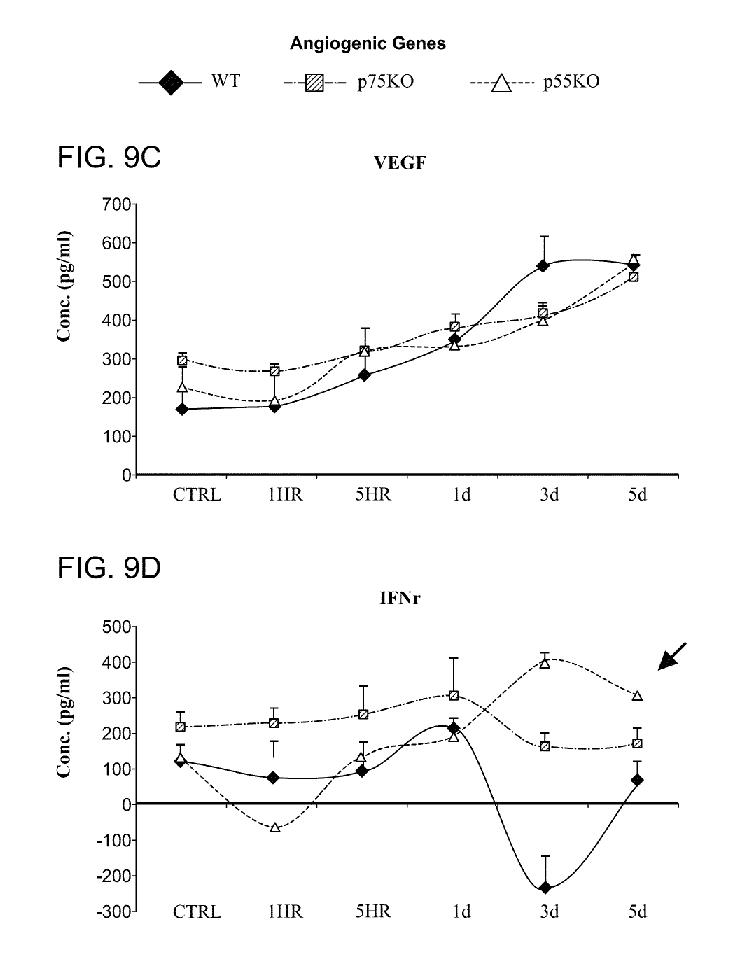

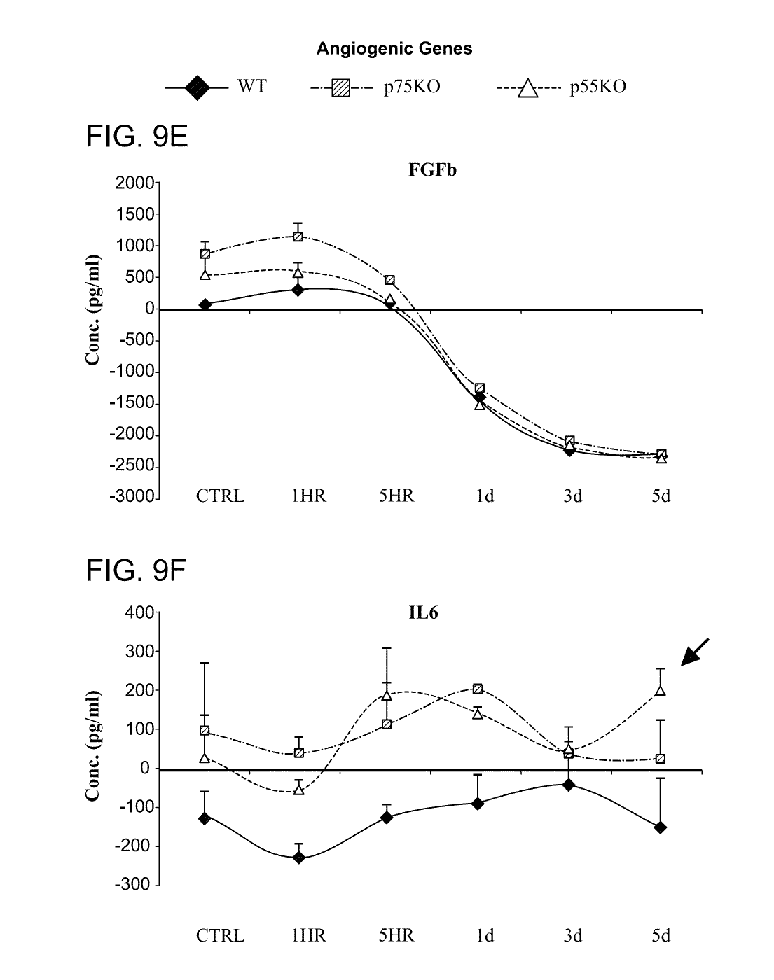

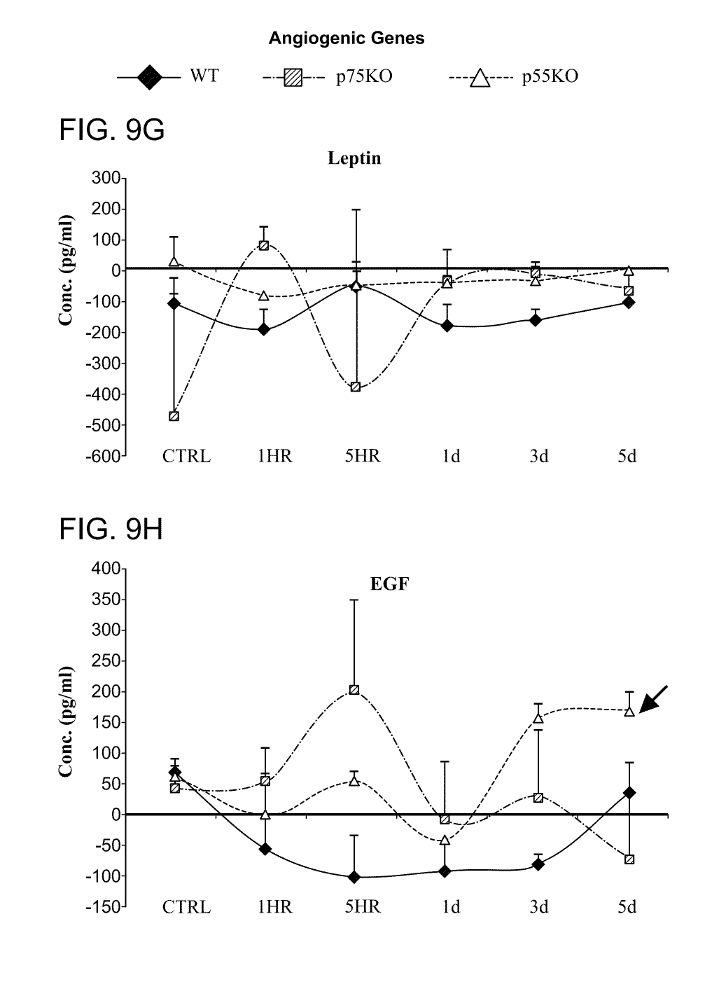

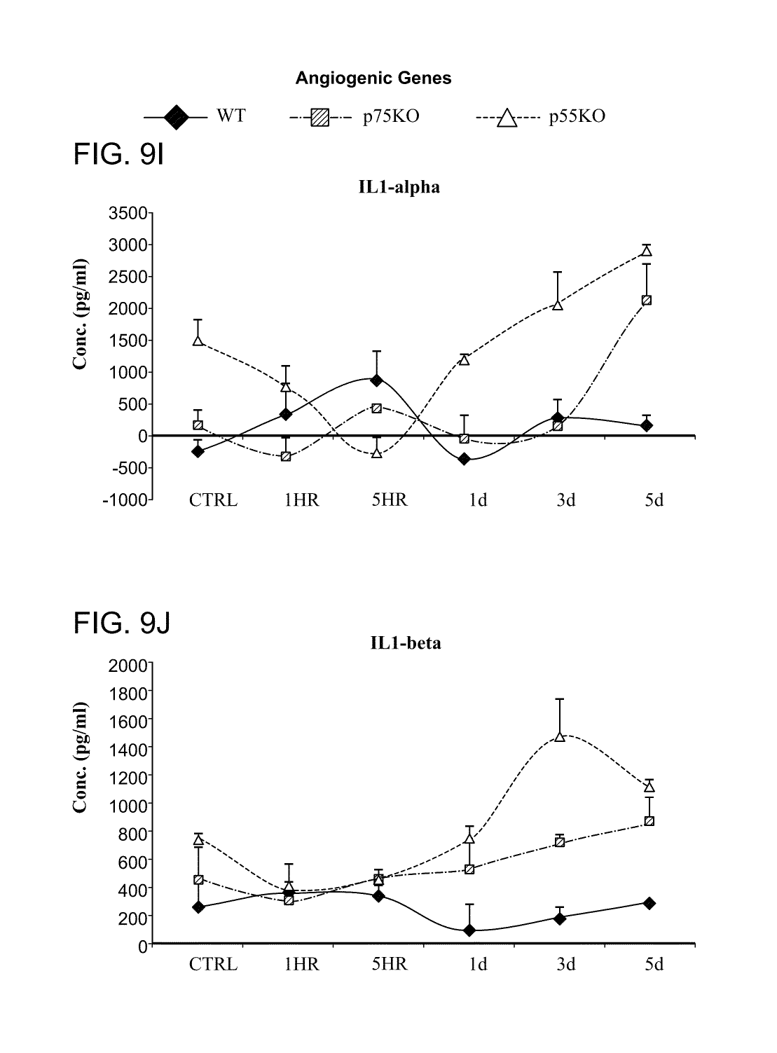

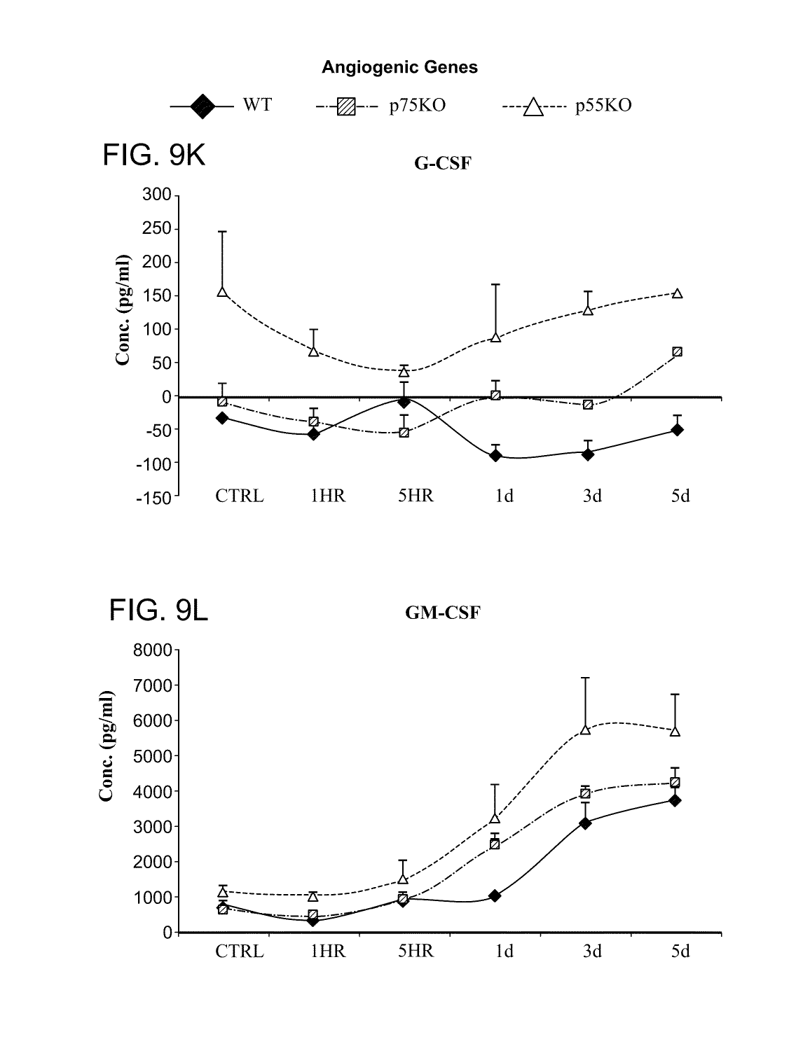

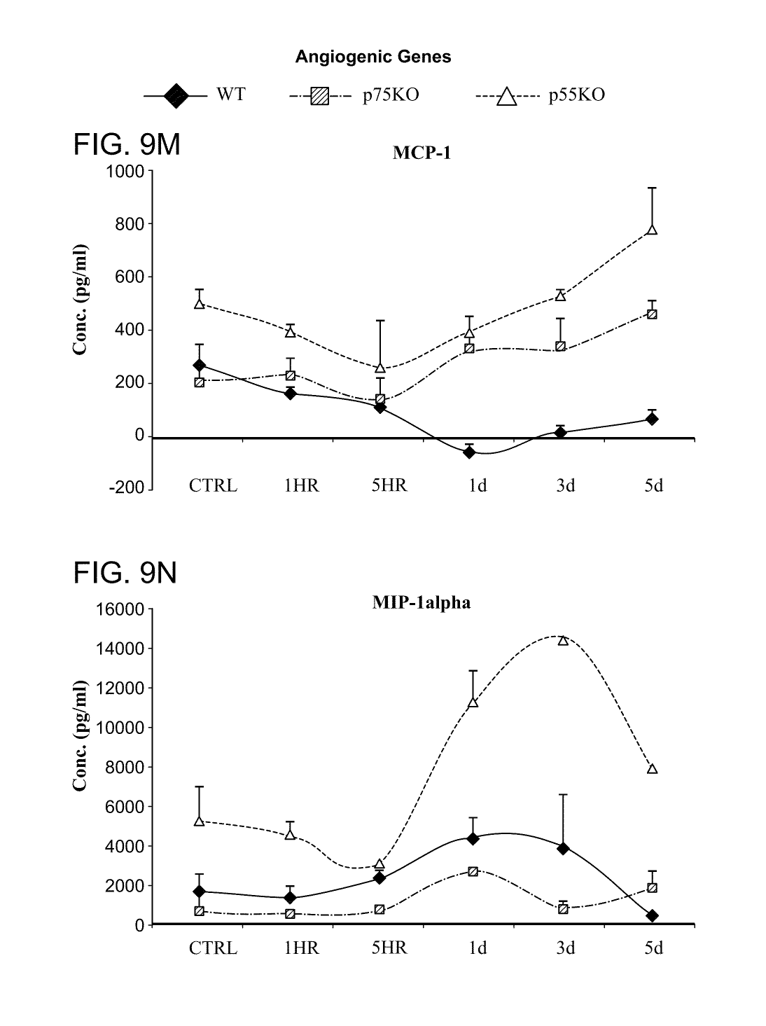

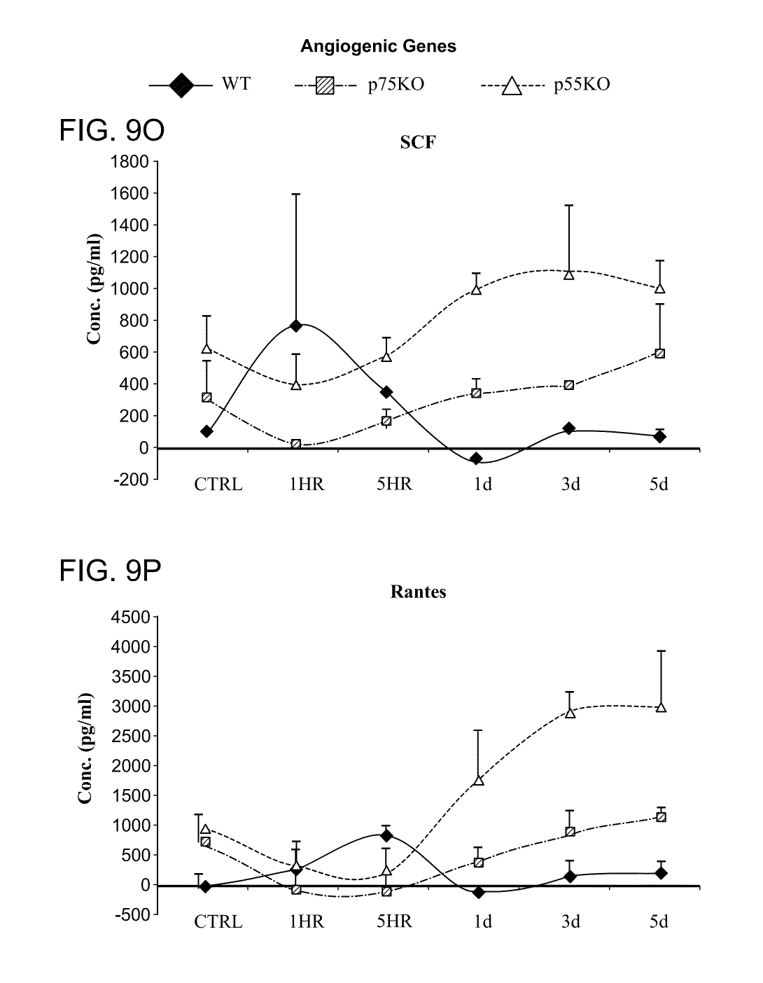

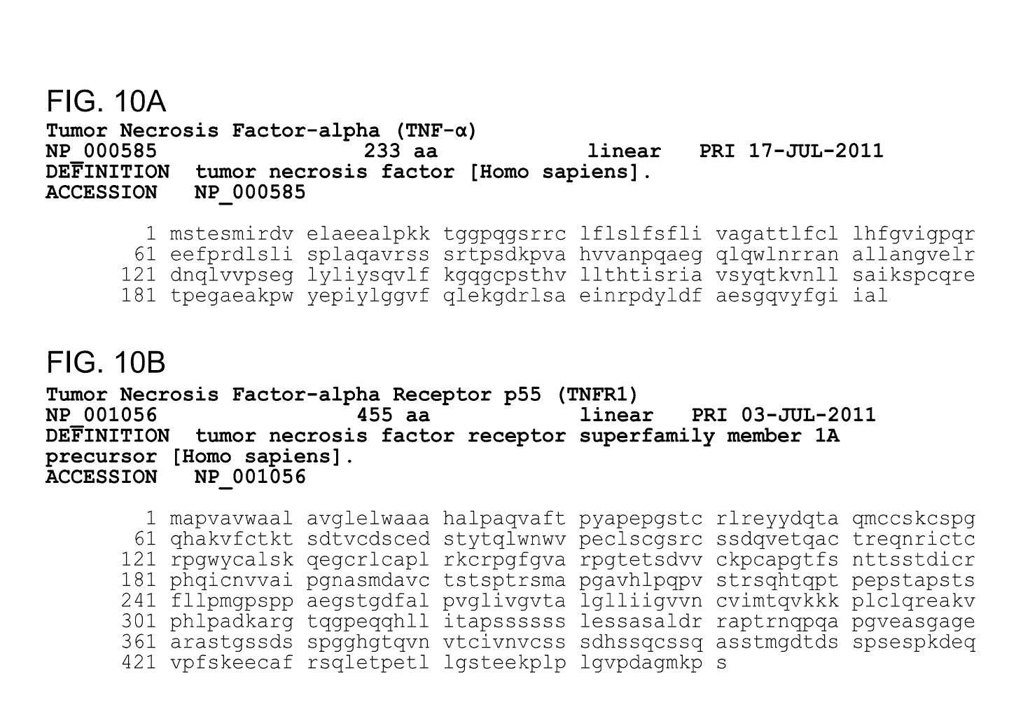

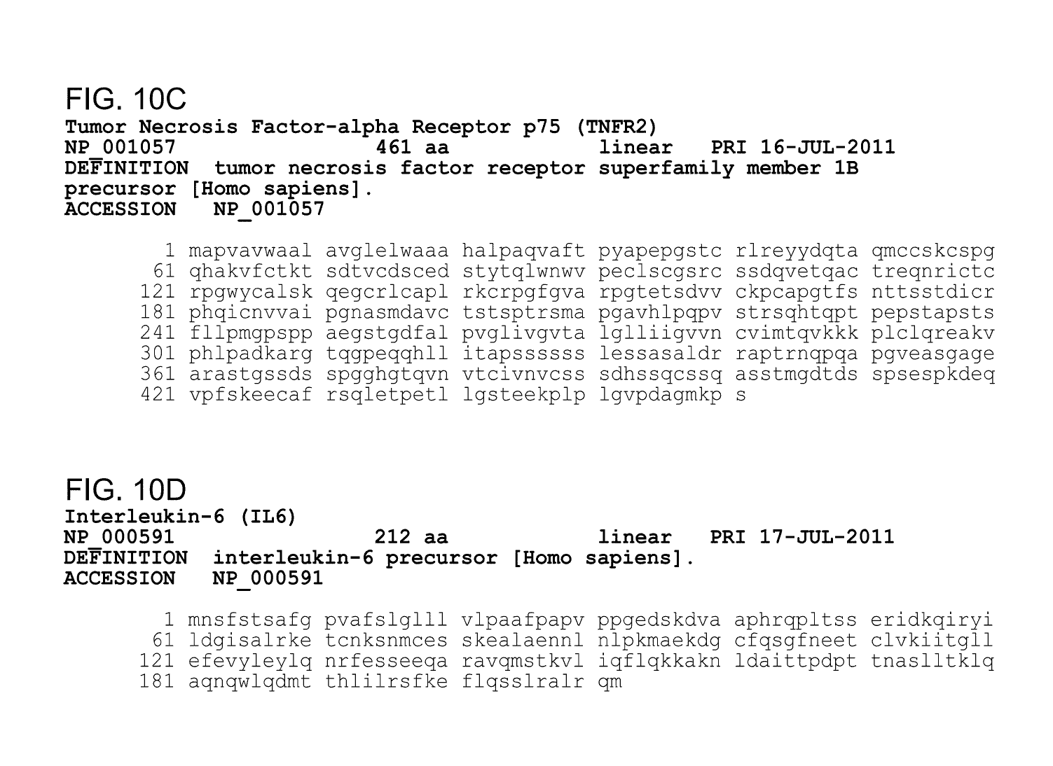

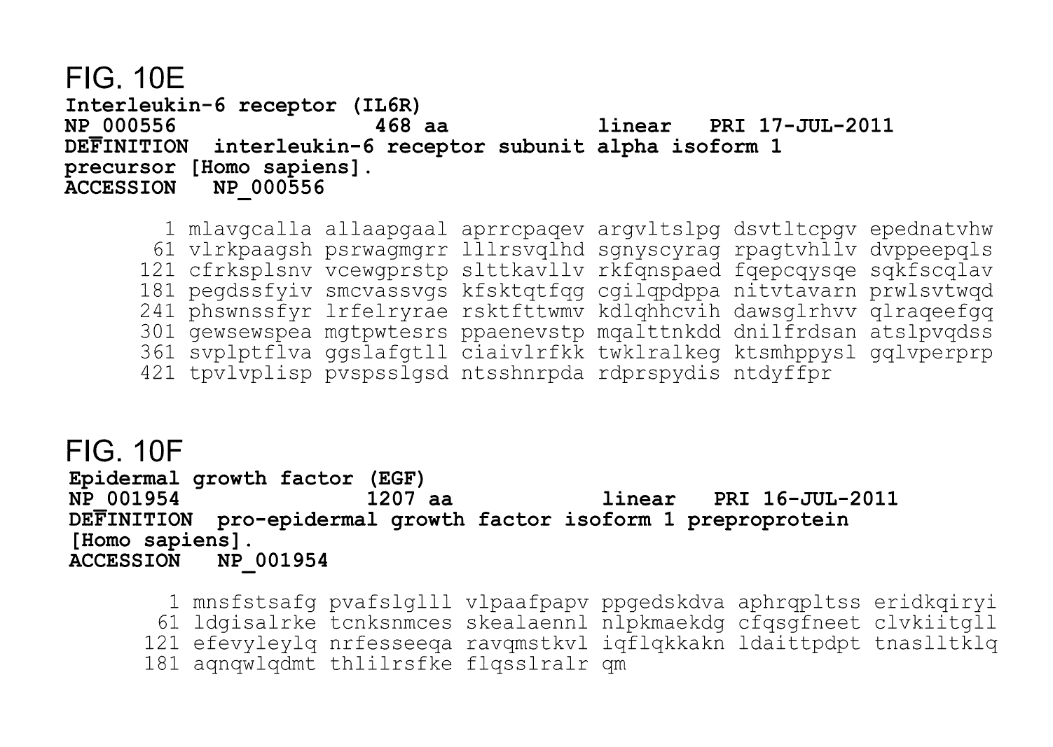

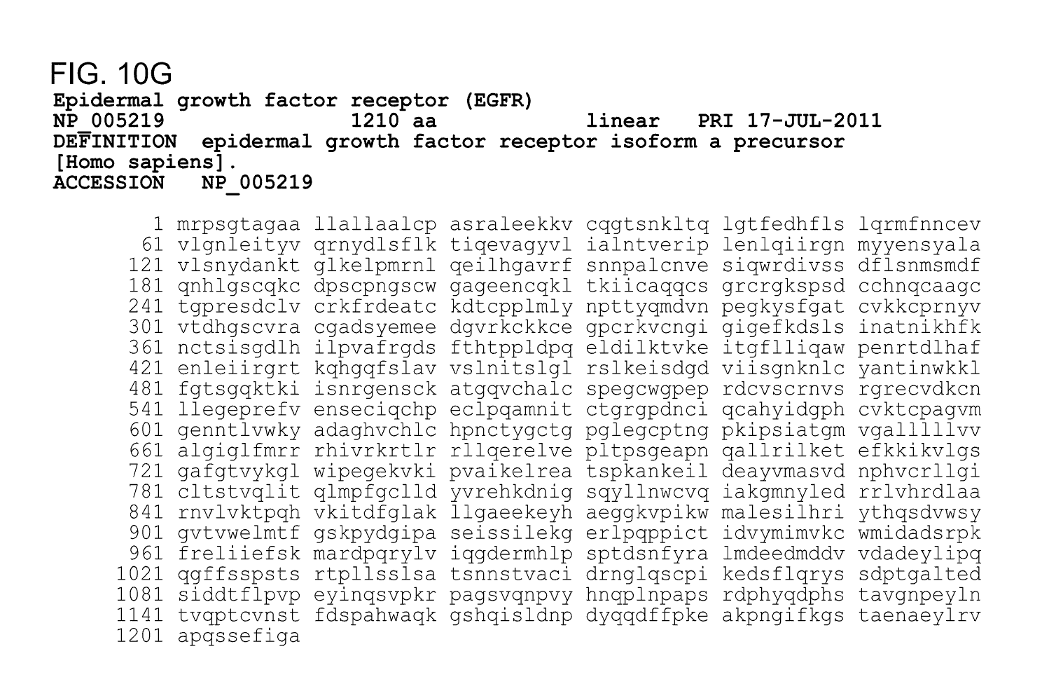

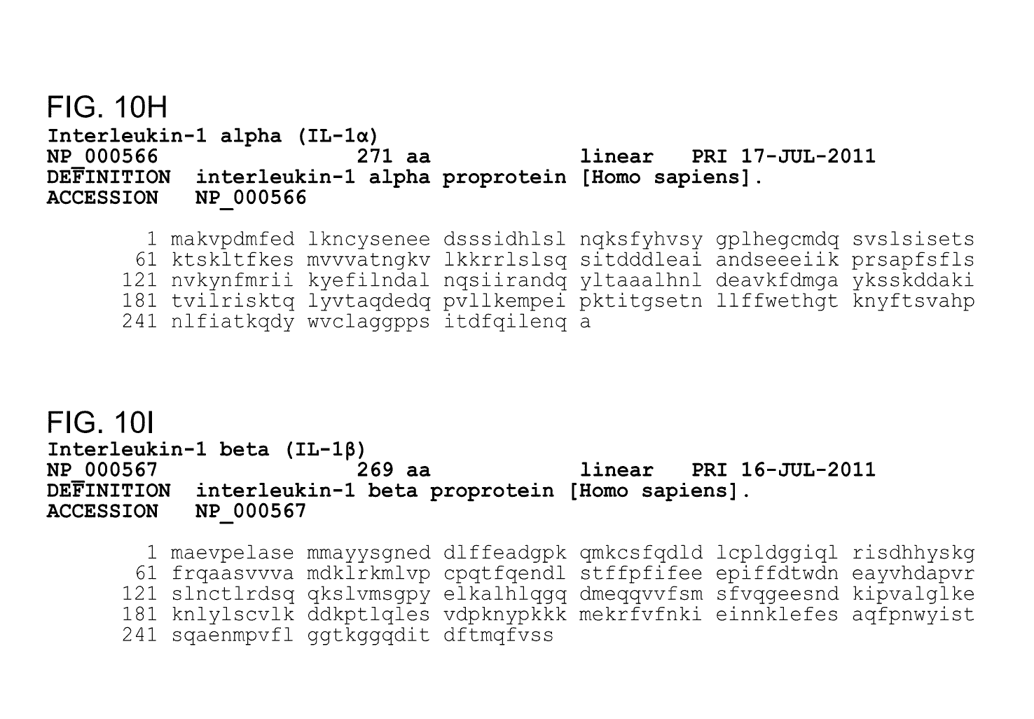

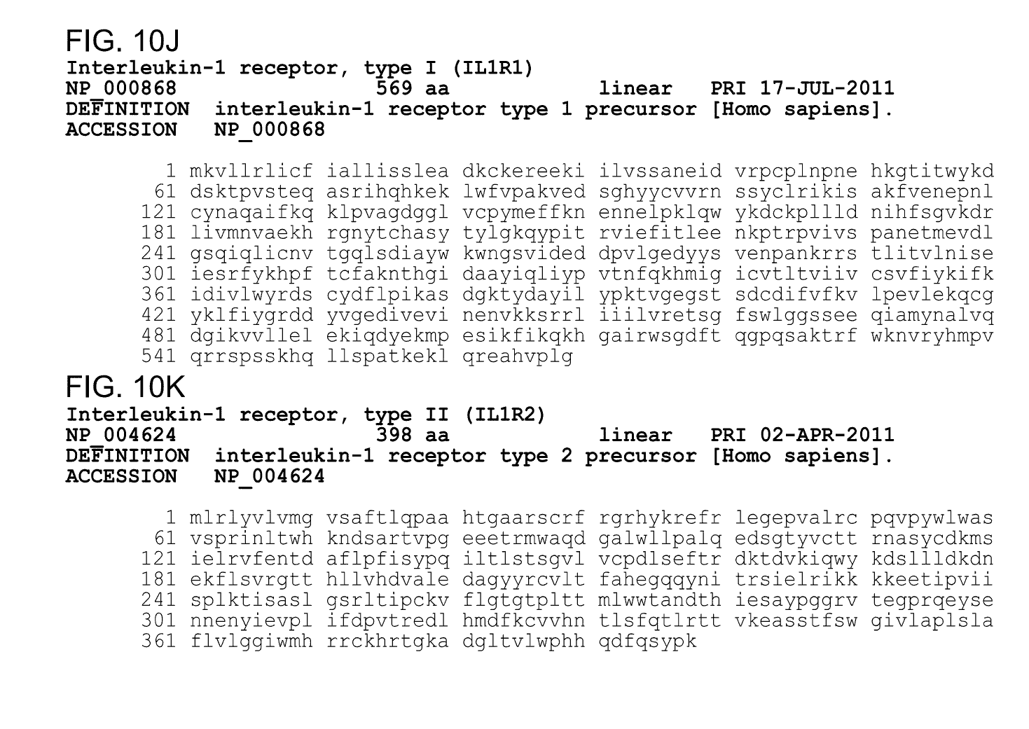

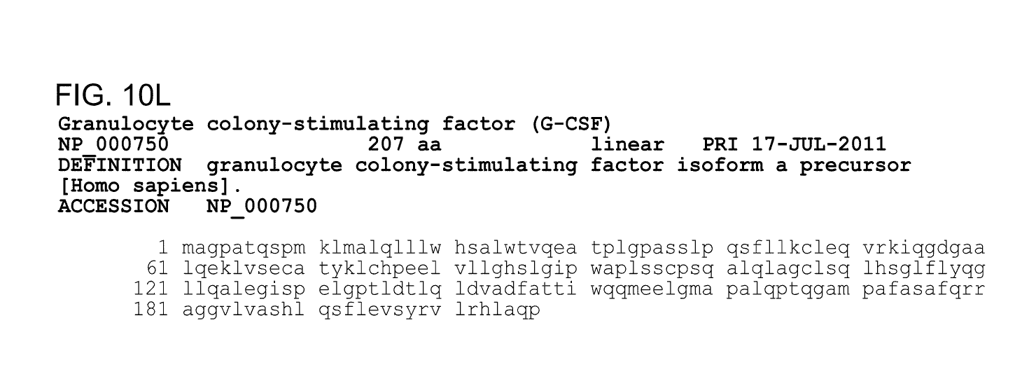

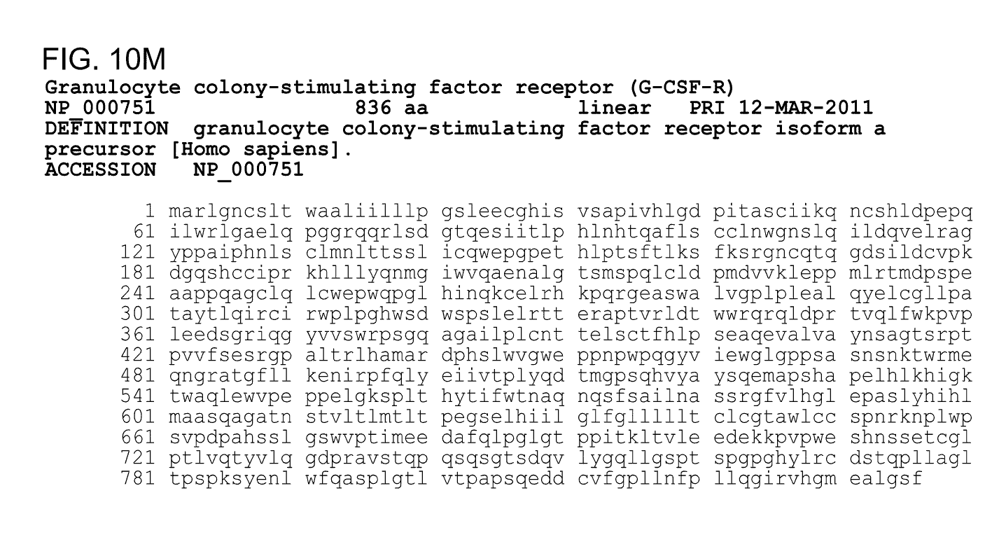

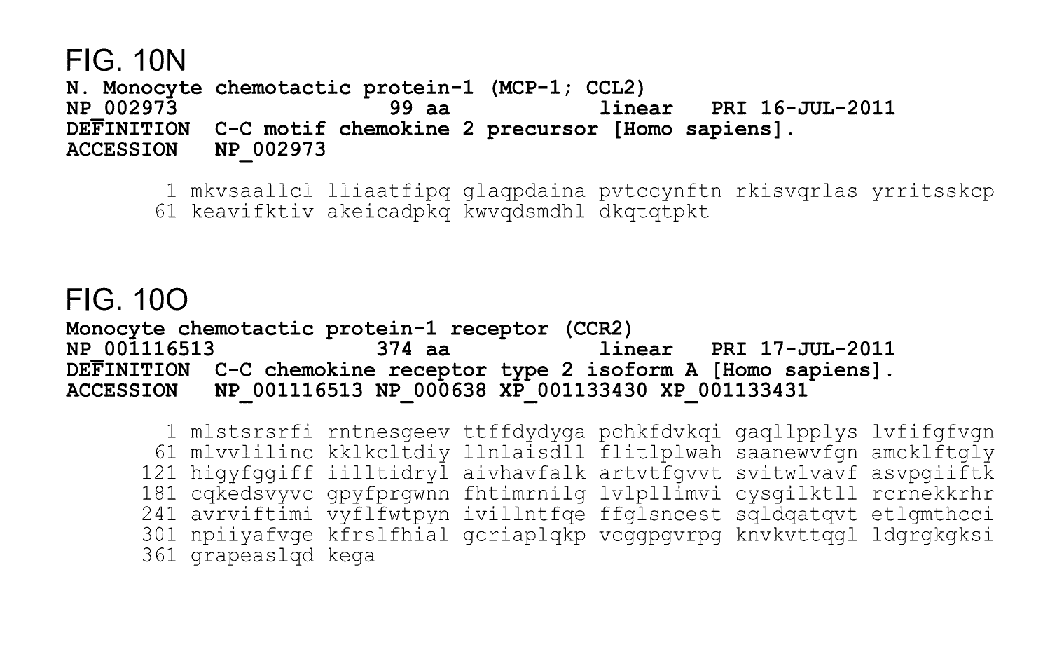

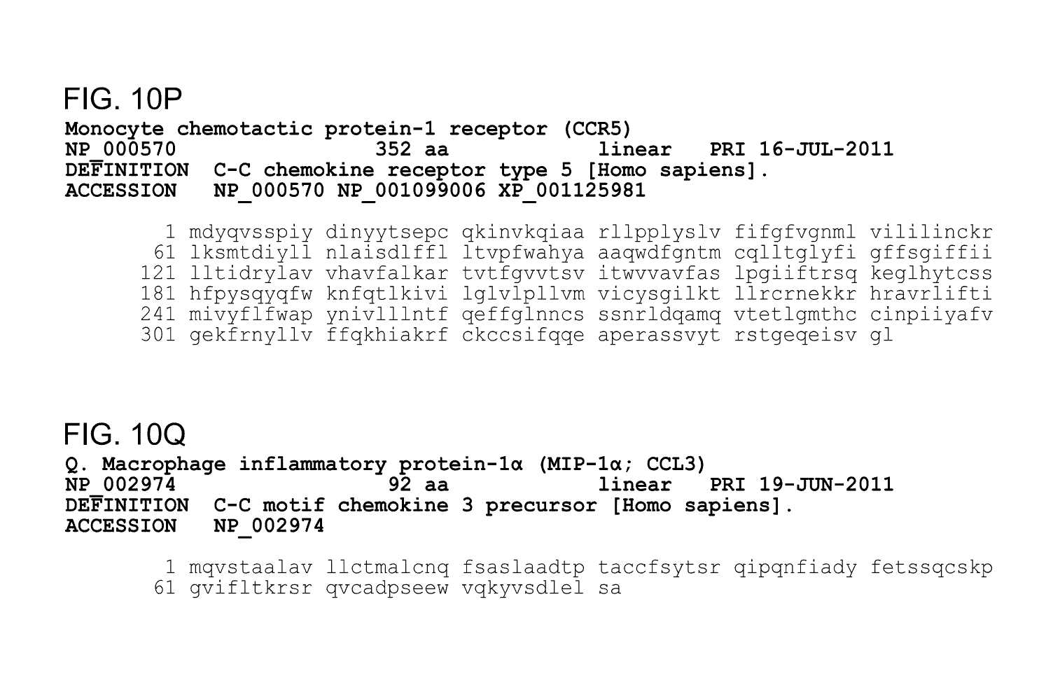

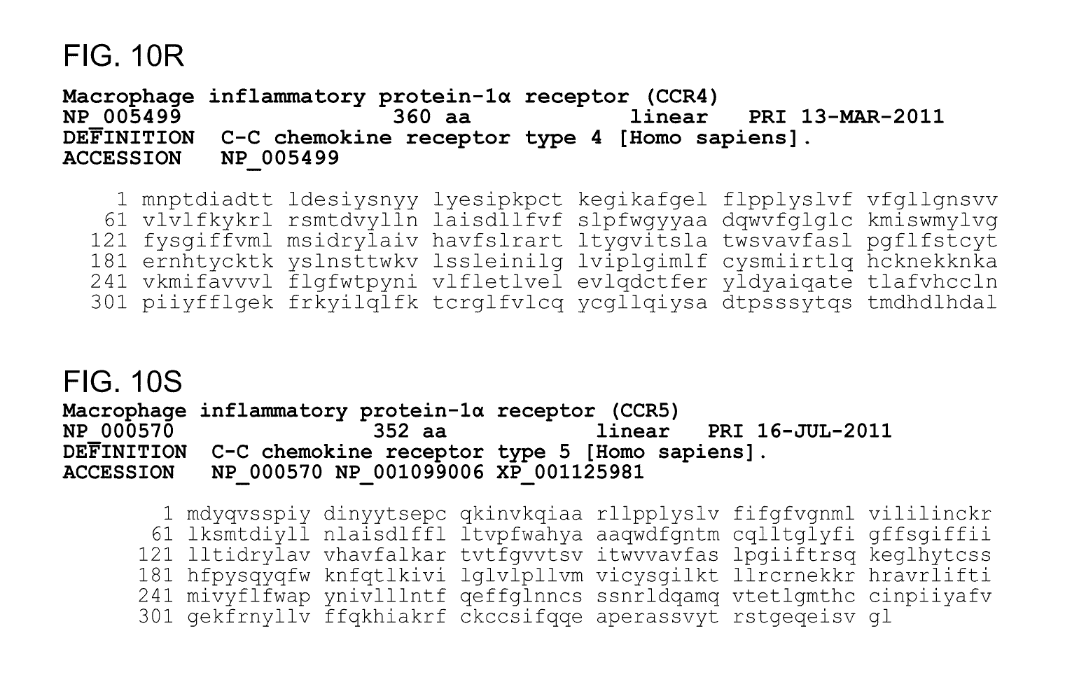

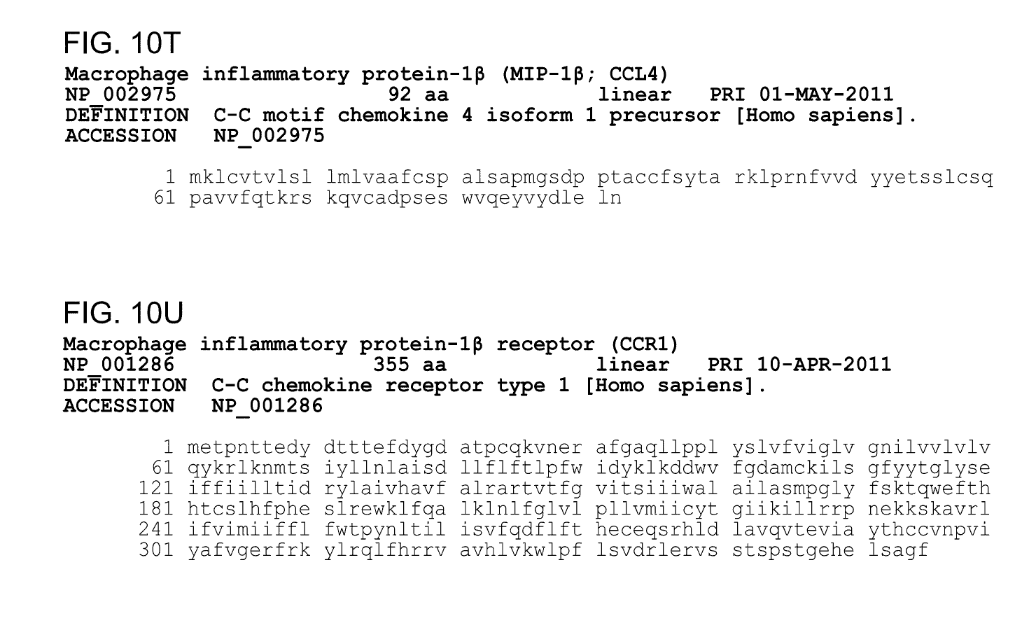

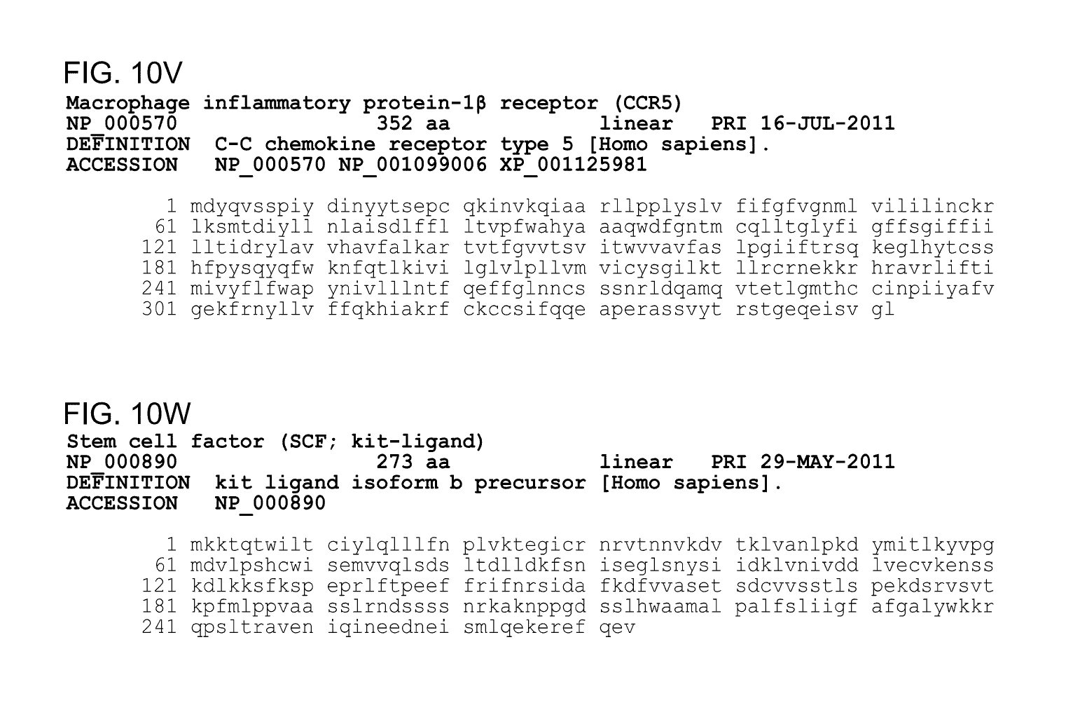

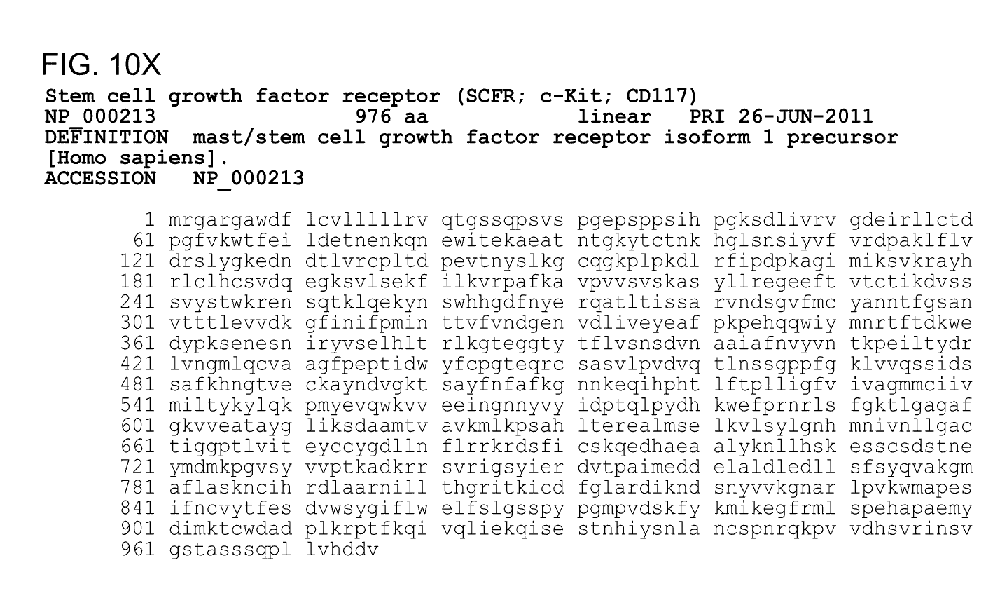

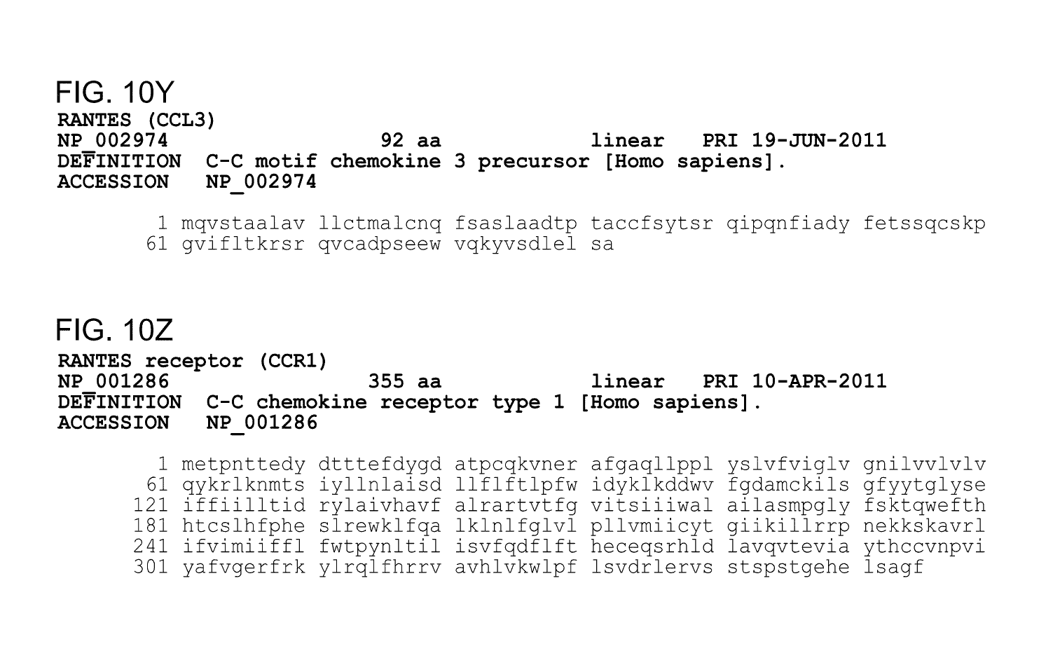

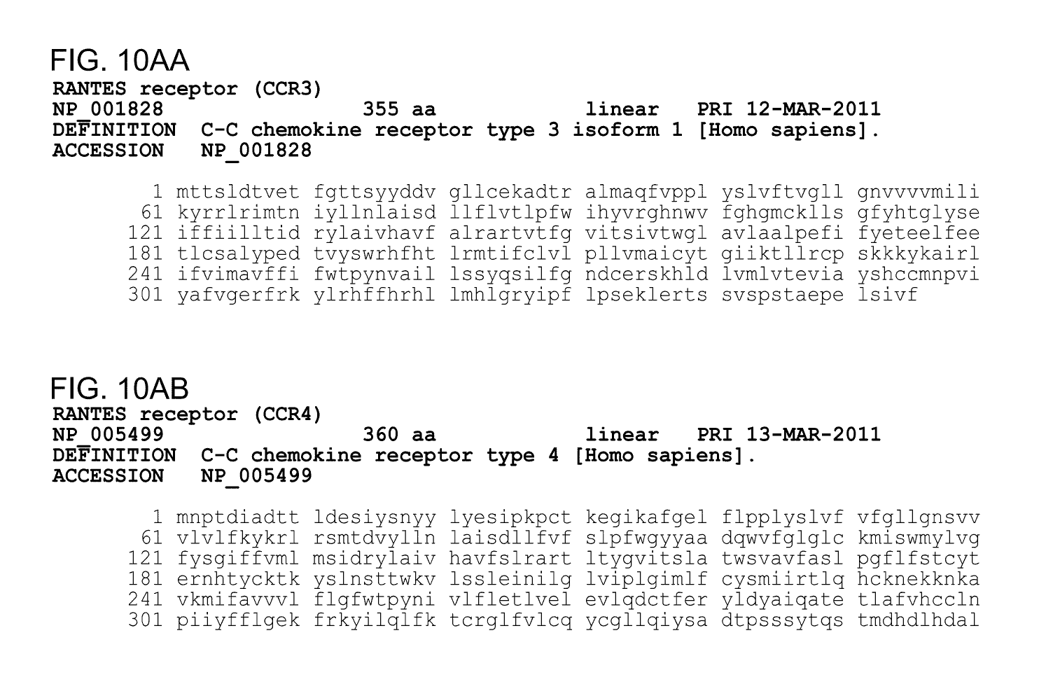

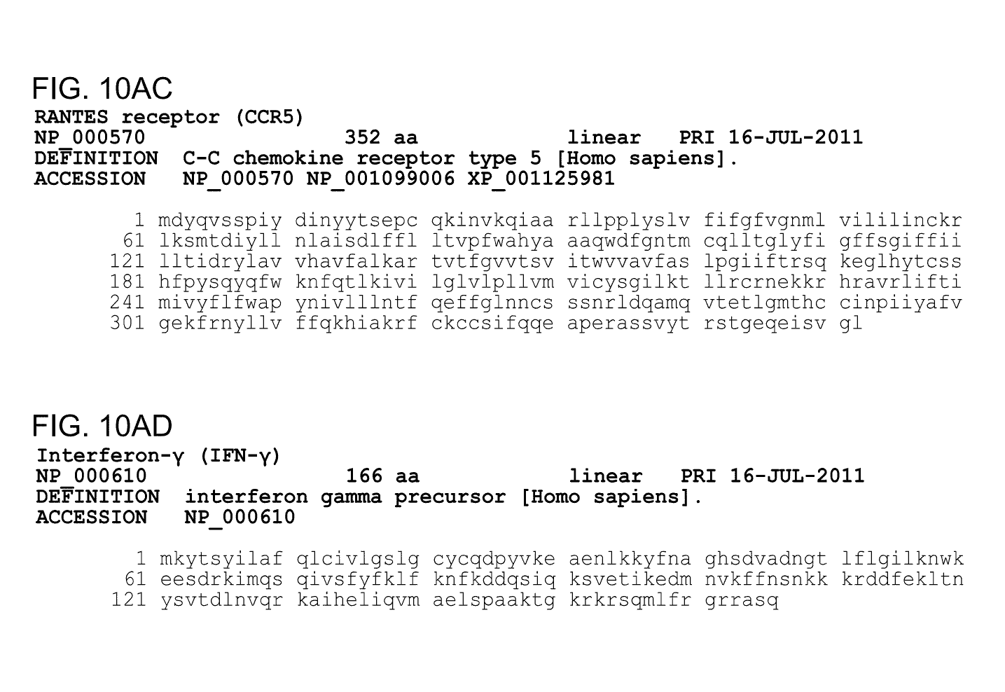

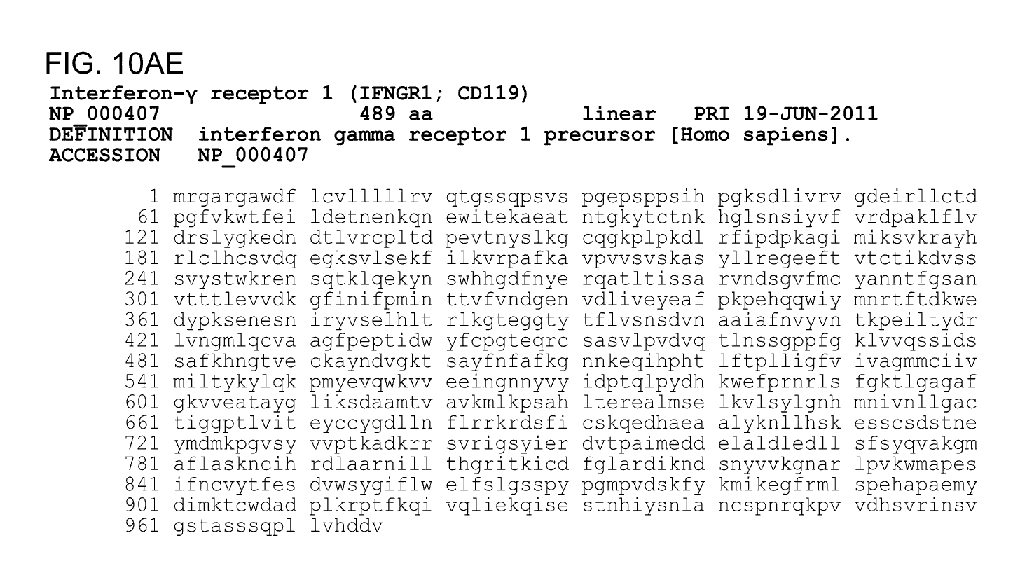

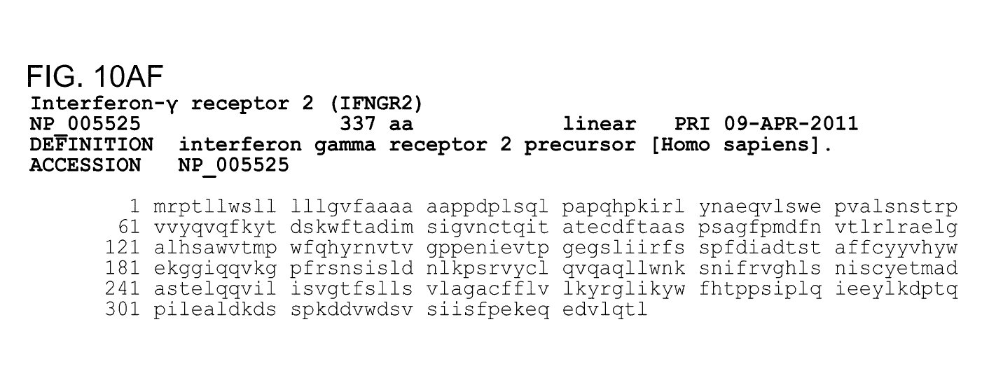

The conditioned media of γ-irradiated (1 Gy) EPCs were also analyzed by ELISA analysis for the expression of cytokines, growth factors, and angiogenic proteins. Increased levels of IL6, EGF, IL-1α, IL-1β, G-CSF, GM-CSF, MCP1, MIP-1, SCF and RANTES were found in conditioned media of p55KO and/or p75KO EPCs, in which signaling TNF signaling occurs via the remaining p75 or p55 receptor (FIG. 9A-FIG. 9D, FIG. 9F, and FIG. 9H-9P). Without intending to be bound by theory, blocking p75 or p55 signaling or inhibiting TNF ligand-receptor interaction (by any means know in the art) reduces production of growth factors and cytokines (e.g., mediators of DNA damage in naïve cells). The increase in cytokines, growth factors, and angiogenic proteins coincided with the increased Double Strand Breaks (DSB) by day 5 (FIG. 6A and FIG. 6B).

Thus, blocking p75 or p55 receptor signaling can be used to reduce harmful effects of damaged and mobilized to the heart and other organs bone marrow derived EPCs and other BM-derived stem and progenitor cells (i.e., HSC, hemangioblasts) in terms of propagating radiobiological bystander effects. The cytokines, growth factors, and angiogenic proteins released in the conditioned medium in vitro or interstitial (intercellular or extracellular space), tissues, organs and blood stream in vivo) and their receptors are targets for treating or preventing the effects of radiobiological bystander responses or effects (e.g., early or delayed).

The results reported herein were obtained using the following methods and materials.

### Kinetics of DNA Damage and Repair in the Heart and BM-Derived EPCs.

DNA double-strand breaks (DSB) are frequently formed by exogenous and endogenous factors including different types of radiation, oxidative damage of the DNA backbone, cellular DNA metabolizing agents and through the process of DNA replication itself⁸¹. Efficient repair of DSBs is necessary, since replication and transcription are blocked at the site of DSBs and the exposed ends of the DNA strands are susceptible to degradation, possibly leading to genetic loss⁸¹. DNA damage and repair studies used fluorescent γ-H2AX nuclear foci formation and decay, optionally with a second co-localizing protein marker tumor suppressor p53 binding protein 1 (p53BP1)⁸² to evaluate the frequency of induction and the rate of repair of DNA double-strand breaks in the heart and in BM-derived EPCs before, 30 min, 1 hr. 24 hr and 7, 15, 30 days post-irradiation.

To determine if BM-derived EPC may have altered expression of one or more DNA repair enzymes (genetic defect) and determine if they may acquired defects (i.e., radiation-induced) in DNA damage repair that may lead to accelerated aging and increased CV risk. EPCs from control and irradiated mice before, 7, 15, 30 days post-irradiation can be profiled for DNA repair enzyme by PCR array. This array profiles the expression of 84 key genes involved in base-excision repair (BER) (Apex1, 2, Lig3, Mutyh, Neil1-3, Ogg1, Parp1-3, Polb, Smug1, Xrcc1, etc.), mismatch repair (MMR) (Mlh1,3, Msh2-6, Pms1,2, Pold3, etc.), double-strand break (DSB) repair (Brca1, 2, Fen1, Lig4, Mre11a, Prkdc, Rad21-54, Xrcc2-6, etc.), and other pathways involved in the repair signaling (Atm, Atr, Exo1, Rfc1, etc.). Controls are included on each array for genomic DNA contamination, RNA quality, and general PCR performance.

### Proliferation and Apoptosis Assays in BM-Derived EPCs.

For evaluation of proliferation and apoptosis in BM-derived EPCs from IR and N-IR mice 30 min, 1 hr, 24 hr and 7, 15, 30 days post-irradiation, MTT assay and active Caspase 3 and Annexin-V immunostaining are utilized. Cells are processed for the MTT assay according to the manufacture's protocol (Roche). A second set are processed for labeling with FITC-conjugated anti-active caspase 3 antibodies (Transduction Laboratories). A third set of similarly treated cells are processed for the detection of apoptosis (Annexin V) using Vibrant Apoptosis Kit (Molecular Probes). Caspase-3 and Annexin-V-labeled cells are then fixed according to manufacturer recommendations and analyzed using a FACScan (Becton Dickinson) flow cytometer.

### Radiation-Induced Inflammatory Responses in the Heart.

Before and 30 min, 1 hr, 24 hr and 7, 15, 30 days post-irradiation, hearts are harvested and preserved in OCT by freezing overnight to process later for serial sectioning. In the hearts of non-irradiated control and irradiated mice, the expression of myeloperoxidase-1 (MPO-1), a neutrophil marker, as well as the expression of CD-68, a glycoprotein normally expressed on macrophages, also known in mice as macrosialin⁸³ are evaluated as described before⁸⁰. Of note, MPO is an antimicrobial enzyme located in the primary granule of neutrophils and MPO-1 is the main MPO isozyme. The number of positive cells are quantified for each staining using appropriate imaging software (Image-J computer software, Wayne-Rasband, NIH).

### Immunodetection of Post-Irradiation Apoptosis in the Heart.

To evaluate apoptotic cells in the heart tissue of irradiated and control mice (before, 1, 3, 7, 15 and 30 days post-irradiation), double immunostaining of Isolectin/B4 (EC-marker) and TUNEL are performed⁸⁴. To evaluate localization of apoptotic cells, an established, commercially available TUNEL kit (ApopTag) is used⁸⁵. Immunohistochemical protocols has been developed and perfected in PI laboratory for at least during last ten years and PI and members of his group are very well prepared in these techniques. Multicolor confocal microscope (Carl Zeiss) will be used for the analyses of immunofluorescent preparations.

### Bystander Responses in Cardiomyocytes and EPCs in Medium Transfer Assay.

Because different cell types respond differently to bystander signaling, bystander phenomenon in cardiomyocytes and BM-derived EPCs were studied to devise an accurate exposure assessment and CV risk characterization of low- and high doses of space, environmental, and therapeutic radiation. Medium Transfer Assay was performed as described⁸⁶. Briefly, EPCs or cardiomyocytes (herein after in this section, “cells”) are irradiated at 60%-70% culture confluence. Two sets of test cultures are prepared as follows: the first are directly irradiated (donor cells) and medium of these cells transferred to a second set of unirradiated recipient cells. Medium from the donor cells are collected at 1, 7, 16 and 24 hrs after irradiation and passed through a 0.22 μm filter before adding to recipient cells to ensure that no irradiated cell is present in the transferred medium. Irradiated medium (500 μl) are collected and processed for ELISA profiling of cytokines and growth factors.

Immediately after media transfer, cells are returned to the incubator and 24 hrs later, recipient cells are fixed and processed for double immunostaining with γ-H2AX/p53BP1 nuclear foci formation and decay to evaluate the frequency of induction and the rate of repair of DSB breaks. For controls, a culture of donor cells are sham-irradiated and the medium at corresponding time points transferred to recipient cells.

### ELISA Strips for Cytokines and Growth Factor Profiling in Media Transfer Experiments.

Cytokines and growth factor are essential molecules that play crucial roles in many biological functions, including inflammation and immunity as well as radiobiological bystander responses. Five hundred ml of medium from the donor cells (see paragraph above) are collected at 1, 7, 16 and 24 hrs post-irradiation and processed for cytokines and growth factor ELISA Profiling Assay (Signosis). The ELISA profiling strips allow simultaneous profiling of 8 cytokines or growth factors (Leptin, TNFα, IGF-1, IL-6, VEGF, IL-1α, IL-1β, and GCSF) in the media of cardiomyocytes and EPCs. Each well of the strip is coated with a primary antibody against a specific cytokine and total 8 wells of a strip target 8 different cytokines.

### Radiation-Induced Mutagenesis in BM-Derived EPCs, Hemangioblasts and Hematopoietic Stem Cells-HSC (e.g., BM-Derived Cells).

An association between low (<0.5 Gy) and moderate doses (<5 Gy) of ionizing radiation and late-occurring cardiovascular disease and a role for somatic mutations has been proposed that would indicate a stochastic effect⁸⁷. For evaluation of mutagenic effects of low-dose space radiation in BM-derived EPCs 15 and 30 days (short-term mutagenesis) and 3, 6, 9 and 12 months (long-term mutagenesis) post-irradiation, a transgenic mouse model is utilized that harbors a bacterial reporter gene for detection of mutation frequencies in virtually all tissues over the lifetime of the animal⁷⁷. This model allows obtaining information on tissue-specificity of spontaneous or radiation-induced mutations and their relationship with causative agent (i.e., radiation)⁸⁸. An advantage of this plasmid-based models compared to the endogenous gene (i.e., Hprt, Aprt) based systems is the ability to recover (large) deletion mutations⁸⁹.

### Measurements of Post-Irradiation ROS Production in BM-Derived Cells.

ROS production are monitored 15 days and 1, 3, 6, 9 and 12 months post-irradiation with the RedOx sensitive probe, 5-(and 6)chloromethyl-2′7′-dichlordihydrofluorescein diacetate (CM-H₂DCFDA) loaded into the cells ex-vivo as described⁹⁰. Irradiated mice are observed for substantial increase in ROS production in the EPCs compared to EPCs from non-irradiated animals.

### Oxidative Stress and Antioxidant Defense PCR Array to Analyze BM-Derived Cells.

To determine if BM-derived EPC have altered expression of one or more oxidative stress and antioxidant defense genes that may lead to accelerated aging and increased CV risk, EPCs from control and irradiated mice 3, 6, 9 and 12 months post-irradiation are used for mouse oxidative stress and antioxidant defense PCR array profiling. Using real-time PCR expression of a focused panel of genes related to oxidative stress is analyzed. The array profiles the expression of 84 genes related to oxidative stress. Peroxidases are represented on this array including glutathione peroxidases (GPx) and peroxiredoxins (TPx). Also included are the genes involved in reactive oxygen species (ROS) metabolism, such as oxidative stress responsive genes and genes involved in superoxide metabolism such as superoxide dismutases (SOD). For each treatment group (3, 6, 9 and 12 months post-irradiation) at least samples are examined.

### Expression of Angiogenic and Pro-Survival Factors in BM-Derived Cells.

Because induction of endothelial growth factors and cytokines (i.e. VEGF, bFGF, PDGF, IL-8, TNF-α, etc.) is involved in initiation of angiogenesis⁹¹ the effect of radiation on gene expression in BM-derived EPCs isolated from non-irradiated and irradiated mice 3, 6, 9 and 12 months post-irradiation was evaluated using commercially available angiogenesis pathway specific gene arrays. Samples are processed according to the protocol provided in Oligo GEArray Microarray Kit from (SABiosciences) as per manufacturer's instructions. For each treatment group (3, 6, 9 and 12 months post-irradiation) least 5 samples each with 4 technical replicates are examined. This allows statistical analysis of the data and the fold increase or decrease in gene expression for the different samples. Part of the isolated RNA is used to perform qRT-PCR using SYBR Green real time PCR reaction to confirm the gene array analysis results.

### EPC Migration.

One of the important functional features of endothelial cells is their ability to migrate towards chemotactic stimuli. Chemotaxis and chemokinesis of BM-derived EPCs isolated from irradiated and non-irradiated mice 3, 6, 9 and 12 months post-irradiation in response to TNF-α (1 and 10 ng/ml, known angiogenic TNF concentrations), and rmVEGF (20 ng/ml) and GCSF (50 ng/ml) are evaluated using a modified checkerboard assay using Coster Transwell chambers (6.5 mm diameter, 5 μm pore) as described previously⁹². Cells migrating into the lower chamber are collected in 50 μl of buffer and counted manually using hemocytometer and Coulter Counter.

### EPC Invasion.

The BioCoat Matrigel Invasion Chamber (BD Biosciences) is used for this assay. BM-derived EPCs isolated from irradiated and non-irradiated mice 3, 6, 9 and 12 months post-irradiation are seeded on the inserts (upper chambers) and allowed to invade through the Matrigel and attach to the membrane. Invasion will be assessed after 22 hours by staining membranes from the bottom of the chambers (containing the invading cells) with Diff-Quick (Fisher).

### EPC Tubulogenesis.

To examine the ability of BM-derived EPCs isolated from irradiated and non-irradiated mice 3, 6, 9 and 12 months post-irradiation to form tube-like structures are evaluated on VEGF-enriched matrigel, another endothelial functional assay. To examine the formation of tube-like structures, cells are seeded at 5×10⁴ cells/well on 4-well chamber slides coated with Matrigel (Collaborative Biomedical Products, MA) and incubated for 12, 16 and 24 hrs in medium containing 5% FBS and supplemented with medium alone or 1 and/or 10 ng/ml of rmTNF-α (BD PharMingen, CA). Cells in the chambers will be examined and photographs are taken after 12, 16 and 24 hrs post-stimulation.

### 2D Targeted M-Mode Echocardiography in Mice.

To evaluate that space, environmental, or therapeutic radiation exposure at early age (young and adult) accelerates age-associated impairment of physiologic homeostasis in the heart, transthoracic echocardiography in irradiated and non-irradiated mice 3, 6, 9, 12 months post-irradiation is performed. Mice are lightly anesthetized by isoflurane inhalation and studied in the conscious condition on a warming pad. 2D guided M-mode echocardiography in the mouse are performed with a 12-MHz transducer (Hewlett Packard) as described⁹³. Left ventricular (LV) wall thickness, LV end-systolic and end-diastolic dimension and endocardial fractional shortening are measured. Echocardiography allows for assessment of cardiac valve areas and function, any abnormal communications between the left and right side of the heart, any leaking of blood through the valves (valvular regurgitation) and calculation of the cardiac output as well as the ejection fraction (fraction of blood pumped out of ventricles with each heartbeat). Other parameters measured include cardiac dimensions (luminal diameters and septal thicknesses).

### Quantification of Circulating Peripheral Blood BM-Derived Cells.

To determine altered regulation of EPC, hemangioblast, and HSC mobilization from bone marrow by radiation during normal aging, the number of circulating peripheral blood (PB) bone marrow-derived cells are calculated 3, 6, 9 and 12 months post-radiation. Circulating PB EPCs are evaluated by fluorescence-activated cell sorter (FACS) analysis, as described¹¹. The mononuclear fraction of PB blood is stained with VEGF receptor Flk1 and Sca1 antibodies (for EPCs) or RAM34 and CD45 (for HSCs) antibodies (and with corresponding IgG antibodies as negative controls) to process cells for FACS analysis. Flk1/Sca1 double-positive cells are used to compare the PB EPCs between different treatment groups. RAM34 and CD45 double positive cells are used to compare HSCs and hemangioblasts.

### Cardiomyocyte Preparation.

Adult cardiomyocytes are isolated from mice 3, 6, 9 and 12 months post-radiation using collagenase perfusion as described previously⁹⁴. Isolated myocytes are superfused with control solution containing (in mM) 137NaCl, 4.0HEPES, 0.5MgSO4, 3.7KCl, 1.5Ca²⁺, 5.6 glucose, and 0.5 probenecid, pH of 7.4.

### Radiation Effects on Resting Cytoplasmic [Ca²⁺].

To determine the effect of low-dose and high-dose space radiation, environmental, and therapeutic on long-term alterations in Ca²⁺ handling in cardiomyocytes, the resting cytoplasmic [Ca²⁺] is determined. Cardiomyocytes from the irradiated (IR) and non-irradiated (N-IR) mice are loaded with Fura-2AM, a ratiometric fluorescent Ca²⁺ dye. The ratio of the fluorescent intensities (340 nm and 380 nm) are used to quantify changes in the cytoplasmic [Ca²⁺]. Fura-2 fluorescence is calibrated in order to quantify the changes in [Ca²⁺]_{c} after the irradiation. The numbers of myocyte experiments are about 10-15 experiments from 5 hearts in each group.

### Radiation Effects on Cardiomyocyte Contractility.

Cardiomyocytes are loaded with the Ca²⁺-sensitive fluorescence indicator Fluo-3. Cell contraction and [Ca²⁺]i transients are measured simultaneously with a video and fluorescence microscopy system following described methods⁹⁵. Under baseline conditions, myocytes are placed with field stimulation at 0.5 Hz at 25° C. To study contractile reserve in response to an increase in pacing frequency, the pacing rate is increased from 1 to 5 Hz, in increments of 1 Hz, with constant extracellular Ca²⁺ concentration ([Ca²⁺] of 1.5 mmol/l. Measurements are performed 1 min after each change in pacing frequency. The numbers of myocyte experiments are about 10-15 experiments from 5 hearts in each group. In all experiments using fluo-3, fluorescence are excited at 480 nm and monitored at 535 nm. Calcium calibration are done using the fluorescence intensity (F) such that [Ca²⁺]i=Kd×(F−Fmin)/(Fmax−F), where Fmin=(Fmax−FBKG)/40+FBKG. Fmax=(F_{Mn}−FBKG)/0.2+FBKG, Fₘₙ is the fluorescence intensity from myocytes superfused with 10 mmol/l Mn solution, and FBKG is fluorescence from the field. The Kd of fluo-3 is 493 nmol/l at 25° C.

Radiation Effects on the Mitochondrial Membrane Potential (ΔΨₘ).

A proper ΔΨₘ is essential for mitochondrial activity and is an indicator for the health of mitochondria. Changes in ΔΨₘ as a measure of mitochondrial functional state are monitored. These experiments determine the effect of radiation on ΔΨₘ changes in resting cardiomyocytes from IR and N-IR mice. In these experiments mitochondria are co-labeled with a MitoTracker green FM (MTG), which accumulates within mitochondria regardless of ΔΨₘ, and TMRE probe, whose accumulation in mitochondria is driven by mitochondrial membrane potential. Simultaneous emission fluorescence of MTG and TMRE fluorescence are captured with laser scanning confocal microscopy. Results demonstrated that resting 1 Gy γ-radiated myocytes exhibit a minor, but statistically significant reduction in TMRE fluorescence, representing a loss of ΔΨₘ. To measure mitochondrial membrane potential, 10-15 experiments from 5 hearts in each group are performed.

Radiation Effects on Mitochondrial Ca²⁺ ([Ca²⁺]ₘ) Overload.

Chronic increase in resting [Ca²⁺]_{c} promote mitochondrial Ca²⁺ overload in irradiated cardiac muscle is determined by monitoring the mitochondrial permeability transition pore (PTP), whose opening rate is proportional to the Ca²⁺ overload. For these experiments cells are loaded with calcein-AM, which gets trapped within mitochondria and whose cytoplasmic signal can be quenched by addition of Co⁹⁶. The increase in the [Ca²⁺]ₘ leads to opening of PTP, which allows calcein to escape from the mitochondria. Monitoring the rate of calcein fluorescence decline in mitochondria in resting N-IR cells allows the establishment of the baseline of PTP opening rate and a benchmark of relative [Ca²⁺]ₘ. Without being bound to theory, irradiation-affected cells have elevated [Ca²⁺]ₘ, which promote an increase in PTP opening rate, resulting in increased rate of calcein efflux from the mitochondria. To measure radiation effects on mitochondrial Ca2+ ([Ca2+]m) overload, 10-15 experiments from 5 hearts/group are performed.

### Acute MI Studies, Post-AMI Survival, Functional Myocardial Recovery.

To evaluate post-acute myocardial infarction (AMI) survival, cardiac function, angiogenesis and recovery in series of experiment in murine model of AMI [ligation of the left anterior descending (LAD) coronary artery] model and age-matched N-IR controls, serial assessments of heart measurements and function are performed. For evaluation of infract size and left ventricular (LV) dimensions, transthoracic echocardiography are performed before surgery (LAD ligation), 7, 14 and 28 days after surgery. LV chamber remodeling and contractile function are assessed by measuring anterior and posterior wall thickness, LV systolic and diastolic dimensions, relative wall thickness and endocardial fractional shortening. Hemodynamic measurements are used to assess LV contractile indices, which include heart rate, LV systolic pressure. LV end-diastolic pressure, dP/dtmax, dP/dtmin and LV developed pressure. Infract size is calculated in the same view by measuring infarct segment length and dividing it by diastolic circumference (×100). Change in fractional area and infarct size is measured three times and the mean of these values used to make comparisons.

Post-AMI BM-Derived Cell (EPC, HSC, Hemanigoblast) Mobilization from Bone Marrow.

To determine alteration of AMI-mediated EPC mobilization from the BM by radiation, the number of circulating PB EPCs are calculated before, 3, 7 and 14 days post-AMI. Mobilized PB EPCs are evaluated by FACS (double Flk1/Sca1+ cells and RAM34/CD45+ cells) as described¹¹.

### Double Immunostaining for TUNEL and Isolectin/B4.

Before and 3, 7 and 14 days post-AMI surgery, hearts are harvested and preserved in OCT by freezing overnight to process later for serial sectioning. To evaluate apoptotic cells in the heart tissue of irradiated and control mice, double immunostaining of Isolectin/B4 (EC-marker) and TUNEL are performed, as described⁸⁴. To evaluate localization of apoptotic cells, an established, commercially available TUNEL kit (ApopTag) is used, as described⁸⁵.

### Post-AMI Inflammatory Responses in the Heart.

Before and 7, 14 and 28 days post-AMI surgery, hearts of non-irradiated control and irradiated mice are evaluated for expression of myeloperoxidase-1 (MPO-1), a neutrophil marker, and CD-68, a macrophages marker. The number of positive cells are quantified for each staining using dedicated software (Image-J, Wayne-Rasband, NIH).

### Measurement of Neovascularization in the Infarct and Infarct Border-Zone.

As a measure of collateral blood flow recovery after ischemia, capillary density is evaluated in myocardium of irradiated and control mice 7, 14 and 28 days after AMI surgery using two EC-specific markers CD31 (PECAM-1)⁹⁷ and Bandeurea simplicifolia (BS)-1 lectin conjugated to Rhodamine (Vector Laboratories)⁹⁸. To measure the functional (perfused) capillary network, 30 minutes before sacrifice a set of mice (5 per treatment group) are anesthetized and perfused with 0.5 mg (in 100 μl of isotonic solution) of Rhodamine-conjugated BS-1 lectin as described^{99,100}. To ensure that every endothelial cell is counted Z-stack mode of confocal microscopy are used and 10 or more images 0.5 μm thick acquired. Computer-assisted Image J (NIH software) is used to calculate the intensity of immunostaining in infarct myocardium.

### Statistical Analyses, Sample Size Calculations.

Results of experiments are expressed as mean±SEM. Comparisons between two means are performed with an unpaired Student's t-Test, while differences among groups are evaluated by ANOVA and Fisher's PLSD post hoc test using StatView software (SAS Institute Inc., Gary, N.C.). Differences will be considered significant at p<0.05. A Power and Sample Size Calculation program (version 2.1.23) are used to calculate the number of experimental animals needed to produce biologically valid and meaningful results. Based on our previous experience, preliminary data and on anticipated differences among groups and previously observed animal-to-animal variability with respect to the endpoints being tested, our sample size calculations suggest that approximately 10-15 mice are needed in each group for each time point in order to have a 90% probability of demonstrating differences at p=0.05, assuming that such differences exist.

### REFERENCES

The following documents are cited herein-above. As should be understood, the citations above to one or more numbers in superscript (e.g., ¹⁻³) refers to the document listed below identified by the same number.
1. Takahashi T, Kalka C, Masuda H, Chen D, Silver M, Kearney M, Magner M, Isner J M, Asahara T. Ischemia- and cytokine-induced mobilization of bone marrow-derived endothelial progenitor cells for neovascularization. Nature medicine. 1999; 5(4):434-438.
2. Orlic D, Kajstura J, Chimenti S, Jakoniuk I, Anderson S M, Li B, Pickel J, Mckay R, Nadal-Ginard B, Bodine D M, Leri A, Aniversa P. Bone marrow cells regenerate infarcted myocardium. Nature. 2001; 401:701-705.
3. Kalka C, Masuda H, Takahashi T, Kalka-Moll W M, Silver M, Kearney M, Li T, Isner J M, Asahara T. Transplantation of ex vivo expanded endothelial progenitor cells for therapeutic neovascularization. Proceedings of the National Academy of Sciences of the United States of America. 2000; 97(7):3422-3427.
4. Kamihata H, Matsubara H, Nishiue T, Fujiyama S, Tsutsumi Y, Ozono R, Masaki H, Mori Y, Iba O, Tateishi E, Kosaki A, Shintani S, Murohara T, Imaizumi T, Iwasaka T. Implantation of bone marrow mononuclear cells into ischemic myocardium enhances collateral perfusion and regional function via side supply of angioblasts, angiogenic ligands, and cytokines. Circulation. 2001; 104(9):1046-1052.
5. Fischer-Rasokat U, Assmus B, Seeger F H, Honold J, Leistner D, Fichtlscherer S, Schachinger V, Tonn T, Martin H, Dimmeler S, Zeiher A M. A pilot trial to assess potential effects of selective intracoronary bone marrow-derived progenitor cell infusion in patients with nonischemic dilated cardiomyopathy: final 1-year results of the transplantation of progenitor cells and functional regeneration enhancement pilot trial in patients with nonischemic dilated cardiomyopathy. Circ Heart Fail. 2009; 2(5):417-423.
6. Tateishi-Yuyama E, Matsubara H, Murohara T, Ikeda U, Shintani S, Masaki H, Amano K, Kishimoto Y, Yoshimoto K, Akashi H, Shimada K, Iwasaka T, Imaizumi T. Therapeutic angiogenesis for patients with limb ischaemia by autologous transplantation of bone-marrow cells: a pilot study and a randomised controlled trial. Lancet. 2002; 360(9331):427-435.
7. Schachinger V, Assmus B, Britten M B, Honold J, Lehmann R, Teupe C, Abolmaali N D, Vogl T J, Hofmann W K, Martin H, Dimmeler S, Zeiher A M. Transplantation of progenitor cells and regeneration enhancement in acute myocardial infarction: final one-year results of the TOPCARE-AMI Trial. J Am Coll Cardiol. 2004; 44(8):1690-1699.
8. van Ramshorst J, Bax J J, Beeres S L, Dibbets-Schneider P, Roes S D, Stokkel M P, de Roos A, Fibbe W E, Zwaginga J J, Boersma E, Schalij M J, Atsma D E. Intramyocardial bone marrow cell injection for chronic myocardial ischemia: a randomized controlled trial. Jama. 2009; 301(19):1997-2004.
9. Goukassian D, Diez-Juan A, Asahara T, Schratzberger P, Silver M, Murayama T, Isner J M, Andres V. Overexpression of p27(Kip1) by doxycycline-regulated adenoviral vectors inhibits endothelial cell proliferation and migration and impairs angiogenesis. Faseb J. 2001; 15(11):1877-1885.
10. Qin G, Ii M, Silver M, Wecker A, Bord E, Ma H, Gavin M, Goukassian D A, Yoon Y S, Papayannopoulou T, Asahara T, Kearney M, Thorne T, Curry C, Eaton L, Heyd L, Dinesh D, Kishore R, Zhu Y, Losordo D W. Functional disruption of alpha4 integrin mobilizes bone marrow-derived endothelial progenitors and augments ischemic neovascularization. The Journal of experimental medicine. 2006; 203(1):153-163.
11. Goukassian D A, Qin G, Dolan C, Murayama T, Silver M, Curry C, Eaton E, Luedemann C, Ma H, Asahara T, Zak V, Mehta S, Burg A, Thorne T, Kishore R, Losordo D W. Tumor necrosis factor-alpha receptor p75 is required in ischemia-induced neovascularization. Circulation. 2007; 115(6):752-762.
12. Kishore R T T, Sasi S P, Silver M, Gilbert H-Y, Yoon Y-S, Park H-Y, Thorne, T, Losordo W, Goukassian D A. Tumor Necrosis Factor-α Signaling Via TNFR1/p55 is Deleterious whereas TNFR2/p75 Signaling is Protective in Adult Infarct Myocardium. Proceeding of 12th International TNF Meeting. 2010; El-Escorial, Madrid, Spain; in press.
13. Kai T, Spradling A. An empty Drosophila stem cell niche reactivates the proliferation of ectopic cells. Proceedings of the National Academy of Sciences of the United States of America. 2003; 100(8):4633-4638.
14. Convertino V A. Status of cardiovascular issues related to space flight: Implications for future research directions. Respiratory physiology & neurobiology. 2009; 169 Suppl 1:S34-37.
15. Bungo M W, Goldwater D J, Popp R L, Sandler H. Echocardiographic evaluation of space shuttle crewmembers. J Appl Physiol. 1987; 62(1):278-283.
16. Convertino V A, Cooke W H, Lurie K G. Inspiratory resistance as a potential treatment for orthostatic intolerance and hemorrhagic shock. Aviation, space, and environmental medicine. 2005; 76(4):319-325.
17. Perhonen M A, Franco F, Lane L D, Buckey J C, Blomqvist C G, Zerwekh J E, Peshock R M, Weatherall P T, Levine B D. Cardiac atrophy after bed rest and spaceflight. J Appl Physiol. 2001; 91(2):645-653.
18. Levine B D, Zuckerman J H, Pawelczyk J A. Cardiac atrophy after bed-rest deconditioning: a nonneural mechanism for orthostatic intolerance. Circulation. 1997; 96(2):517-525.
19. Spaak J, Montmerle S, Sundblad P, Linnarsson D. Long-term bed rest-induced reductions in stroke volume during rest and exercise: cardiac dysfunction vs. volume depletion. J Appl Physiol. 2005; 98(2):648-654.
20. Fleissner F, Thum T. The IGF-1 receptor as a therapeutic target to improve endothelial progenitor cell function. Molecular medicine (Cambridge, Mass. 2008; 14(5-6):235-237.
21. Watson G E, Lorimore S A, Macdonald D A, Wright E G. Chromosomal instability in unirradiated cells induced in vivo by a bystander effect of ionizing radiation. Cancer research. 2000; 60(20):5608-5611.
22. Watson G E, Pocock D A, Papworth D, Lorimore S A, Wright E G. In vivo chromosomal instability and transmissible aberrations in the progeny of hemopoietic stem cells induced by high- and low-LET radiations. International journal of radiation biology. 2001; 77(4):409-417.
23. Todd P, Pecaut M J, Fleshner M. Combined effects of space flight factors and radiation on humans. Mutation research. 1999; 430(2):211-219.
24. Pant G S, Kamada N. Chromosome aberrations in normal leukocytes induced by the plasma of exposed individuals. Hiroshima journal of medical sciences. 1977; 26(2-3):149-154.
25. Emerit I, Levy A, Cernjavski L, Arutyunyan R, Oganesyan N, Pogosian A, Mejlumian H, Sarkisian T, Gulkandanian M, Quastel M, et al. Transferable clastogenic activity in plasma from persons exposed as salvage personnel of the Chernobyl reactor. Journal of cancer research and clinical oncology. 1994; 120(9):558-561.
26. Bose A S, Shetty V, Sadiq A, Shani J, Jacobowitz I. Radiation induced cardiac valve disease in a man from Chernobyl. J Am Soc Echocardiogr. 2009; 22(8):973 e971-973.
27. Gyenes G, Fornander T, Carlens P, Rutqvist L E. Morbidity of ischemic heart disease in early breast cancer 15-20 years after adjuvant radiotherapy. International journal of radiation oncology, biology, physics. 1994; 28(5):1235-1241.
28. Taylor C W, McGale P, Darby S C. Cardiac risks of breast-cancer radiotherapy: a contemporary view. Clinical oncology (Royal College of Radiologists (Great Britain)). 2006; 18(3):236-246.
29. Gayed I W, Liu H H, Yusuf S W, Komaki R, Wei X, Wang X, Chang J Y, Swafford J, Broemeling L, Liao Z. The prevalence of myocardial ischemia after concurrent chemoradiation therapy as detected by gated myocardial perfusion imaging in patients with esophageal cancer. J Nucl Med. 2006; 47(1):1756-1762.
30. Asahara T, Murohara T, Sullivan A, Silver M, van der Zee R, Li T, Witzenbichler B, Schatteman G, Isner J M. Isolation of putative progenitor endothelial cells for angiogenesis. Science (New York, N.Y. 1997; 275(5302):964-967.
31. Vasa M, Fichtischerer S, Adler K, Aicher A, Martin H, Zeiher A M, Dimmeler S. Increase in circulating endothelial progenitor cells by statin therapy in patients with stable coronary artery disease. Circulation. 2001.
32. Tepper O M, Galiano R D, Capla J M, Kalka C, Gagne P J, Jacobowitz G R, Levine J P, Gurtner G C. Human endothelial progenitor cells from type II diabetics exhibit impaired proliferation, adhesion, and incorporation into vascular structures. Circulation. 2002; 106(22):2781-2786.
33. Hill J M, Zalos G, Halcox J P, Schenke W H, Waclawiw M A, Quyyumi A A, Finkel T. Circulating endothelial progenitor cells, vascular function, and cardiovascular risk. The New England journal of medicine. 2003; 348(7):593-600.
34. Loomans C J, de Koning E J, Staal F J, Rookmaaker M B, Verseyden C, de Boer H C, Verhaar M C, Braam B, Rabelink T J, van Zonneveld A J. Endothelial progenitor cell dysfunction: a novel concept in the pathogenesis of vascular complications of type 1 diabetes. Diabetes. 2004; 53(1):195-199.
35. Ivanova N B, Dimos J T, Schaniel C, Hackney J A, Moore K A, Lemischka I R. A stem cell molecular signature. Science (New York, N.Y. 2002; 298(5593):601-604.
36. Ramalho-Santos M, Yoon S, Matsuzaki Y, Mulligan R C, Melton D A. “Stemness”: transcriptional profiling of embryonic and adult stem cells. Science (New York, N.Y. 2002; 298(5593):597-600.
37. Dernbach E, Urbich C, Brandes R P, Hofmann W K, Zeiher A M, Dimmeler S. Antioxidative stress-associated genes in circulating progenitor cells: evidence for enhanced resistance against oxidative stress. Blood. 2004; 104(12):3591-3597.
38. He T, Peterson T E, Holmuhamedov E L, Terzic A, Caplice N M, Oberley L W, Katusic Z S. Human endothelial progenitor cells tolerate oxidative stress due to intrinsically high expression of manganese superoxide dismutase. Arteriosclerosis, thrombosis, and vascular biology. 2004; 24(11):2021-2027.
39. Raes M, Michiels C, Remacle J. Comparative study of the enzymatic defense systems against oxygen-derived free radicals: the key role of glutathione peroxidase. Free radical biology & medicine. 1987; 3(1):3-7.
40. Forgione M A, Weiss N, Heydrick S, Cap A, Klings E S, Bierl C, Eberhardt R T, Farber H W, Loscalzo J. Cellular glutathione peroxidase deficiency and endothelial dysfunction. American journal of physiology. 2002; 282(4):H1255-1261.
41. Cramer T, Yamanishi Y, Clausen B E, Forster I, Pawlinski R, Mackman N, Haase V H, Jaenisch R, Corr M, Nizet V, Firestein G S, Gerber H P, Ferrara N, Johnson R S. HIF-1alpha is essential for myeloid cell-mediated inflammation. Cell. 2003; 112(5):645-657.
42. Pandey R, Shankar B S, Sharma D, Sainis K B. Low dose radiation induced immunomodulation: effect on macrophages and CD8+ T cells. International journal of radiation biology. 2005; 81(11):801-812.
43. Wright E G, Coates P J. Untargeted effects of ionizing radiation: implications for radiation pathology. Mutation research. 2006; 597(1-2):119-132.
44. Lorimore S A, Coates P J, Scobie G E, Milne G, Wright E G. Inflammatory-type responses after exposure to ionizing radiation in vivo: a mechanism for radiation-induced bystander effects? Oncogene. 2001; 20(48):7085-7095.
45. Mungrue I N, Gros R, You X, Pirani A, Azad A, Csont T, Schulz R, Butany J, Stewart D J, Husain M. Cardiomyocyte overexpression of iNOS in mice results in peroxynitrite generation, heart block, and sudden death. The Journal of clinical investigation. 2002; 109(6):735-743.
46. Kojda G, Laursen J B, Ramasamy S, Kent J D, Kurz S, Burchfield J, Shesely E G, Harrison D G. Protein expression, vascular reactivity and soluble guanylate cyclase activity in mice lacking the endothelial cell nitric oxide synthase: contributions of NOS isoforms to blood pressure and heart rate control. Cardiovascular research. 1999; 42(1):206-213.
47. Aikawa R, Komuro I, Yamazaki T, Zou Y, Kudoh S, Tanaka M, Shiojima I, Hiroi Y, Yazaki Y. Oxidative stress activates extracellular signal-regulated kinases through Src and Ras in cultured cardiac myocytes of neonatal rats. The Journal of clinical investigation. 1997; 100(7):1813-1821.
48. Denton R M, McCormack J G. Physiological role of Ca2+ transport by mitochondria. Nature. 1985; 315(6021):635.
49. Denton R M, McCormack J G. The calcium sensitive dehydrogenases of vertebrate mitochondria. Cell Calcium. 1986; 7(5-6):377-386.
50. Rutter G A, Burnett P, Rizzuto R, Brini M, Murgia M, Pozzan T, Tavare J M, Denton R M. Subcellular imaging of intramitochondrial Ca2+ with recombinant targeted aequorin: significance for the regulation of pyruvate dehydrogenase activity. Proc Natl Acad Sci USA. 1996; 93(11):5489-5494.
51. Wan B, LaNoue K F, Cheung J Y, Scaduto R C, Jr. Regulation of citric acid cycle by calcium. J Biol Chem. 1989; 264(23):13430-13439.
52. Lawlis V B, Roche T E. Regulation of bovine kidney alpha-ketoglutarate dehydrogenase complex by calcium ion and adenine nucleotides. Effects on S0.5 for alpha-ketoglutarate. Biochemistry. 1981; 20(9):2512-2518.
53. Das A M, Byrd D J, Brodehl J. Regulation of the mitochondrial ATP-synthase in human fibroblasts. Clin Chim Acta. 1994; 231(1):61-68.
54. Das A M. Regulation of mitochondrial ATP synthase activity in human myocardium. Clin Sci (Lond). 1998; 94(5):499-504.
55. Mildaziene V, Baniene R, Nauciene Z, Bakker B M, Brown G C, Westerhoff H V, Kholodenko B N. Calcium indirectly increases the control exerted by the adenine nucleotide translocator over 2-oxoglutarate oxidation in rat heart mitochondria. Arch Biochem Biophys. 1995; 324(1):130-134.
56. Brookes P S, Yoon Y, Robotham J L, Anders M W, Sheu S S. Calcium, ATP, and ROS: a mitochondrial love-hate triangle. Am J Physiol Cell Physiol. 2004; 287(4):C817-833.
57. Crompton M. The mitochondrial permeability transition pore and its role in cell death. Biochem J. 1999; 341 (Pt 2):233-249.
58. Huser J, Blatter L A. Fluctuations in mitochondrial membrane potential caused by repetitive gating of the permeability transition pore. Biochem J. 1999; 343 Pt 2:311-317.
59. O'Reilly C M, Fogarty K E, Drummond R M, Tuft R A, Walsh J V, Jr. Quantitative analysis of spontaneous mitochondrial depolarizations. Biophys J. 2003; 85(5):3350-3357.
60. Ichas F, Mazat J P. From calcium signaling to cell death: two conformations for the mitochondrial permeability transition pore. Switching from low- to high-conductance state. Biochim Biophys Acta. 1998; 1366(1-2):33-50.
61. Wang W, Fang H, Groom L, Cheng A, Zhang W, Liu J, Wang X, Li K, Han P, Zheng M, Yin J, Wang W, Mattson M P, Kao J P, Lakatta E G, Sheu S S, Ouyang K, Chen J, Dirksen R T, Cheng H. Superoxide flashes in single mitochondria. Cell. 2008; 134(2):279-290.
62. Chen Q, Chai Y C, Mazumder S, Jiang C, Macklis R M, Chisolm G M, Almasan A. The late increase in intracellular free radical oxygen species during apoptosis is associated with cytochrome c release, caspase activation, and mitochondrial dysfunction. Cell Death Differ. 2003; 10(3):323-334.
63. Rothkamm K, Lobrich M. Evidence for a lack of DNA double-strand break repair in human cells exposed to very low x-ray doses. Proceedings of the National Academy of Sciences of the United States of America. 2003; 100(9):5057-5062.
64. Lorimore S A, Coates P J, Wright E G. Radiation-induced genomic instability and bystander effects: inter-related nontargeted effects of exposure to ionizing radiation. Oncogene. 2003; 22(45):7058-7069.
65. Morgan W F, Sowa M B. Non-targeted bystander effects induced by ionizing radiation. Mutation research. 2007; 616(1-2):159-164.
66. Hei T K, Zhou H, Ivanov V N, Hong M, Lieberman H B, Brenner D J, Amundson S A, Geard C R. Mechanism of radiation-induced bystander effects: a unifying model. The Journal of pharmacy and pharmacology. 2008; 60(8):943-950.
67. Little J B. Cellular radiation effects and the bystander response. Mutation research. 2006; 597(1-2):113-118.
68. Rzeszowska-Wolny J, Przybyszewski W M, Widel M. Ionizing radiation-induced bystander effects, potential targets for modulation of radiotherapy. European journal of pharmacology. 2009; 625(1-3):156-164.
69. Azzam E I, de Toledo S M, Little J B. Direct evidence for the participation of gap junction-mediated intercellular communication in the transmission of damage signals from alpha-particle irradiated to nonirradiated cells. Proceedings of the National Academy of Sciences of the United States of America. 2001; 98(2):473-478.
70. Azzam E I, de Toledo S M, Little J B. Expression of CONNEXIN43 is highly sensitive to ionizing radiation and other environmental stresses. Cancer research. 2003; 63(21):7128-7135.
71. Little J B, Lauriston S. Taylor lecture: nontargeted effects of radiation: implications for low-dose exposures. Health physics. 2006; 91(5):416-426.
72. Morgan W F. Non-targeted and delayed effects of exposure to ionizing radiation: I. Radiation-induced genomic instability and bystander effects in vitro. Radiation research. 2003; 159(5):567-580.
73. Ballarini F, Alloni D, Facoetti A, Mairani A, Nano R, Ottolenghi A. Modelling radiation-induced bystander effect and cellular communication. Radiation protection dosimetry. 2006; 122(1-4):244-251.
74. Sowa M B, Goetz W, Baulch J E, Pyles D N, Dziegielewski J, Yovino S, Snyder A R, de Toledo S M, Azzam E I, Morgan W F. Lack of evidence for low-LET radiation induced bystander response in normal human fibroblasts and colon carcinoma cells. International journal of radiation biology. 86(2):102-113.
75. Tang W, Willers H, Powell S N. p53 directly enhances rejoining of DNA double-strand breaks with cohesive ends in gamma-irradiated mouse fibroblasts. Cancer research. 1999; 59(11):2562-2565.
76. Mendiola-Cruz M T, Morales-Ramirez P. Repair kinetics of gamma-ray induced DNA damage determined by the single cell gel electrophoresis assay in murine leukocytes in vivo. Mutation research. 1999; 433(1):45-52.
77. Boerrigter M E, Dolle M E, Martus H J, Gossen J A, Vijg J. Plasmid-based transgenic mouse model for studying in vivo mutations. Nature. 1995; 377(6550):657-659.
78. Goukassian D A, Helms E, van Steeg H, van Oostrom C, Bhawan J, Gilchrest B A. Topical DNA oligonucleotide therapy reduces UV-induced mutations and photocarcinogenesis in hairless mice. Proc Natl Acad Sci USA. 2004; 101(11):3933-3938.
79. Goukassian D A, Gilchrest B A. The interdependence of skin aging, skin cancer, and DNA repair capacity: a novel perspective with therapeutic implications. Rejuv Res. 2004; 7(3):175-185.
80. Zattra E, Coleman C, Arad S, Helms E, Levine D, Bord E, Guillaume A, El-Hajahmad M, Zwart E, van Steeg H, Gonzalez S, Kishore R, Goukassian D A. Polypodium leucotomos extract decreases UV-induced Cox-2 expression and inflammation, enhances DNA repair, and decreases mutagenesis in hairless mice. The American journal of pathology. 2009; 175(5):1952-1961.
81. van Gent D C, Hoeijmakers J H, Kanaar R. Chromosomal stability and the DNA double-stranded break connection. Nature reviews. 2001; 2(3):196-206.
82. Rappold I, Iwabuchi K, Date T, Chen J. Tumor suppressor p53 binding protein 1 (53BP1) is involved in DNA damage-signaling pathways. The Journal of cell biology. 2001; 153(3):613-620.
83. Kurushima H, Ramprasad M, Kondratenko N, Foster D M, Quehenberger O, Steinberg D. Surface expression and rapid internalization of macrosialin (mouse CD68) on elicited mouse peritoneal macrophages. Journal of leukocyte biology. 2000; 67(1):104-108.
84. Botchkareva N V, Khlgatian M, Longley B J, Botchkarev V A, Gilchrest B A. SCF/c-kit signaling is required for cyclic regeneration of the hair pigmentation unit. Faseb J. 2001; 15(3):645-658.
85. Botchkarev V A, Welker P, Albers K M, Botchkareva N V, Metz M, Lewin G R, Bulfone-Paus S, Peters E M, Lindner G, Paus R. A new role for neurotrophin-3: involvement in the regulation of hair follicle regression (catagen). Am J Pathol. 1998; 153(3):785-799.
86. Mothersill C. Seymour C. Survival of human epithelial cells irradiated with cobalt 60 as microcolonies or single cells. International journal of radiation biology. 1997; 72(5):597-606.
87. Little M P, Tawn E J, Tzoulaki I, Wakeford R, Hildebrandt G, Paris F, Tapio S, Elliott P. A systematic review of epidemiological associations between low and moderate doses of ionizing radiation and late cardiovascular effects, and their possible mechanisms. Radiation research. 2008; 169(1):99-109.
88. Vijg J. Somatic mutations and aging: a re-evaluation. Mutation research. 2000; 447(1):117-135.
89. Dolle M E, Martus H J, Novak M, van Orsouw N J, Vijg J. Characterization of color mutants in lacZ plasmid-based transgenic mice, as detected by positive selection. Mutagenesis. 1999; 14(3):287-293.
90. Quijano C, Castro L, Peluffo G, Valez V, Radi R. Enhanced mitochondrial superoxide in hyperglycemic endothelial cells: direct measurements and formation of hydrogen peroxide and peroxynitrite. American journal of physiology. 2007; 293(6):H3404-3414.
91. Yoshida S, Ono M, Shono T, Izumi H, Ishibashi T, Suzuki H, Kuwano M. Involvement of interleukin-8, vascular endothelial growth factor, and basic fibroblast growth factor in tumor necrosis factor alpha-dependent angiogenesis. Mol Cell Biol. 1997; 17(7):4015-4023.
92. Asahara T, Takahashi T, Masuda H, Kalka C, Chen D, Iwaguro H, Inai Y, Silver M, Isner J M. VEGF contributes to postnatal neovascularization by mobilizing bone marrow-derived endothelial progenitor cells. EMBO J. 1999; 18:3964-3972.
93. Hamada H, Kim M K, Iwakura A, Ii M, Thorne T, Qin G, Asai J, Tsutsumi Y, Sekiguchi H, Silver M, Wecker A, Bord E, Zhu Y, Kishore R, Losordo D W. Estrogen receptors alpha and beta mediate contribution of bone marrow-derived endothelial progenitor cells to functional recovery after myocardial infarction. Circulation. 2006; 114(21):2261-2270.
94. Tajima M, Weinberg E O, Bartunek J, Jin H, Yang R, Paoni N F, Lorell B H. Treatment with growth hormone enhances contractile reserve and intracellular calcium transients in myocytes from rats with postinfarction heart failure. Circulation. 1999; 99(1):127-134.
95. Ito K, Yan X, Tajima M, Su Z, Barry W H, Lorell B H. Contractile reserve and intracellular calcium regulation in mouse myocytes from normal and hypertrophied failing hearts. Circulation research. 2000; 87(7):588-595.
96. Petronilli V, Miotto G, Canton M, Brini M, Colonna R, Bernardi P, Di Lisa F. Transient and long-lasting openings of the mitochondrial permeability transition pore can be monitored directly in intact cells by changes in mitochondrial calcein fluorescence. Biophys J. 1999; 76(2):725-734.
97. Qin G, Kishore R, Dolan C M, Silver M, Wecker A, Luedemann C N, Thorne T, Hanley A, Curry C, Heyd L, Dinesh D, Kearney M, Martelli F, Murayama T, Goukassian D A, Zhu Y, Losordo D W. Cell cycle regulator E2F1 modulates angiogenesis via p53-dependent transcriptional control of VEGF. Proc Natl Acad Sci USA. 2006; 103(29):11015-11020.
98. Goukassian D A, Diez-Juan, A., Asahara, T., Schratzber er. P., Silver, M., Murayama, T., Isner, J. M., & Andres, V. Overexpression of p27^{Kip1} by doxycycline-regulated adenoviral vectors inhibits endothelial cell proliferation and migration and impairs angiogenesis. FASEB J. 2001; 15(In press):1877-1885.
99. Alroy J, Goyal V, Skutelsky E. Lectin histochemistry of mammalian endothelium. Histochemistry. 1987; 86(6):603-607.
100. Asahara T, Chen D, Takahashi T, Fujikawa K, Kearney M, Magner M, Yancopoulos G D, Isner J M. Tie2 receptor ligands, angiopoietin-1 and angiopoietin-2, modulate VEGF-induced postnatal neovascularization. Circ Res. 1998; 83(3):233-240.

### Example 6

### TNF-α and IL-1α but not MCP-1 and Rantes Increase Significantly the Formation of p-H2AX Foci in Naïve BM-Derived TNFR1/p55KO EPC

Tumor necrosis factor-α (TNF) binds two receptors TNFR1/p55 and TNFR2/p75 and activates several signaling cascades. Ionizing radiation (IR) increases tissue levels of TNF. TNF signaling regulates numerous cytokines and chemokines that are known to mediate radiation-induced non-targeted effects (NTEs), a phenomenon where cells that are not directly ‘hit’ by IR exhibit IR effects as a result of signals received from nearby or distant IR cells. Little is known about the role of p55 or p75 in regulating NTE in bone marrow (BM) cells, specifically in BM-derived endothelial progenitor cells (EPCs). In media transfer experiments, we have shown that compared with WT EPCs, early NTEs (within 1-5 h) are inhibited in p55KO and p75KO EPCs, whereas delayed NTEs (within 3-5 days) are amplified in p55KO and to a lesser degree in p75KO EPCs, suggesting significant role of TNFR/p75 signaling (the remaining active receptor in p55KO EPCs) in mediating delayed NTEs. It was hypothesized that signaling through TNFR2/p75 may alter radiation-induced TNF-mediated inflammatory response increasing tissue levels of various cytokines, chemokines and growth factors that could then induce NTE, possibly, via activation of NFkB and other stress response transcription factors.

### Methods:

To test the hypothesis ex vivo, expanded p55KO EPCs were irradiated with 1 Gy of γ-IR, then IR-conditioned medium (CM) was collected at 1, 5, 24 h, and 3, 5 days post-IR. CM from IR p55KO EPCs were processed for multiplex ELISA (12 proteins). After determining concentrations of each of 12 proteins in control and IR-CM media of p55KO EPCs over 5 days, we treated naïve p55KO EPCs with various concentrations of four mouse recombinant (rm) proteins that were steadily increased in IR-CM between Days 3-5. After 24 h incubation, naïve p55KO EPCs were stained with anti-p-H2AX antibodies and the formation of p-H2AX foci was visualized at ×100 magnification using laser scanning confocal microscopy. The p-H2AX foci were quantified manually by a single investigator blindfolded to the treatment conditions and were confirmed using computer-assisted algorithm.

### Results

ELISA profiling of 12 proteins in IR-CM over 5 days post-IR showed 200-1600% increases (P<0.02, at least, p55KO vs WT, Days 3-5) in cumulative levels of TNF, IFNr, IL1α, IL1β, IL6, EGF, MIP-1α, MCP-1, GCSF, GM-CSF, Rantes and Leptin. The steadiest and the highest increases between Days 3 and 5 were observed in IL-1α, MCP-1 and Rantes.

Naïve p55KO EPCs were then treated ex vivo with concentrations determined in the ELISA: IL-1α (290, 580 pg/ml), MCP-1 (580, 1160, 2900 pg/ml), Rantes (600, 1500 pg/ml) and TNF (100 pg/ml, 1, 40 ng/ml). After 24 h incubation with rm proteins, p55KO EPCs were stained with anti-p-H2AX antibodies. The cells were imaged and the quantification of the p-H2AX foci was performed as described above.

Results showed that the mean p-H2AX foci count of MCP-1 and Rantes was not significantly different from control which had a mean of 0.98 p-H2AX foci/cell count, with the exception of MCP-1 at 1160 pg/ml (P<0.03, mean foci count of 1.9). TNF-treated naïve p55KO EPCs showed a significant increase in the mean p-H2AX foci/cell count at all concentration compared with the control, MCP-1 and Rantes (P<0.0001 with the mean p-H2AX foci ranging from 2.8 to 3.9).

IL1α-treated p55KO EPCs showed the greatest increase in p-H2AX foci with the mean foci count of 7.1 at 290 pg/ml and 9.3 at 580 pg/ml, and was significantly different from all tested mouse recombinant proteins. Analysis of p-H2AX foci distribution of EPCs with one or more foci showed that in control p55KO EPCs, <1% of cells had a maximum of 4-9 p-H2AX foci/cell. Whereas in TNF- and IL-1α-treated p55KO EPCs, >2% and >4% of cells had 9-18 foci/cell, respectively. Remarkably, 1% of cells had as many as 18-31 foci/cell for TNF-treated cells and as many as 19-51 foci/cell for IL-1α-treated p55KO EPCs.

Thus, TNF-TNFR2/p75 axis induces NTEs in naïve BM-EPCs, which suggests that blocking/neutralizing TNFR2/p75 signaling represent a mitigating measure for prevention of delayed NTEs, specifically, in BM-derived EPCs and, conceivably, in BM milieu in general.

### Example 7

### TNF-TNFR2/p75 Signaling Inhibits Early and Increases Delayed Non-Targeted Effects in Bone Marrow-Derived Endothelial Progenitor Cells

Ionizing radiation can induce DNA damage in non-irradiated (N-IR) cells via non-targeted effects (NTE). As described in detail below, TNF-α and IL1-α mediate NTE in N-IR bone marrow-derived EPCs and neutralizing TNF-α diminishes NTE in WT and p55 knockout BM-derived EPCs. These results indicate that TNF-TNFR2/p75 signaling alters accumulation of inflammatory cytokines that attenuate NTE in N-IR EPCs. Thus, TNFR2/p75 is a gene target for mitigation of delayed RBR in BM-derived EPCs.

TNF-α, a pro-inflammatory cytokine, is highly expressed after ionizing radiation (IR) and is implicated in mediating radiobiological bystander responses (RBR). Little is known about specific TNF receptors in regulating TNF-induced RBR in bone marrow-derived endothelial progenitor cells (BM-EPCs). Full-body γ-IR wild type (WT) BM-EPCs showed biphasic response: slow decay of p-H2AX foci during initial 24 h and increase between 24 h and 7 days post-IR, indicating a significant RBR in BM-EPCs in vivo. Individual TNF receptor signaling in RBR was evaluated in BM-EPCs from WT, TNFR1/p55KO and TNFR2/p75KO mice, in-vitro. Compared to WT, early RBR (1-5 h) were inhibited in p55KO and p75KO EPCs, whereas delayed RBR (3-5 d) were amplified in p55KO EPCs, suggesting possible role for TNFR2/p75 signaling in delayed RBR. Neutralizing TNF in γ-IR conditioned media (CM) of WT and p55KO BM-EPCs largely abolished RBR in both cell types. ELISA protein profiling of WT and p55KO EPC γ-IR-CM over 5 d showed significant increases in several pro-inflammatory cytokines, including TNF-α, IL-1α, Rantes and MCP1. In-vitro treatments with murine recombinant (rm) rmTNF-α and rmIL-1α, but not rmMCP-1 or rmRantes increased the formation of p-H2AX foci in N-IR p55KO EPCs. It is concluded that TNF-TNFR2 signaling may induce RBR in naïve BM-EPCs and that blocking TNF-TNFR2 signaling may prevent delayed RBR in BM-derived EPCs, conceivably, in bone marrow milieu in general.

Radiation-induced bystander responses (RBR) are defined as the induction of biological effects in cells that were not directly traversed by an ionized particle, but were affected due to their proximity to cells that were directly exposed to radiation (1-7). RBR effects are well documented in vitro in variety of cell cultures (8-11). These responses have been shown by various methodologies, such as, media transfer experiments (12,13), co-cultures of irradiated (IR) and Non-Irradiated (N-LR) cells (14,15), microbeam studies (16) and animal models in vivo (11). It has been proposed that RBR is mediated by an initiating event near the cell surface that activates and integrates numerous intracellular signaling pathways followed by activation of transcription factors and expression of genes that mediate RBR (7). Based on the previous investigations, it is evident that there appears to be a significant cell specificity in both, the ability to induce the RBR (11) and to receive the secreted signals (8). This suggests that in addition to the ability of IR cells to release cytokines, chemokines and growth factors, the ligand-receptor interaction on N-IR cells may also play an important role in propagation of the bystander response (3,8-10).

Low linear energy transfer (low-LET) radiation such as gamma-irradiation (γ-IR), has been reported to induce a bystander effect in glioblastoma cells (3). A more recent report found no evidence for low-LET induction of bystander responses in normal human fibroblast and colon carcinoma cells (17). Therefore, it is apparent that in addition to many factors that may influence bystander responses, including, but not limited to production and release of inflammatory cytokines and chemokines, such as tumor necrosis factor-α (TNF-α), interleukin-1α (IL-α) and others (9), there is a large intrinsic variability for bystander responses in different primary and tumor cells. Full body low-dose radiation such as X-ray and gamma-IR has been found to induce apoptotic and immunological responses in various organ and tissues, including bone marrow (18). The acute phase is usually characterized by neutrophil infiltration of the affected area, whereas macrophages are responsible for the phagocytic clearance of the apoptotic cells (19,20). It was shown that phagocytosis of IR-induced apoptotic cells can activate macrophages, leading to their induction of an inflammatory response in the surrounding tissue (21). This is mediated by a release of various cytokines, superoxide and nitric oxide (8). All of which are capable of causing tissue damage (22) by signaling through pro-apoptosis mediator TNF-α, Fas ligand (FasL), nitric oxide and superoxide (23,24).

TNF-α is a pro-inflammatory cytokine whose expression is known to be highly up-regulated in many tissues and cells after IR (23,25). TNF-α is a 17 kDa polypeptide that specifically binds and exerts its function via two cell surface receptors, TNFR1 (p55) and TNFR2 (p75). Each TNF receptor has been shown to activate distinct signaling pathways with a small degree of overlap (26,27). Functions of TNFR1/p55 have been well-studied and described (28,29). TNFR1/p55 is responsible for signaling a variety of responses predominantly cytotoxic, such as apoptosis and cell death, but also regulates inflammatory responses including cytokine secretion (30-33). In contrast. TNFR2/p75 is generally pro-survival and pro-angiogenic and responsible for cell protective effects of TNF, but regulates inflammatory signaling, as well (30,31,33-35). Both TNF receptors are ubiquitously expressed on nearly all cell types, but the p75 receptor is predominantly expressed by lymphoid cells as well as other hematopoietic and endothelial lineage cells, including endothelial progenitor cells (EPCs) (27,36,37). TNF induces inflammation via activation of transcription factor NF-κB and its downstream targets—COX-2, MMP1, IL1-α, IL1-β, IL6, IL8, IL33, IGF1 and TNF itself, along with many other cytokines (9). Many of these cytokines, chemokines and inflammatory enzymes (e.g., COX-2) are implicated in mediating RBR in variety of cells (38). However, the role of TNF receptors, p55 or p75, in regulating RBR in endothelial lineage cells, specifically in endothelial progenitor cells (EPCs), is largely unknown.

A growing body of evidence indicates that neovascularization involves both, the proliferation of local endothelial cells (EC) as well as mobilization, recruitment and proliferation of the endothelial progenitor cells (EPC) (39-43). EPCs have been shown to be proliferating clonally and be capable of migrating and differentiating into ECs (44-48). In various animal models (48-52) and human clinical trials (53-56) it has been shown that transplantation of EPCs leads to migration and homing of these cells to the areas of injury the ischemic injury such as acute myocardial infarction (AMI). Improved neovascularization and increased blood flow was observed in surgically-induced hind limb ischemia (HLI) model with infusion of ex vivo expanded EPCs (39,41,48,54,57). In green fluorescent protein (GFP) bone marrow transplanted (BMT) wild type mice using HLI model our laboratory has shown that 60-70% of GFP-positive cells mobilized from the BM to ischemic limbs were BM-derived endothelial lineage cells (35). Thus, without being bound to a particular theory, if EPCs are critical to endothelial maintenance and repair, radiation-induced EPC dysfunction could impair the recovery after ischemic injury or trauma.

We hypothesized that inhibition of TNF-ligand-receptor interactions may alter TNF-mediated downstream signaling, thereby affecting regulation of inflammatory cytokines and chemokines. Increased levels of IR-induced cytokines, chemokines and growth factors may then augment non-targeted effects in nearby cells not traversed directly by ionizing radiation, as well as in cells and tissues distant from the initial irradiation site, hence propagate bystander responses.

### Experimental Procedures

Animal Model—Mice used in this study were all 8-12 weeks old. They included WT (C57BL/6J-control of the mixed C57BL/6 and 129 background strains defined by the vendor as N10F34, meaning that these two strains were backcrossed 10 times ([N10 is number of backcross generations] and inbreed 34 times [F34 is number of filial or inbreeding generations]), TNFR2/p75 knockout (KO) (B6.129S2-Tnfrsf1btm1Mwm/J) and TNFR1/p55KO (B6.129-Tnfrsf1atm1Mak/J) were purchased from the Jackson Laboratory (Bar Harbor, Me., USA). Mice were fed standard laboratory chow diet (Harlan Teklad), and given water access ad libitum and kept in the temperature-controlled and light controlled (12 hour light/dark cycles) environment.

Isolation, Culture and Characterization of Bone-marrow derived Endothelial Progenitor Cells—Young WT mice were euthanized and EPCs were isolated from mononuclear faction of bone marrow cells using density gradient centrifugation and cultured in 6-well dishes (Corning Inc., Corning, N.Y., USA) on 22 mm×22 mm square glass coverslips (Fisher HealthCare, Houston, Tex., USA) coated with 0.2% gelatin (SIGMA, St Louis, Mo., USA). EPCs were expanded ex vivo in selective EBM2 growth medium (Lonza, Walkersville, Pa., USA) supplemented with growth factor as described previously (35,41,58). Upon attaining 70-80% confluence, cells were trypsinized using 0.25% Trypsin (Genesee Scientific, San Diego, Calif., USA) and processed for FACS analysis using EPC markers.

Immunoflourescent Characterization of Bone-marrow derived Endothelial Progenitor Cells—BM-derived EPCs expanded ex vivo to ˜70-80% confluence on glass cover slips as described in the paragraph above. To characterize our bone marrow-derived cell cultures, glass coverslips on which EPCs were cultured were fixed in 4% paraformaldehyde (PFA, FD NeuroTechnologies Inc., Columbia, Md., USA) for 15 min at room temperature (RT) and washed with ice-cold 1× phosphate buffered saline (PBS, MediaTech, Herndon, Va., USA) for 5 min. Fixed cells were permeabilized with 0.1% Triton X-100 (SIGMA) for 15 min at RT and washed three times in 1×PBS for 5 min. To determine the stem or progenitor nature and to confirm endothelial cell lineage of cells in our ex-vivo expanded BM marrow-derived cultures cells on cover slips were triple stained with rat anti-mouse Sca-1 (Cat.sc-52601, Santa Cruz Biotechnology Inc., Dallas, Tex., USA) or rat anti-mouse c-kit (Cat. 553868, BD Pharmingen, San Jose, Calif., USA) and Biotinylated Isolectin-B4 (Cat.I21414, Life Technologies. Carlsbad, Calif., USA) along with TopRo3 (Cat.T3605; Life Technologies) to visualize nuclei. Alexa-488 Goat anti-rat (Cat.A11006, Life Technologies) secondary antibody was used for both Sca-1 and c-kit, while Alexa-594 labeled streptavidin (Cat.S11227, Life Technologies) was used as secondary antibody for Isolectin-B4. In addition, to determine the purity and a possible hematopoietic “contamination” of our BM-derived EPCs cultures we have performed immunofluorescent staining of our BM-derived cultures with a panel of mouse hematopoietic anti-mouse antibodies for. Alexa-488 conjugated rat anti-mouse Gr1/Ly-6G (neutrophils) (Cat. 108419, BioLegend, San Diego, Calif., USA), rat anti-mouse F4/80 (macrophages and blood monocytes) (Cat. 123101, BioLegend). B220 (B lymphocytes), CD3ε (T lymphocytes), TER-119 (erythrocytes and erythroid precursors) (Cat. 88-7774-75, eBiosciences, San Diego, Calif., USA) and endothelial cell marker Isolectin-B4 (Life Technologies) (staining), as well as uptake while still in culture of the Acetylated Low Density Lipoprotein, labeled with 1,1′-dioctadecyl-3,3,3′,3′-tetramethyl-indocarbocyanine perchlorate (DiI-ac-LDL, Cat.BT-902, Biomedical Technologies Inc., Stoughton, Mass., USA). Please note that DiI-ac-LDL is reported to label both vascular endothelial cells and bone marrow-derived macrophages. Alexa-488 Goat anti-rat (Life Technologies) secondary antibody was used for F4/80, while FITC-labeled streptavidin (Cat. 11-4317-87, eBiosciences) was used as secondary antibody for staining mouse hematopoietic lineage panel and Alexa-594 labeled streptavidin (Life Technologies) was used as secondary antibody for Isolectin-B4. Corresponding IgG antibodies were also used as negative control to confirm the specificity of the primary antibodies. Images were obtained using laser scanning confocal microscope (LSM 510 Meta, ZEISS, Thornwood, N.Y., USA) at ×20 magnification.

Irradiation and Dosimetry—Prior to full-body irradiation (IR) each animals was placed individually into a rectangular polypropylene box with multiple air holes (3 mm in diameter) for stress free environment. Typically eight un-anesthetized mice were irradiated simultaneously. All gamma-irradiation (γ-IR) experiments were performed at Steward St Elizabeth's Medical Center using a Cesium (137Cs) source irradiator to yield a total single full-body dose of 1 Gy at an average dose rate of 46.6 cGy/min.

BM-derived EPC culture from full-body irradiated WT mice—Young WT mice were exposed to 1 Gy full-body T-IR as described above. Both, N-IR control and γ-IR WT mice were euthanized 30 min, 24 hours or 7 days post-IR. EPCs were isolated from mononuclear fraction of bone marrow cells and expanded ex vivo until the cells attained ˜70% confluence as described previously (35,41,58). Upon attaining confluence, cells were fixed on glass coverslips and processed for p-H2AX immunostaining.

Media Transfer Experiments in WT BM-derived EPCs—Two sets of ˜70% confluent EPCs from WT mice were derived from each animal. Within each preparation, one set of EPCs were γ-IR with 1 Gy. The conditioned media (CM) from irradiated EPCs (IR-CM) were collected at 30 min, 5 h and 24 h post-IR. The second set of cells was not irradiated (N-IR) and was used as naïve (e.g., non-irradiated) EPCs for media transfer study. Media was changed in all wells, including control N-IR wells, with fresh 3 ml of EBM2 media on the day of study and EPCs were incubated in fresh media for 1 h prior to IR. IR-CM was filtered through a sterile 0.22 μm membrane syringe filter (Corning Inc.) and 2 ml of IR-CM collected from EPCs 30 min, 5 h and 24 h post-IR was added to N-IR EPCs. Control medium from N-IR cells (N-IR-CM) were also collected and filtered similarly. After 24 h incubation with IR-CM and N-IR-CM naïve EPCs at each time point were fixed and stained for the formation and decay of p-H2AX foci.

Media Transfer Experiments in WT, p55KO and p75KO BM-derived EPCs—Two sets of sub-confluent ex vivo expanded EPCs from WT, p55KO and p75KO mice were prepared from the same animals for each genotype. At ˜70% confluence one set of 6-well dishes of WT, p55KO and p75KO EPCs were 7-IR with 1 Gy and conditioned media (CM) from irradiated EPCs (IR-CM) of all genotypes were collected before, 1 h, 5 h, 24 h, 3 and 5 days post-IR. The second set of non-irradiated (N-IR) WT, p55KO and p75KO cells was used as corresponding genotype naïve EPCs for media transfer study. Media was changed in all wells, including control N-IR wells, with fresh 3 ml of EBM2 media on the day of study and EPCs were incubated in fresh media for 1 h prior to IR. IR-CM was filtered through a sterile 0.22 μm membrane syringe filter (Corning Inc.) and 2 ml of IR-CM collected from WT, p55KO and p75KO EPCs at 1 h, 5 h, 24 h, 3 and 5 days post-IR was added to corresponding genotype naïve N-IR EPCs. Control media from N-IR WT, p55KO and p75KO EPCs were also collected and filtered similarly. After 24 h incubation with IR-CM and N-IR-CM collected from WT, p55KO and p75KO EPCs at 1 h, 5 h, 24 h, 3 and 5 days post-IR naïve EPCs on the cover slips were fixed and processed for p-H2AX immunostaining. In addition, both IR-CM and N-IR-CM from all three genotypes were collected and saved for ELISA profiling. WT, p55KO and p75KO IR EPC pellets were harvested at each time point and snap-frozen for future processing. All studies involved three biological replicates of WT, p75KO and p55KO mice for each time point.

Media Transfer Experiments in WT and p55KO BM-derived EPCs post TNF-α neutralization—As described in the previous section two sets of sub-confluent ex vivo expanded EPCs from WT and p55KO mice were prepared from same animal and one set was γ-IR with 1 Gy. At various time points before, 1 h, 5 h, 24 h, 3 and 5 days post-IR CM from irradiated WT and p55KO EPCs were collected and filtered as described earlier. CM was incubated with TNF-α neutralizing antibody (Cat. 506309, BioLegend, San Diego, Calif., USA) at a final concentration of 10 ng/ml for 1 h at room temperature and then transferred on to corresponding genotype naïve N-IR EPCs at respective time points. Control media from N-IR WT and p55KO EPCs were also treated the same way. After 24 h incubation of N-IR EPCs with IR-CM and N-IR-CM collected from respective genotypes at 1 h, 5 h, 24 h, 3 days and 5 days post-IR, naïve EPCs on the cover slips were fixed and processed for p-H2AX immunostaining.

Immunostaining. Imaging and Analysis—To assess the formation and decay of p-H2AX foci in CM-treated and in untreated WT, p55KO and p75KO EPCs treated with CM from corresponding genotype γ-IR EPCs, glass coverslips on which EPCs from each genotype were cultured were fixed in 4% paraformaldehyde (PFA, FD NeuroTechnologies Inc., Columbia, Md., USA) for 15 min at room temperature (RT) and washed with ice-cold 1× phosphate buffered saline (PBS, MediaTech, Herndon, Va., USA) for 5 min. Fixed cells were permeabilized with 0.1% Triton X-100 (SIGMA) for 15 min at RT and washed three times in 1×PBS for 5 min. Primary anti-p-H2AX rabbit monoclonal antibody (Cat. 9718S; Cell Signaling Technology, Danvers, Mass., USA) and Alexa-488 goat anti-rabbit secondary antibody (Cat.A11008; Life Technologies) were used to assay p-H2AX foci formation and decay over time. Topro-3 was used to visualize nuclei (Cat.T3605; Life Technologies). Images were obtained using laser scanning confocal microscope (LSM 510 Meta, ZEISS, Thornwood, N.Y., USA) at ×100 magnification. Cells with apoptotic features or micronuclei were not considered for p-H2AX analysis. Data were obtained from three replicate samples for both IR and N-IR treatment conditions at each time point totaling 300-400 cells each. Using a computer assisted image analysis algorithm based on pixel and color distribution the p-H2AX foci were evaluated by quantifying all cells with ≧1 p-H2AX foci. Graphs were plotted for mean foci/cell and for a percent of cells with an N of p-H2AX foci.

Enzyme-Linked Immunosorbent Assay (ELISA)—Aliquots of the IR-CM and N-IR-CM collected from WT and p55KO EPCs at 1 h, 5 h, 24 h, 3 days and 5 days post-IR were processed for mouse multiplex cytokine ELISA according to manufacturer protocols (Signosis, Sunnyvale Calif. USA). Following 14 cytokines, chemokines and growth factors were analyzed—interleukin-1 alpha (IL-1α), interleukin-1 beta (IL-1β), interleukin 6 (IL-6), monocyte chemoattractant protein-1 (MCP-1). Rantes, microphage inflammatory protein-1 alpha (MIP-1α), granulocyte colony-stimulating factor (G-CSF), granulocyte macrophage colony-stimulating factor (GM-CSF), epidermal growth factor (EGF), insulin growth factor 1 (IGF-1), vascular endothelial growth factor (VEGF), stem cell factor (SCF), basic fibroblast growth factor (bFGF) and Tumor Necrosis Factor-α (TNF-α). The plates were read using Tecan Spectra model 96 Well Microplate Reader (MTX Lab Systems, Vienna, Va., USA). The ELISA assay was also performed after TNF-α neutralization of γ-IR conditioned for three cytokines—TNF-α, IL-1α and IL-1β.

Mouse Recombinant Cytokine and Chemokine Treatments—To determine the role of TNF-α, IL-1α, MCP-1 and Rantes in the formation of p-H2AX foci p55KO EPCs were treated with various concentrations of these mouse recombinant (rm) proteins. Recombinant, lyophilized IL-1α, MCP-1 and Rantes (Cat.D-61112, D-64030 and D-6413; PromoKine, Heidelberg, Germany) were reconstituted with sterile, ultra-pure water to a stock concentration of 0.1 mg/ml and stored as aliquots according to manufacturer recommendations. Recombinant protein for TNF-α used for the study was available as a 0.5 mg/ml stock from the vendor (Cat. 34-8321-82; eBiosciences). Based on the results of multiplex ELISA of CM from 1 Gy γ-IR p55KO EPC; naïve p55KO EPCs were treated with the following concentrations of single recombinant protein: IL-1α—290 pg/ml, 580 pg/ml, MCP-1—580 pg/ml, 1160 pg/ml, 2900 pg/ml, and Rantes—600 pg/ml, 1500 pg/ml, for 24 h under normal growth conditions. Based on our previously published work we used three significantly different concentrations for TNF-α: two angiogenic—0.1 ng/ml and 1 ng/ml, and one cytotoxic—40 ng/ml) (35). Formation and decay of p-H2AX foci were quantified as described in the section for Immunostaining. Imaging and Analysis.

Statistical Analysis—All results are expressed as mean±SEM. Statistical analysis was performed using one-way ANOVA (Stat View Software, SAS Institute Inc.; Gary, N.C.). Differences were considered significant at p<0.05.

### Results

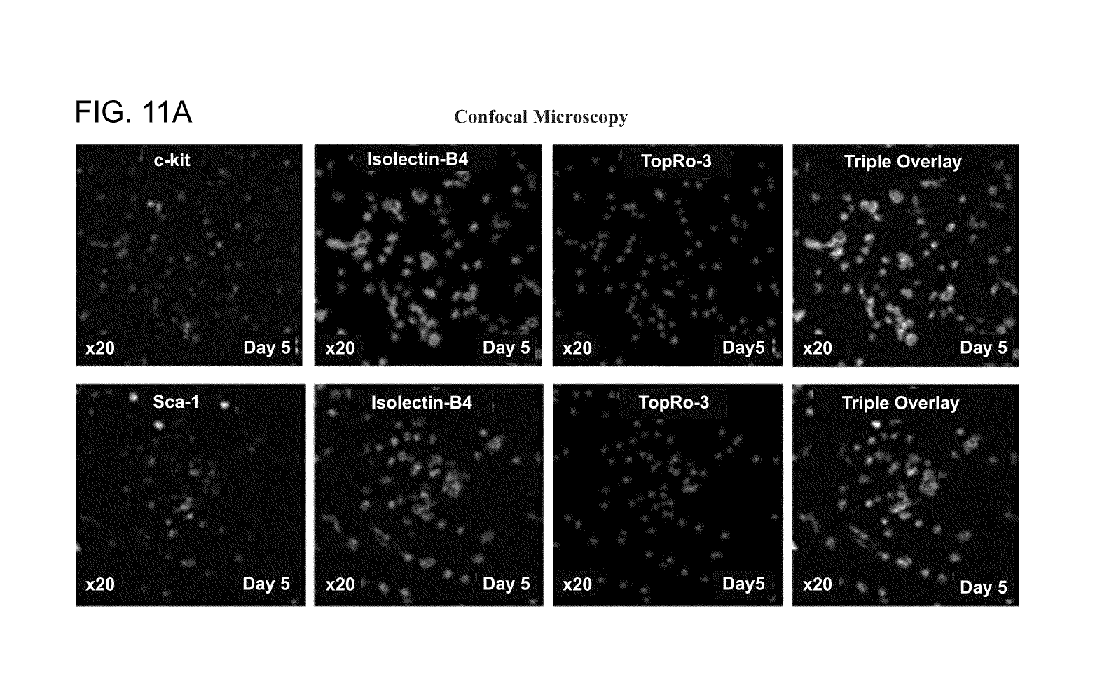

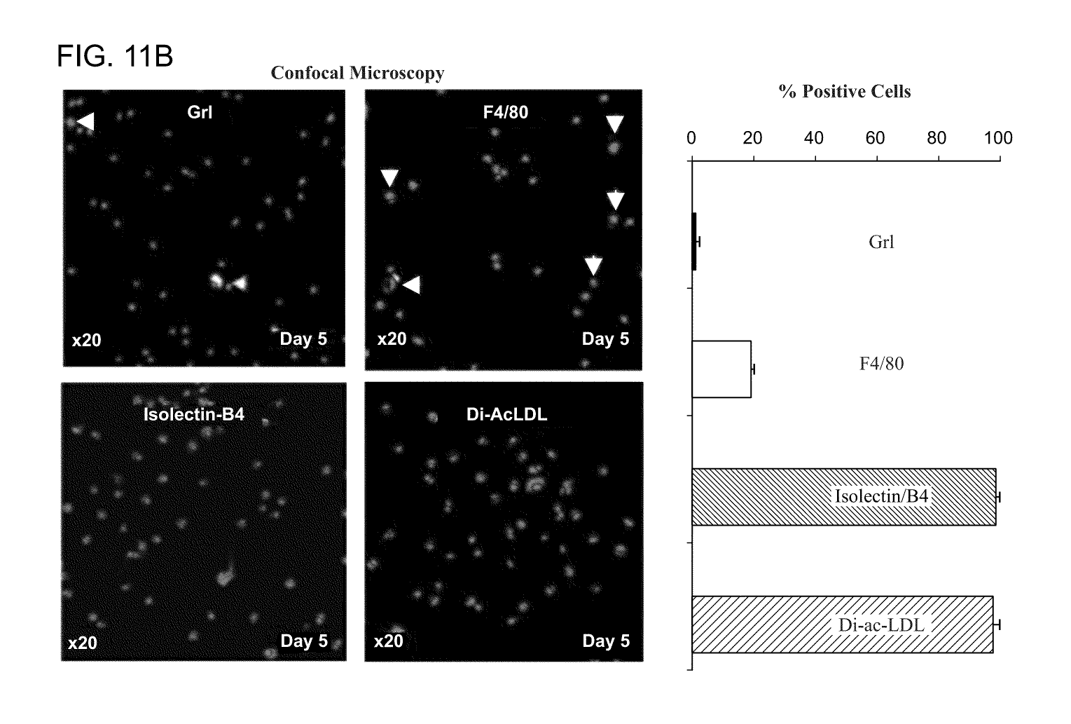

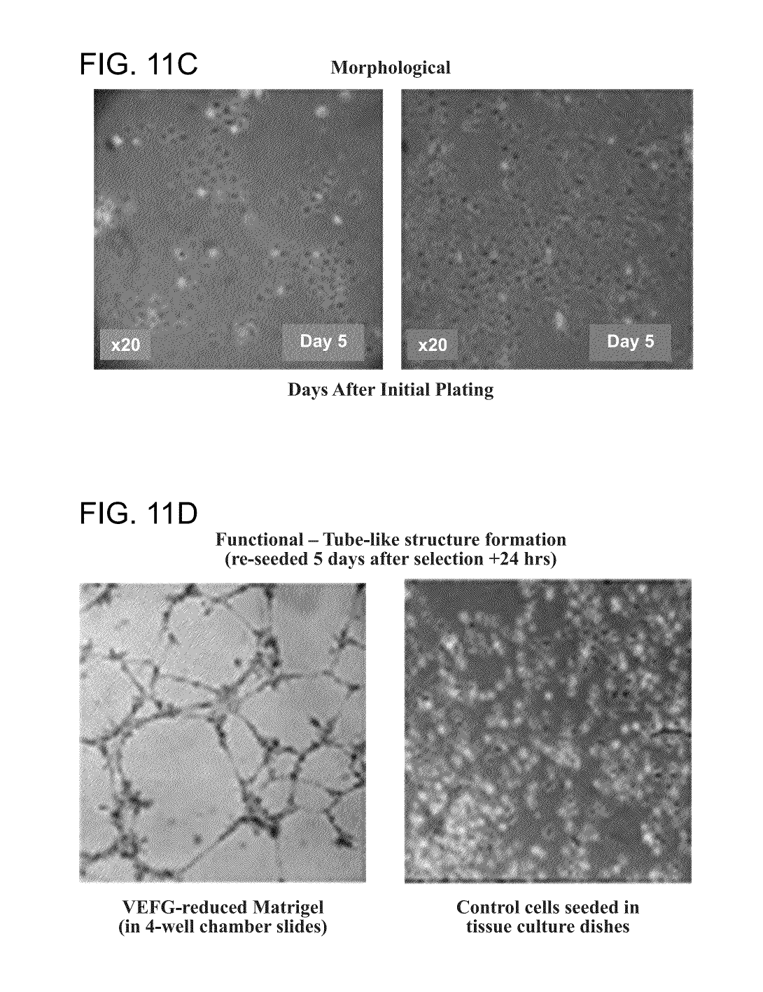

Characterization of ex vivo Expanded BM-derived EPC Culture—We have previously characterized and publishes our BM-derived EPCs cultures (35). Briefly, in our previous work BM-derived EPCs were stained with β-gal (biological EC marker-cells were grown from Tie2/LacZ mice) and c-kit (stem/progenitor cell marker) and we demonstrated that 95-100% of cells by day 4 and 6 were double positive for both markers (35). Two additional markers for endothelial cell lineage—Isolectin-B4 and Flk-1 also showed similar results by Day 6 in culture (35). In spite of extensive EPC culture characterization over the years we have tested but have not published a possibility that at early stages of ex-vivo BM-derived EPCs selection there may be hematopoietic “contamination” of the BM-derived EPC culture. Accordingly, we performed immunofluorescent staining of our BM-derived cultures with antibodies for c-kit and Sca-1 (stem/progenitor cell markers) combined with the endothelial cell marker Isolectin-B4. Nearly 100% of cells were double positive for c-kit/Isolectin-B4 and ˜95% were positive for Sca-1/Isolectin-B4 confirming stem/progenitor nature and endothelial lineage of cells in our BM-derived cultures (FIG. 11A).

To determine whether our BM-derived EPCs cultures may contain other lineage specific hematopoietic cells, we have performed immunofluorescent staining of our BM-derived cultures with antibodies for—Gr1/Ly-6G (neutrophils), F4/80 (macrophages and blood monocytes), CD45R/B220 (B lymphocytes), CD3ε (T lymphocytes), TER-119 (erythrocytes and erytroid precursors) and endothelial cell marker Isolectin-B4 (staining), as well as uptake while still in culture of the acetylated low density lipoprotein, labeled with 1,1′-dioctadecyl-3,3,3′,3′-tetramethyl-indocarbocyanine perchlorate (DiI-ac-LDL). Please note that DiI-ac-LDL is reported to label both vascular endothelial cells and bone marrow-derived macrophages (59-61). On day 5 no staining was detected for B220, Cd3ε and TER-119, suggesting that our culture was free of B and T lymphocyte and erythroid precursor “contamination”. On day 5 there was a negligible 1.17±0.7% positivity for Gr1 (neutrophils) staining (FIG. 11B, upper left panel). Further, ˜19% of cells were positive for F4/80 staining (FIG. 11B, upper right panel), a macrophage-specific marker, suggesting that by day 5 approximately ⅕ of the cells in our culture could be macrophages/monocytes. However, several lines of evidence from different groups have suggested that various macrophage markers, such as F4/80, Mac-3, CD68 or CD11b may be expressed in bone marrow-derived cultures during selection and especially during maturation/differentiation (62,63). More importantly, Modari et al., using “chimera” animal model where bone marrow (BM) of WT mice were transplanted with GFP/Tie2 BM cells (Tie2 is a tyrosine kinase receptor that is expressed on endothelial cells), have shown that many of BM-derived Tie2/GFP-expressing cells that were recruited into the thrombus during its resolution also expressed Mac-3, CD68 and/or F4/80, suggesting that these Tie2 positive cells have retain macrophage phenotype. These authors suggested that these cells with macrophage phenotype could be a population of plastic stem cells, but this was not validated experimentally.

Phase contrast images (×20) of ex vivo expanded BM-derived EPCs on days 3 and 5 showed that already by day 3 after initial plating EPC clusters formed well defined colonies (FIG. 11C). By both 3 and 5 days there was no significant morphological differences was observed in EPC culture and by day 5 cells attained ˜70% confluence (FIG. 11C). To further confirm endothelial lineage of cells in our culture we performed an additional functional characterization of using tube-like structure formation assay. EPCs collected on day 5 and re-seeded to 4-well chambers coated with phenol free and VEGF-reduced matrigel formed tube-like structures starting at 16 hrs after re-seeding (FIG. 11D), confirming an important EC functional characteristic of BM-derived EPCs. Phase contrast images (×20) of EPCs in culture at 3 and 5 days after initial plating were taken using Nikon Eclipse TE 200 microscope (Nikon Instruments Inc., Melville, N.Y., USA).

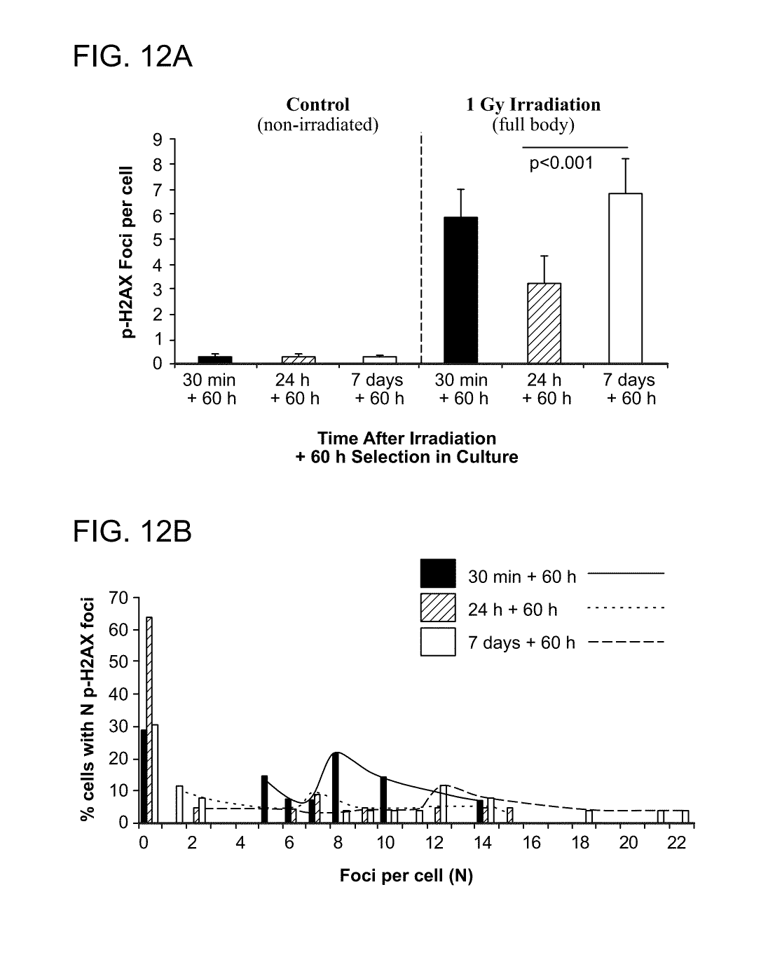

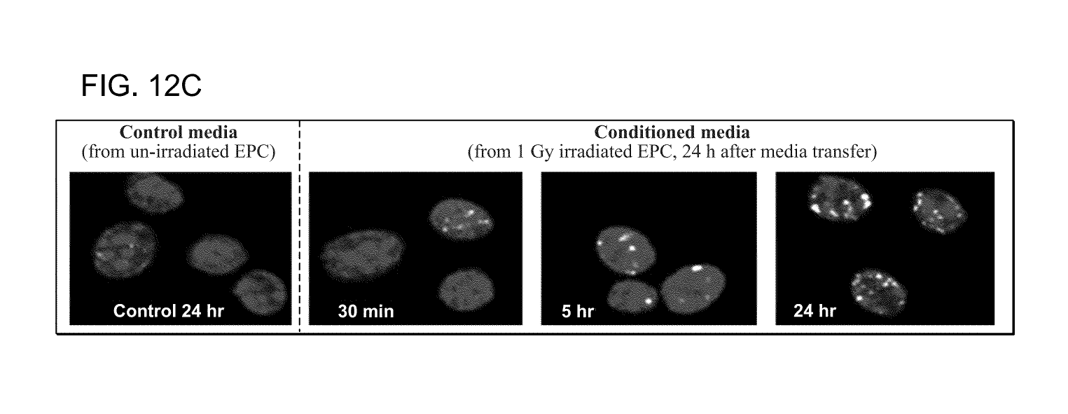

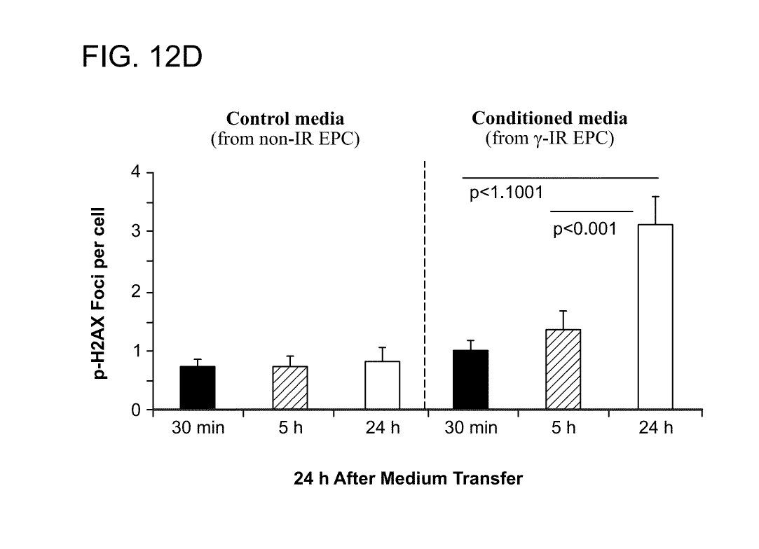

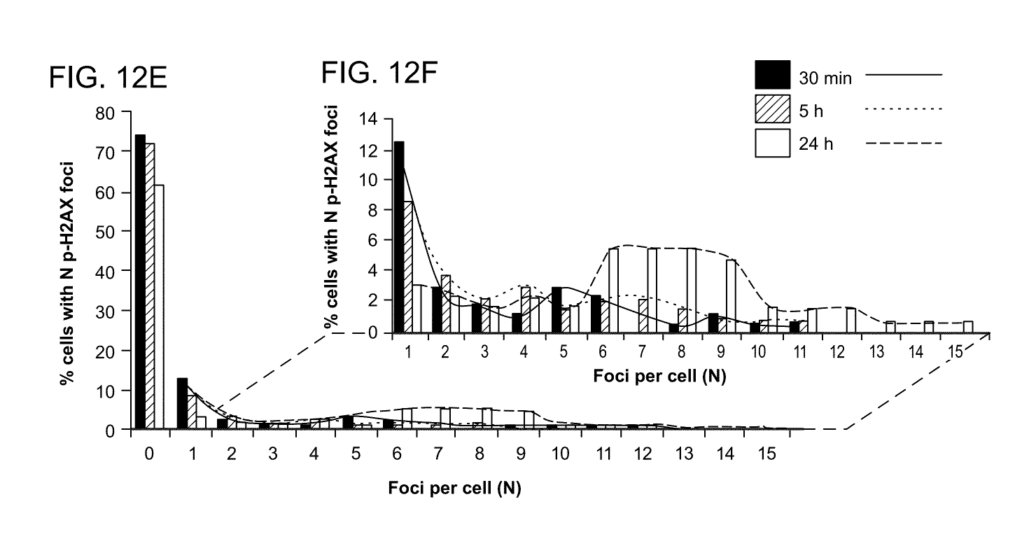

Within 24 h the Decay of p-H2AX foci in BM-EPCs is Slow and the Number of p-H2AX Foci is doubled by 7 Days—DNA damage-induced p-H2AX foci occur specifically at sites of double strand breaks (DSB) and the time-dependent decline in p-H2AX foci correlates well with DSB repair (64). We sought to determine the effect of low dose full body γ-IR on the formation and decay of p-H2AX foci in BM-derived EPCs from WT mice. BM-derived EPCs were isolated 30 min, 24 h and 7 days after full body γ-IR and selected in the culture ex vivo for 60 h. In control, N-IR EPCs there was a negligible number of p-H2AX (+) EPCs over a 7-day period (FIG. 12A). Compared to N-IR EPCs analysis of γ-IR samples revealed a significant >20-fold increase in the p-H2AX foci at 30 min (0.27±0.15 vs. 5.8±1.2 foci/cell, p<0.0003) that was followed by a ˜43% statistically not significant reduction (5.8±1.2 vs. 3.3±1.1 foci/cell, p=NS) at 24 h post-IR (FIG. 12A). However, compared to 24 h time point, the number of p-H2AX foci/cell at 7 days was increased more than 200% (3.3±1.1 vs. 6.8±1.4, p<0.006), to the level of the pH2AX foci at 30 min (FIG. 12A).

Analysis of p-H2AX foci quantity distribution at each time point showed that ˜5% of BM-EPCs showed an increase in the percent of cells with ≧18-22 p-H2AX foci/cell on day 7 (FIG. 12B, dotted line). These data indicate that decay of p-H2AX foci in WT BM-derived EPCs ex vivo is slow within first 24 h, which may be indicative of delayed or inefficient radiation-induced DNA damage repair. In addition, significant increase in mean foci/cell on day 7 post-IR along with an increase in the percent of p-H2AX foci/cell may be indicative of significant radiobiological bystander responses in BM-derived EPCs (3,4,6,7,65).

BM-derived EPCs Exhibit Significant Bystander Responses in vitro—To determine whether BM-derived EPCs show evidence of a bystander response in media transfer experiments in vitro. N-IR WT EPC cells were treated with conditioned medium (CM) collected from γ-IR WT EPCs. Formation of p-H2AX foci over 24 h post-treatment was used to evaluate the bystander effect. In control N-IR media-treated EPCs there was negligible number of detectable p-H2AX (FIG. 12C, 12D). There was no significant change in the mean foci/cell when comparing 30 min and 5 h IR-CM-treated cells (1±0.2 vs. 1.4±0.3, p=N.S) (FIG. 12C, 12D). BM-EPCs treated with 24 h IR-CM had a significant 320% (p<0.0001) and 228% (p<0.001) increases in the mean number of foci/cell, when compared to N-IR EPCs treated with 30 min and 5 h IR-CM, respectively (FIG. 12D, 12C). Quantification of p-H2AX foci distribution with a given number (N) of foci/cell showed an increase in the percent of cells with more than 6 to 15 p-H2AX foci/cell 24 h after addition IR-CM (FIG. 12E, 12F), suggests that BM-derived EPC exhibit significant bystander responses in vitro.

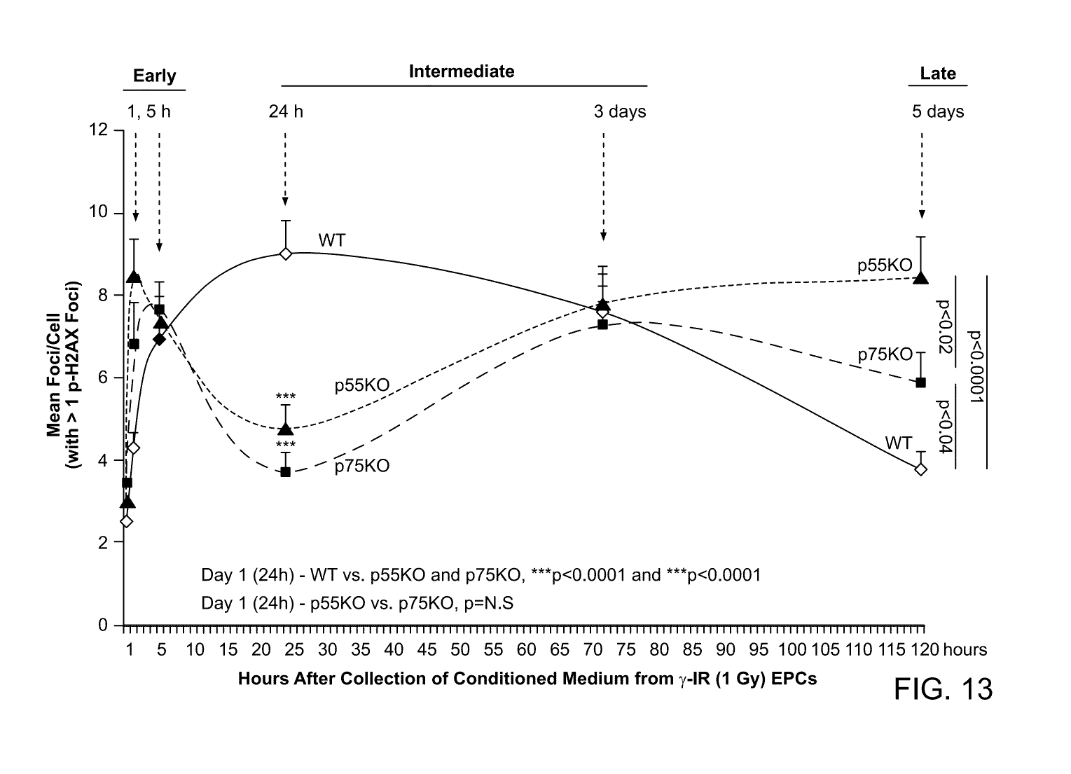

TNF Ligand-Receptor Interactions Modify Formation of p-H2AX Foci in BM-derived EPCs in vitro—It has been reported that early IR-induced vascular damage can be diminished by anti-TNF antibody (66) and elevated tissue TNF levels after IR are associated with significant increase in p-H2AX foci and genomic instability (67). Moreover, TNFR2/p75 signaling was suggested to have protective effects against 1R-induced demyelination in the brain (68). Therefore, we sought to examine whether TNF ligand-receptor interactions mediate RBR in BM-EPCs. To determine the involvement of TNFRs, media transfer experiments were carried out with in N-IR BM-derived EPCs isolated from WT, TNFR1/p55 and TNFR2/p75 knockout (KO) animals. Both p55KO and p75KO EPCs treated with 1 h IR-CM showed a significant increase in the mean pH2AX foci/cell with respect to the WT cells (p<0.002 and p<0.0001, respectively) (FIG. 13). For the clarity of data presentation only cells with 21 p-H2AX positive foci were considered for the graphs presented in FIG. 13.

There was no significant difference in the formation of p-HA2X foci in N-IR WT, p55KO and p75KO EPC treated with 5 h IR-CM (FIG. 13). Compared to WT, in the absence TNF receptors (p55 or p75) there was at least 2-fold decrease (p<0.0001) in the formation of p-H2AX foci/cell in N-IR p55KO and p75KO EPCs treated for 24 h with 1 day IR-CM from the respective genotype EPCs (FIG. 13). This was not the case for the WT cells which exhibited the peak increase (9.0±0.8 p-H2AX foci/cell) post treatment with 1 day IR-CM media. There was no difference in the formation of p-HA2X foci between N-IR p55KO and p75KO EPC treated with 1 day IR-CM (FIG. 13). These findings indicate that in γ-IR EPCs the presence of both TNF receptors (p55 and p75) is necessary for 1 day CM to increase the formation of p-H2AX foci in corresponding genotype N-IR EPCs, suggesting that by blocking either p55 or p75 TNF receptors one could inhibit formation of p-H2AX foci in N-IR radiated BM-EPCs within a day after radiation exposure.

Although, there was no significant difference in the formation of p-H2AX foci in N-IR WT, p55KO and p75KO EPC treated with 3-day IR-CM (FIG. 13), over 5-day period IR-CM treated WT N-IR EPCs showed a significant decrease (p<0.0001) in the mean p-H2AX foci/cell (FIG. 13). In contrast, there was a significant, steady increase over time in the formation of p-H2AX foci in N-IR p55KO treated with 1, 3 and 5-days IR-CM from corresponding genotype EPCs and, to a lesser degree, in p75KO EPCs (FIG. 13). Both WT and p75KO EPCs treated with 5-day IR-CM, exhibited a decreasing trend in mean p-H2AX foci/cell from 3 to 5 days with a small but significant (p<0.04) difference between them at 5 days (FIG. 13). There was a much larger >2-fold increase in the mean p-H2AX foci/cell for N-IR p55KO EPCs treated with 5-day IR-CM compared to both p75KO and WT EPCs (p<0.02 and p<0.0001, respectively) with p55KO EPCs having the highest and WT having the lowest mean p-H2AX foci/cell (FIG. 13). Foci distribution of % EPCs with N Foci count (data not shown) showed an increased 0.5-2.2% of p55KO EPCs with 14-27 foci at 5 h and 5 days compared to respective control, while peaks for WT and p75KO EPCs at 1 and 3 days demonstrated a decreasing trend normalizing to respective controls by day 5. These findings indicate that TNF signaling via TNFR1/p55 and TNFR2/p75 is necessary for development of early RBR in N-IR naïve EPCs. More importantly, our results demonstrate significant increase of delayed bystander response seen in naïve p55KO EPCs at 5 day time point, when compared to WT and p75KO EPCs, suggesting that signaling though remaining TNFR2/p75 in p55KO cells plays an important role in mediating radiobiological bystander responses in BM-EPCs. Due to significant and continued (from day 1 throughout day 5) increase in delayed bystander response in p55KO BM-EPCs we decide to focus or next set of studies on p55KO BM-EPCs.

Neutralization of TNF-α in γ-IR-CM Resulted in Significant Decrease in the Formation p-H2AX Foci in TNFR1/p55KO EPCs—Due to the role of elevated TNF levels post-IR in tissue resulting in increased p-H2AX foci formation (67) we determined the effects of TNF-α neutralization in IR-CM on p-H2AX foci formation in vitro for both p55KO and WT EPCs over 5 days. Media transfer experiments were performed in N-IR BM-derived EPCs from both WT and TNFR1/p55KO mice, where in the IR-CM at respective time points were filtered and then incubated in TNF-α neutralizing antibody before transferring on to respective naïve N-IR BM-EPCs.

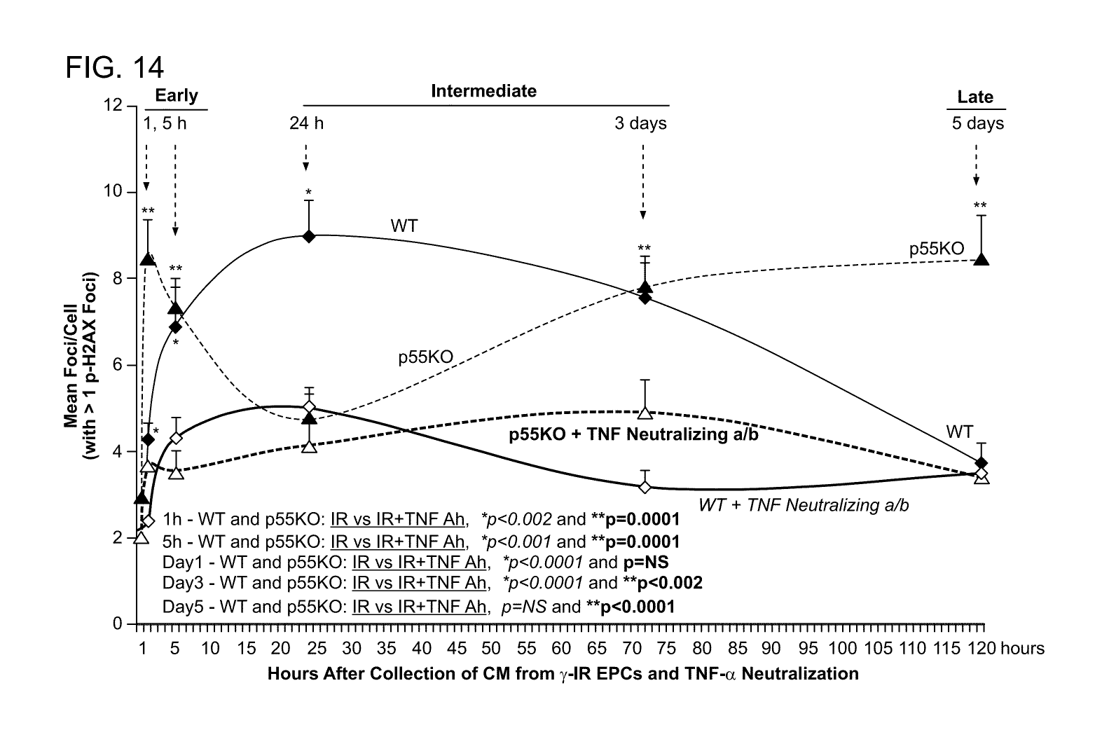

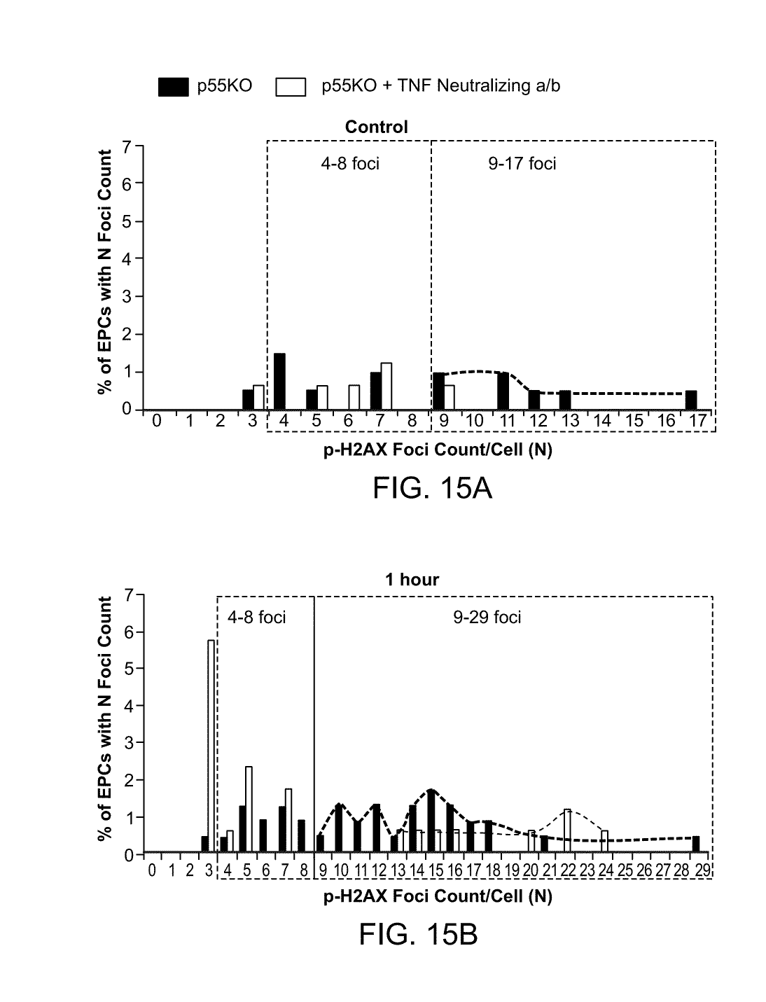

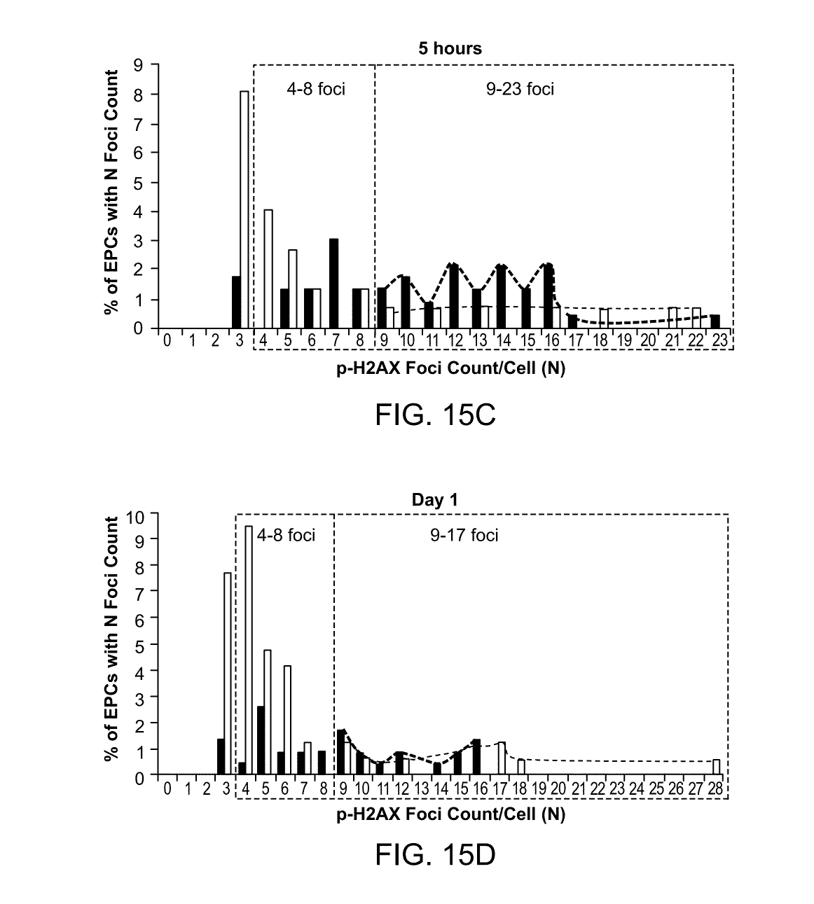

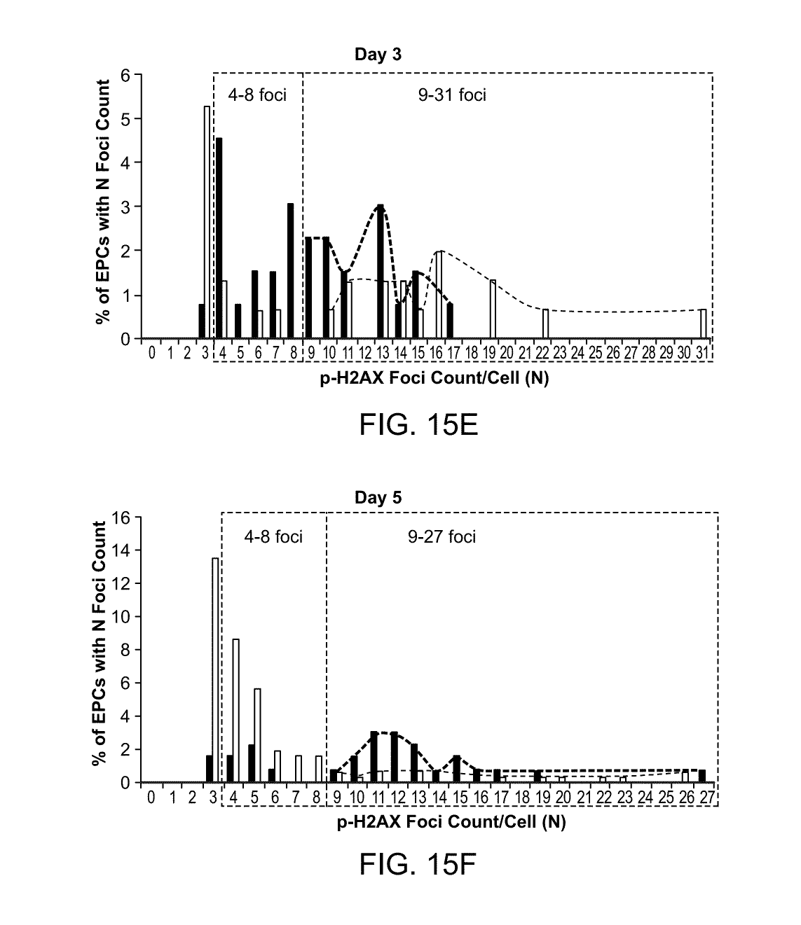

Non-irradiated WT EPCs treated with 1 h TNF-α neutralized IR-CM showed a significant increase in the mean pH2AX foci/cell with respect to N-IR p55KO EPCs (p<0.05) (FIG. 14, red vs. blue dashed lines). There was no significant difference in the formation of p-HA2X foci between N-IR WT and p55KO EPCs treated with respective 5 h, 24 h, 3 and 5 days TNF-α neutralized IR-CM (FIG. 14). Even though N-IR WT EPCs treated with TNF-α neutralized IR-CM still showed a gradual increase in p-H2AX foci formation over time with the peak increase at 24 hours, it was not significant compared to the number of p-H2AX foci in p55KO EPCs at this time point (FIG. 14). Foci distribution of naïve p55KO EPCs with N number of foci with and without TNF-α neutralization of 1 Gy γ-IR conditioned media at various time points—before (control), 1 h, 5 h, 24 h, days 3 and 5 is presented (FIG. 15A-15F). Taken together with the data of mean p-H2AX foci/cell the distribution of the N number of foci/cell confirms significant decrease in foci formation after TNF-α neutralization at 1, 5 hours and 5 days.

The continuous increase of p-H2AX foci formation over time in N-IR p55KO treated with 1, 3 and 5-days IR-CM from corresponding genotype EPCs (FIG. 14, red solid line) was significantly inhibited in p55KO EPCs treated with IR-CM after incubation with TNF-α neutralizing antibody before the media transfer (FIG. 14). These significant decreases in the formation of p-H2AX foci in N-IR p55KO EPCs treated with TNF-α neutralized IR-CM at all time points examined, substantiate the significant role of TNF-TNFR2/p75 signaling axis in delayed radiobiological responses in p55KO EPCs. Moreover, the incubation of γ-IR-CM from WT and p55KO EPCs with TNF-α neutralizing antibody showed that the formation of p-H2AX foci was significantly inhibited not only in p55KO but also in WT BM-EPCs up to 3 days indicating that inhibition of TNF-α may represent a therapeutic modality for the prevention of early and intermediate radiobiological bystander responses in WT tissue.

Increased Radiation Induced Accumulation of Cytokines and Growth Factors in TNFR1/p55KO EPCs in vitro—Radiation-mediated effects converge with increased levels of various cytokines and chemokines in that both generate reactive oxygen and nitrogen species that may lead to inflammation (69). In the cell growth media of p55KO EPCs, we sought to determine the effect of γ-IR on secretion and accumulation of cytokines, chemokines and growth factors, such as TNF-α, IL-1α, IL-1β, IL6, MCP1, MIP-1α, G-CSF, GM-CSF, EGF, VEGF, etc., all of which are known to be elevated within minutes to hours after ionizing radiation and other exogenous signals without the need of de novo protein synthesis (8).

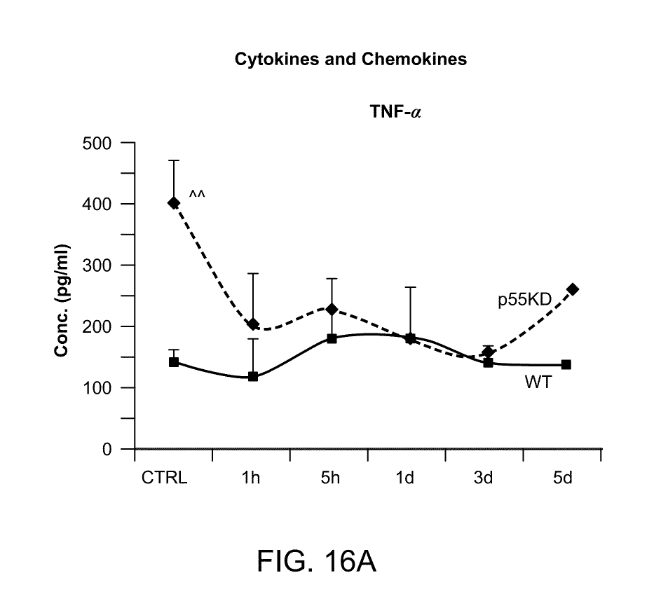

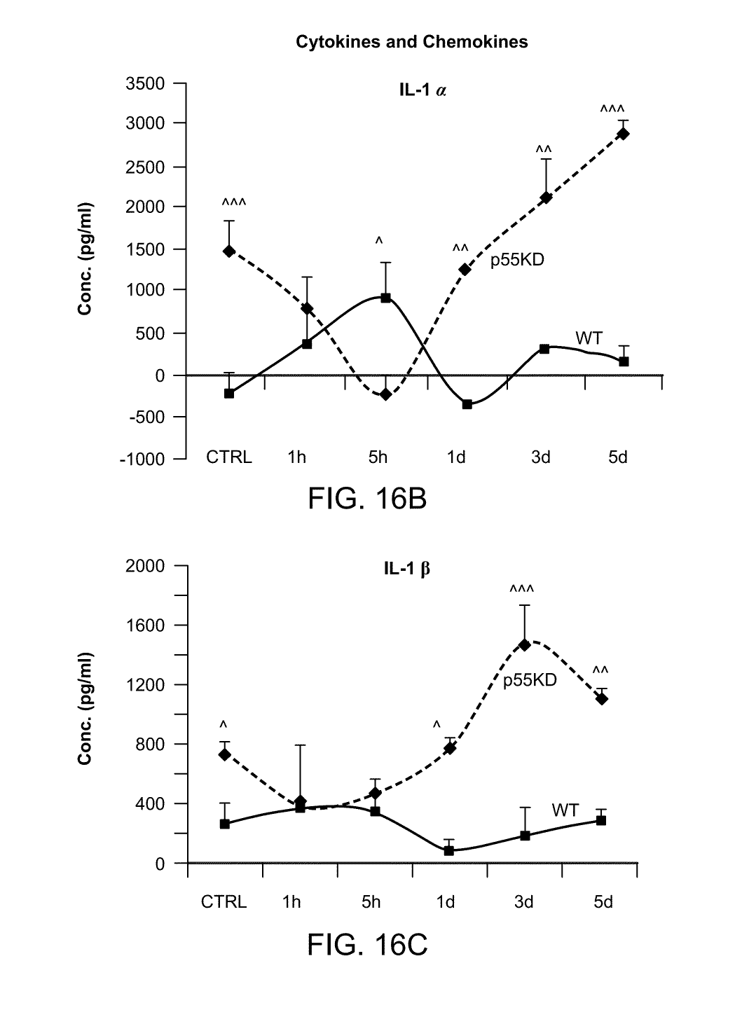

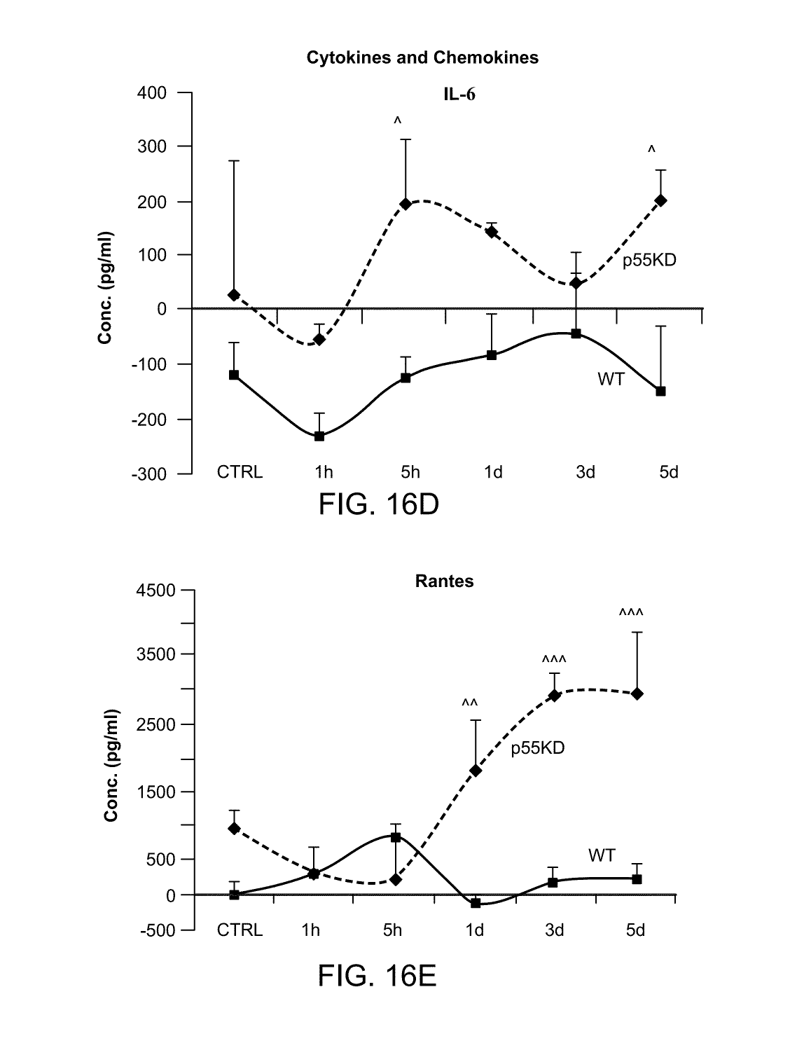

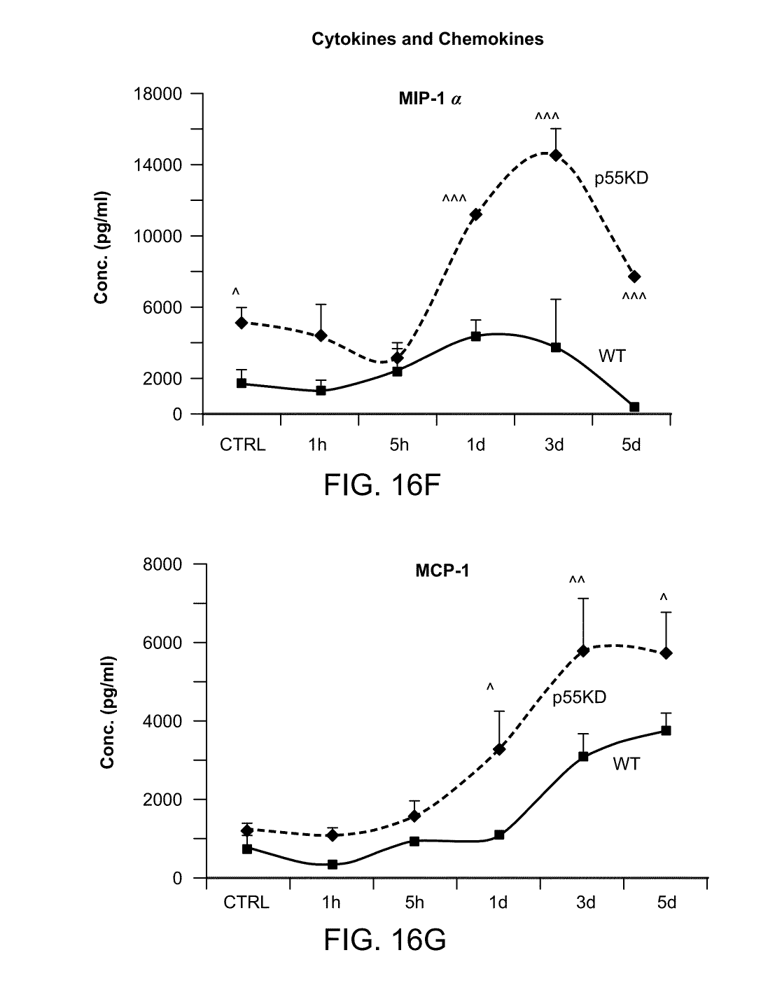

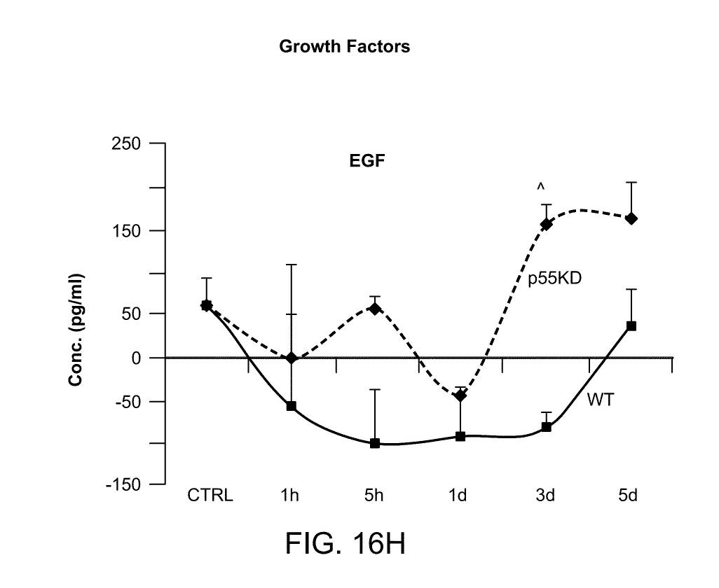

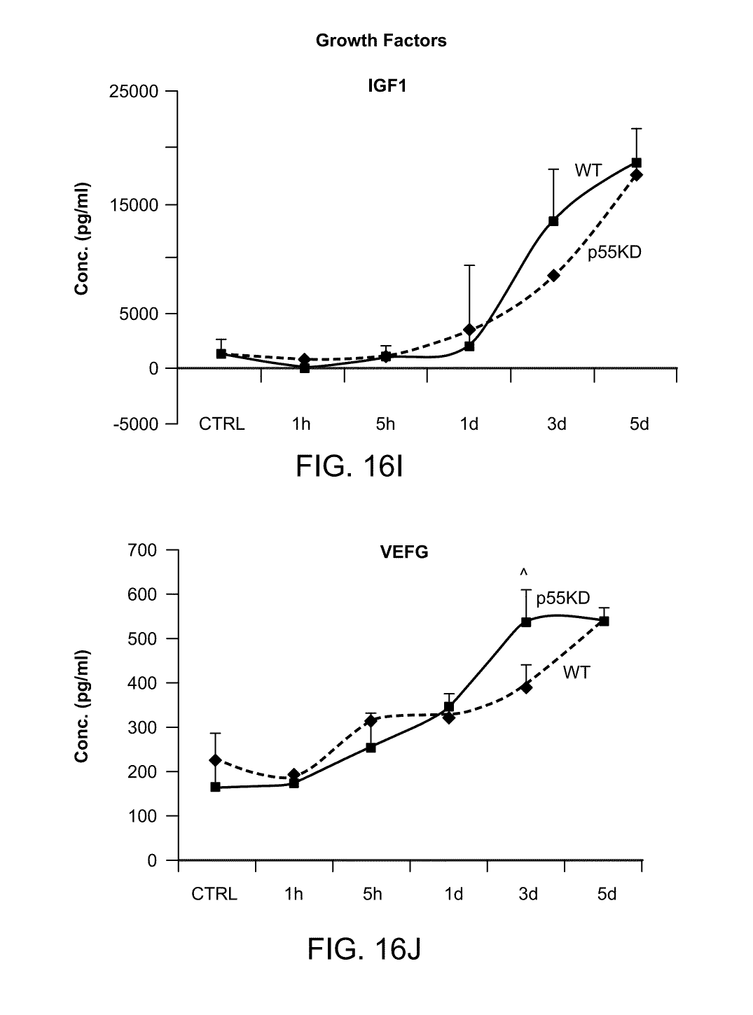

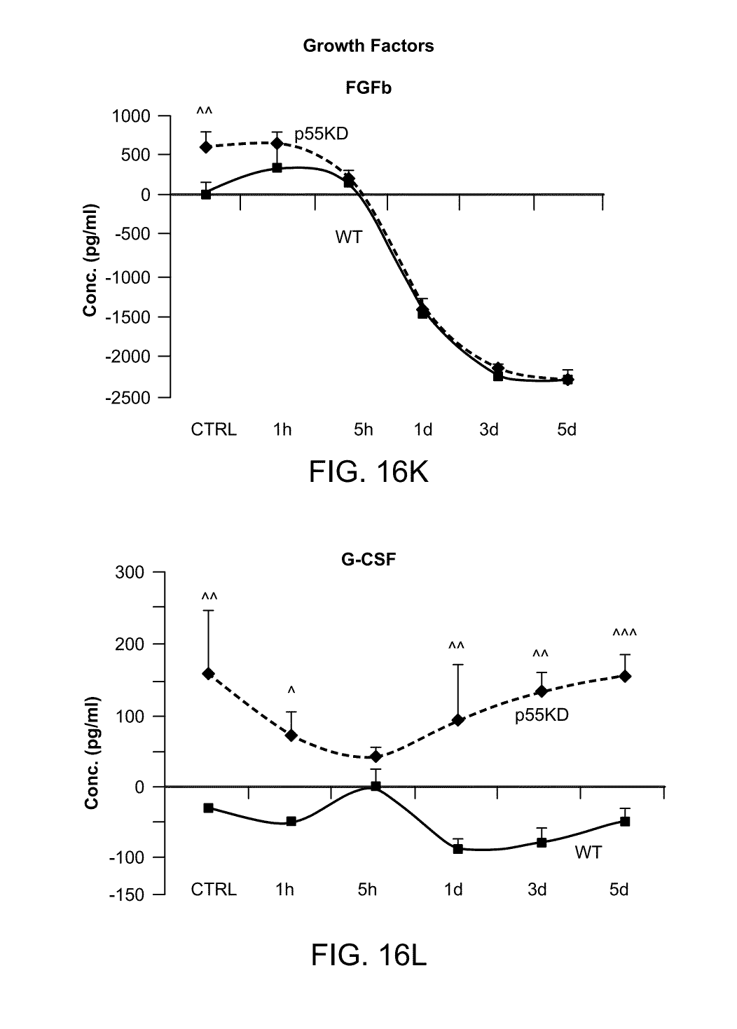

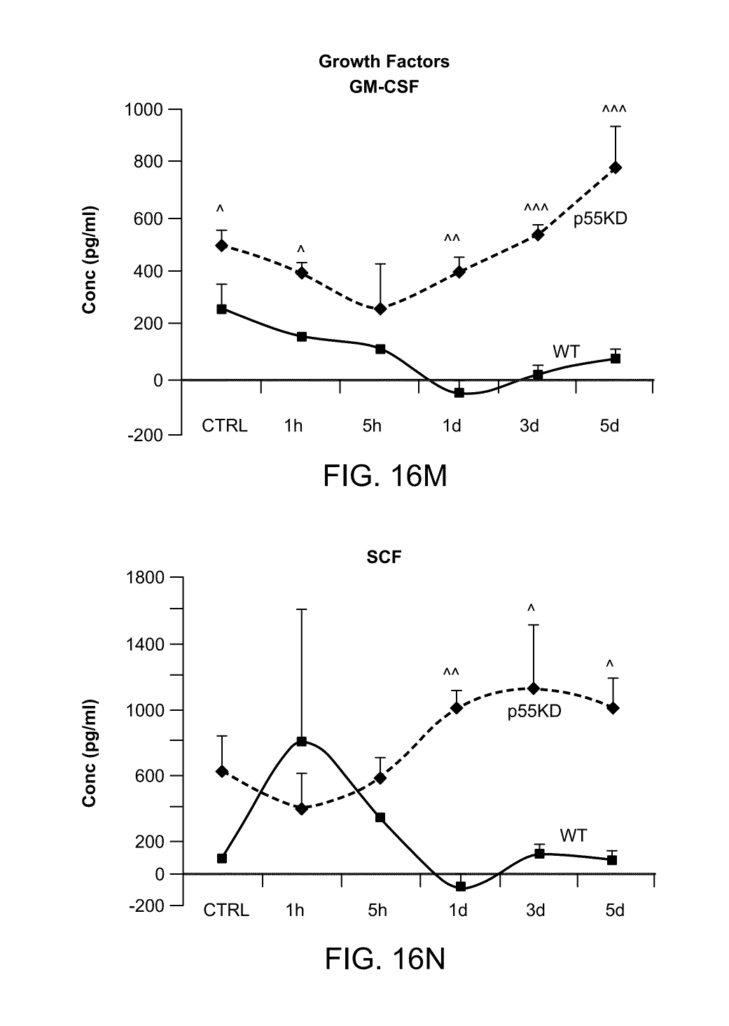

Conditioned media from γ-IR WT and p55KO EPCs were collected before (control), 1 h, 5 h or 24 h, 3 and 5 days post-IR and processed for multiplex (14-gene) ELISA. There was no significant difference in TNF-α level in IR-CM from p55KO and WT EPCs at 1 h and up to 3 days. However, compared to WT EPCs, there was a significant 175% increase (p<0.04) in TNF-α level in p55KO EPCs on day 5 (FIG. 16A). Compared to the media from IR WT EPCs, conditioned media from IR p55KO EPCs showed significant (200-1600%) increases in the secretion of IL-1α, IL-1β, Rantes, MIP-1α, MCP-1, G-CSF, GM-CSF and SCF as compared to IR-CM WT media (*p<0.05, **p<0.001 and ***p<0.0009) (FIG. 16B-16C, 16E-16G, 16L-16N). There was no significant difference in the concentration of IGF1, VEGF and bFGF between γ-IR-CM-treated WT and p55KO (FIG. 16I-16K), except a small but statistically significant (p<0.05) increase in VEGF concentration at day 3 in p55KO vs. WT IR-CM.

In addition to comparing increases in the concentration of cytokines and growth factors between IR-CM of WT and p55KO EPCs at each time point, we also analyzed the kinetics of changes for each protein over time when compared to corresponding genotype N-IR control levels (Table 2A, B).

This analysis revealed that in WT IR-CM statistically significant increases that were observed over 5 days were primarily in growth factors such (e.g., IGF1, VEGF, bFGF, GM-CSF and G-CSF) (Table 2A). In contrast, in p55KO IR-CM statistically significant increases (when comparing control levels with any one time point post-IR) were predominantly in cytokine levels such as TNF-α, IL-1α, IL-1β, Rantes, MIP-1α and MCP-1 (Table 2B). Taken together, our findings in multiplex ELISA studies suggest that the signaling through TNFR2/p75 in irradiated TNFR1/p55KO EPCs may lead to significant increases in the accumulation of the several cytokines and chemokines such as IL-1α, IL-1β, Rantes and MIP-1α (with the exception of MCP-1 at 3 and 5 days and IL-1α at 5 h, which were also increased in WT irradiated EPCs). Please note that cytokine levels for TNF-α, IL-1α and IL-1β after neutralization of TNF-α in IR-CM were not detectable in our ELISA assay.

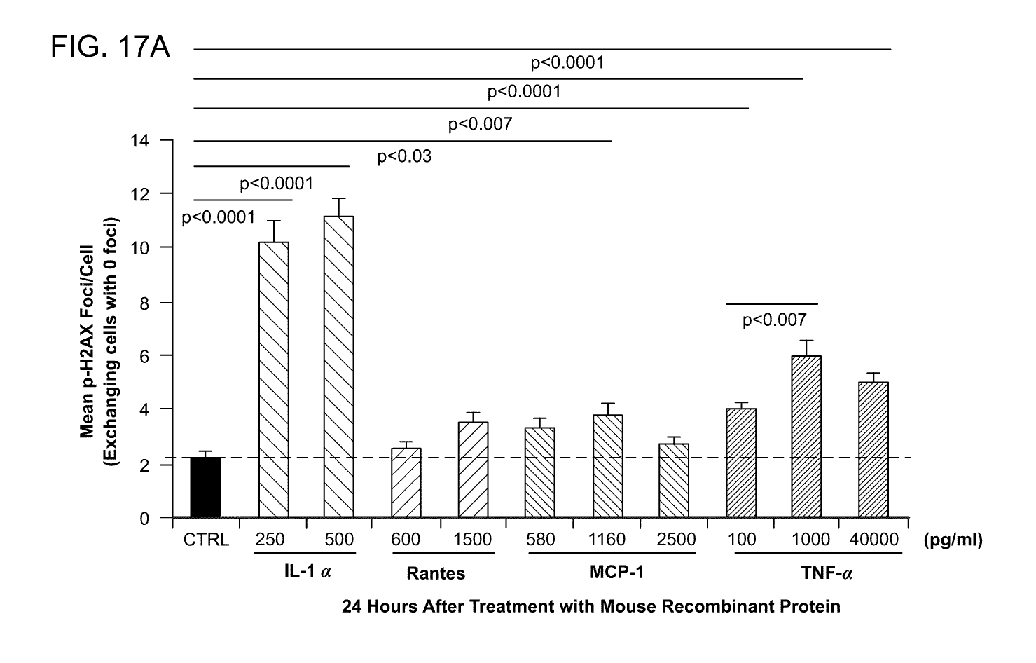

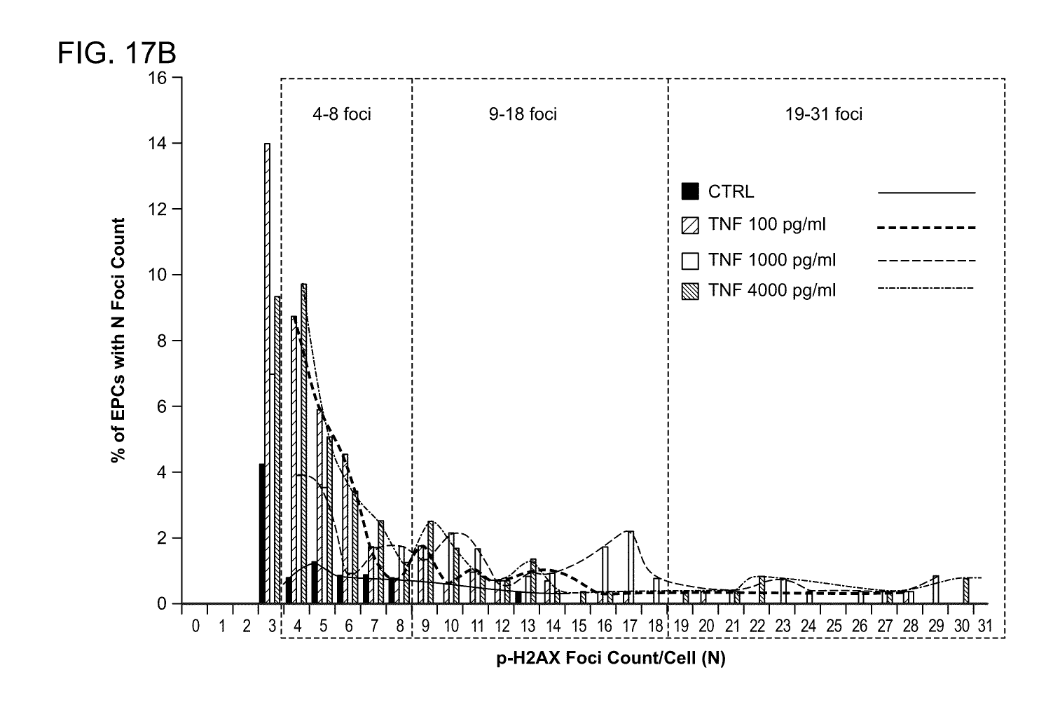

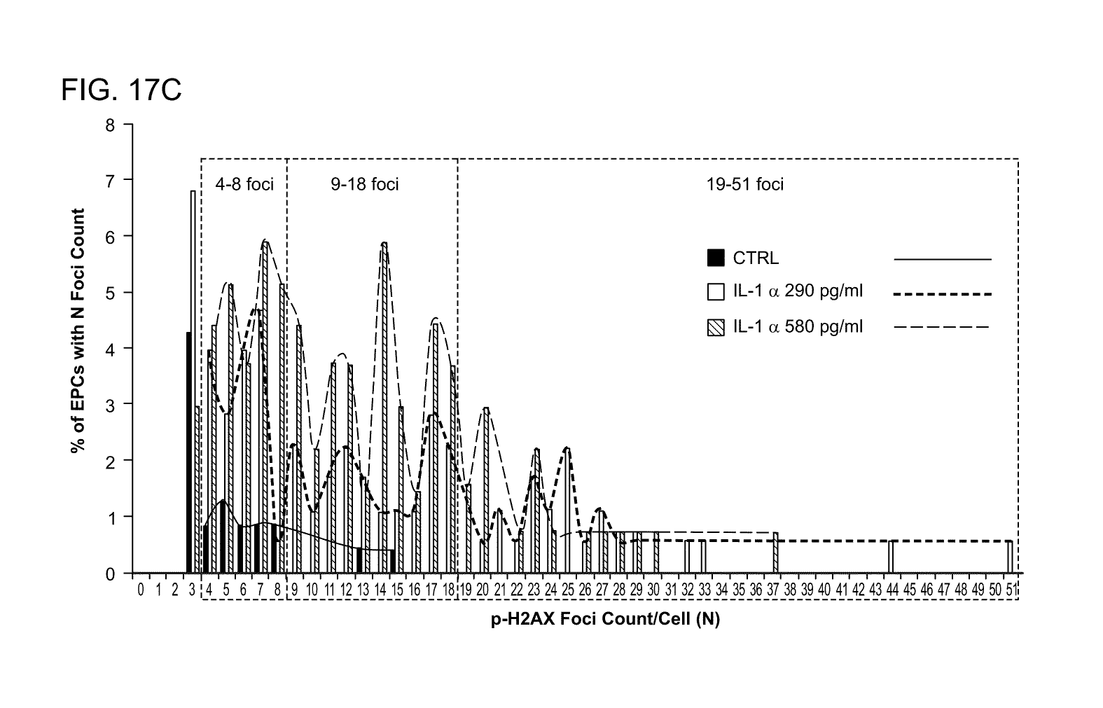

Treatment with Mouse Recombinant TNF-α and IL-1α, but not MCP-1 or Rantes Increases the Formation of p-H2AX Foci in N-IR TNFR1/p55KO EPCs in vitro—Based on our findings that p55KO EPCs exhibited the continuous increases in the levels TNF-α, IL-1α, Rantes and MCP-1 between days 1-5 (FIG. 16A, 16B, 16E, 16G and Table 2A, B) and the continuous increase in the mean p-H2AX foci/cell in N-IR p55KO EPCs treated with 1-5-day p55KO EPC IR-CM (FIG. 13, dotted line and 4, red solid line) we sought to determine whether treatment of N-IR p55KO EPCs with mouse recombinant IL-1α, MCP-1, Rantes and TNF-α could lead to the formation of p-H2AX foci. N-IR p55KO EPCs were treated with mouse recombinant TNF-α (100 pg/ml, 1,000 pg/ml, 40,000 pg/ml), IL-1α (290 pg/ml, 580 pg/ml), MCP-1 (580 pg/ml, 1.160 pg/ml, 2,900 pg/ml) or Rantes (600 pg/ml, 1,500 pg/ml). For the clarity of data presentation only cells with 21 p-H2AX positive foci were considered for the graphs presented in FIG. 17A. The data with inclusion of cells with zero p-H2AX foci was also quantified and plotted, which showed a similar trend in mean p-H2AX foci formation between treatment conditions (not shown).

After 24-hr treatment N-IR p55KO EPCs were stained with anti-p-H2AX antibodies. Quantification showed that the mean p-H2AX foci count of MCP-1 and Rantes were not significantly different from control which had a mean of 2.2±0.2 p-H2AX foci/cell, with the exception of MCP-1 at 1160 pg/ml (p<0.03, mean foci count of 3.7±0.5) (FIG. 17A). TNF-treated N-IR p55KO EPCs showed significant increase (p<0.0001) in mean p-H2AX foci/cell at all concentration (with mean p-H2AX foci in TNF-treated ranging from 4±0.3 to 6±0.6) compared to the control, MCP-1, and Rantes (FIG. 17A). IL-1α-treated p55KO EPCs showed the greatest increase in p-H2AX foci/cell with mean foci count of 10.1±0.9 at 290 pg/ml and 11.1±0.7 at 580 pg/ml, and was significantly (p<0.0001) different from all tested mouse recombinant proteins (FIG. 17A). Analysis of p-H2AX foci distribution of EPCs with more than 3 foci showed that in control p55KO EPCs less than 1-1.3% of cells had a maximum of 4-8 p-H2AX foci/cell and less than 0.5% of cells had 13 and 15 p-H2AX foci/cell (FIGS. 17B and 17C). Whereas in TNF-α and IL-1α-treated p55KO EPCs >2% and >4% of cells had 9-18 foci/cell, respectively (FIGS. 17B and 17C). Remarkably, 0.5-1% of cells had as many as 19-31 foci/cell for TNF-treated cells (FIG. 17B) and as many as 0.5-3% of cells had 19-51 foci/cell for IL-1α-treated p55KO EPCs (FIG. 17C). These findings suggest that TNF-α and IL-1α but not MCP-1 and Rantes increase significantly the formation of p-H2AX foci in naïve BM-derived TNFR1/p55KO EPCs.

### Discussion

The hematopoietic endothelial progenitor cells (EPCs) originate from the bone marrow and are part of the subpopulation of hematopoietic stem cells (HSC), which have been shown to be sensitive to radiobiological bystander response (20,24,70). EPCs are recruited to areas of neovascularization in the process of vasculogenesis to repair tissues with ischemia and cardiovascular diseases (44,48,57). EPCs are critical also to endothelial maintenance, and thus it is possible that radiation-induced EPC dysfunction could contribute to the pathogenesis of coronary and peripheral vascular diseases. Studies have demonstrated that in patients with CV risk factors, the number and migratory ability of EPCs isolated from peripheral blood is reduced (71) and EPC function is impaired (72). In 2006, Fadini et al. (73) demonstrated that early sub-clinical atherosclerosis in healthy subjects arises due to depletion of CD34+/KDR+ EPCs. In addition, a strong inverse correlation was reported between the number of circulating EPCs, vascular function and the subject's combined Framingham cardiovascular factor score (74). Furthermore, measurements of flow-mediated brachial-artery reactivity also revealed a significant relation between endothelial function and the number of EPCs, supporting a role for BM-derived EPCs in the maintenance of endothelial integrity (75).

Dose response curves for γ-radiation has been found through medium transfer experiments using ranges of 0.01-5.0 Gy on a human epithelial cell line. The degree of bystander effect seemed to saturate at ranges 0.03-0.05 Gy as measured by clonogenic death (76,77). In experiments using γ-radiation, a low-LET (linear energy transfer) radiation, it has been found that not all cell types have the same bystander response to radiation. In experiments with keratinocytes (epithelial cells), fibroblasts and radio-sensitive carcinoma cells, fibroblasts did not show induction of bystander effect through irradiated conditioned media transfer (17,76,77). In particular, the role of the bystander responses in bone marrow-derived EPC remains largely unknown.

Radiation-induced chromosomal instability was demonstrated in the bone marrow for up to 24 months after full body irradiation with either X-rays or neutrons, indicating that chromosomal instability can be initiated and maintained in-vivo (78,79). In addition, it was shown for myeloid and lymphoid bone marrow stem and progenitor cells that after space flight the numbers of these cells are reduced to just one-half of their normal levels (80), suggesting that EPCs may be similarly reduced in the normal EPC population after space flights. Unfortunately, neither data on EPC survival or mobilization, nor DNA damage responses of EPCs during and after space flights nor after lower doses of terrestrial radiation were available prior to the invention described herein.

Current understanding of low dose space and terrestrial radiation and its biological effects is that direct damage of DNA in the nucleus causes cell death and mutations (3). However, there have been numerous studies, especially in the past two decades, which suggest that radiation can cause damage in non-irradiated cells through radiobiological effects currently not fully understood (81,82). The term “bystander effect” was used to describe the ability of a cell, affected by radiation, to cause damage in other cells not directly traversed by the initial radiation (83). This radiobiological bystander effect was observed in radiation dosage as low as 0.31 mGy in an experiment by Nagasawa and Little, where Chinese hamster ovary cells (CHO) were irradiated with α-particles in G1 phase and measured for the induction of sister chromatid exchanges (SCE) (84). Results showed that 30% of cells had increased SCE even though only 1% of cell nuclei were traversed by α-particles.

It has been demonstrated that ionizing radiation at high doses can suppress the immune system; however, the response at low doses was less well-known prior to the invention described herein. Present risk assessment of high and low dose radiation is extrapolated from epidemiological studies of atomic bomb survivors in Hiroshima and Nagasaki; however there is some uncertainty in the assessment of risks at lower doses (11,85). Full body low-dose radiation is known to induce apoptotic and immunological responses in the organ-tissues, including the bone marrow (18,86,87). Full body low-dose radiation in mice has been found to enhance the function of macrophages (and CD8+ T cells) in several studies (18-20). Current evidence suggests that the immune response is enhanced at lower doses and influences the production and the release of inflammatory cytokines and chemokines such as tumor necrosis factor-α (TNF-α), interleukin-1-α (IL-1α), and others (18). The main goal of this study was to elucidate the mechanisms of the signal transmission for IR-induced radiobiological bystander responses in bone marrow-derived EPCs and determine the role of TNF ligand-receptor interaction in mediating RBR in these cells.

TNF-α is thought to be an important factor in the immune response of non-irradiated cells. ELISA of lung fibroblasts irradiated with low doses of α-particles showed a dosage dependent increase in IL-8 mRNA levels from 30 minutes to 24 hours post-irradiation (88). IL-8 expression is under the control of cis-elements of nuclear factor NF-κB, which is associated with the major pro-inflammatory pathway for TNF-α. (89). TNF-α was also directly implicated as one of the damaging signals in in vivo bystander experiments, as well as Fas ligand (FasL), nitric oxide, and super oxide (24,90). Hematopoietic cells from bone marrow were treated with radiation and measured for cells in sub-G0 region as a screening method for dead/damaged cells. The experiment showed an increasing trend in the number of sub-G0 cells at 1, 2, and 3 hour time points post-irradiation. When TNF-α neutralizing antibody was added to the irradiated bone marrow medium, the number of sub-G0 cells was significantly decreased in all three time points (24,90). Reduction was also noted for FasL neutralizing antibody, which might be another signal of interest.

Our findings here complement a recent gene expression study in directly irradiated and bystander cells that revealed NF-κB as the dominant transcription factor in mediating bystander responses (91,92). TNF is one of the key cytokines that activates the NF-κB (93). In turn NF-κB activation may lead to increased expression of NF-κB-dependent genes (94) in irradiated and Non-IR bystander cells. These include NF-κB-dependent cytokines, IL-6 and IL-8 (16), and cytokines that induce the NF-κB signaling pathway via paracrine or autocrine mechanisms—IL-1β, TNF-α and IL-33 (11, 17).

The results presented herein suggest that by blocking TNFR2/p75 signaling one can reduce production of growth factors and cytokines after ionizing radiation via reduced activation of NF-κB and other stress response transcription factors. Moreover our findings indicate that unopposed signaling via TNFR2/p75 in TNFR1/p55KO in EPCs plays a significant role in inhibiting early and increasing delayed (5 days) formation of p-H2AX foci, which correlate well with the induction of double strand breaks (95.96).

In summary, in (1) BM-derived EPCs in vivo and ex-vivo we report (a) slow decay of p-H2AX foci after full body 1 Gy γ-IR over 24 hours and increase over 7 days; (b) significant bystander responses in BM-derived EPCs in media transfer experiments; (2) in BM-derived WT, TNFR2/p75KO and TNFR1/p55KO EPCs ex-vivo we report that (a) compared to WT, in the absence of either of TNF receptors (p55 or p75) there is a significant decrease in the formation of p-H2AX foci at 5 and 24 h after adding IR-conditioned medium to naïve BM-derived EPCs, indicating that TNF and signaling via either of TNF receptors is necessary for development of radiobiological bystander responses in N-IR radiated BM-derived EPC; (b) compared to WT, in the absence of the either of TNF receptors (p55 or p75) there is a significant increase in the formation of p-H2AX foci on days 1, 3 and 5 after adding IR-conditioned medium to naïve BM-derived EPCs, indicating that TNF and signaling via either of TNF receptors increases delayed bystander responses in non-irradiated BM-derived EPCs; (c) continuous increase in the number (N) p-H2AX foci/cell between 1-5 days in naïve TNFR1/p55KO BM-derived EPCs may indicate that unopposed (by p55) signaling via TNFR2/p75 in TNFR1/p55KO EPCs plays a significant role in delayed bystander responses; (3) the specificity of the role of TNF in mediating bystander responses in BM-EPCs was confirmed in two experiments (a) the formation of p-H2AX foci was decreased more than twice in EPCs treated with γ-IR conditioned media after neutralizing TNF; (b) treatment of non-irradiated naïve EPCs with recombinant murine TNF led to significant increase in the formation of p-H2AX.

Thus, TNF plays significant role in mediating radiobiological bystander responses in BM-EPCs and these effects may be regulated (decreased or increased) through modification of TNF signaling via TNFR1/p55 or TNFR2/75. As described herein, blocking TNF or the signaling of TNF through one of it two receptors may be used to prevent radiation-induced delayed bystander effects in naïve “non-hit” BM-derived EPC. Additionally, blocking/neutralizing TNFR2/p75 (vs. TNFR1/p55) signaling may more effective strategy to inhibit the formation of p-H2AX foci in non-IR radiated EPCs, and conceivably other cells in bone marrow milieu.

Table 2A and Table 2B show statistical analysis of accumulation of cytokines, chemokines and growth factors in WT and p55KO γ-IR EPC media in control vs. all other time points. (A) Statistical analysis of protein concentrations using ELISA for different cytokines and chemokine and growth factors of γ-IR-CM from WT EPCs compared to respective control N-IR medium. Categorized into cytokines, chemokines and growth factors showed that in WT EPC medium most statistical significance at any time point compared to control was mostly growth factors with the exception of just two cytokines, IL-1α and MCP-1. (B) Statistical analysis of protein concentrations using ELISA for different cytokines, chemokine and growth factors of γ-IR-CM from p55KO EPCs compared to respective control N-IR medium. Compared to WT, IR-CM from p55KO EPCs demonstrated significantly higher levels of predominantly cytokine at any time point compared to control with the exception of just one growth factor, G-CSF. Red font indicates cytokines and chemokines that were significantly different in IR-CM from WT vs. p55KO EPCs and blue font indicates growth factors that were significantly different in IR-CM from WT vs. p55KO EPCs.

TABLE 2A and 2B

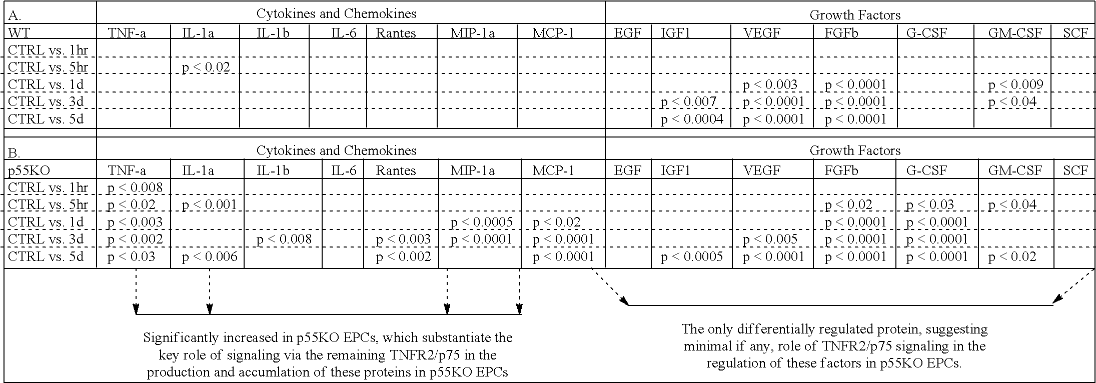

### REFERENCES

The following documents are cited in Example 7. As should be understood, the citations above to one or more numbers in parentheses (e.g., (1-3)) refers to the document listed below identified by the same number.
1. Azzam, E. I., de Toledo, S. M., and Little, J. B. (2001) Direct evidence for the participation of gap junction-mediated intercellular communication in the transmission of damage signals from alpha-particle irradiated to nonirradiated cells. Proceedings of the National Academy of Sciences of the United States of America 98, 473-478
2. Azzam, E. I., de Toledo, S. M., and Little, J. B. (2003) Expression of CONNEXIN43 is highly sensitive to ionizing radiation and other environmental stresses. Cancer research 63, 7128-7135
3. Lorimore, S. A., Coates, P. J., and Wright, E. G. (2003) Radiation-induced genomic instability and bystander effects: inter-related nontargeted effects of exposure to ionizing radiation. Oncogene 22, 7058-7069
4. Little, J. B. (2006) Cellular radiation effects and the bystander response. Mutation research 597, 113-118
5. Little, J. B. (2006) Lauriston S. Taylor lecture: nontargeted effects of radiation: implications for low-dose exposures. Health physics 91, 416-426
6. Morgan, W. F., and Sowa, M. B. (2007) Non-targeted bystander effects induced by ionizing radiation. Mutation research 616, 159-164
7. Hei, T. K., Zhou, H., Ivanov, V. N., Hong, M., Lieberman, H. B., Brenner, D. J., Amundson, S. A., and Geard, C. R. (2008) Mechanism of radiation-induced bystander effects: a unifying model. The Journal of pharmacy and pharmacology 60, 943-950
8. Schaue, D., Kachikwu, E. L., and McBride, W. H. (2012) Cytokines in radiobiological responses: a review. Radiation research 178, 505-523
9. Ivanov, V. N., Zhou, H., Ghandhi, S. A., Karasic, T. B., Yaghoubian, B., Amundson, S. A., and Hei, T. K. (2010) Radiation-induced bystander signaling pathways in human fibroblasts: a role for interleukin-33 in the signal transmission. Cellular signalling 22, 1076-1087
10. Hubackova, S., Krejcikova, K., Bartek, J., and Hodny, Z. (2012) IL1- and TGFbeta-Nox4 signaling, oxidative stress and DNA damage response are shared features of replicative, oncogene-induced, and drug-induced paracrine ‘Bystander senescence’. Aging 4, 932-951
11. Morgan, W. F. (2003) Non-targeted and delayed effects of exposure to ionizing radiation: 1. Radiation-induced genomic instability and bystander effects in vitro. Radiation research 159, 567-580
12. Yang, H., Asaad, N., and Held, K. D. (2005) Medium-mediated intercellular communication is involved in bystander responses of X-ray-irradiated normal human fibroblasts. Oncogene 24, 2096-2103
13. Yang, H., Anzenberg, V., and Held, K. D. (2007) The time dependence of bystander responses induced by iron-ion radiation in normal human skin fibroblasts. Radiation research 168, 292-298
14. Mitchell, S. A., Marino, S. A., Brenner, D. J. and Hall, E. J. (2004) Bystander effect and adaptive response in C3H 10(T(1/2) cells. International journal of radiation biology 80, 465-472
15. Yang, H., Magpayo, N., and Held, K. D. (2011) Targeted and non-targeted effects from combinations of low doses of energetic protons and iron ions in human fibroblasts. International journal of radiation biology 87, 311-319
16. Burdak-Rothkamm, S., Short, S. C., Folkard, M., Rothkamm, K., and Prise, K. M. (2007) ATR-dependent radiation-induced gamma H2AX foci in bystander primary human astrocytes and glioma cells. Oncogene 26, 993-1002
17. Sowa, M. B., Goetz, W., Baulch, J. E., Pyles, D. N., Dziegielewski, J., Yovino, S., Snyder, A. R., de Toledo, S. M., Azzam, E. I., and Morgan, W. F. (2010) Lack of evidence for low-LET radiation induced bystander response in normal human fibroblasts and colon carcinoma cells. International journal of radiation biology 86, 102-113
18. Pandey, R., Shankar, B. S., Sharma, D., and Sainis, K. B. (2005) Low dose radiation induced immunomodulation: effect on macrophages and CD8+ T cells. International journal of radiation biology 81, 801-812
19. Wright, E. G., and Coates, P. J. (2006) Untargeted effects of ionizing radiation: implications for radiation pathology. Mutation research 597, 119-132
20. Lorimore, S. A., Coates, P. J., Scobie, G. E., Milne, G., and Wright, E. G. (2001) Inflammatory-type responses after exposure to ionizing radiation in vivo: a mechanism for radiation-induced bystander effects? Oncogene 20, 7085-7095
21. Mungrue, I. N., Gros, R., You, X., Pirani, A., Azad, A., Csont, T., Schulz, R., Butany, J., Stewart, D. J., and Husain, M. (2002) Cardiomyocyte overexpression of iNOS in mice results in peroxynitrite generation, heart block, and sudden death. The Journal of clinical investigation 109, 735-743
22. Kojda, G., Laursen, J. B., Ramasamy, S., Kent, J. D., Kurz, S., Burchfield, J., Shesely, E. G., and Harrison, D. G. (1999) Protein expression, vascular reactivity and soluble guanylate cyclase activity in mice lacking the endothelial cell nitric oxide synthase: contributions of NOS isoforms to blood pressure and heart rate control. Cardiovascular research 42, 206-213
23. Meng, Y., Beckett, M. A., Liang, H., Mauceri, H. J., van Rooijen, N., Cohen, K. S., and Weichselbaum, R. R. (2010) Blockade of tumor necrosis factor alpha signaling in tumor-associated macrophages as a radiosensitizing strategy. Cancer research 70, 1534-1543
24. Burr, K. L., Robinson, J. I., Rastogi, S., Boylan, M. T., Coates, P. J., Lorimore, S. A., and Wright, E. G. (2010) Radiation-induced delayed bystander-type effects mediated by hemopoietic cells. Radiation research 173, 760-768
25. Sherman, M. L., Datta, R., Hallahan, D. E., Weichselbaum, R. R., and Kufe, D. W. (1991) Regulation of tumor necrosis factor gene expression by ionizing radiation in human myeloid leukemia cells and peripheral blood monocytes. The Journal of clinical investigation 87, 1794-1797
26. Tartaglia, L. A., Weber, R. F., Figari, I. S., Reynolds, C., Palladino, M. A., Jr., and Goeddel, D. V. (1991) The two different receptors for tumor necrosis factor mediate distinct cellular responses. Proceedings of the National Academy of Sciences of the United States of America 88, 9292-9296
27. Higuchi, Y., McTiernan, C. F., Frye, C. B., McGowan, B. S., Chan, T. O., and Feldman, A. M. (2004) Tumor necrosis factor receptors 1 and 2 differentially regulate survival, cardiac dysfunction, and remodeling in transgenic mice with tumor necrosis factor-alpha-induced cardiomyopathy. Circulation 109, 1892-1897
28. Chen, G., and Goeddel, D. V. (2002) TNF-R1 signaling: a beautiful pathway. Science (New York, N.Y 296, 1634-1635
29. Ihnatko, R., and Kubes, M. (2007) TNF signaling: early events and phosphorylation. General physiology and biophysics 26, 159-167
30. Jacobsen, S. E., Jacobsen, F. W., Fahlman, C., and Rusten, L. S. (1994) TNF-alpha, the great imitator: role of p55 and p75 TNF receptors in hematopoiesis. Stem Cells 12 Suppl 1, 111-126; discussion 126-118
31. Barbara, J. A., Van ostade, X., and Lopez, A. (1996) Tumour necrosis factor-alpha (TNF-alpha): the good, the bad and potentially very effective. Immunol Cell Biol 74, 434-443
32. Pastorino, F., Mumbengegwi, D. R., Ribatti, D., Ponzoni, M., and Allen, T. M. (2008) Increase of therapeutic effects by treating melanoma with targeted combinations of c-myc antisense and doxorubicin. J Control Release 126, 85-94
33. Sasi, S. P., Yan, X., Enderling, H., Park, D., Gilbert, H. Y., Curry, C., Coleman, C., Hlatky, L., Qin, G., Kishore, R., and Goukassian, D. A. (2012) Breaking the ‘harmony’ of TNF-alpha signaling for cancer treatment. Oncogene 31, 4117-4127
34. Vandenabeele, P., Declercq, W., Beyaert, R., and Fiers, W. (1995) Two tumour necrosis factor receptors: structure and function. Trends in cell biology 5, 392-399
35. Goukassian, D. A., Qin, G., Dolan, C., Murayama, T., Silver, M., Curry, C., Eaton, E., Luedemann, C., Ma, H., Asahara, T., Zak, V., Mehta, S., Burg, A., Thorne, T., Kishore, R., and Losordo, D. W. (2007) Tumor necrosis factor-alpha receptor p75 is required in ischemia-induced neovascularization. Circulation 115, 752-762
36. Ksontini, R., MacKay, S. L., and Moldawer, L. L. (1998) Revisiting the role of tumor necrosis factor alpha and the response to surgical injury and inflammation. Arch Surg 133, 558-567
37. Brockhaus, M., Schoenfeld, H. J., Schlaeger, E. J., Hunziker, W., Lesslauer, W., and Loetscher, H. (1990) Identification of two types of tumor necrosis factor receptors on human cell lines by monoclonal antibodies. Proceedings of the National Academy of Sciences of the United States of America 87, 3127-3131
38. Zhou, H., Ivanov, V. N., Gillespie, J., Geard, C. R., Amundson, S. A., Brenner, D. J., Yu, Z., Lieberman, H. B., and Hei, T. K. (2005) Mechanism of radiation-induced bystander effect: role of the cyclooxygenase-2 signaling pathway. Proceedings of the National Academy of Sciences of the United States of America 102, 14641-14646
39. Asahara, T., Murohara, T., Sullivan, A., Silver, M., van der Zee, R., Li, T., Witzenbichler, B., Schatteman, G., and Isner, J. M. (1997) Isolation of putative progenitor endothelial cells for angiogenesis. Science (New York, N.Y 275, 964-967
40. Lin, Y., Weisdorf, D. J., Solovey, A., and Hebbel, R. P. (2000) Origins of circulating endothelial cells and endothelial outgrowth from blood. The Journal of clinical investigation 105, 71-77
41. Kalka, C., Masuda, H., Takahashi, T., Kalka-Moll, W. M., Silver, M., Kearney, M., Li, T., Isner, J. M., and Asahara, T. (2000) Transplantation of ex vivo expanded endothelial progenitor cells for therapeutic neovascularization. Proceedings of the National Academy of Sciences of the United States of America 97, 3422-3427.
42. Shi, Q., Rafii, S., Wu, M. H.-D., Wijelath, E. S., Yu, C., Ishida, A., Fujita, Y., Kothari, S., Mohle, R., Sauvage, L. R., Moore, M. A. S., Storb, R. F., and Hammond, W. P. (1998) Evidence for circulating bone marrow-derived endothelial cells. Blood 92, 362-367
43. Asahara, T., Masuda, H., Takahashi. T., Kalka, C., Pastore, C., Silver, M., Kearne, M., Magner, M., and Isner, J. M. (1999) Bone marrow origin of endothelial progenitor cells responsible for postnatal vasculogenesis in physiological and pathological neovascularization. Circulation research 85, 221-228
44. Ribatti, D. (2007) The discovery of endothelial progenitor cells. An historical review. Leuk Res 31, 439-444
45. Urbich, C., and Dimmeler, S. (2004) Endothelial progenitor cells: characterization and role in vascular biology. Circulation research 95, 343-353
46. Folkman, J., and Shing, Y. (1992) Angiogenesis. Journal of Biological Chemistry 267, 10931-10934
47. Risau, W. (1995) Differentiation of endothelium. FASEB J. 9, 926-933
48. Yang, J., Ii, M., Kamei, N., Alev, C., Kwon, S. M., Kawamoto, A., Akimaru, H., Masuda, H., Sawa, Y., and Asahara, T. (2011) CD34+ cells represent highly functional endothelial progenitor cells in murine bone marrow. PLoS One 6, e20219
49. Gill, M., Dias, S., Hattori, K., Rivera, M. L., Hicklin, D., Witte, L., Girardi, L., Yurt, R., Himel, H., and Rafii, S. (2001) Vascular trauma induces rapid but transient mobilization of VEGFR(+)AC133(+) endothelial precursor cells Circulation research 88, 167-174
50. Shintani, S., Murohara, T., Ikeda, H., Ueno, T., Honma, T., Katoh, A., Sasaki, K., Shimada, T., Oike, Y., and Imaizumi, T. (2001) Mobilization of endothelial progenitor cells in patients with acute myocardial infarction. Circulation 103, 2776-2779
51. Kawamoto, A., Gwon, H. C., Iwaguro, H., Yamaguchi, J. I., Uchida, S., Masuda, H., Silver, M., Ma, H., Kearney, M., Isner, J. M., and Asahara, T. (2001) Therapeutic potential of ex vivo expanded endothelial progenitor cells for myocardial ischemia. Circulation 103, 634-637.
52. Kocher, A. A., Schuster, M. D., Szabolcs, M. J., Takuma, S., Burkhoff, D., Wang, J., Homma, S., Edwards, N. M., and Itescu, S. (2001) Neovascularization of ischemic myocardium by human bone-marrow-derived angioblasts prevents cardiomyocyte apoptosis, reduces remodeling and improves cardiac function. Nature medicine 7, 430-436.
53. Fischer-Rasokat, U., Assmus, B., Seeger, F. H., Honold, J., Leistner, D., Fichtlscherer, S., Schachinger, V., Tonn, T., Martin, H., Dimmeler, S., and Zeiher, A. M. (2009) A pilot trial to assess potential effects of selective intracoronary bone marrow-derived progenitor cell infusion in patients with nonischemic dilated cardiomyopathy: final 1-year results of the transplantation of progenitor cells and functional regeneration enhancement pilot trial in patients with nonischemic dilated cardiomyopathy. Circ Heart Fail 2, 417-423
54. Tateishi-Yuyama, E., Matsubara, H., Murohara, T., Ikeda, U., Shintani, S., Masaki, H., Amano, K., Kishimoto, Y., Yoshimoto, K., Akashi, H., Shimada, K., Iwasaka, T., and Imaizumi, T. (2002) Therapeutic angiogenesis for patients with limb ischaemia by autologous transplantation of bone-marrow cells: a pilot study and a randomised controlled trial. Lancet 360, 427-435
55. Schachinger, V., Assmus, B., Britten, M. B., Honold, J., Lehmann, R., Teupe, C., Abolmaali, N. D., Vogl, T. J., Hofmann, W. K., Martin, H., Dimmeler, S., and Zeiher, A. M. (2004) Transplantation of progenitor cells and regeneration enhancement in acute myocardial infarction: final one-year results of the TOPCARE-AMI Trial. J Am Coil Cardiol 44, 1690-1699
56. van Ramshorst, J., Bax, J. J., Beeres, S. L., Dibbets-Schneider, P., Roes, S. D., Stokkel, M. P., de Roos, A., Fibbe, W. E., Zwaginga, J. J., Boersma, E., Schalij, M. J., and Atsma, D. E. (2009) Intramyocardial bone marrow cell injection for chronic myocardial ischemia: a randomized controlled trial. Jama 301, 1997-2004
57. Iwaguro, H., Yamaguchi, J., Kalka, C., Murasawa, S., Masuda, H., Hayashi, S., Silver, M., Li, T., Isner, J. M., and Asahara, T. (2002) Endothelial progenitor cell vascular endothelial growth factor gene transfer for vascular regeneration. Circulation 105, 732-738.
58. Asahara, T., Takahashi, T., Masuda, H., Kalka, C., Chen, D., Iwaguro, H., Inai, Y., Silver, M., and Isner, J. M. (1999) VEGF contributes to postnatal neovascularization by mobilizing bone marrow-derived endothelial progenitor cells. The EMBO journal 18, 3964-3972
59. Goldstein, J. L, Ho, Y. K., Basu, S. K., and Brown, M. S. (1979) Binding site on macrophages that mediates uptake and degradation of acetylated low density lipoprotein, producing massive cholesterol deposition. Proceedings of the National Academy of Sciences of the United States of America 76, 333-337
60. Stein, O., and Stein, Y. (1980) Bovine aortic endothelial cells display macrophage-like properties towards lipoacetylated 125I-labelled low density lipoprotein. Biochem. Biophys. Acta 620, 631-635
61. Voyta, J. C., Via, D. P., Butterfield, C. E., and Zetter, B. R. (1984) Identification and isolation of endothelial cells based on their increased uptake of acetylated-low density lipoprotein. The Journal of Cell Biology 99, 2034-2040
62. Modarai, B., Burnand, K. G., Sawyer, B., and Smith, A. (2005) Endothelial progenitor cells are recruited into resolving venous thrombi. Circulation 111, 2645-2653
63. Lee, C. M., and Hu, J. (2013) Cell density during differentiation can alter the phenotype of bone marrow-derived macrophages. Cell & bioscience 3, 30
64. Rothkamm, K., and Lobrich, M. (2003) Evidence for a lack of DNA double-strand break repair in human cells exposed to very low x-ray doses. Proceedings of the National Academy of Sciences of the United States of America 100, 5057-5062
65. Rzeszowska-Wolny, J., Przybyszewski, W. M., and Widel, M. (2009) Ionizing radiation-induced bystander effects, potential targets for modulation of radiotherapy. European journal of pharmacology 625, 156-164
66. Ansari, R., Gaber, M. W., Wang, B., Pattillo, C. B., Miyamoto, C., and Kiani, M. F. (2007) Anti-TNFA (TNF-alpha) treatment abrogates radiation-induced changes in vascular density and tissue oxygenation. Radiation research 167, 80-86
67. Westbrook, A. M., Wei, B., Hacke, K., Xia, M., Braun, J., and Schiestl, R. H. (2012) The role of tumour necrosis factor-alpha and tumour necrosis factor receptor signalling in inflammation-associated systemic genotoxicity. Mutagenesis 27, 77-86
68. Daigle, J. L., Hong, J. H., Chiang, C. S., and McBride, W. H. (2001) The role of tumor necrosis factor signaling pathways in the response of murine brain to irradiation. Cancer research 61, 8859-8865
69. Bubici, C., Papa, S., Dean, K., and Franzoso, G. (2006) Mutual cross-talk between reactive oxygen species and nuclear factor-kappa B: molecular basis and biological significance. Oncogene 25, 6731-6748
70. Wright, E. G. (1998) Radiation-induced genomic instability in haemopoietic cells. International journal of radiation biology 74, 681-687
71. Vasa, M., Fichtischerer, S., Adler, K., Aicher, A., Martin, H., Zeiher, A. M., and Dimmeler, S. (2001) Increase in circulating endothelial progenitor cells by statin therapy in patients with stable coronary artery disease. Circulation
72. Tepper, O. M., Galiano, R. D., Capla, J. M., Kalka, C., Gagne, P. J., Jacobowitz, G. R., Levine. J. P., and Gurtner, G. C. (2002) Human endothelial progenitor cells from type II diabetics exhibit impaired proliferation, adhesion, and incorporation into vascular structures. Circulation 106, 2781-2786
73. Fadini, G. P., Coracina, A., Baesso, I., Agostini, C., Tiengo, A., Avogaro, A., and de Kreutzenberg, S. V. (2006) Peripheral blood CD34+KDR+ endothelial progenitor cells are determinants of subclinical atherosclerosis in a middle-aged general population. Stroke 37, 2277-2282
74. Hill, J. M., Zalos, G., Halcox, J. P., Schenke, W. H., Waclawiw, M. A., Quyyumi, A. A., and Finkel, T. (2003) Circulating endothelial progenitor cells, vascular function, and cardiovascular risk. The New England journal of medicine 348, 593-600
75. Loomans, C. J., de Koning, E. J., Staal, F. J., Rookmaaker, M. B., Verseyden, C., de Boer, H. C., Verhaar, M. C., Braam, B., Rabelink, T. J., and van Zonneveld, A. J. (2004) Endothelial progenitor cell dysfunction: a novel concept in the pathogenesis of vascular complications of type 1 diabetes. Diabetes 53, 195-199
76. Mothersill, C., and Seymour, C. (1997) Medium from irradiated human epithelial cells but not human fibroblasts reduces the clonogenic survival of unirradiated cells. International journal of radiation biology 71, 421-427
77. Mothersill, C., and Seymour, C. B. (1998) Cell-cell contact during gamma irradiation is not required to induce a bystander effect in normal human keratinocytes: evidence for release during irradiation of a signal controlling survival into the medium. Radiation research 149, 256-262
78. Watson, G. E., Lorimore, S. A., Macdonald, D. A., and Wright, E. G. (2000) Chromosomal instability in unirradiated cells induced in vivo by a bystander effect of ionizing radiation. Cancer research 60, 5608-5611
79. Watson, G. E., Pocock, D. A., Papworth, D., Lorimore, S. A., and Wright, E. G. (2001) In vivo chromosomal instability and transmissible aberrations in the progeny of haemopoietic stem cells induced by high- and low-LET radiations. International journal of radiation biology 77, 409-417
80. Todd, P., Pecaut, M. J., and Fleshner, M. (1999) Combined effects of space flight factors and radiation on humans. Mutation research 430, 211-219
81. Mothersill, C., Seymour, R. J., and Seymour, C. B. (2006) Increased radiosensitivity in cells of two human cell lines treated with bystander medium from irradiated repair-deficient cells. Radiation research 165, 26-34
82. Averbeck, D. (2010) Non-targeted effects as a paradigm breaking evidence. Mutation research 687, 7-12
83. Djordjevic, B. (2000) Bystander effects: a concept in need of clarification. BioEssays: news and reviews in molecular, cellular and developmental biology 22, 286-290
84. Nagasawa, H., and Little, J. B. (1992) Induction of sister chromatid exchanges by extremely low doses of alpha-particles. Cancer research 52, 6394-6396
85. Pierce, D. A., and Preston, D. L. (2000) Radiation-related cancer risks at low doses among atomic bomb survivors. Radiation research 154, 178-186
86. van Os, R., Konings, A. W., and Down, J. D. (1993) Compromising effect of low dose-rate total body irradiation on allogeneic bone marrow engraftment. International journal of radiation biology 64, 761-770
87. Seed, T. M., Fritz, T. E., Tolle, D. V., and Jackson, W. E., 3rd. (2002) Hematopoietic responses under protracted exposures to low daily dose gamma irradiation. Adv Space Res 30, 945-955
88. Narayanan, P. K., LaRue, K. E., Goodwin, E. H., and Lehnert, B. E. (1999) Alpha particles induce the production of interleukin-8 by human cells. Radiation research 152, 57-63
89. Sethi, G., Sung, B., and Aggarwal, B. B. (2008) TNF: a master switch for inflammation to cancer. Front Biosci 13, 5094-5107
90. Rastogi, S., Coates, P. J., Lorimore, S. A., and Wright, E. G. (2012) Bystander-type effects mediated by long-lived inflammatory signaling in irradiated bone marrow. Radiation research 177, 244-250
91. Amundson, S. A., Do, K. T., Vinikoor, L. C., Lee, R. A., Koch-Paiz, C. A., Ahn, J., Reimers, M., Chen, Y., Scudiero, D. A., Weinstein, J. N., Trent, J. M., Bittner, M. L., Meltzer, P. S., and Fornace, A. J., Jr. (2008) Integrating global gene expression and radiation survival parameters across the 60 cell lines of the National Cancer Institute Anticancer Drug Screen. Cancer research 68, 415-424
92. Ghandhi, S. A., Yaghoubian, B., and Amundson, S. A. (2008) Global gene expression analyses of bystander and alpha particle irradiated normal human lung fibroblasts: synchronous and differential responses. BMC medical genomics 1, 63
93. Coward, W. R., Okayama, Y., Sagara, H., Wilson, S. J., Holgate. S. T., and Church, M. K. (2002) NF-kappa B and TNF-alpha: a positive autocrine loop in human lung mast cells? J Immunol 169, 5287-5293
94. Pahl, H. L. (1999) Activators and target genes of Rel/NF-kappaB transcription factors. Oncogene 18, 6853-6866
95. Sharma, A., Singh, K., and Almasan, A. (2012) Histone H2AX phosphorylation: a marker for DNA damage. Methods Mol Biol 920, 613-626
96. Valdiglesias, V., Giunta, S., Fenech, M., Neri, M., and Bonassi, S. (2013) gammaH2AX as a marker of DNA double strand breaks and genomic instability in human population studies. Mutation research

### Example 8

### Cyclical Responses of Ionizing Particle Radiation in Bone Marrow-Derived Endothelial Progenitor Cells

During future exploration-type space missions astronauts will be exposed to space IR composed of a spectrum of low-fluence protons (¹H) and high charge and energy (HZE) nuclei (e.g. ⁵⁶Fe) for extended time. Bone-marrow (BM) derived endothelial progenitor cells (EPCs) are critical for endothelial cell (EC) maintenance and repair. The data on effects of low-dose ionizing space-type particle radiation (IR) on survival and proliferation of BM-EPCs is limited. As described in detail below, in media transfer experiments, it was examined in vitro whether treatment of non-irradiated (N-IR) BM-derived EPCs with ¹H- and/or ⁵⁶Fe-irradiated conditioned media (CM) may induce radiobiological bystander responses in naïve N-IR EPCs. Between 2-24 h, both, ¹H- and ⁵⁶Fe-IR CM-treated nave EPC showed significant increase in the number of p-H2AX foci, a well-accepted marker of DNA double strand break (DSB) formation and decay. This was associated with 2-15-fold increase in the levels of TNF-α, IL-1α, IL-1β, MCP-1 and MIP-1α in ¹H- and ⁵⁶Fe-IR EPC CM. The effect of a single, low dose, full-body 90 cGy, 1 GeV ¹H-IR and 15 cGy, 1 GeV/nucleon (n) ⁵⁶Fe-IR on survival and proliferation of BM-EPCs in C57BL/6NT WT mice was also evaluated ex-vivo. There was a cyclical (early 2-5 h and delayed 28 days) increase in BM-EPC apoptosis and decreased proliferation after a single low dose ¹H- or ⁵⁶Fe-IR. The data indicate that early, within hours, increase in BM-EPC apoptosis may be the effect of direct IR exposure, whereas late increase in apoptosis and decrease in proliferation could be a result of non-targeted effects in the cells that were not traversed by IR directly. As described herein, identifying the role of specific cytokines responsible for IR-induced non-targeted effects in BM milieu allows for the development of mitigating factors to reduced long-term effects of ionizing particle IR.

Radiation (IR)-induced chromosomal instability was demonstrated in the bone-marrow (BM) for up to 24 months after full body IR with either X-rays or neutrons, indicating that chromosomal instability can be initiated and maintained in-vivo [1, 2]. However, prior to the invention described there is a significant gap in studies to date assessing full body IR-induced survival and function of BM-derived EPCs (BM-EPCs) and effects of space IR on the heart. It was shown for myeloid and lymphoid BM-derived stem and progenitor cells that after space flights the numbers of these cells are reduced to just one-half of their normal levels [3], suggesting that EPCs may be similarly reduced in the normal EPC population. Prior to the invention described herein, neither data on BM-derived EPCs survival and proliferation during and after space flights, nor DNA damage responses of EPCs or heart tissue to space IR, were available. The results presented herein indicate that non-targeted/radiobiological bystander effects may be responsible for late/delayed harmful effects of radiation in the BM milieu.

The role of EPCs in repair and regeneration and post-natal angiogenesis (neovascularization) processes after injury or in the ischemic tissue has been well-documented. In various animal models [4-7] and human clinical trials [8-11] it has been shown that transplantation of BM cells and BM-EPCs leads to migration and homing of these cells to the areas of damage, where EPCs contribute to the processes of neovascularization leading to the development of collateral vessels, which then contribute to the recovery of blood flow in the damaged tissue [12-15]. Consequently, as described herein, a decrease in the total number of BM-EPCs or their dysfunction could contribute to the pathogenesis of ischemic and/or peripheral vascular diseases, recovery after tissue injury, as well as affect the maintenance of normal vascular homeostasis in the organs and tissue in general.

NASA Human Research Program (HRP) identified space radiation as one of the space flight risk factors to the vascular/circulatory system which is vastly unknown and limited to information collected days to weeks after space missions [16]. This will be accompanied by significant growth of the associated vascular network and will likely involve mobilization of BM-EPCs.

Since astronauts will be exposed to radiation composed of a spectrum of low-fluence (¹H and ²He) and high charge and energy (HZE) nuclei (e.g., ¹²C, ⁵⁶Fe) between few days through months [20, 21], a significant risk may exist for the potential development of acute impairments during space travel and long-term degenerative risks later in life following IR exposure. Prior to the invention described herein, the degree and specifically the dose-response relationship on BM-EPCs and its bearing on eventual disease risk was unknown. As described herein, low-dose space radiation-induced DNA damage responses in BM progenitor cell populations, including EPCs, may be of long duration and this may lead to significant decrease in the number of these cells, as well as long-term loss of EC function of BM-EPCs. This may then pose significant degenerative risk on physiologic homeostasis in the organs and tissue under conditions of normal aging and on repair and regeneration processes in the under pathologic conditions, such as injury or ischemia. As described in detail below, it was determined whether BM-derived EPCs may exhibit radiobiological bystander responses in vitro. As described below, the effect of low dose full body ionizing particle IR on the survival and proliferation of BM-derived EPCs was also determined in vivo.

### Material and Methods

### Animal Models

To determine low-dose full-body space-type ¹H-IR and ⁵⁶Fe-IR induced effects on survival and proliferation of BM-derived EPCs, adult 8-10 months old male C57Bl/6N mice were shipped directly from Taconic (Hudson, N.Y.) to Brookhaven National Laboratory (BNL) to be irradiated at NASA Space Radiation Laboratory (NSRL). Mice were kept in the temperature- and light-controlled environment and handled in accordance with IACUC guidelines and protocols approved by GeneSys Research Institute (GRI) and BNL. Since these adult mice were retired male breeders that tend to fight and induce skin lesions animals were housed one per cage.

### Radiation and Dosimetry

Full-body low dose space-type radiation experiments for both low-linear energy transfer (LET) proton (¹H) and high-LET iron (⁵⁶Fe) exposures were performed at the BNL in the NSRL according to standardized procedures. For both proton and iron full body IR mice were placed in individual polypropylene boxes with 4 mm holes drilled to produce a stress-free environment. LET levels for both particle radiations were held constant and the average dose-rate of 16.7±5 cGy/min for ¹H-IR and 5±0.5 cGy/min for ⁵⁶Fe-IR to deliver a cumulative dose of 90 cGy for ¹H and 15 cGy for ⁵⁶Fe, respectively. Constant energy of 1,000 MeV/nucleon (n) was used to deliver both, ¹H- and ⁵⁶Fe-IRs. Mice exposed to low dose space-IR were driven back to GRI animal facility from BNL for housing and experimental analysis. Control N-IR mice for each type of ion was sham irradiated—mice were placed in the same individual polypropylene boxes, taken to the irradiation “cave”, placed on beam line platform for the same duration of the time for each ion, but not irradiated.

BM-Derived EPC Ex-Vivo Culture and Ex-Vivo Selection from Full-Body Irradiated Mice

BM-EPCs were isolated from mononuclear (MNC) faction of total bone marrow isolated from tubular bones by flushing tibiae and femurs of ⁵⁶Fe-IR, ¹H-IR and N-IR mice using density gradient centrifugation. MNCs were then cultured on 22×22 mm square glass coverslips (Fisher Scientific, Pittsburgh, Pa.) pre-coated with 0.2% gelatin (Sigma, St Louis, Mo.) in 6-well dishes (Corning Inc., Corning, N.Y.). BM-EPCs were expanded ex-vivo in selective EBM-2 growth medium (Lonza, Hopkinton, Mass.) supplemented with growth factor until they attained ˜70-80% confluence as described previously [5, 14, 23]. All experiments using BM-EPCs and data analyses were performed in triplicate biological samples.

Medium Transfer Experiments in BM-EPCs after ⁵⁶Fe-IR and ¹H-IR

BM-EPCs were isolated from full-body ⁵⁶Fe- and ¹H-IR mice as described above. Based on earlier work performed by Emerit et al. [24] for IR-conditioned media transfer studies, two sets of BM-EPCs from the same WT mice that were IR with ¹H or ⁵⁶Fe and N-IR controls were prepared described previously (Sasi et al., 2014 Journal of Radiation Research, 55:i122-i123). Upon attaining ˜70% confluence one set of BM-EPCs was exposed to 15 cGy, 1 GeV/n ⁵⁶Fe-IR and 90 cGy, 1 GeV ¹H-IR. After irradiations conditioned media (CM) from ¹H- or ⁵⁶Fe-IR-EPCs (¹H- or ⁵⁶Fe-IR-CM) and control N-IR EPCs (N-IR-CM) were collected at 2, 5 and 24-hour time points. Prior to IR exposures, media was changed in all wells of both sets with fresh 3 ml of EBM-2 media without growth factors and incubated for 1 hour. The second set of N-IR cells from same mice was used as naïve EPCs for media transfer study from their respective ⁵⁶Fe-IR and ¹H-IR exposed EPCs. IR-CM was filtered through a sterile 0.22 μm membrane syringe filter and 2 ml of IR-CM collected at 2, 5 and 24-hours post-IR was added onto corresponding mice N-IR EPCs. N-IR-CM from N-IR cells were also collected, filtered and transferred similarly. Naïve EPCs were incubated for 24-hours with ¹H-, ⁵⁶Fe-IR and N-IR conditioned media collected 2, 5 and 24 hours post-IR and were processed for p-H2AX immunostaining as described below. The remaining 1 ml of ¹H-IR-, ⁵⁶Fe-IR- and N-IR-CM was aliquoted and snap frozen in liquid nitrogen for protein analyses.

### Immunofluorescent Staining

The formation and decay of p-H2AX foci was assessed in naïve EPCs treated with 2-, 5- and 24-hour ¹H-IR, ⁵⁶Fe-IR and N-IR EPC conditioned media for 24 hours after treatment with CM. Cell on cover slips were washed with 1×PBS, fixed, and then incubated with primary anti-p-H2AX rabbit monoclonal antibody (Cat. 9718S; Cell Signaling Technology, Danvers, Mass.). Alexa-488 goat anti-rabbit secondary antibody (Cat.A11008; Life Technologies) and Alexa-594 labeled streptavidin (Cat.S11227, Life Technologies) were used respectively to assay p-H2AX foci formation and decay over time in N-IR, ¹H-IR and 56Fe-IR-CM-treated ex-vivo expanded EPCs. Topro-3 was used to visualize nuclei (Cat.T3605; Life Technologies).

### Confocal Microscopy and Analysis

Laser scanning confocal images (LSM 510 Meta, ZEISS, Thornwood, N.Y.) at ×200 magnification were obtained and p-H2AX foci analysis was performed using a computer assisted image analysis algorithm based on pixel and color distribution. Data analysis was performed using stringent constrain of not including cells with apoptotic features or micronuclei for p-H2AX analysis. Graphs were plotted as percent cells with an N of p-H2AX foci for controls N-IR and all time point after ¹H and ⁵⁶Fe-IR by quantifying cells with ≧1 p-H2AX foci/cell.

### Enzyme-Linked Immunosorbent Assay (ELISA)

Frozen aliquots of conditioned media from BM-EPCs after ⁵⁶Fe-IR, ¹H-IR and N-IR were collected at 2, 5 and 24-hours post-IR and processed for mouse multiplex cytokine ELISA using manufacturer protocol (Signosis, Santa Clara, Calif.). Following 9 cytokines, chemokines and growth factors were analyzed: interleukin-1 alpha (IL-1α), interleukin-beta (IL-1β), monocyte chemoattractant protein-1 (MCP-1), Rantes, microphage inflammatory protein-1 alpha (MIP-1α), granulocyte colony-stimulating factor (G-CSF), granulocyte macrophage colony-stimulating factor (GM-CSF), stem cell factor (SCF) and Tumor Necrosis Factor-α (TNF-α). Absorbance readings at 450 nm were read using Tecan Spectra model 96-well Microplate Reader (MTX Lab Systems, Vienna, Va.) and data plotted using respective standard graphs obtained for each protein. Data analyzed was categorized into two separate groups—cytokines/chemokines and growth factors.

Apoptosis Assay of Ex-Vivo Expanded BM-EPCs from ⁵⁶Fe and ¹H Full-Body Irradiated Mice Over 28 Days

To assess the effects of ⁵⁶Fe-IR and ¹H-IR on survival and proliferation of EPCs ex-vivo, BM-EPCs were isolated from the bone marrow of the full-body IR mice for short-term time points of 2, 5 and 24-hours and long-term time points of 7, 14 and 28-days post-IR. Isolated BM-EPCs from each ⁵⁶Fe-IR and ¹H-IR mice were cultured for 72 hours ex-vivo in EPC selective EBM-2 media supplemented with bullet kit growth factors, on 15 mm circular glass coverslips (Electron Microscopy Sciences. Hatfield, Pa.) pre-coated with 0.2% gelatin in 24-well dishes (Corning Inc.). At the end of 72 hours after initial seeding for both short and long-term time points, BM-EPCs were trypsinized and harvested along with the supernatant growth media. No media change was done while BM-EPCs were in culture for 72 hours after seeding. Harvested cells were stained using Annexin V-FITC Apoptosis detection kit (eBiosciences Inc., San Diego, Calif.) as per manufacturer protocol and Propidium Iodide and analyzed by FACS analysis to evaluate ¹H- or ⁵⁶Fe-IR induced apoptosis in BM-EPCs. Cells in early stages of apoptosis are stained with Annexin V-FITC and Propidium Iodide (PI) was used to visualize nuclei. Data analyzed was plotted as percent (%) change in double Annexin V/PI (+) cells for both full-body ⁵⁶Fe-IR and ¹H-IR ex-vivo selected BM-EPCs compared to N-IR BM-EPCs that were set at 100%.

MTT Proliferation Assay of Ex-Vivo Expanded BM-EPCs from ⁵⁶Fe and ¹H Full-Body Irradiated Mice Over 28 Days

The proliferative capacity of BM-EPCs ex-vivo was evaluated after low and high-LET, ¹H and ⁵⁶Fe full-body IR for short-term time points of 2, 5 and 24 hours and long-term time points of 7, 14 and 28 days. Isolated and selected ex-vivo BM-EPCs from N-IR, ¹H-IR and ⁵⁶Fe-IR mice for all time points were counted using trypan blue exclusion and serial dilution were prepared and seeded at 5×10⁵ and 2.5×10⁵ cells/well into multiple wells of 96-well microplate (Corning Inc.) with 5 mm glass coverslips (Electron Microscopy Sciences) coated with 0.2% gelatin. After 72 hour incubation in 96-well microplares, media was removed from the wells and microplates were washed with PBS. CyQUANT cell proliferation kit (Life Technologies) was used to assay the proliferative capacity of BM-EPCs by incubating each well with 200 μl of CyQUANT GR dye for 2-5 minutes at RT as per manufacturer protocol. At the end of incubation, fluorescence of each well was measured using a microplate reader at 485/530 nm (excitation/emission) and O.D. values were compared to cell number standard curve obtained using the same dye concentration for a 12 standard serial dilution range. Data analyzed was plotted as total cell count for each seeding density over time as well as average cell count after 72-hour incubation in 96-well microplates for both ions.

### Statistical Analysis

All results were expressed as mean±SEM and plots were obtained. Statistical analysis was performed on the data by one-way ANOVA (Stat View Software, SAS Institute Inc.; Gary, N.C.). Differences were considered significant at p<0.05.

### Results

The Number of p-H2AX Foci is Increased in Non-Irradiated BM-Derived EPCs Treated with ¹H-IR In-Vitro

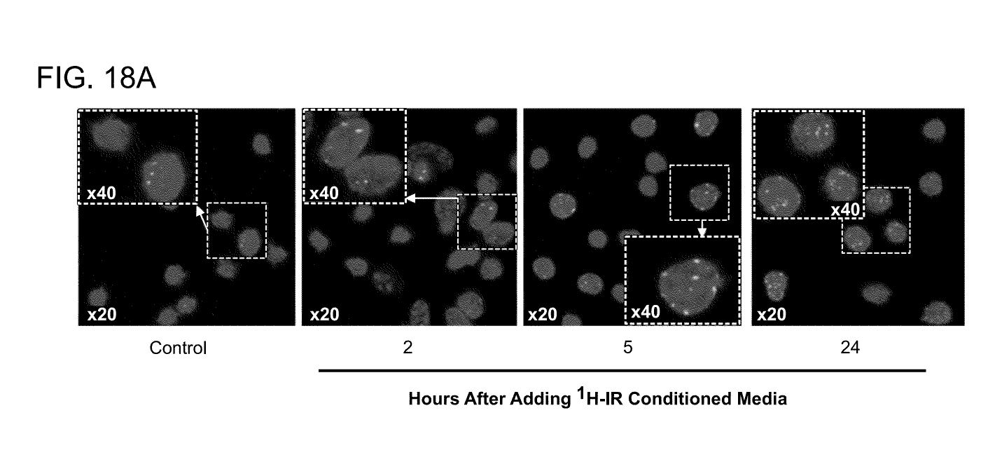

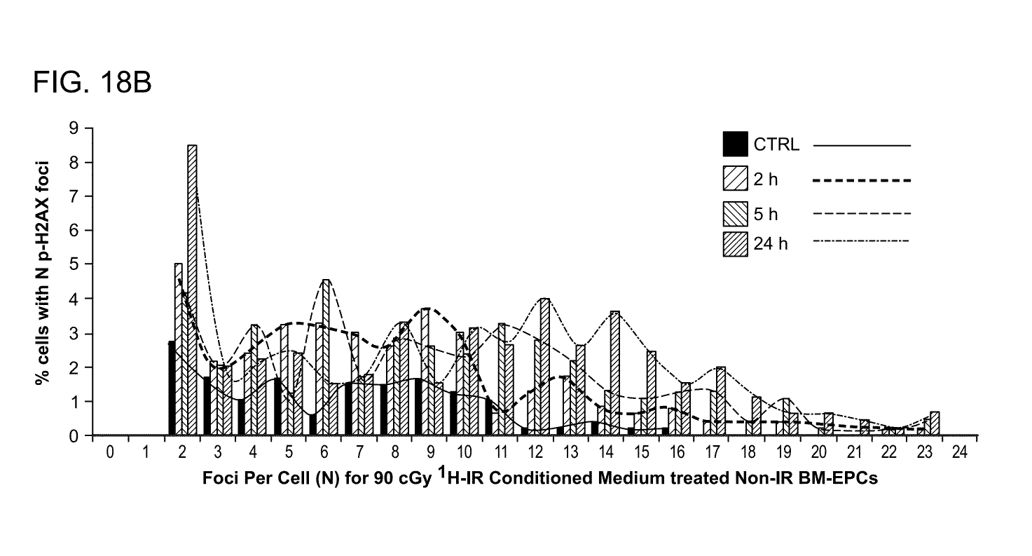

It was determined whether non-IR BM-EPCs may show evidence of bystander responses in media transfer experiment after treatment with ¹H-IR conditioned BM-EPCs media as described before (Sasi et al., 2014 Journal of Radiation Research, 55:i122-i123). The formation of p-H2AX foci in non-IR BM-EPCs was quantified 24 hours after treatment with media collected 2, 5 and 24-hours from BM-EPCs after ¹H-IR (IR-CM media). There was a steady and significant increase in the mean p-H2AX foci/cell for non-IR BM-EPCs treated with ¹H-IR BM-EPCs. Compared to control CM-treated non-IR BM-EPCs, there was 2-4-fold increase in the percent of cells with more than 4-11 p-H2AX foci/cell for ¹H-IR-CM-treated cells (FIG. 18A and FIG. 18B). There was a negligible less than 0.3% of cells with 12-16 p-H2AX foci/cell in control CM-treated BM-EPC; whereas 1.5-4% of ¹H-IR CM-treated BM-EPC had 12-16 p-H2AX foci/cell. Further, only ¹H-IR CM-treated BM-EPCs revealed 0.3-2% of cells with more than 17-23 p-H2AX foci/cell at 2, 5 and 24 hours after treatment (FIG. 18A and FIG. 18B). These findings suggest that non-IR BM-EPCs treated ¹H-IR-CM exhibit significant bystander responses in vitro up to 24 hours.

### Candidate Inflammatory Cytokines are Significantly Increased in ¹H-IR Conditioned Medium

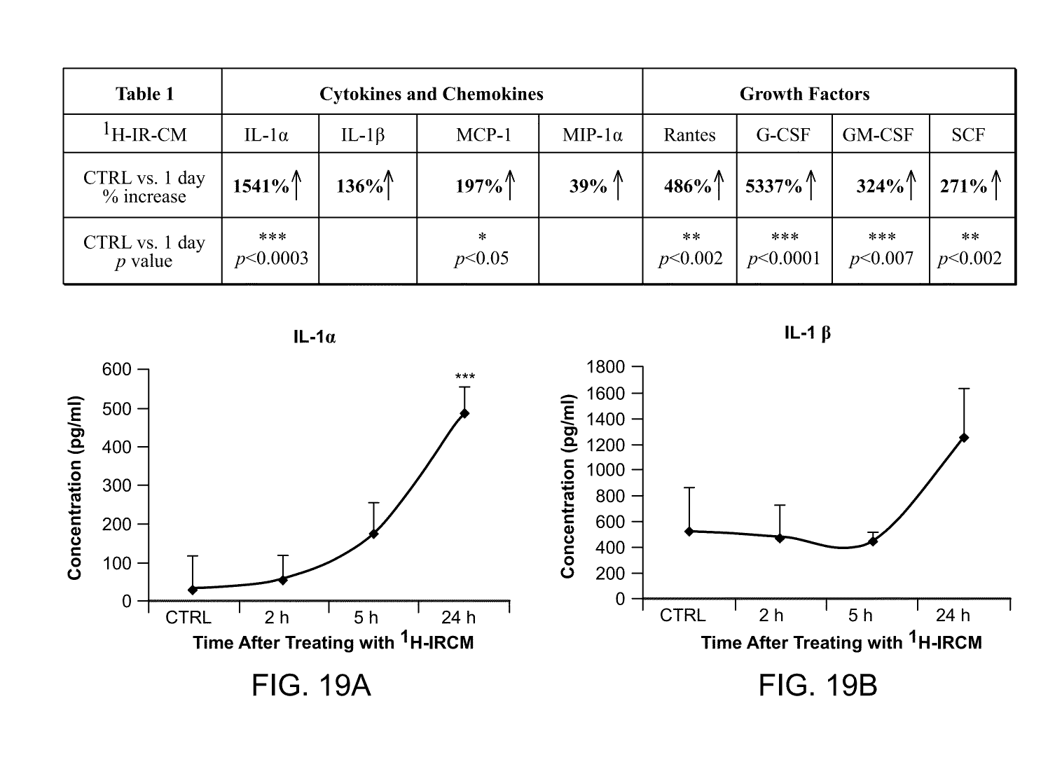

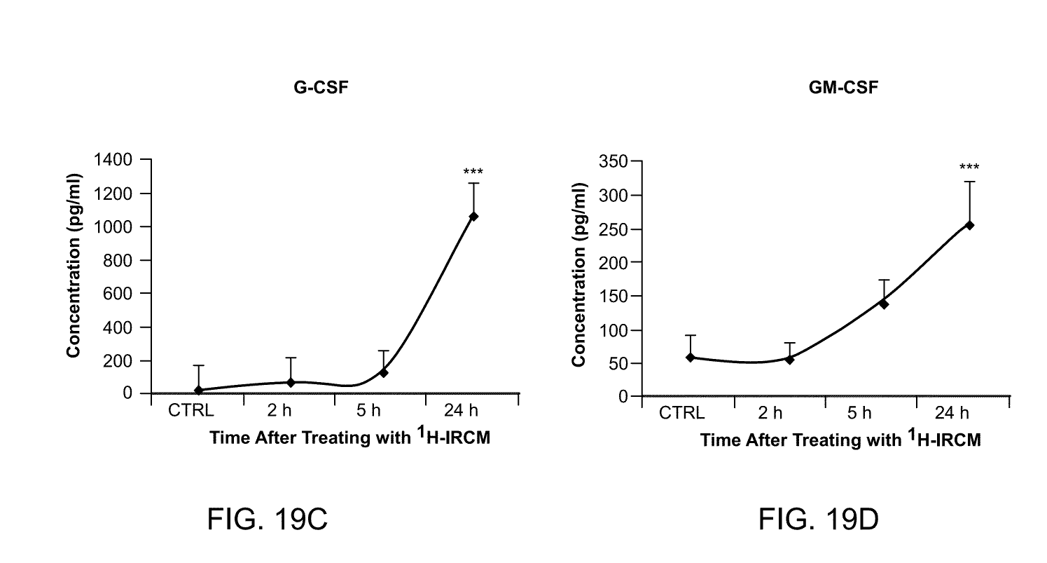

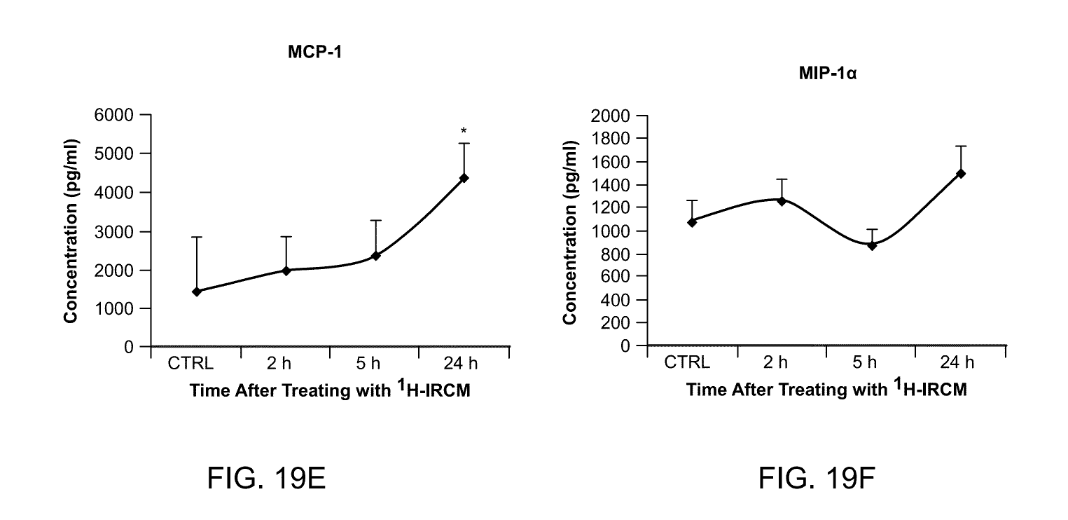

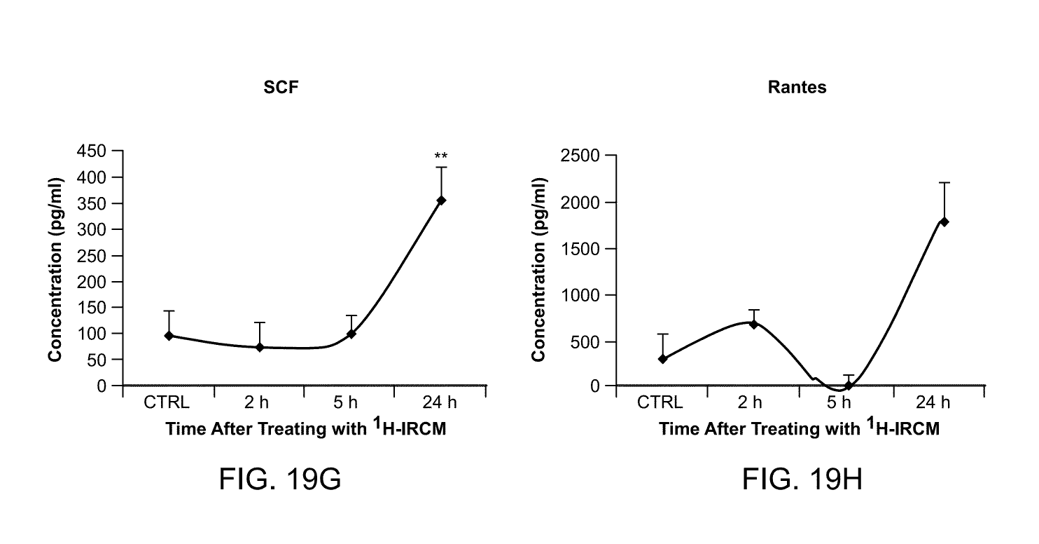

In 2006, Bubici et al., demonstrated that the convergence of radiation-mediated effect results in inflammation due to increased levels of various cytokines and chemokines that generate reactive oxygen and nitrogen species [25]. As described herein, the effect of ¹H-IR on production and accumulation of cytokines, chemokines and growth factors, such as IL-1α, IL-1β, MCP1, MIP-1α, Rantes, G-CSF, GM-CSF and SCF in BM-EPCs, all of which are known to be elevated within minutes to hours after ionizing radiation, was analyzed [26]. ELISA analysis of conditioned media from ¹H-IR BM-EPCs showed a gradual increase in the levels of several cytokines, chemokines and growth factor, when compared to non-JR-CM. The maximum and statistically significant increase (2-53-fold) in IL-1α, MCP-1, Rantes, G-CSF, GM-CSF and SCF were observed by 24 hours (FIG. 19A, FIG. 19C-E, FIG. 19G, FIG. 19H and Table 3). Although, IL-1β and MIP-1α levels in ¹H-IR EPC media were slightly elevated (˜39-136%) by 24 hours it was not significant when compared to non-IR EPC media (FIG. 19B, FIG. 19F and Table 3). These findings suggest that ¹H-IR at 90 cGy induces accumulation of several cytokines and growth factors that have been directly implicated in mediating bystander responses in BM-derived EPCs (Sasi et al., 2014 Journal of Radiation Research, 55:i122-i123) as well as other cell types.

### Table 3A-Table 3B. Statistical Analysis for Accumulation of Cytokines, Chemokines and Growth Factors in BM-EPC ⁵⁶Fe- and ¹H-IR-CM

Table 3A shows statistical analysis of protein concentrations using ELISA for different cytokines, chemokines and growth factors for ⁵⁶Fe-IR-CM from IR-EPCs compared to respective control N-IR-CM. Categorized into cytokines, chemokines and growth factors IR-CM revealed statistical significance, in comparison to control only for 1 day post-IR time point with the exception of TNF-α. Table 3B shows statistical analysis of protein concentrations using ELISA for different cytokines, chemokines and growth factors for ⁵⁶Fe-IR-CM from IR-EPCs compared to respective control N-IR-CM. Categorized into cytokines, chemokines and growth factors IR-CM revealed statistical significance, in comparison to control only for 1 day post-IR time point. Table 3C shows statistical analysis of protein concentrations using ELISA for different cytokines, chemokines and growth factors at each time point compared between ⁵⁶Fe-IR-CM and ¹H-IR-CM from EPCs. Categorized into cytokines, chemokines and growth factors IR-CM from both ions revealed statistical significance, at each time point mostly in Cytokines and Chemokines and no significant change in growth factors.

Statistical analysis of cumulative levels of proteins in ⁵⁶Fe-IR-CM and ¹H-IR-CM in
control vs. all time points post-IR and between Ions for all time points (ANOVA)

| | Cytokines and Chemokines | | | | | Growth factors | | | |
|---|---|---|---|---|---|---|---|---|---|
| | TNF-a | IL-1a | IL-1b | MCP-1 | MIP-1a | Rantes | G-CSF | GM-CSF | SCF |
| A. | | | | | | | | | |
| ⁵⁶Fe-IR-CM | | | | | | | | | |
| CTRL vs. 2 hr | | | | | | | | | |
| CTRL vs. 5 hr | p < 0.03 | | | | | | | | |
| CTRL vs. 1 d | | p < 0.02 | | p < 0.04 | p < 0.04 | p < 0.0001 | p < 0.02 | p < 0.04 | p < 0.02 |
| B. | | | | | | | | | |
| ¹H-IR-CM | | | | | | | | | |
| CTRL vs. 2 hr | | | | | | | | | |
| CTRL vs. 5 hr | p < 0.0003 | | | | | | | | |
| CTRL vs. 1 d | p < 0.0001 | p < 0.0003 | | p < 0.05 | | p < 0.002 | p < 0.0001 | p < 0.007 | p < 0.002 |
| C. | | | | | | | | | |
| ⁵⁶Fe-IR vs. ¹H-IR | | | | | | | | | |
| 2 hr | | | | | | | | | |
| 5 hr | p < 0.0001 | | p < 0.04 | | | | | | |
| 1 day | p < 0.0001 | p < 0.003 | p < 0.03 | | p < 0.05 | | | | |

Full Body ¹H-IR Induce Cyclical Increases in BM-Derived EPCs Apoptosis and No Change in Proliferation Capacity of BM-EPCs over 28 Days Post-IR

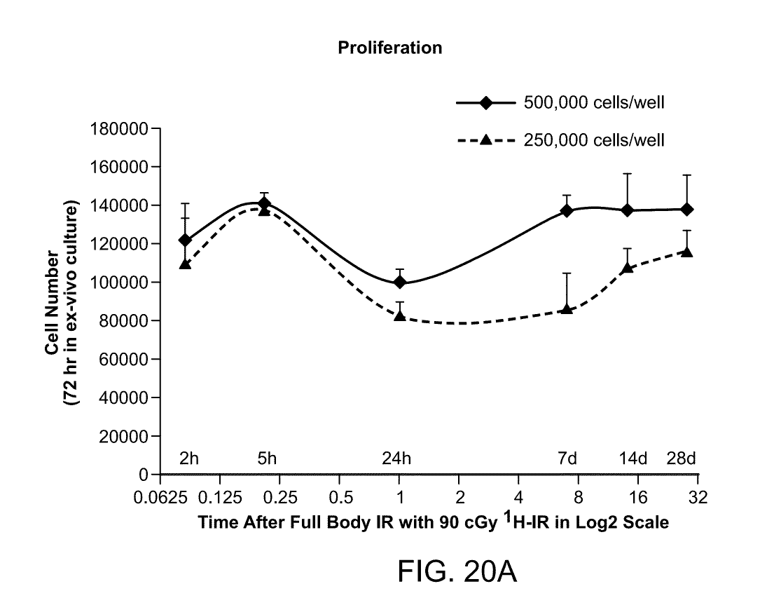

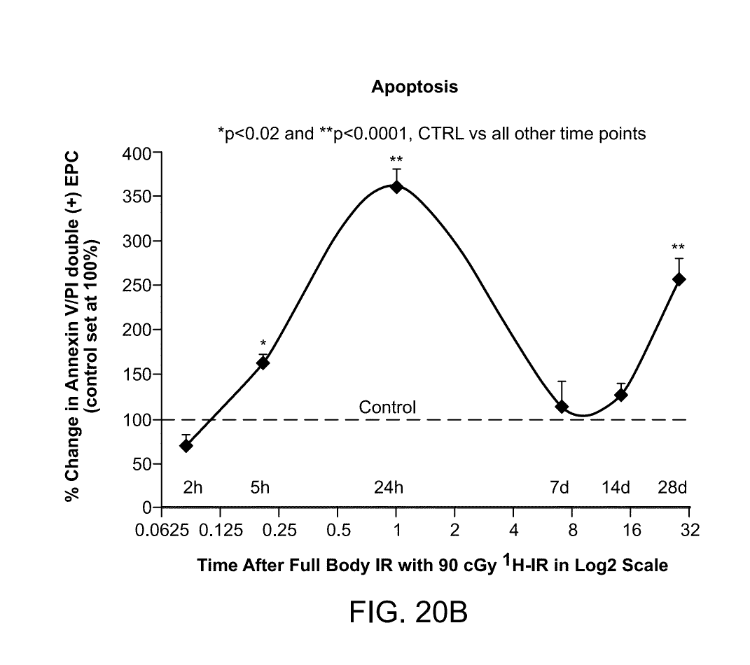

To determine the effect of full-body ¹H-IR on ex-vivo proliferation, BM-derived EPCs were plated in 96-well plates (two different densities 5×10⁵ and 2.5×10⁵) at 2, 5 24 hours and 7, 14, 28 days after isolation from bone marrow. BM-EPC proliferation capacity was determined 72 hours after plating using the MTT assay. The number of cell in control non-IR BM-EPCs 72 hours after plating, collected at various time points up to 28 days, for either of the plating densities was not changed significantly at any time (1.3×10⁵±6.799 and 1.04×10⁵±11,050 cells, for 5×10⁵ and 2.5×10⁵ plating densities, respectively). Compared to control non-IR BM-EPCs at any collection time point, there was a small 20-30% up or down variation in the number of BM-EPCs over time, however, this was not significant statistically (FIG. 20A).

To determine the effect of full-body ¹H-IR on ex-vivo apoptosis, BM-derived EPCs were plated in 24-well plates at 2, 5 24 hours and 7, 14, 28 days after isolation from bone marrow. BM-EPC apoptosis was determined 72 hours after plating using FACS analysis of BM-derived EPCS double stained with Annexin V and Propidium Iodide. The results revealed that after full-body ¹H-IR there was a 50% and 350% increase in BM-EPC apoptosis collected at 5 and 24 hours, respectively, and cultured for 72 hours ex-vivo (FIG. 20B). By day 7 the apoptosis was decreased to near control non-IR levels. However, there was a second 250% increase in BM-EPC apoptosis in ¹H-IR on day 28 (FIG. 20B). This data indicates that there is a cyclical increase, early at 5 hours and delayed at 28 days, in BM-EPC apoptosis after a single low dose ¹H-IR.

The Number of p-H2AX Foci is Increased in Non-Irradiated BM-Derived EPCs Treated with ⁵⁶Fe-IR In-Vitro

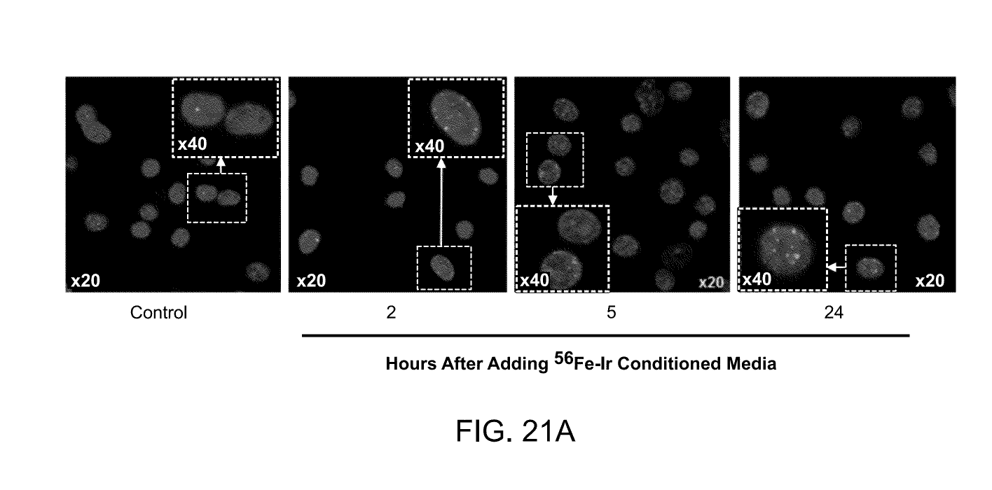

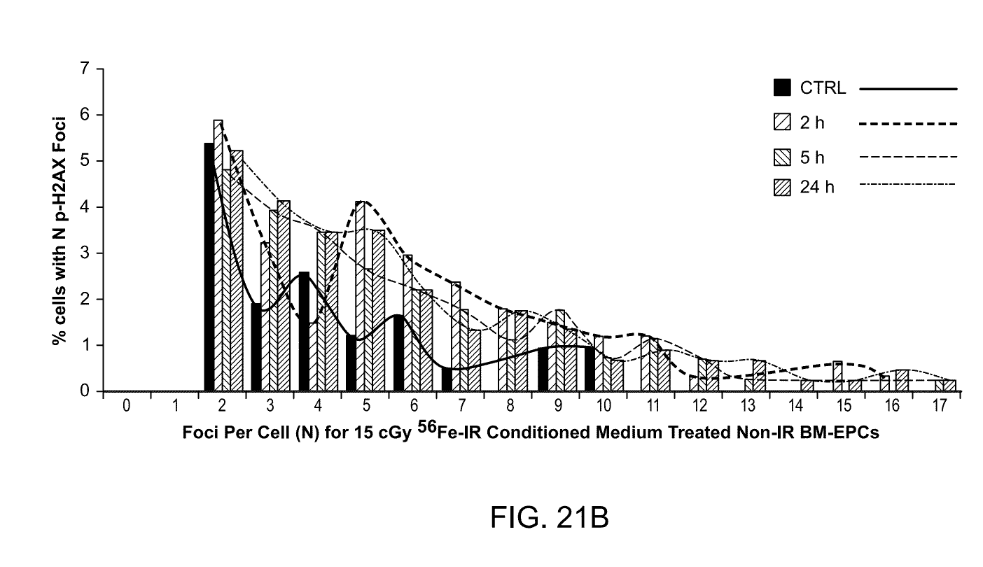

It was next determined whether non-IR BM-EPCs may show evidence of bystander responses in media transfer experiment after treatment with ⁵⁶Fe-IR conditioned BM-EPCs media as described before (Sasi et al., 2014 Journal of Radiation Research, 55:i122-i123). The formation of p-H2AX foci in non-IR BM-EPCs was quantified 24 hours after treatment with media collected 2, 5 and 24-hours from BM-EPCs after ⁵⁶Fe-IR (⁵⁶Fe-IR-CM media). There was a steady and significant increase in the mean p-H2AX foci/cell for non-IR BM-EPCs treated with ⁵⁶Fe-IR BM-EPCs. Compared to control CM-treated non-IR BM-EPCs, there was 1-4-fold increase in the percent of cells with more than 4-10 p-H2AX foci/cell for ⁵⁶Fe-IR-CM-treated cells (FIG. 21A and FIG. 21B). Further, only ⁵⁶Fe-IR CM-treated BM-EPCs revealed 0.3-1.3% of cells with more than 11-17 p-H2AX foci/cell at 2, 5 and 24 hours after treatment (FIG. 21A and FIG. 21B). These findings suggest that non-IR BM-EPCs treated ⁵⁶Fe-IR-CM exhibit significant bystander responses in vitro up to 24 hours.

### Inflammatory Cytokines are Significantly Increased in ⁵⁶Fe-IR Conditioned Medium

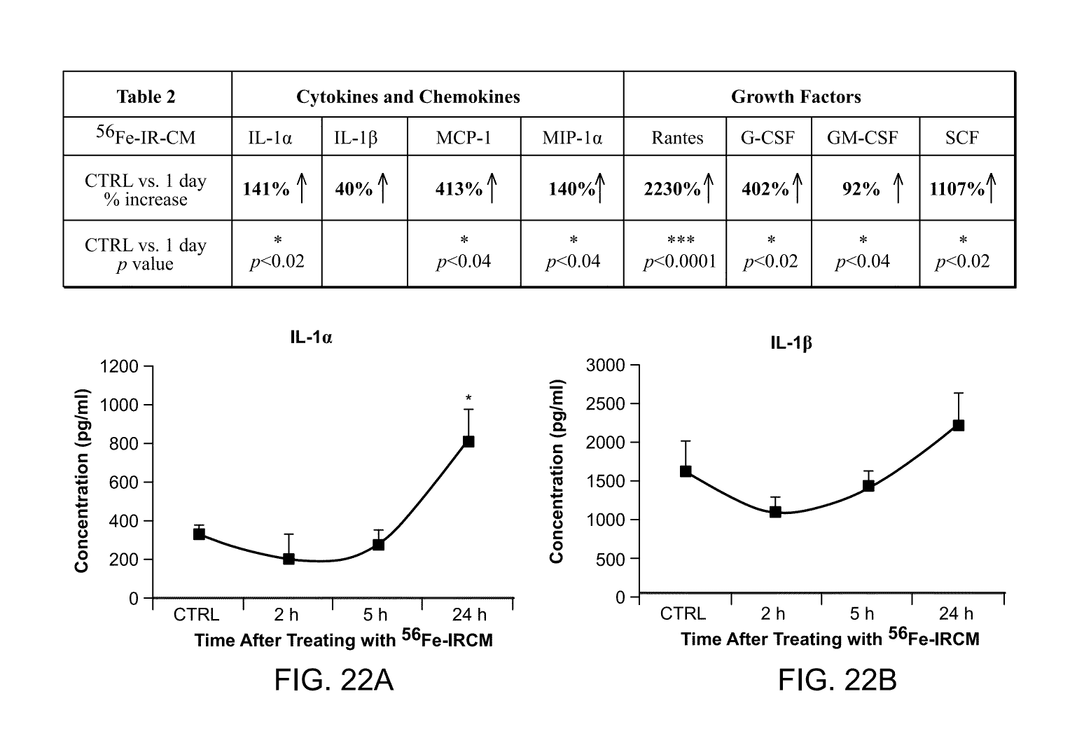

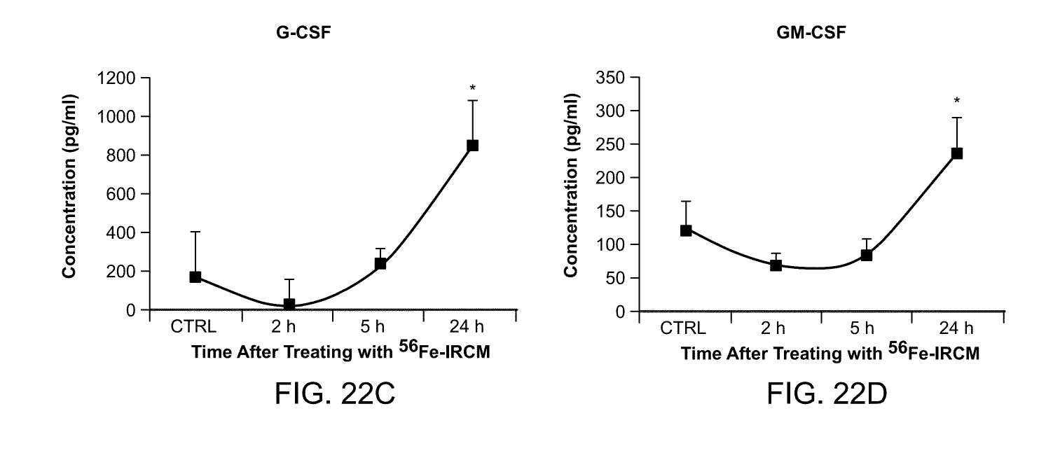

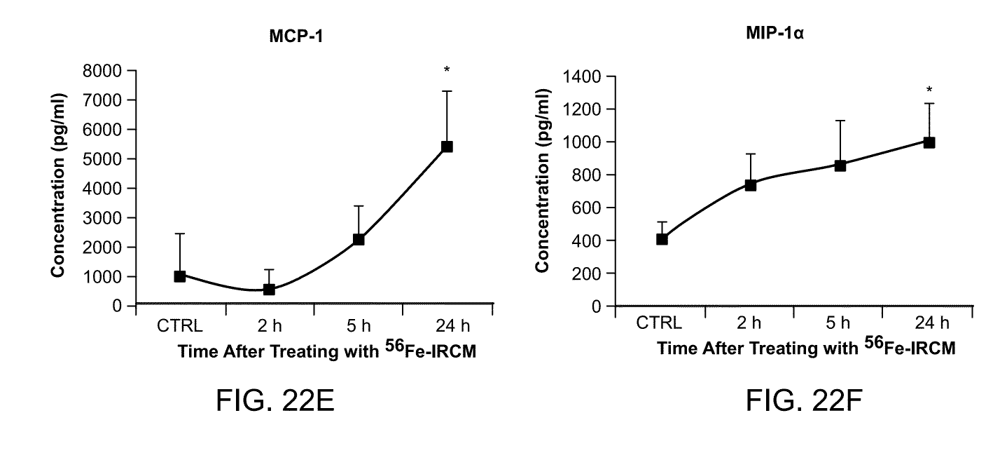

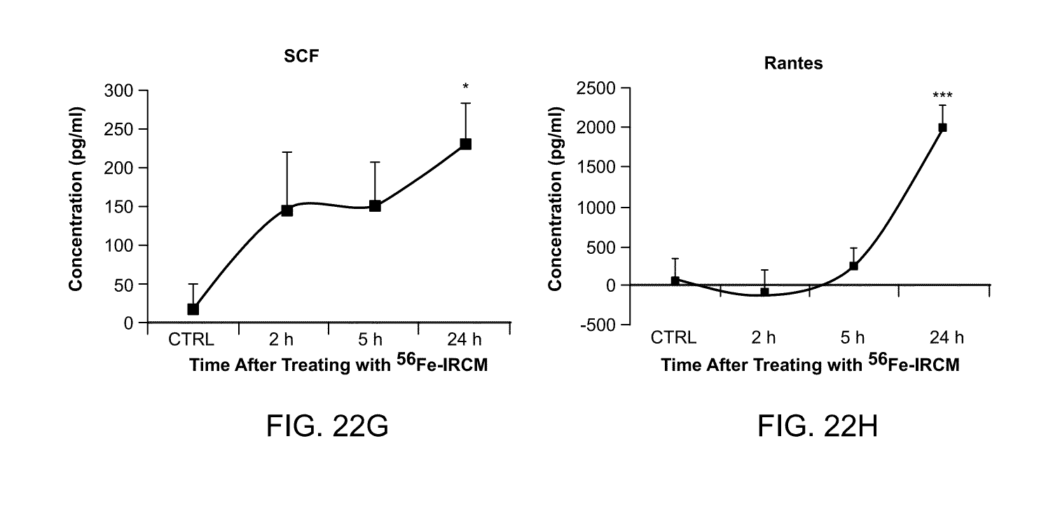

Similar to studies with ¹H-IR BM-EPCs the production and accumulation of cytokines, chemokines and growth factors in the media of BM-EPCs irradiated with 15 cGy, 1 GeV/n of ⁵⁶Fe-IR was evaluated. ELISA analysis of conditioned media from ⁵⁶Fe-IR BM-EPCs showed a gradual increase in the levels of several cytokines, chemokines and growth factor, when compared to non-IR-CM. The maximum and statistically significant increase (1.4-22-fold) in IL-1α, MCP-1, MIP-1α, Rantes, G-CSF, GM-CSF and SCF were observed by 24 hours (FIG. 22A, FIG. 22C-H, and Table 4). Although, IL-1β level in ⁵⁶Fe-IR EPC media were slightly elevated (˜40%) by 24 hours it was not significant when compared to non-IR EPC media (FIG. 22B and Table 4). These findings suggest that, like in ¹H-IR media, ⁵⁶Fe-IR at 15 cGy induces accumulation of several cytokines and growth factors that have been directly implicated in mediating bystander responses in various cell types (Sasi et al., 2014 Journal of Radiation Research, 55:i122-i123).

Table 4A-Table 4B. Percent change in Cumulative Levels of Cytokines, Chemokines and Growth Factors in BM-EPC ⁵⁶Fe- and ¹H-IR-CM

Table 4A shows the percent change in cumulative protein concentrations by 24 hours post-IR for different cytokines, chemokines and growth factors on ⁵⁶Fe-IR-CM from IR-EPCs compared to respective control N-IR-CM. Categorized into cytokines, chemokines and growth factors IR-CM revealed a significant increase (in red) for all protein analyzed using ELISA with the exception of TNF-α which showed a decrease by 24 hours post-IR (in blue). Table 4B shows the percent change in cumulative protein concentrations by 24 hours post-IR for different cytokines, chemokines and growth factors on ¹H-IR-CM from IR-EPCs compared to respective control N-IR-CM. Categorized into cytokines, chemokines and growth factors IR-CM revealed a significant increase (in red) for all protein analyzed using ELISA. Table 4C shows the percent change in cumulative protein concentrations by 24 hours post-IR for different cytokines, chemokines and growth factors compared between ⁵⁶Fe-IR-CM and ¹H-IR-CM from IR-EPCs at 24 hours post-IR. Categorized into cytokines, chemokines and growth factors IR-CM revealed a significant increase (in red) and significant decrease (in blue) for respective proteins analyzed using ELISA. P values are also provided in the Table below.

Percent Change in Cumulative levels of Cytokine, Chemokines and
Growth Factors in ⁵⁶Fe-IR-CM and ¹H-IR-CM by 24 hours vs. Control

| | Cytokines and Chemokines | | | | | Growth Factors | | | |
|---|---|---|---|---|---|---|---|---|---|
| | TNF-a | IL-1a | IL-1b | MCP-1 | MIP-1a | Rantes | G-CSF | GM-CSF | SCF |
| A. | | | | | | | | | |
| ⁵⁶Fe-IR-CM | | | | | | | | | |
| CTRL vs. 1 d | 90% ↓ | 141%↑ | 40%↑ | 413%↑ | 140%↑ | 2230%↑ | 402%↑ | 92% ↑ | 1107%↑ |
| B. | | | | | | | | | |
| ¹H-IR-CM | | | | | | | | | |
| CTRL vs. 1 d | 663%↑ | 1541%↑ | 136%↑ | 197%↑ | 39%↑ | 486%↑ | 5337%↑ | 324%↑ | 271%↑ |
| C. | | | | | | | | | |
| ⁵⁶Fe-IR vs. | | | | | | | | | |
| ¹H-IR | | | | | | | | | |
| 1 day | 98%↓ | 67%↑ | 80%↑ | 26%↑ | 34%↓ | 11%↑ | 21%↓ | 5% ↓ | 36%↓ |

| | Cytokines and Chemokines | | | | Growth Factors | | | |
|---|---|---|---|---|---|---|---|---|
| | IL-1α | 1L-1β | MCP-1 | MIP-1α | Rantes | G-CSF | GM-CSF | SCF |
| ¹H-IR-CM | | | | | | | | |
| CTRL vs. 1 day | 1541%↑ | 136%↑ | 197%↑ | 39% ↑ | 486%↑ | 5337%↑ | 324%↑ | 271%↑ |
| % increase | | | | | | | | |
| CTRL vs. 1 day | *** | | * | | ** | *** | *** | ** |
| p value | p < 0.0003 | | p < 0.05 | | p < 0.002 | p < 0.0001 | p < 0.007 | p < 0.002 |
| ⁵⁶Fe-IR-CM | | | | | | | | |
| CTRL vs. 1 day | 141% ↑ | 40% ↑ | 413%↑ | 140% ↑ | 2230%↑ | 402% ↑ | 92% ↑ | 1107%↑ |
| % increase | | | | | | | | |
| CTRL vs. 1 day | * | | * | * | *** | * | * | * |
| p value | p < 0.02 | | p < 0.04 | p < 0.04 | p < 0.0001 | p < 0.02 | p < 0.04 | p < 0.02 |

### Full Body ⁵⁶Fe-IR Induce Cyclical Increases in BM-EPCs Apoptosis and Affects Significantly Proliferation Capacity of BM-EPCs Ex-Vivo Over 28 Days Post-IR

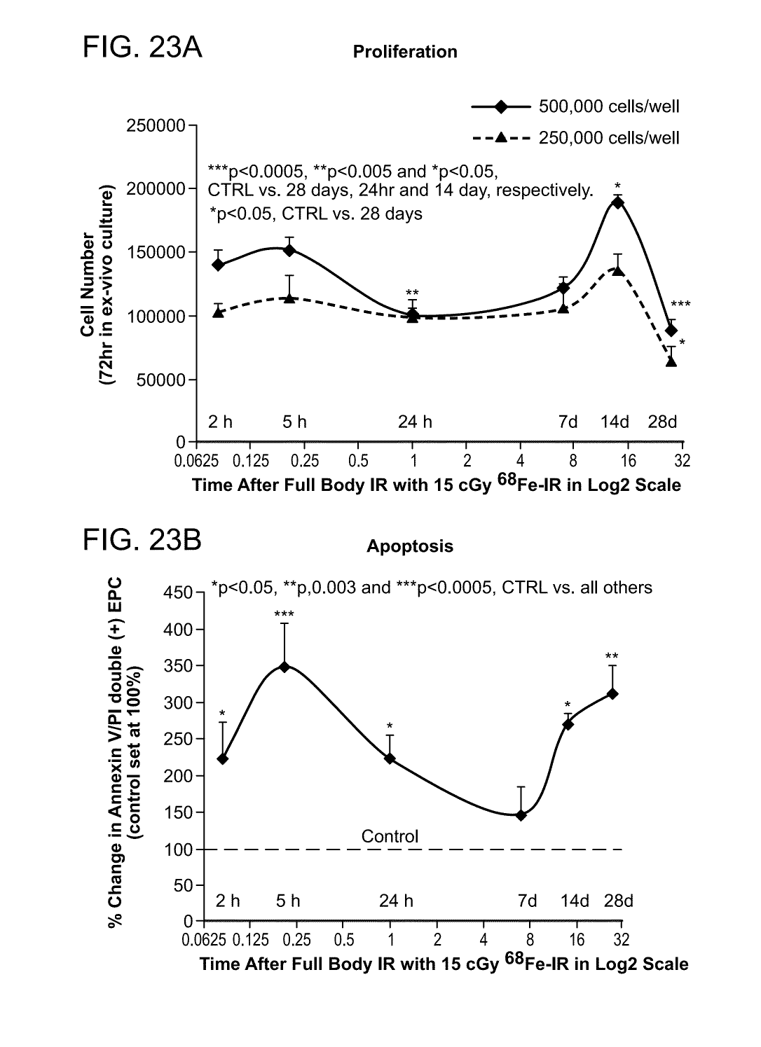

MTT assay performed after 72 hours ex-vivo expansion of BM-EPCs at two plating densities (5×10⁵ and 2.5×10⁵ cells per well) showed no significant change in average cell proliferation for full-body ⁵⁶Fe-IR mice up to 7 days post-IR (FIG. 23A). However compared to 7 days post-IR there was ˜41% increase in proliferation on day 14, but the rate of BM-EPC proliferation dropped significantly (to ˜55% of day 14) below control levels on day 28 for ⁵⁶Fe-IR mice (FIG. 23A). The significant decrease in cell number by 28 days was significant compared to all time points and be an indication long lasting effect of a single low dose ⁵⁶Fe-IR on survival and proliferation of BM-EPCs ex-vivo.

Accordingly, FASC analysis of Annexin V/PI double positive cells revealed that 2, 5 and 24 hours after full-body ⁵⁶Fe-IR there was 2.5-3.5-fold increase in BM-EPC apoptosis, with the peak 3.5-fold increases in apoptosis after ⁵⁶Fe-IR at 5 hours (FIG. 23B). By day 7 the apoptosis was decreased to near control non-IR levels. However, there was a gradual increase in EPC apoptosis in ⁵⁶Fe-IR mice between days 7-28, with maximum 3-fold increase in apoptosis on day 28 (FIG. 23B). This data indicates that there is a cyclical increase, early 5 hours and delayed 28 days, in BM-EPC apoptosis after a single low dose ⁵⁶Fe-IR.

### Discussion

A growing body of evidence indicates that in the heart and other organ-tissues vascular homeostasis does not exclusively rely on proliferation of local endothelial cells (ECs) but also involves BM-EPCs [27]. Indeed, studies have demonstrated that in patients with CV risk factors, the number and migratory ability of EPCs isolated from peripheral blood is reduced [28] and EPC function is impaired [29]. In addition, a strong inverse correlation was reported between the number of circulating EPCs, vascular function and the subject's combined Framingham cardiovascular factor score [30]. Furthermore, measurements of flow-mediated brachial-artery reactivity also revealed a significant relation between endothelial function and the number of EPCs, supporting a role for EPCs in the maintenance of endothelial integrity [31].

DNA damage caused by ionizing radiation in the form of a broad spectrum of lesions in DNA, includes single-strand breaks (SSBs) and DSBs [32]. Radiation induced DSB in DNA is a major toxic component in cell apoptosis induction [32-34] and improper DSB repair can result in mutations [34]. Cells respond to DNA-damage by initiating slow cell cycle program and in some cases where damage is to a great extent cells can enter apoptotic cycle [34-36]. Of the several known factors involved in DNA-repair of DSB, the phosphorylation of histone H2A isoform (H2AX) at serine 139 site results in alterations in chromatin structure at the site of DNA-damage for efficient DSB repair [35-38]. This phosphorylation of H2AX takes place within minutes of DNA damage [35] and the number of ionizing radiation induced foci (IRIF) is observed to increase proportionally with the number of DSBs which indicates the severity of damage [35, 38-41]. There is a significant decay of p-H2AX foci in γ-IR mouse heart resident ECs and non-ECs, which is indicative of considerable DNA DSB repair, however with slower than usual repair kinetics reported for other primary cells, i.e., fibroblasts, leukocytes [42, 43]. The slow decay of p-H2AX foci in the heart ECs and decreased survival of BM-EPCs ex-vivo 7 days after low dose full-body 1 Gy γ-IR along with earlier findings [44, 45] further highlights the emphasis and involvement of BM-EPCs in endothelial function and survival.

The magnitude of ionizing radiation induced DNA damage reflected by p-H2AX foci [46] is measured in terms of LET along the track of irradiation. Heavy ion particles such as ⁵⁶Fe (high-LET) that can pass deeper within a cell or tissue tend to cause dense localized ionization and lose more energy along their straight track [47, 48] resulting in severe, complex and obdurate to repair clustered damage [46, 48, 49] when compared to low-LET particles such as γ and ¹H that diffuse DNA damage produced by sparse ionization [36, 46, 48, 50-52]. As described herein, both low dose ⁵⁶Fe- and ¹H-IR induces radiobiological bystander responses (RBR) in naïve EPC in-vitro that are of longer duration with increased presence of p-H2AX foci over 24 hours post-IR which signifies the detrimental effects of radiation on EPCs. Since the formation of foci depends on phosphorylation of H2AX and precedes repair mechanisms it is considered as a necessary step in initiation of DNA damage repair [35] which is evident for both ions within 2 hours post-IR. The normal DNA repair capacity of hematopoietic stem and progenitor cells have been known to be overwhelmed under extreme damaging conditions such as in tobacco users resulting in un-repairable cluster damage [53] which is comparable to the response seen in ⁵⁶Fe-IR BM-EPCs. Cells deficient in DNA-repair tend to exhibit reduced p-H2AX foci post ionizing-IR [35] and since high-LETs such as ⁵⁶Fe-IR induce more serious DNA DBSs than low-LETs such as ¹H-IR [50, 54]. This can result in increased cell death and cell cycle arrest and our findings of lower mean p-H2AX foci in ⁵⁶Fe-IR BM-EPCs due to clustering of DNA damage foci [48] when compared to ¹H-IR cells, but increasing for both ions can be considered as an indirect acute effect of such ionizing-IR on naïve BM-EPCs.

BM-EPCs which are mobilized from the bone marrow into circulation in response to injury or stress are aided by numerous chemokines and growth factors [55] that are known to be elevated within minutes to hours after ionizing radiation [26, 56]. Pro-inflammatory cytokines such as TNF-α, IL-1α and IL-6 have been well documented to be regulated as a direct effect of γ-IR in murine hematopoietic cells [57] as well after ionizing-IR in human epithelial cells [58]. Inflammation and fibrotic response as a result of injury in human lungs or mammalian cells is mediated by IL-1α, IL-1β and TNF-α and these cytokines are also known to be increased after irradiation [59, 60]. Fibroblasts exposed to X-rays [61] and human endothelial cells [62] exposed to ionizing radiation have shown to induce increased levels of IL-6 and IL-8. Similar observations were made in the bystander study described herein where in BM-EPCs exposed to low dose ionizing-IR resulted in ⁵⁶Fe-IR-CM having elevated levels of IL-1α, IL-1β and MCP-1 while cumulative levels for G-CSF, GM-CSF and SCF were decreased when compared to ¹H-IR-CM by 24 hours post-IR except for TNF-α.

BM-EPCs are mobilized into circulation by G-CSF [63] and G-CSF has also been identified for its radioprotective role in mammals [64, 65] along with VEGF. VEGF treatment of human dermal endothelial cells significantly increased its radioprotectivity to γ-IR and VEGF also plays a crucial role in protecting ECs against ionizing-IR [44]. Since VEGF is also known to promote angiogenesis and a key survival factor for ECs [44], undetectable levels of VEGF in CM from ⁵⁶Fe-IR from BM-EPCs along with other growth factors such as basic fibroblast growth factor (bFGF) and epidermal growth factor (EGF) when compared to ¹H-IR-CM is a significant indicator of high-LET ⁵⁶Fe ionizing-IR induced severe damage to BM-EPCs which can eventually result in EC dysfunction causing tissue damage and delayed effects. FGF is known to aid in repair of radiation induced damage in ECs [66, 67] and proliferation of ECs and it is safe to assume that undetectable levels of FGF in our CM from ⁵⁶Fe-IR BM-EPCs when compared to ¹H-IR can be indicative of extensive un-repairable radiation induced DNA damage in EPCs thus hampering its survival and proliferative capacity. Also slightly increased accumulation of G-CSF over 24 hour post-IR in ¹H-IR-CM might be indicative of immediate initiation of survival response to low-LET ionizing-IR induced DNA damage. A recently published report showed linear relationship between changes in the concentration of MCP-1 and cumulative doses of small fractionated IR doses [68]. The results described herein showed similar findings for BM-EPCs exposed to both ionizing-IR in-vitro with ⁵⁶Fe-IR-CM having constitutively higher concentrations by 24 hours. Enhanced cytokine and chemokines levels after ⁵⁶Fe-IR appears to be a result of significant increase in cell death and any surviving cells entering the apoptotic cycle while the increased release of growth factors along with cytokines and chemokines by BM-EPCs post ¹H-IR appears to be suggestive of cells being maintained at a metabolic activity over 24 hours post-IR.

High levels of pro-inflammatory cytokines after radiation exposure can cause profound effects by such as cell death and perpetuate further DNA damage. DSBs induced by high-LET are hard to repair and extremely lethal with the potential to undergo mutagenesis [50, 53] which is evident from the initial increase in BM-EPCs within 5 hours post ⁵⁶Fe-IR and significantly lower proliferative capacity over 28 days. This reduced proliferation works in tandem with the increased apoptosis at later time points (7-28 days). In comparison to low-LET where complex damage is uncommon [48] initial increase in apoptosis for BM-EPCs was only by 24 hours post ¹H-IR which eventually increased by 28 days. However this increased cell death was compensated by the initial proliferative response at 5 hours that was maintained till later time points. Taken together, this data suggests that early increase in BM-EPC apoptosis may be a direct “hit” radiation-mediated effect, whereas later increase in apoptosis and decrease in proliferation could be a consequence of non-targeted radiobiological effects. Thus, it is concluded that a single low dose ⁵⁶Fe-IR may have long-lasting effect on survival and proliferation of BM-EPCs and may induce delayed non-targeted effects when compared to ¹H-IR. Growth arrest by 3-6 days in culture after a single dose 10 Gy γ-IR has been underlined in human dermal microvascular endothelial cells previously [44]. Significantly higher growth inhibition along with progressive cell death of ECs exposed to a dose response of 2.5-20 Gy γ-IR was evaluated by Johnson et al. [45].

Stem cells express at high levels genes associated with DNA repair and protection from stress, including oxidative stress [69, 70]. It has been shown that EPCs express lower levels of basal and stress-induced intracellular reactive oxygen species (ROS) than primary ECs because EPCs express higher levels of catalase, manganese superoxide dismutase (MnSOD) and glutathione peroxidase-1 (GPx-1) [71, 72] and collective inhibition of catalase, MnSOD, and GPx-1[73] increases ROS levels in EPCs and that in turn impairs EPC survival and migration [71]. As ischemic/damaged tissue is characterized by high levels of inflammatory cytokines which activate ROS production [74], it has been proposed that high levels of ROS metabolizing enzymes in EPCs are essential to maintain their survival during tissue regeneration after injury. Conversely, these findings suggest that an imbalance in ROS can contribute to EPC dysfunction and that oxidative stress may impair neovascularization, thereby contributing to the pathogenesis and the progression of CV risks. Since ionizing radiation induce oxidative stress in target tissue through the generation of reactive oxygen and nitrogen species mediated by increase in cytokines and chemokines eventually resulting in cell death [25, 75-77] our cumulative ELISA findings are a good indicator of impaired BM-EPC function post ionizing-IR. These findings in BM-EPCs can be corroborated with radiation induced inflammatory changes in ECs resulting in modification of homeostasis and endothelial dysfunction [78].

A recent retrospective study in cancer survivor patients diagnosed with CV diseases after radiotherapy has shown a 2.5 fold increase in mortality after cardiac surgery [79], where in only 5% death is associated with recurrent malignancy and an astounding 49% mortality is cardiothoracic associated [79]. Epidemiologic data on IR-induced circulatory disease from radiotherapy patients [80-82], non-occupational exposure [24, 83, 84] and occupational exposure has demonstrated that CV morbidity may occur within months or years, and CV mortality may occur within decades, after initial IR exposure. Since EPCs are embedded in the microenvironment of bone marrow stroma which is considered as the most concentrated reservoir [85], as well as ECs and are mobilized to the circulation in response to activation of several mobilizing signaling pathways [55, 86], ionizing radiation induced dysfunction in BM-EPCs can ultimately result in degenerative CV risks. Although there is no quantifiable data available on the contribution of EPCs during adaptive cardiac growth in healthy individuals, EPC contribution to the process of post-natal neovascularization is well documented over the years [8-11].

Reduced cell survival is accompanied by the persistence of DNA damage [87] as seen with high-LETs which can be fatal to glioma stem-like cells [32]. Also, radiation induced cytokines are known to hunt in packs and with them altering radiosensitivity of tumor cells [88]. Such alteration of intrinsic radiosensitivity can be linked to damage in cells due to DNA repair, secretion of biomolecules such as chemokines, cytokines and growth factors, cell proliferation, differentiation and death [88, 89] as seen in the results presented herein with BM-EPCs exposed to ionizing-IR. Since the transition from pro-inflammatory to more anti-inflammatory environment is crucial for proper tissue recovery it is of the utmost importance that cell proliferation and resistance to radiation induced cell death in bystander cells is enhanced [26, 89].

Longitudinal studies using low-dose heavy ion (HZE) radiation studies are utilized to determine radiation-induced long-term CV risks. Taken together, in the presence of extreme damaging conditions such as low- and high-LET ionizing radiation, BM-EPCs demonstrate slow DNA-repair characteristics along with increased cell apoptosis and decreased proliferation. Such BM-EPC dysfunction can ultimately culminate in the form of fibrosis in the heart, loss of cardiac of functions, and eventually increased CV degenerative risks.

### REFERENCES

The following documents are cited in Example 8. As should be understood, the citations above to one or more numbers in brackets (e.g., [1-3]) refers to the document listed below identified by the same number.
1. Watson G E, Pocock D A, Papworth D, Lorimore S A and Wright E G (2001) In vivo chromosomal instability and transmissible aberrations in the progeny of haemopoietic stem cells induced by high- and low-LET radiations. International journal of radiation biology 77: 409-417.
2. Watson G E, Lorimore S A, Macdonald D A and Wright E G (2000) Chromosomal instability in unirradiated cells induced in vivo by a bystander effect of ionizing radiation, Cancer research 60: 5608-5611.
3. Todd P, Pecaut M J and Fleshner M (1999) Combined effects of space flight factors and radiation on humans. Mutation research 430: 211-219.
4. Orlic D, Kajstura J, Chimenti S, Jakoniuk I, Anderson S M, et al. (2001) Bone marrow cells regenerate infarcted myocardium. Nature 410: 701-705.
5. Kalka C, Masuda H, Takahashi T, Kalka-Moll W M, Silver M, et al. (2000) Transplantation of ex vivo expanded endothelial progenitor cells for therapeutic neovascularization. Proceedings of the National Academy of Sciences of the United States of America 97: 3422-3427.
6. Kamihata H, Matsubara H, Nishiue T, Fujiyama S, Tsutsumi Y, et al. (2001) Implantation of bone marrow mononuclear cells into ischemic myocardium enhances collateral perfusion and regional function via side supply of angioblasts, angiogenic ligands, and cytokines. Circulation 104: 1046-1052.
7. Takahashi T, C. Kalka, H. Masuda, D. Chen, M. Silver, M. Kearney, M. Magner, J. M. Isner & T. Asahara (1999) Ischemia- and cytokine-induced mobilization of bone marrow-derived endothelial progenitor cells for neovascularization. Nature medicine 5: 434-438.
8. Fischer-Rasokat U, Assmus B, Seeger F H, Honold J, Leistner D, et al. (2009) A pilot trial to assess potential effects of selective intracoronary bone marrow-derived progenitor cell infusion in patients with nonischemic dilated cardiomyopathy: final 1-year results of the transplantation of progenitor cells and functional regeneration enhancement pilot trial in patients with nonischemic dilated cardiomyopathy. Circ Heart Fail 2: 417-423.
9. Tateishi-Yuyama E, Matsubara H, Murohara T, Ikeda U, Shintani S, et al. (2002) Therapeutic angiogenesis for patients with limb ischaemia by autologous transplantation of bone-marrow cells: a pilot study and a randomised controlled trial. Lancet 360: 427-435.
10. Schachinger V, Assmus B, Britten M B, Honold J, Lehmann R, et al. (2004) Transplantation of progenitor cells and regeneration enhancement in acute myocardial infarction: final one-year results of the TOPCARE-AMI Trial. J Am Coil Cardiol 44: 1690-1699.
11. van Ramshorst J, Bax J J, Beeres S L, Dibbets-Schneider P, Roes S D, et al. (2009) Intramyocardial bone marrow cell injection for chronic myocardial ischemia: a randomized controlled trial. Jama 301: 1997-2004.
12. Goukassian D, Diez-Juan A, Asahara T, Schratzberger P, Silver M, et al. (2001) Overexpression of p27(Kip1) by doxycycline-regulated adenoviral vectors inhibits endothelial cell proliferation and migration and impairs angiogenesis. Faseb J 15: 1877-1885.
13. Qin G, Ii M, Silver M, Wecker A, Bord E. et al. (2006) Functional disruption of alpha4 integrin mobilizes bone marrow-derived endothelial progenitors and augments ischemic neovascularization. The Journal of experimental medicine 203: 153-163.
14. Goukassian D A, Qin G, Dolan C, Murayama T, Silver M, et al. (2007) Tumor necrosis factor-alpha receptor p75 is required in ischemia-induced neovascularization. Circulation 115: 752-762.
15. Kishore R, Tkebuchava T., Sasi S P, Silver M, Gilbert H Y, et al. (2011) Tumor necrosis factor-alpha signaling via TNFR1/p55 is deleterious whereas TNFR2/p75 signaling is protective in adult infarct myocardium. Adv Exp Med Biol 691: 433-448.
16. Convertino V A (2009) Status of cardiovascular issues related to space flight: Implications for future research directions. Respiratory physiology & neurobiology 169 Suppl 1: S34-37.
17. Bungo M W, Goldwater D J, Popp R L and Sandier H (1987) Echocardiographic evaluation of space shuttle crewmembers. J Appl Physiol 62: 278-283.
18. Convertino V A, Cooke W H and Lurie K G (2005) Inspiratory resistance as a potential treatment for orthostatic intolerance and hemorrhagic shock. Aviation, space, and environmental medicine 76: 319-325.
19. Perhonen M A, Franco F, Lane L D, Buckey J C, Blomqvist C G, et al. (2001) Cardiac atrophy after bed rest and spaceflight. J Appl Physiol 91: 645-653.
20. Reitz G (2008) Characteristic of the radiation field in low Earth orbit and in deep space. Z Med Phys 18: 233-243.
21. NCRP (2006) Information needed to make radiation protection recommendations for space missions beyond low-Earth orbit. National Council on Radiation Protection and Measurements.
22. Kai T and Spradling A (2003) An empty Drosophila stem cell niche reactivates the proliferation of ectopic cells. Proceedings of the National Academy of Sciences of the United States of America 100: 4633-4638.
23. Asahara T, Takahashi T, Masuda H, Kalka C, Chen D, et al. (1999) VEGF contributes to postnatal neovascularization by mobilizing bone marrow-derived endothelial progenitor cells. The EMBO journal 18: 3964-3972.
24. Emerit I, Levy A, Cernjavski L, Arutyunyan R, Oganesyan N, et al. (1994) Transferable clastogenic activity in plasma from persons exposed as salvage personnel of the Chernobyl reactor. Journal of cancer research and clinical oncology 120: 558-561.
25. Bubici C. Papa S, Dean K and Franzoso G (2006) Mutual cross-talk between reactive oxygen species and nuclear factor-kappa B: molecular basis and biological significance. Oncogene 25: 6731-6748.
26. Schaue D. Kachikwu E L and McBride W H (2012) Cytokines in radiobiological responses: a review. Radiation research 178: 505-523.
27. Asahara T, Murohara T, Sullivan A, Silver M, van der Zee R, et al. (1997) Isolation of putative progenitor endothelial cells for angiogenesis. Science (New York, N.Y. 275: 964-967.
28. Vasa M, Fichtischerer S, Adler K, Aicher A, Martin H. et al. (2001) Increase in circulating endothelial progenitor cells by statin therapy in patients with stable coronary artery disease. Circulation.
29. Tepper O M, Galiano R D, Capla J M, Kalka C, Gagne P J, et al. (2002) Human endothelial progenitor cells from type II diabetics exhibit impaired proliferation, adhesion, and incorporation into vascular structures. Circulation 106: 2781-2786.
30. Hill J M, Zalos G, Halcox J P, Schenke W H, Waclawiw M A, et al. (2003) Circulating endothelial progenitor cells, vascular function, and cardiovascular risk. The New England journal of medicine 348: 593-600.
31. Loomans C J, de Koning E J, Staal F J, Rookmaaker M B, Verseyden C, et al. (2004) Endothelial progenitor cell dysfunction: a novel concept in the pathogenesis of vascular complications of type 1 diabetes. Diabetes 53: 195-199.
32. Hirota Y. Masunaga S I, Kondo N, Kawabata S, Hirakawa H, et al. (2013) High linear-energy-transfer radiation can overcome radioresistance of glioma stem-like cells to low linear-energy-transfer radiation. Journal of radiation research.
33. Rosander K and Zackrisson B (1995) DNA damage in human endothelial cells after irradiation in anoxia. Acta oncologica 34: 111-116.
34. Jackson S P (2002) Sensing and repairing DNA double-strand breaks. Carcinogenesis 23: 687-696.
35. Paull T T, Rogakou E P, Yamazaki V, Kirchgessner C U, Gellert M, et al. (2000) A critical role for histone H2AX in recruitment of repair factors to nuclear foci after DNA damage. Current biology: CB 10: 886-895.
36. Staaf E, Brehwens K, Haghdoost S, Czub J and Wojcik A (2012) Gamma-H2AX foci in cells exposed to a mixed beam of X-rays and alpha particles. Genome integrity 3: 8.
37. Rogakou E P, Boon C, Redon C and Bonner W M (1999) Megabase chromatin domains involved in DNA double-strand breaks in vivo. J Cell Biol 146: 905-916.
38. Rogakou E P, Pilch D R, Orr A H, Ivanova V S and Bonner W M (1998) DNA double-stranded breaks induce histone H2AX phosphorylation on serine 139. The Journal of biological chemistry 273: 5858-5868.
39. Rothkamm K and Lobrich M (2003) Evidence for a lack of DNA double-strand break repair in human cells exposed to very low x-ray doses. Proceedings of the National Academy of Sciences of the United States of America 100: 5057-5062.
40. Fernandez-Capetillo O, Lee A, Nussenzweig M and Nussenzweig A (2004) H2AX: the histone guardian of the genome. DNA repair 3: 959-967.
41. Sedelnikova O A, Rogakou E P, Panyutin I G and Bonner W M (2002) Quantitative detection of (125)IdU-induced DNA double-strand breaks with gamma-H2AX antibody. Radiation research 158: 486-492.
42. Mendiola-Cruz M T and Morales-Ramirez P (1999) Repair kinetics of gamma-ray induced DNA damage determined by the single cell gel electrophoresis assay in murine leukocytes in vivo. Mutation research 433: 45-52.
43. Tang W, Willers H and Powell S N (1999) p53 directly enhances rejoining of DNA double-strand breaks with cohesive ends in gamma-irradiated mouse fibroblasts. Cancer research 59: 2562-2565.
44. Kumar P, Miller A I and Polverini P J (2004) p38 MAPK mediates gamma-irradiation-induced endothelial cell apoptosis, and vascular endothelial growth factor protects endothelial cells through the phosphoinositide 3-kinase-Akt-Bcl-2 pathway. The Journal of biological chemistry 279: 43352-43360.
45. Johnson L K, Longenecker J P and Fajardo L F (1982) Differential radiation response of cultured endothelial cells and smooth myocytes. Analytical and quantitative cytology 4: 188-198.
46. Goodhead D T, Thacker J and Cox R (1993) Weiss Lecture. Effects of radiations of different qualities on cells: molecular mechanisms of damage and repair. International journal of radiation biology 63: 543-556.
47. Goodhead D T (1994) Initial events in the cellular effects of ionizing radiations: clustered damage in DNA. International journal of radiation biology 65: 7-17.
48. Cucinotta F A and Durante M (2006) Cancer risk from exposure to galactic cosmic rays: implications for space exploration by human beings. The lancet oncology 7: 431-435.
49. Hada M and Sutherland B M (2006) Spectrum of complex DNA damages depends on the incident radiation. Radiation research 165: 223-230.
50. Leatherbarrow E L, Harper J V, Cucinotta F A and O'Neill P (2006) Induction and quantification of gamma-H2AX foci following low and high LET-irradiation. International journal of radiation biology 82: 111-118.
51. Karlsson K H and Stenerlow B (2004) Focus formation of DNA repair proteins in normal and repair-deficient cells irradiated with high-LET ions. Radiation research 161: 517-527.
52. Desai N, Davis E, O'Neill P, Durante M, Cucinotta F A, et al. (2005) Immunofluorescence detection of clustered gamma-H2AX foci induced by HZE-particle radiation. Radiation research 164: 518-522.
53. Bennett P, Ishchenko A A, Laval J, Paap B and Sutherland B M (2008) Endogenous DNA damage clusters in human hematopoietic stem and progenitor cells. Free radical biology & medicine 45: 1352-1359.
54. Kinashi Y, Takahashi S, Kashino G, Okayasu R, Masunaga S, et al. (2011) DNA double-strand break induction in Ku80-deficient CHO cells following boron neutron capture reaction. Radiation oncology 6: 106.
55. Ray S, Kulkarni S S, Chakraborty K, Pessu R. Hauer-Jensen M. et al. (2013) Mobilization of progenitor cells into peripheral blood by gamma-tocotrienol: a promising radiation countermeasure. International immunopharmacology 15: 557-564.
56. Petit-Frere C, Capulas E, Lyon D A, Norbury C J, Lowe J E, et al. (2000) Apoptosis and cytokine release induced by ionizing or ultraviolet B radiation in primary and immortalized human keratinocytes. Carcinogenesis 21: 1087-1095.
57. Chang C M, Limanni A, Baker W H, Dobson M E, Kalinich J F, et al. (1997) Sublethal gamma irradiation increases IL-1alpha, IL-6, and TNF-alpha mRNA levels in murine hematopoietic tissues. Journal of interferon & cytokine research: the official journal of the International Society for Interferon and Cytokine Research 17: 567-572.
58. Beetz A, Messer G, Oppel T, van Beuningen D, Peter R U, et al. (1997) Induction of interleukin 6 by ionizing radiation in a human epithelial cell line: control by corticosteroids. International journal of radiation biology 72: 33-43.
59. O'Brien-Ladner A, Nelson M E, Kimler B F and Wesselius L J (1993) Release of interleukin-1 by human alveolar macrophages after in vitro irradiation. Radiation research 136: 37-41.
60. Hallahan D E, Spriggs D R, Beckett M A, Kufe D W and Weichselbaum R R (1989) Increased tumor necrosis factor alpha mRNA after cellular exposure to ionizing radiation. Proceedings of the National Academy of Sciences of the United States of America 86: 10104-10107.
61. Brach M A, Gruss H J, Kaisho T, Asano Y, Hirano T, et al. (1993) Ionizing radiation induces expression of interleukin 6 by human fibroblasts involving activation of nuclear factor-kappa B. The Journal of biological chemistry 268: 8466-8472.
62. Meeren A V, Bertho J M, Vandamme M and Gaugler M H (1997) Ionizing radiation enhances IL-6 and IL-8 production by human endothelial cells. Mediators of inflammation 6: 185-193.
63. Petit I, Szyper-Kravitz M, Nagler A, Lahav M, Peled A, et al. (2002) G-CSF induces stem cell mobilization by decreasing bone marrow SDF-1 and up-regulating CXCR4. Nat Immunol 3: 687-694.
64. Cui Y F, Yang H, Luo Q L, Dong B, Liu X L, et al. (2004) [Radioprotection of recombinant human interleukin-3 and granulocyte-macrophage colony-stimulating factor on peripheral lymphocytes of rhesus monkey irradiated by 3.0 Gy gamma-rays]. Zhongguo wei zhong bing ji jiu yi xue=Chinese critical care medicine=Zhongguo weizhongbingjijiuyixue 16: 22-25.
65. Drouet M and Herodin F (2010) Radiation victim management and the haematologist in the future: time to revisit therapeutic guidelines? International journal of radiation biology 86: 636-648.
66. Witte L, Fuks Z, Haimovitz-Friedman A, Vlodavsky I, Goodman D S, et al. (1989) Effects of irradiation on the release of growth factors from cultured bovine, porcine, and human endothelial cells. Cancer research 49: 5066-5072.
67. Haimovitz-Friedman A, Vlodavsky I, Chaudhuri A, Witte L and Fuks Z (1991) Autocrine effects of fibroblast growth factor in repair of radiation damage in endothelial cells. Cancer research 51: 2552-2558.
68. Little M P, Gola A and Tzoulaki I (2009) A model of cardiovascular disease giving a plausible mechanism for the effect of fractionated low-dose ionizing radiation exposure. PLoS computational biology 5: e1000539.
69. Ivanova N B, Dimos J T, Schaniel C, Hackney J A, Moore K A, et al. (2002) A stem cell molecular signature. Science (New York, N.Y. 298: 601-604.
70. Ramalho-Santos M, Yoon S, Matsuzaki Y, Mulligan R C and Melton D A (2002) “Sternness”: transcriptional profiling of embryonic and adult stem cells. Science (New York, N.Y. 298: 597-600.
71. Dernbach E, Urbich C, Brandes R P, Hofmann W K, Zeiher A M, et al. (2004) Antioxidative stress-associated genes in circulating progenitor cells: evidence for enhanced resistance against oxidative stress. Blood 104: 3591-3597.
72. He T, Peterson T E, Holmuhamedov E L, Terzic A, Caplice N M, et al. (2004) Human endothelial progenitor cells tolerate oxidative stress due to intrinsically high expression of manganese superoxide dismutase. Arteriosclerosis, thrombosis, and vascular biology 24: 2021-2027.
73. Raes M, Michiels C and Remacle J (1987) Comparative study of the enzymatic defense systems against oxygen-derived free radicals: the key role of glutathione peroxidase. Free radical biology & medicine 3: 3-7.
74. Cramer T, Yamanishi Y, Clausen B E, Forster I, Pawlinski R, et al. (2003) HIF-1alpha is essential for myeloid cell-mediated inflammation. Cell 112: 645-657.
75. Boerma M and Hauer-Jensen M (2010) Preclinical research into basic mechanisms of radiation-induced heart disease. Cardiology research and practice 2011.
76. Lee T K, O'Brien K F, Wang W, Johnke R M, Sheng C, et al. (2010) Radioprotective effect of American ginseng on human lymphocytes at 90 minutes postirradiation: a study of 40 cases. Journal of alternative and complementary medicine 16: 561-567.
77. Soucy K G, Lim H K, Attarzadeh D O, Santhanam L, Kim J H, et al. (2010) Dietary inhibition of xanthine oxidase attenuates radiation-induced endothelial dysfunction in rat aorta. Journal of applied physiology 108: 1250-1258.
78. Wang J, Zheng H, Ou X, Fink L M and Hauer-Jensen M (2002) Deficiency of microvascular thrombomodulin and up-regulation of protease-activated receptor-1 in irradiated rat intestine: possible link between endothelial dysfunction and chronic radiation fibrosis. The American journal of pathology 160: 2063-2072.
79. Wu W, Masri A, Popovic Z B, Smedira N G, Lytle B W, et al. (2013) Long-term survival of patients with radiation heart disease undergoing cardiac surgery: a cohort study. Circulation 127: 1476-1484.
80. Gyenes G, Fornander T, Carlens P and Rutqvist L E (1994) Morbidity of ischemic heart disease in early breast cancer 15-20 years after adjuvant radiotherapy. International journal of radiation oncology, biology, physics 28: 1235-1241.
81. Taylor C W, McGale P and Darby S C (2006) Cardiac risks of breast-cancer radiotherapy: a contemporary view. Clinical oncology (Royal College of Radiologists (Great Britain)) 18: 236-246.
82. Gayed I W, Liu H H, Yusuf S W, Komaki R, Wei X, et al. (2006) The prevalence of myocardial ischemia after concurrent chemoradiation therapy as detected by gated myocardial perfusion imaging in patients with esophageal cancer. J Nucl Med 47: 1756-1762.
83. Bose A S, Shetty V, Sadiq A, Shani J and Jacobowitz I (2009) Radiation induced cardiac valve disease in a man from Chernobyl. J Am Soc Echocardiogr 22: 973 e971-973.
84. Pant G S and Kamada N (1977) Chromosome aberrations in normal leukocytes induced by the plasma of exposed individuals. Hiroshima journal of medical sciences 26: 149-154.
85. Pitchford S C, Furze R C, Jones C P, Wengner A M and Rankin S M (2009) Differential mobilization of subsets of progenitor cells from the bone marrow. Cell stem cell 4: 62-72.
86. Fleissner F and Thum T (2008) The IGF-1 receptor as a therapeutic target to improve endothelial progenitor cell function. Molecular medicine 14: 235-237.
87. Prevo R, Deutsch E, Sampson O, Diplexcito J, Cengel K, et al. (2008) Class I P13 kinase inhibition by the pyridinylfuranopyrimidine inhibitor PI-103 enhances tumor radiosensitivity. Cancer research 68: 5915-5923.
88. McBride W H and Dougherty G J (1995) Radiotherapy for genes that cause cancer. Nature medicine 1: 1215-1217.
89. Rodemann H P and Blaese M A (2007) Responses of normal cells to ionizing radiation. Seminars in radiation oncology 17: 81-88.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of ameliorating the effects of radiation exposure on a cell, the method comprising contacting the cell with an agent that selectively reduces the expression or activity of one or more of a p75 TNF-α, p55 TNF-α, IL6, EGF, IL1-beta, G-CSF, MCP-1, MIP-1, SCF, or RANTES receptor in the cell relative to an untreated control cell, thereby ameliorating the effects of radiation exposure on the cell.

2. A method of ameliorating the effects of radiation exposure on a cell, the method comprising contacting the cell with an agent that selectively reduces the expression or activity of one or more of a p75 TNF-α, p55 TNF-α, IL6, EGF, IL1-alpha, IL1-beta, G-CSF, MCP-1, MIP-1, SCF, or RANTES peptide in the cell relative to an untreated control cell, thereby ameliorating the effects of radiation exposure on the cell.

3. The method of claim 1, wherein the cell is contacted with a cytokine produced by radiation exposure.

4. The method of claim 3, wherein the cytokine is one or more of TNF-α, IL6, EGF, IL1-beta, G-CSF, MCP-1, MIP-1, SCF, and RANTES.

5. The method of claim 1, wherein the radiation is one or more of low-dose or high-dose of terrestrial or hadron radiation, or high charge and energy (HZE) heavy ion particle radiation.

6. The method of claim 1, wherein the cell is an endothelial progenitor cell, hemangioblast, hematopoietic stem cell, endothelial cell, cardiac cell, cardiomyocyte, muscle cell, vascular smooth muscle cell, satellite-cell, myoblast, or differentiated skeletal muscle cell.

7. The method of claim 1, wherein the effect of radiation exposure is direct or indirect.

8. The method of claim 7, wherein the cell is not exposed to radiation.

9. The method of claim 7, wherein the cell is contacted with a cell or product of a cell that has been exposed to radiation.

10. The method of claim 9, wherein the contacting is via a gap junction.

11. The method of claim 7, wherein the cell is in the immediate vicinity of and not in direct contact with a cell that has been exposed to radiation.

12. The method of claim 1, wherein the cell and cell exposed to radiation are present in a subject.

13. The method of claim 1, wherein the cell exposed to radiation is a BM-derived endothelial progenitor cell, hemangioblast, hematopoietic stem cell, endothelial cell, cardiomyocyte, vascular smooth muscle cell, satellite-cell, myoblast, or differentiated skeletal muscle cell.

14. The method of claim 1, wherein the effect of radiation exposure is one or more of a DNA double-strand break, gene inactivating mutation in a somatic or stem cell, increase in cytoplasmic Ca²⁺ signaling, reduction in mitochondrial action potential, decreased ATP production, increased production of reactive oxygen and nitrogen species (ROS and NOS), or decreased cardiomyocyte or skeletal muscle contractility.

15. The method of claim 14, wherein the effect occurs within 24 hrs, 1-28 days, 1-24 months 1-40 years after radiation exposure.

16. The method of claim 1, wherein the agent is an inhibitory nucleic acid molecule that is complementary to at least a portion of a p75 TNF-α, p55 TNF-α, IL6, EGF, IL1-beta, G-CSF, MCP-1, MIP-1, SCF, or RANTES receptor nucleic acid molecule; or a TNF-α, EGF, IL1-alpha, IL1-beta, G-CSF, MCP-1, MIP-1, SCF, or RANTES nucleic acid molecule.

17. The method of claim 16, wherein the inhibitory nucleic acid molecule is selected from the group consisting of an antisense molecule, an siRNA, and an shRNA.

18. The method of claim 17, wherein the inhibitory nucleic acid molecule comprises or consists essentially of a nucleic acid molecule with a sequence selected from the group consisting SEQ ID NO: 1, SEQ ID NO: 2, SEQ ID NO: 3, and SEQ ID NO: 4.

19. The method of claim 1, wherein the agent is an antibody or fragment thereof that selectively binds to a p75 TNF-α, p55 TNF-α, IL6, EGF, IL1-beta, G-CSF, MCP-1, MIP-1, SCF, or RANTES receptor; or a TNF-α, EGF, IL1-alpha, IL1-beta, G-CSF, MCP-1, MIP-1, SCF, or RANTES peptide.

20. The method of claim 19, wherein the antibody is a monoclonal or polyclonal antibody.

21. The method of claim 1, wherein the method reduces cell death, reduces DNA damage, or increases DNA repair.

22. A method of ameliorating the effects of radiation exposure on a subject, the method comprising administering to the subject an agent that selectively reduces the expression or activity of one or more of a receptor for p75 TNF-α, p55 TNF-α, IL6, EGF, IL1-beta, G-CSF, MCP-1, MIP-1, SCF, or RANTES in a cell relative to an untreated control cell, thereby ameliorating the effects of radiation exposure on the subject.

23. A method of ameliorating the effects of radiation exposure on a subject, the method comprising administering to the subject an agent that selectively reduces the expression or activity of one or more of a TNF-α, IL6, EGF, IL1-beta, G-CSF, MCP-1, MIP-1, SCF, or RANTES peptide in a cell relative to an untreated control cell, thereby ameliorating the effects of radiation exposure on the subject.

24. The method of claim 22, wherein the cell is contacted with a cytokine produced by radiation exposure.

25. The method of claim 24, wherein the cytokine is one or more of TNF-α, IL6, EGF, IL1-beta, G-CSF, MCP-1, MIP-1, SCF, and RANTES.

26. The method of claim 22, wherein the cell is a BM-derived endothelial progenitor cell, hemangioblast, hematopoietic stem cell, endothelial cell, cardiac cell, cardiomyocyte, muscle cell, vascular smooth muscle cell, satellite-cell, myoblast, or differentiated skeletal muscle cell.

27. The method of claim 22, wherein the radiation exposure is direct or indirect.

28. The method of claim 27, wherein the cell is not exposed to radiation.

29. The method of claim 27, wherein the cell is contacted with a cell that has been exposed to radiation.

30. The method of claim 29, wherein the contacting is via a gap junction.

31. The method of claim 27, wherein the cell is in the immediate vicinity of and not in direct contact with a cell that has been exposed to radiation.

32. The method of claim 29, wherein the cell and cell exposed to radiation are present in the subject.

33. The method of claim 29, wherein the cell exposed to radiation is an endothelial progenitor cell, hemangioblast, hematopoietic stem cell, endothelial cell, cardiomyocyte, vascular smooth muscle cell, or a satellite-cell, myoblast, differentiated skeletal muscle cell.

34. The method of claim 22, wherein the effect is one or more of a DNA double-strand break, gene inactivating mutation in a somatic or stem cell, increase in cytoplasmic Ca²⁺ signaling, reduction in mitochondrial action potential, decreased ATP production, increased production of reactive oxygen and nitrogen species (ROS and NOS), or decreased cardiomyocyte or skeletal muscle contractility.

35. The method of claim 34, wherein the effect occurs within 24 hrs, 1-28 days, 1-24 months 1-40 years after radiation exposure.

36. The method of claim 22, wherein the agent is an inhibitory nucleic acid molecule that is complementary to at least a portion of a p75 TNF-α, p55 TNF-α, IL6, EGF, IL1-beta, G-CSF, MCP-1, MIP-1, SCF, or RANTES receptor nucleic acid molecule; or a TNF-α, IL6, EGF, IL1-beta, G-CSF, MCP-1, MIP-1, SCF, or RANTES nucleic acid molecule.

37. The method of claim 36, wherein the inhibitory nucleic acid molecule is selected from the group consisting of an antisense molecule, an siRNA, and an shRNA.

38. The method of claim 37, wherein the inhibitory nucleic acid molecule comprises or consists essentially of a nucleic acid molecule with a sequence selected from the group consisting SEQ ID NO: 1, SEQ ID NO: 2, SEQ ID NO: 3, and SEQ ID NO: 4.

39. The method of claim 22, wherein the agent is an antibody or fragment thereof that selectively binds to a p75 or p55 TNF-α receptor; an IL6, EGF, IL1-beta, G-CSF, MCP-1, MIP-1, SCF, or RANTES receptor; or an IL6, EGF, IL1-beta, G-CSF, MCP-1, MIP-1, SCF, or RANTES peptide.

40. The method of claim 39, wherein the antibody is a monoclonal or polyclonal antibody.

41. The method of claim 22, wherein the method reduces cell death, reduces DNA damage, or increases DNA repair.

42. A pharmaceutical composition for the treatment of radiation exposure, the composition comprising an effective amount of two or more agents that selectively reduce the expression or activity of two or more of a p75 TNF-α, p55 TNF-α, IL6, EGF, IL1-beta, G-CSF, MCP-1, MIP-1, SCF, or RANTES receptor in a cell, relative to a reference cell.

43. A pharmaceutical composition for the treatment of radiation exposure, the composition comprising an effective amount of two or more agents that selectively reduce the expression or activity of two or more of a TNF-α, IL6, EGF, IL1-beta, G-CSF, MCP-1, MIP-1, SCF, or RANTES peptide in a cell, relative to a reference cell.

44. The pharmaceutical composition of claim 42, wherein at least one agent is an inhibitory nucleic acid molecule siRNA that is complementary to at least a portion of a p75 TNF-α, p55 TNF-α, IL6, EGF, IL1-beta, G-CSF, MCP-1, MIP-1, SCF, or RANTES receptor nucleic acid molecule; or a TNF-α, IL6, EGF, IL1-beta, G-CSF, MCP-1, MIP-1, SCF, or RANTES nucleic acid molecule.

45. The pharmaceutical composition of claim 44, wherein the inhibitory nucleic acid molecule is selected from the group consisting of an antisense molecule, an siRNA, and an shRNA.

46. The pharmaceutical composition of claim 45, wherein the inhibitory nucleic acid molecule comprises a nucleic acid molecule with a sequence selected from the group consisting SEQ ID NO: 1, SEQ ID NO: 2, SEQ ID NO: 3, and SEQ ID NO: 4.

47. The pharmaceutical composition of claim 42, wherein at least one agent is an antibody or fragment thereof that selectively binds to a p75 TNF-α, p55 TNF-α, IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide.

48. The pharmaceutical composition of claim 47, wherein the antibody is monoclonal or polyclonal.

49. The pharmaceutical composition of claim 42, wherein the agent reduces cell death, reduces DNA damage, or increases DNA repair in the subject.

50. A kit for treating radiation exposure comprising an effective amount of an agent that selectively reduces the expression or activity of a p75 TNF-α, p55 TNF-α, IL6, EGF, IL1-beta, G-CSF, MCP-1, MIP-1, SCF, or RANTES receptor; or a TNF-α, IL6, EGF, IL1-beta, G-CSF, MCP-1, MIP-1, SCF, or RANTES peptide in a cell and instructions for using the kit to treat radiation exposure.

51. The kit of claim 50, wherein the agent is an inhibitory nucleic acid molecule shRNA that is complementary to at least a portion of a p75 TNF-α, p55 TNF-α, IL6, EGF, IL1-alpha, IL1-beta, G-CSF, MCP-1, MIP-1, SCF, or RANTES receptor nucleic acid molecule; or a TNF-α, IL6, EGF, IL1-alpha, IL1-beta, G-CSF, MCP-1, MIP-1, SCF, or RANTES nucleic acid molecule.

52. The kit of claim 51, wherein the inhibitory nucleic acid molecule is selected from the group consisting of an antisense molecule, an siRNA, and an shRNA.

53. The kit of claim 52, wherein the inhibitory nucleic acid molecule comprises a nucleic acid molecule with a sequence selected from the group consisting SEQ ID NO: 1, SEQ ID NO: 2, SEQ ID NO: 3, and SEQ ID NO: 4.

54. The kit of claim 50, wherein the agent is an antibody or fragment thereof that selectively binds to p75 TNF-α, p55 TNF-α, IL6, EGF, IL1-alpha, IL1-beta, G-CSF, MCP-1, MIP-1, SCF, or RANTES receptor; or a TNF-α, IL6, EGF, IL1-alpha, IL1-beta, G-CSF, MCP-1, MIP-1, SCF, or RANTES peptide.

55. The kit of claim 54, wherein the antibody is monoclonal or polyclonal.

56. The kit of claims 50, wherein the agent reduces cell death, reduces DNA damage, or increases DNA repair.
